SEQUENCE LISTING (146 sequences)

SEQ ID NO: 1 - DNA, 2239 bp, Homo sapiens
  CDS (25)..(2076)
tcgccgagcccgtccgccgccgccatggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatggatggatgacatcccagggctgctgccacccccacctgtggggagacaccagactgggggtggtgtggagatactcttagagaagaggctgctgggccacgggctcggcatggcagggcagtggctagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaatgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggtgcctgtgtctgcagaaaaacaagaccagatgtgatttgttaaaaaaaaaa

SEQ ID NO: 2 - protein, 684 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPWMDDIPGLLPPPPVGRHQTGGGVEILLEKRLLGHGLGMAGQWLARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 3 - DNA, 2014 bp, Homo sapiens
  CDS (14)..(2011)
caccggatccaccatggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccaatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtga

SEQ ID NO: 4 - protein, 666 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPSRKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 5 - DNA, 1998 bp, Homo sapiens
  CDS (1)..(1998)
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccaatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcag

SEQ ID NO: 6 - protein, 666 aa, Homo sapiens; identical to SEQ ID NO: 4

SEQ ID NO: 7 - DNA, 1809 bp, Homo sapiens
  CDS (1)..(1809)
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcag

SEQ ID NO: 8 - protein, 603 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAKSLLNKKADGVKPQTNSTKNSAAATSPKGTLPPAALEPQTTVIHNPVDGIKESSDSANTTIEDEDAKAPRISDILNSVRRGSGTPEAEGPLSAGPPPCLSPALLGPLSSPSPRISDILNSVRRGSGTPEAEGPSPVGPPPCPSPTIPGPLPTPSRKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 9 - DNA, 1923 bp, Homo sapiens
  CDS (1)..(1923)
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctttttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtacggcaagcccgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccaatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaatgtgcacttccactgctcgggcgcgcctgtggccccgctgcag

SEQ ID NO: 10 - protein, 641 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPSRKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 11 - DNA, 840 bp, Homo sapiens
  CDS (1)..(840)
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatcc

SEQ ID NO: 12 - protein, 280 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVAS

SEQ ID NO: 13 - DNA, 1824 bp, Homo sapiens
  CDS (433)..(1671)
gcggcccgcgtcgaccgagcgcacgccgagcccgtccgccgccgccatggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttgtgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctgaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtacggcaagcccgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagtaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggttcctgtgtctgcagaaaaacaagaccagatgtgatttgtt

SEQ ID NO: 14 - protein, 413 aa, Homo sapiens
MGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 15 - DNA, 1676 bp, Homo sapiens
  CDS (25)..(1533)
tcgccgagcccgtccgccgccgccatggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtacggcaagcccgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagcagccaagagtttactcaacaagaaagcagatggagtcaagccccatacgaatagcaccaaaaacagtgcagccgccaccagccccaaagggacgcttcctcctgccgccctggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggtgcctgtgtctgcagaaaaacaagcccgact

SEQ ID NO: 16 - protein, 503 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAAKSLLNKKADGVKPHTNSTKNSAAATSPKGTLPPAALESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 17 - DNA, 2251 bp, Homo sapiens
  CDS (1)..(2088)
atggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagggccactcgcactaacccacctgctgtttgccacagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatggatggatgacatcccagggctgctgccacccccacctgtggggagacaccagactgggggtggtgtggagatactcttagagaagaggctgctgggccacgggctcggcatggcagggcagtggctagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaatgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggtgcctgtgtctgcagaaaaacaagaccagatgtgatttgttaaaaaaaaaa

SEQ ID NO: 18 - protein, 696 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADARATRTNPPAVCHSHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPWMDDIPGLLPPPPVGRHQTGGGVEILLEKRLLGHGLGMAGQWLARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 19 - DNA, 1727 bp, Homo sapiens
  CDS (4)..(1629)
gccatggccaccacggtgacctgcacccgcttcaccgacgagtaccagctatacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagtgggcagacagaccaccgctccggccacaatgtccaccgcggcctccggcaccaccatggggctggtggaacaagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccaatccacacgaccatcctgaacccacacgtgcacgtcattggagaggatgtcgcctgcaccgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctc

SEQ ID NO: 20 - protein, 542 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSVGRQTTAPATMSTAASGTTMGLVEQAKSLLNKKADGVKPQTNSTKNSAAATSPKGTLPPAALEPQTTVIHNPVDGIKESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDVACTAYIRLTQYIDGQGRPRTSQSEETRVWHRRDGKWQNVHFHCSGAPVAPLQ

SEQ ID NO: 21 - DNA, 1689 bp, Homo sapiens
  CDS (2)..(1546)
catggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagggccactcgcactaacccacctgctgtttgccacagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtacggcaagcccgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagcagccaagagtttactcaacaagaaagcagatggagtcaagccccatacgaatagcaccaaaaacagtgcagccgccaccagccccaaagggacgcttcctcctgccgccctggagtcttctgacagtgccaataccaccatagaggatgaagacgctaaagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatggacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaacgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggtgcctgtgtctgcagaaaaacaagcccgact

SEQ ID NO: 22 - protein, 515 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADARATRTNPPAVCHSHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAAKSLLNKKADGVKPHTNSTKNSAAATSPKGTLPPAALESSDSANTTIEDEDAKARKQEIIKTTEQLIEAVNNGDFEAYAKICDPGLTSFEPEALGNLVEGMDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 23 - DNA, 2239 bp, Homo sapiens
  CDS (25)..(2076)
tcgccgagcccgtccgccgccgccatggccaccacggtgacctgcacccgcttcaccgacgagtaccagctctacgaggatattggcaagggggctttctctgtggtccgacgctgtgtcaagctctgcaccggccatgagtatgcagccaagatcatcaacaccaagaagctgtcagccagagatcaccagaagctggagagagaggctcggatctgccgccttctgaagcattccaacatcgtgcgtctccacgacagcatctccgaggagggcttccactacctggtcttcgatctggtcactggtggggagctctttgaagacattgtggcgagagagtactacagcgaggctgatgccagtcactgtatccagcagatcctggaggccgttctccattgtcaccaaatgggggtcgtccacagagacctcaagccggagaacctgcttctggccagcaagtgcaaaggggctgcagtgaagctggcagacttcggcctagctatcgaggtgcagggggaccagcaggcatggtttggtttcgctggcacaccaggctacctgtcccctgaggtccttcgcaaagaggcgtatggcaagcctgtggacatctgggcatgtggggtgatcctgtacatcctgctcgtgggctacccacccttctgggacgaggaccagcacaagctgtaccagcagatcaaggctggtgcctatgacttcccgtcccctgagtgggacaccgtcactcctgaagccaaaaacctcatcaaccagatgctgaccatcaaccctgccaagcgcatcacagcccatgaggccctgaagcacccgtgggtctgccaacgctccacggtagcatccatgatgcacagacaggagactgtggagtgtctgaaaaagttcaatgccaggagaaagctcaagggagccatcctcaccaccatgctggccacacggaatttctcagccaagagtttactcaacaagaaagcagatggagtcaagccccagacgaatagcaccaaaaacagtgcagccgccaccagccccaaagggacgcttcctcctgccgccctggagcctcaaaccaccgtcatccataacccagtggacgggattaaggagtcttctgacagtgccaataccaccatagaggatgaagacgctaaagcccccagggtccccgacatcctgagctcagtgaggaggggctcgggagccccagaagccgaggggcccctgccctgcccatctccggctccctttagccccctgccagccccatcccccaggatctctgacatcctgaactctgtgagaaggggttcaggaaccccagaagccgagggccccctctcagcggggcccccgccctgcctgtctccggctctcctaggccccctgtcctccccgtcccccaggatctctgacatcctgaactctgtgaggaggggctcagggaccccagaagccgagggcccctcgccagtggggcccccgccctgcccatctccgactatccctggccccctgcccaccccatggatggatgacatcccagggctgctgccacccccacctgtggggagacaccagactgggggtggtgtggagatactcttagagaagaggctgctgggccacgggctcggcatggcagggcagtggctagcccggaagcaggagatcattaagaccacggagcagctcatcgaggccgtcaacaacggtgactttgaggcctacgcgaaaatctgtgacccagggctgacctcgtttgagcctgaagcactgggcaacctggttgaagggatagacttccacagattctacttcgagaacctgctggccaagaacagcaagccgatccacacgaccatcctgaacccacacgtgcacgtcattggagaggatgccgcctgcatcgcttacatccggctcacgcagtacattgacgggcagggccggccccgcaccagccagtctgaggagacccgcgtgtggcaccgccgcgacggcaagtggcagaatgtgcacttccactgctcgggcgcgcctgtggccccgctgcagtgaagagctgcgccctggtttcgccggacagagttggtgtttggagcccgactgccctcgggcacacggcctgcctgtcgcatgtttgtgtctgcctcgttccctcccctggtgcctgtgtctgcagaaaaacaagaccagatgtgatttgttaaaaaaaaaa

SEQ ID NO: 24 - protein, 684 aa, Homo sapiens
MATTVTCTRFTDEYQLYEDIGKGAFSVVRRCVKLCTGHEYAAKIINTKKLSARDHQKLEREARICRLLKHSNIVRLHDSISEEGFHYLVFDLVTGGELFEDIVAREYYSEADASHCIQQILEAVLHCHQMGVVHRDLKPENLLLASKCKGAAVKLADFGLAIEVQGDQQAWFGFAGTPGYLSPEVLRKEAYGKPVDIWACGVILYILLVGYPPFWDEDQHKLYQQIKAGAYDFPSPEWDTVTPEAKNLINQMLTINPAKRITAHEALKHPWVCQRSTVASMMHRQETVECLKKFNARRKLKGAILTTMLATRNFSAKSLLNKKADGVKPQTNSTKNSAAATSPKGTLPPAALEPQTTVIHNPVDGIKESSDSANTTIEDEDAKAPRVPDILSSVRRGSGAPEAEGPLPCPSPAPFSPLPAPSPRISDILNSVRRGSGTPEAEGPLSAGPPPCLSPALLGPLSSPSPRISDILNSVRRGSGTPEAEGPSPVGPPPCPSPTIPGPLPTPWMDDIPGLLPPPPVGRHQTGGGVEILLEKRLLGHGLGMAGQWLARKQEIIKTTEQLIEAVNNGDFEAYAKICDPGLTSFEPEALGNLVEGIDFHRFYFENLLAKNSKPIHTTILNPHVHVIGEDAACIAYIRLTQYIDGQGRPRTSQSEETRVWHRRDGKWQNVHFHCSGAPVAPLQ

SEQ ID NO: 25 - DNA, 3309 bp, Homo sapiens
  CDS (295)..(1983)
gcggccgcgctgcctggcagcccgggaagccgcggcacagctgctcggcgcctgcagctccggctcgggggctggaaccgaagcgggggcggcgggagcgcggagaccacagcccccggggagaggcggagggggtccctggcctgggcggagaggctgagctgagtgcgcgtgagaaagagggctgcaccgctgctcggcgcggactctgccagccccagcttcagccccggctcaggtcgccgcagcccgggagcctccccgcttgcgccccaaggcacgcgcggcacagccatgaacaccaacgatgccaaggagtatctggcccggagggaaatccctcagctttttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgtaatgcctgaaaactcaaactttccatatcggcggtatgaccggctccctccaatccatcaattctccatagaaagtgacacggatctctctgagactgcagagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgattttgtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacagctacacaagataaatgcagagggaacaccagaggacgtttttcttcaactctgcacagctattgactctattattttctgaaggcaaaaatgcatgtttgttagaatggaaacagaaaaacattaaaaagttcattccttaacacaatgtttcaagttaaaccttttgtgtcaccgccccccaccaaccaccacctcctaaatcctgacagcactgtttgcttcccagctagacctgtgtgagaggtgtctggaaatcatgcatggtgtatttgggactatatcaacctattctccacacttcagacaactgtctgcactcacggcacgcacactttgtatcatgcaggccacactcagagctagtcagtacatgaacagtggtgcggtgccagtctgtgtccgttgtgatcacaggccttgctagaccctgatcatctggttctcctctcattaagcatccctaacccccagtcacaccttcctcttacatactgttccccaatggaggcccctggcataggggacagccctgggcatcttcctttggtgtctggctgttttgtcaactctcatccactggtggctcagagccataaggtgggttgattacacaatgccttgtacatgatatagaggcatcaagcaagtaaaatttgacagaaattttaaaatatgaagatgtatagctttcccaagatgatggtaaaacccaggttagtcatcagtaaccttctctattattattattttttagaaacttggaatactgtcattatggctaagagaacaaatctgataaattgtgtaacctagtctcttctctacatggtgatgcatttcagcaattataaattaatataaatgaccaaaagtaacttaaaagcatgagatatttgctatttcattcattgggcacatatcaaattataattttgattttaaatggtcacccatgtatttgttgccaagcaagtaaaaaaataccctaacaaacctgatgtgggtgggaggggcatgtcagtaagtggtgtgttcaatgtgtttgtttcatatgggccctttccaggagtttgcaaaccttgtcatacccatatgcaaaactgtgtttccttgcattaaaccagtgaagtttgggttctcttttgtgctatcaatcagttgtaaaatcagagcttcttatatattctactggaataactgcatcttccactcagtcactacaaaaaagcatagtttcagtttgcatgaatttttttttttttcttcaatggttgtgcagataaggatccatttctgggatagaattgtattttttaagtcatttttttttcttgaaatggatatgtacaaataaaattaaatggaagacaggaaaaaaaaaaaaaaaaaaaaaaaaaaaaaa

SEQ ID NO: 26 - protein, 563 aa, Homo sapiens
MNTNDAKEYLARREIPQLFESLLNGLMCSKPEDPVEYLESCLQKVKELGGCDKVKWDTFVSQEKKTLPPLNGGQSRRSFLRNVMPENSNFPYRRYDRLPPIHQFSIESDTDLSETAELIEEYEVFDPTRPRPKIILVIGGPGSGKGTQSLKIAERYGFQYISVGELLRKKIHSTSSNRKWSLIAKIITTGELAPQETTITEIKQKLMQIPDEEGIVIDGFPRDVAQALSFEDQICTPDFVVFLACANQRLKERLLKRAEQQGRPDDNVKATQRRLMNFKQNAAPLVKYFQEKGLIMTFDADRDEDEVFYDISMAVDNKLFPNKEAAAGSSDLDPSMILDTGEIIDTGSDYEDQGDDQLNVFGEDTMGGFMEDLRKCKIIFIIGGPGSGKGTQCEKLVEKYGFTHLSTGELLREELASESERSKLIRDIMERGDLVPSGIVLELLKEAMVASLGDTRGFLIDGYPREVKQGEEFGRRIGDPQLVICMDCSADTMTNRLLQRSRSSLPVDDTTKTIAKRLEAYYRASIPVIAYYETKTQLHKINAEGTPEDVFLQLCTAIDSIIF

SEQ ID NO: 27 - DNA, 490 bp, Homo sapiens
  CDS (2)..(490)
caccagatctataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacaggtcgacggc

SEQ ID NO: 28 - protein, 163 aa, Homo sapiens
TRSIIGGPGSGKGTQCEKLVEKYGFTHLSTGELLREELASESERSKLIRDIMERGDLVPSGIVLELLKEAMVASLGDTRGFLIDGYPREVKQGEEFGRRIGDPQLVICMDCSADTMTNRLLQRSRSSLPVDDTTKTIAKRLEAYYRASIPVIAYYETKTQVDG

SEQ ID NO: 29 - DNA, 493 bp, Homo sapiens
  CDS (2)..(493)
caccagatctgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgatttggtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcgtcgacggc

SEQ ID NO: 30 - protein, 164 aa, Homo sapiens
TRSVIGGPGSGKGTQSLKIAERYGFQYISVGELLRKKIHSTSSNRKWSLIAKIITTGELAPQETTITEIKQKLMQIPDEEGIVIDGFPRDVAQALSFEDQICTPDLVVFLACANQRLKERLLKRAEQQGRPDDNVKATQRRLMNFKQNAAPLVKYFQEKGLVDG

SEQ ID NO: 31 - DNA, 1714 bp, Homo sapiens
  CDS (2)..(1714)
caccagatctcccaccatgaacaccaacgatgccaaggagtatctggcccggagggaaatccctcagctttttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgtaatgcctgaaaactcaaactttccatatcggcggtatgaccggctccctccaatccatcaattctccatagaaagtgacacggatctctctgagactgcagagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgatttggtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacagctacacaagataaatgcagagggaacaccagaggacgtttttcttcaactctgcacagctattgactctattattttcgtcgacggc

SEQ ID NO: 32 - protein, 571 aa, Homo sapiens
TRSPTMNTNDAKEYLARREIPQLFESLLNGLMCSKPEDPVEYLESCLQKVKELGGCDKVKWDTFVSQEKKTLPPLNGGQSRRSFLRNVMPENSNFPYRRYDRLPPIHQFSIESDTDLSETAELIEEYEVFDPTRPRPKIILVIGGPGSGKGTQSLKIAERYGFQYISVGELLRKKIHSTSSNRKWSLIAKIITTGELAPQETTITEIKQKLMQIPDEEGIVIDGFPRDVAQALSFEDQICTPDLVVFLACANQRLKERLLKRAEQQGRPDDNVKATQRRLMNFKQNAAPLVKYFQEKGLIMTFDADRDEDEVFYDISMAVDNKLFPNKEAAAGSSDLDPSMILDTGEIIDTGSDYEDQGDDQLNVFGEDTMGGFMEDLRKCKIIFIIGGPGSGKGTQCEKLVEKYGFTHLSTGELLREELASESERSKLIRDIMERGDLVPSGIVLELLKEAMVASLGDTRGFLIDGYPREVKQGEEFGRRIGDPQLVICMDCSADTMTNRLLQRSRSSLPVDDTTKTIAKRLEAYYRASIPVIAYYETKTQLHKINAEGTPEDVFLQLCTAIDSIIFVDG

SEQ ID NO: 33 - DNA, 1642 bp, Homo sapiens
  CDS (2)..(1642)
caccagatctcccaccatgaacaccaacgatgccaaggagtatctggcccggagggaaatccctcagctttttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgaaagtgacacggatctctctgagactgcagagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgatttggtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacagctacacaagataaatgcagagggaacaccagaggacgtttttcttcaactctgcacagctattgactctattattttcgtcgacggc

SEQ ID NO: 34 - protein, 547 aa, Homo sapiens
TRSPTMNTNDAKEYLARREIPQLFESLLNGLMCSKPEDPVEYLESCLQKVKELGGCDKVKWDTFVSQEKKTLPPLNGGQSRRSFLRNESDTDLSETAELIEEYEVFDPTRPRPKIILVIGGPGSGKGTQSLKIAERYGFQYISVGELLRKKIHSTSSNRKWSLIAKIITTGELAPQETTITEIKQKLMQIPDEEGIVIDGFPRDVAQALSFEDQICTPDLVVFLACANQRLKERLLKRAEQQGRPDDNVKATQRRLMNFKQNAAPLVKYFQEKGLIMTFDADRDEDEVFYDISMAVDNKLFPNKEAAAGSSDLDPSMILDTGEIIDTGSDYEDQGDDQLNVFGEDTMGGFMEDLRKCKIIFIIGGPGSGKGTQCEKLVEKYGFTHLSTGELLREELASESERSKLIRDIMERGDLVPSGIVLELLKEAMVASLGDTRGFLIDGYPREVKQGEEFGRRIGDPQLVICMDCSADTMTNRLLQRSRSSLPVDDTTKTIAKRLEAYYRASIPVIAYYETKTQLHKINAEGTPEDVFLQLCTAIDSIIFVDG

SEQ ID NO: 35 - DNA, 1842 bp, Homo sapiens
  CDS (375)..(1625)
gcggccgcgctgcctggcagcccgggaagccgcggcacagctgctcggcgcctgcagctccggctcgggggctggaaccgaagcgggggcggcgggagcgcggagaccacagcccccggggagaggcggagggggtccctggcctgggcggagaggctgagctgagtgcgcgtgagaaagagggctgcaccgctgctcggcgcggactctgccagccccagcttcagccccggctcaggtcgccgcagcccgggagcctccccgcttgcgccccaaggcacgcgcggcacagccatgaacaccaacgatgccaaggagtatctggcccggagggaaatccctcagttatatttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgtaatgcctgaaaactcaaactttccatatcggcggtatgaccggctccctccaatccatcaattctccatagaaagtgacacggatctctctgagactgcggagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccagatctgtacccccgatttggtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgtttggagctcctgaaggaggcatggtggcagctcggggacaccaggggcttctgatgacggttatctcgggaggggaagcaggggaagagtcggacggcagatggagaccaacagtgggactggatggactgcccgaaacaccagacaacgcttccaaaggacggagattgctgggagaacacaaaaacaggaacgcaagcaaacgaaggaaccagggagcccagaaa

SEQ ID NO: 36 - protein, 417 aa, Homo sapiens
MCSKPEDPVEYLESCLQKVKELGGCDKVKWDTFVSQEKKTLPPLNGGQSRRSFLRNVMPENSNFPYRRYDRLPPIHQFSIESDTDLSETAELIEEYEVFDPTRPRPKIILVIGGPGSGKGTQSLKIAERYGFQYISVGELLRKKIHSTSSNRKWSLIAKIITTGELAPQETTITEIKQKLMQIPDEEGIVIDGFPRDVAQALSFEDQICTPDLVVFLACANQRLKERLLKRAEQQGRPDDNVKATQRRLMNFKQNAAPLVKYFQEKGLIMTFDADRDEDEVFYDISMAVDNKLFPNKEAAAGSSDLDPSMILDTGEIIDTGSDYEDQGDDQLNVFGEDTMGGFMEDLRKCKIIFIIGGPGSGKGTQCEKLVEKYGFTHLSTGELLREELASESERSKLIRDIMERGDLVPSGIVWSS

SEQ ID NO: 37 - DNA, 1714 bp, Homo sapiens; identical to SEQ ID NO: 31
  CDS (17)..(1705)

SEQ ID NO: 38 - protein, 563 aa, Homo sapiens [sequence omitted]

SEQ ID NO: 39 - DNA, 1714 bp, Homo sapiens
  CDS (14)..(1702)
caccggatccaccatgaacaccaacgatgccaaggagtatctggcccggagggaaatccctcagctttttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgtaatgcctgaaaactcaaactttccatatcggcggtatgaccggctccctccaatccatcaattctccatagaaagtgacacggatctctctgagactgcagagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgatttggtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacagctacacaagataaatgcagagggaacaccagaggacgtttttcttcaactctgcacagctattgactctattattttctagctcgagggc

SEQ ID NO: 40 - protein, 563 aa, Homo sapiens [sequence omitted]

SEQ ID NO: 41 - DNA, 3309 bp, Homo sapiens
  CDS (295)..(1983)
gcggccgcgctgcctggcagcccgggaagccgcggcacagctgctcggcgcctgcagctccggctcgggggctggaaccgaagcgggggcggcgggagcgcggagaccacagcccccggggagaggcggagggggtccctggcctgggcggagaggctgagctgagtgcgcgtgagaaagagggctgcaccgctgctcggcgcggactctgccagccccagcttcagccccggctcaggtcgccgcagcccgggagcctccccgcttgcgccccaaggcacgcgcggcacagccatgaacaccaacgatgccaaggagtatctggcccggagggaaatccctcagctttttgagagccttttgaatggactgatgtgttctaagcccgaagatccagtagaatacttggaaagttgtttacaaaaagtaaaggaactgggtggctgtgacaaggtgaaatgggatacatttgtaagccaggaaaagaagaccttacctccactaaatggaggacagtcacggagatcctttctaagaaatgtaatgcctgaaaactcaaactttccatatcggcggtatgaccggctccctccaatccatcaattctccatagaaagtgacacggatctctctgagactgcggagttgattgaggagtatgaggtttttgatcctaccagacctcgaccaaaaatcattcttgttataggtggtccaggaagtggaaagggtactcagagtttgaaaattgcagaacgatatggattccaatacatttctgtgggagaattattaagaaagaagatccacagtaccagcagcaataggaaatggagtcttattgccaagataattacaactggagaattggccccacaggaaacaacaattacagagataaaacaaaaattgatgcaaatacctgatgaagagggcattgttattgatggatttccaagagatgttgcccaggctctatcttttgaggaccaaatctgtacccccgattttgtggtattcctggcttgtgctaatcagagactcaaagaaagattactgaagcgtgcagaacagcagggccgaccagacgacaatgtaaaagctacccaaaggagactaatgaacttcaagcagaatgctgctccattggttaaatacttccaggaaaaggggctcatcatgacatttgatgccgaccgcgatgaggatgaggtgttctatgacatcagcatggcagttgacaacaagttatttccaaacaaagaggctgcagcaggttcaagtgaccttgatccttcgatgatattggacactggagagatcattgatacaggatctgattatgaagatcagggtgatgaccagttaaatgtatttggagaggacactatgggaggtttcatggaagatttgagaaagtgtaaaattattttcataattggtggtcctggctctggcaaaggcacacagtgtgaaaagctggtggaaaaatatggatttacacatctctcaactggcgagctcctgcgtgaggaactggcatcagaatctgaaagaagcaaattgatcagagacattatggaacgtggagacctggtgccctcaggcatcgttttggagctcctgaaggaggccatggtggccagcctcggggacaccaggggcttcctgattgacggctatcctcgggaggtgaagcaaggggaagagttcggacgcaggattggagacccacagttggtgatctgtatggactgctcggcagacaccatgaccaaccgccttctccaaaggagccggagcagcctgcctgtggacgacaccaccaagaccatcgccaagcgcctagaagcctactaccgagcgtccatccccgtgatcgcctactacgagacaaaaacacagctacacaagataaatgcagagggaacaccagaggacgtttttcttcaactctgcacagctattgactctattattttctgaaggcaaaaatgcatgtttgttagaatggaaacagaaaaacattaaaaagttcattccttaacacaatgtttcaagttaaaccttttgtgtcaccgccccccaccaaccaccacctcctaaatcctgacagcactgtttgcttcccagctagacctgtgtgagaggtgtctggaaatcatgcatggtgtatttgggactatatcaacctattctccacacttcagacaactgtctgcactcacggcacgcacactttgtatcatgcaggccacactcagagctagtcagtacatgaacagtggtgcggtgccagtctgtgtccgttgtgatcacaggccttgctagaccctgatcatctggttctcctctcattaagcatccctaacccccagtcacaccttcctcttacatactgttccccaatggaggcccctggcataggggacagccctgggcatcttcctttggtgtctggctgttttgtcaactctcatccactggtggctcagagccataaggtgggttgattacacaatgccttgtacatgatatagaggcatcaagcaagtaaaatttgacagaaattttaaaatatgaagatgtatagctttcccaagatgatggtaaaacccaggttagtcatcagtaaccttctctattattattattttttagaaacttggaatactgtcattatggctaagagaacaaatctgataaattgtgtaacctagtctcttctctacatggtgatgcatttcagcaattataaattaatataaatgaccaaaagtaacttaaaagcatgagatatttgctatttcattcattgggcacatatcaaattataattttgattttaaatggtcacccatgtatttgttgccaagcaagtaaaaaaataccctaacaaacctgatgtgggtgggaggggcatgtcagtaagtggtgtgttcaatgtgtttgtttcatatgggccctttccaggagtttgcaaaccttgtcatacccatatgcaaaactgtgtttccttgcattaaaccagtgaagtttgggttctcttttgtgctatcaatcagttgtaaaatcagagcttcttatatattctactggaataactgcatcttccactcagtcactacaaaaaagcatagtttcagtttgcatgaatttttttttttttcttcaatggttgtgcagataaggatccatttctgggatagaattgtattttttaagtcatttttttttcttgaaatggatatgtacaaataaaattaaatggaagacaggaaaaaaaaaaaaaaaaaaaaaaaaaaaaaa

SEQ ID NO: 42 - protein, 563 aa, Homo sapiens; identical to SEQ ID NO: 26

SEQ ID NO: 43 - DNA, 3205 bp, Homo sapiens
  CDS (75)..(1145)
gacaagagctcagacctgaggagagtgactagcttctctgtgtcccaggtggccaccttccactgtggaagctcatggactccattgggtcttcagggttgcggcagggggaagaaaccctgagttgctctgaggagggcttgcccgggccctcagacagctcagagctggtgcaggagtgcctgcagcagttcaaggtgacaagggcacagctacagcagatccaagccagcctcttgggttccatggagcaggcgctgaggggacaggccagccctgcccctgcggtccggatgctgcctacatacgtggggtccaccccacatggcactgagcaaggagacttcgtggtgctggagctgggggccacaggggcctcactgcgtgttttgtgggtgactctaactggcattgaggggcatagggtggagcccagaagccaggagtttgtgatcccccaagaggtgatgctgggtgctggccagcagctctttgactttgctgcccactgcctgtctgagttcctggatgcgcagcctgtgaacaaacagggtctgcagcttggcttcagcttctctttcccttgtcaccagacgggcttggacaggagcaccctcatttcctggaccaaaggttttaggtgcagtggtgtggaaggccaggatgtggtccagctgctgagagatgccattcggaggcagggggcctacaacatcgacgtggttgctgtggtgaacgacacagtgggcaccatgatgggctgtgagccgggggtcaggccgtgtgaggttgggctagttgtagacacgggcaccaacgcgtgttacatggaggaggcacggcatgtggcagtgctggacgaagaccggggccgcgtctgcgtcagcgtcgagtggggctccttaagcgatgatggggcgctgggaccagtgctgaccaccttcgaccataccctggaccatgagtccctgaatcctggtgctcagaggtttgagaagatgatcggaggcctgtacctgggtgagctggtgcggctggtgctggctcacttggcccggtgtggggtcctctttggtggctgcacctcccctgccctgctgagccaaggcagcatcctcctggaacacgtggctgagatggaggagtgagtcggggagatggtggtttagtgggggattcttggcttggaggaaggggatgatactctgttcccaaggtagccatggggctttagtgggatggggagcttctgggctgagccccaaaccacttccctttcccctccagcccctctactggggcagcccgtgtccatgctatcctgcaggacttgggcctgagccctggggcttcggatgttgagcttgtgcagcacgtctgtgcggccgtgtgcacgcgggctgcccagctctgtgctgccgccctggccgctgttctctcctgcctccagcacagccgggagcaacaaacactccaggttgctgtggccaccggaggccgagtgtgtgagcggcaccccaggttctgcagcgtcctgcaggggacagtgatgctcctggccccggaatgcgatgtctccttaatcccctctgtggatggtggtggccggggagtggcgatggtgactgctgtggctgcccgtctggctgcccaccggcgcctgctggaggagaccctggccccattccggttgaaccatgatcaactggctgcggttcaggcacagatgcggaaggccatggccaaggggctccgaggggaggcctcctcccttcgcatgctgcccactttcgtccgggccacccctgacggcagcgagcgaggggatttcctggccctggacctcgggggcacgaacttccgtgtcctcctggtacgtgtgaccacaggcgtgcagatcaccagcgagatctactccattcccgagactgtggcccagggttctgggcagcagctctttgaccacatcgtggactgcatcgtggacttccagcagaagcagggcctgagcgggcagagcctcccactgggttttaccttctccttcccatgtaggcagcttggcctagaccagggcatcctcctgaactggaccaagggtttcaaggcatcagactgcgagggccaagatgtcgtgagtctgttgcgggaagccatcactcgcagacaggcagtggagctgaatgtggttgccattgtcaatgacacggtggggaccatgatgtcctgtggctatgaggacccccgttgcgagataggcctcattgtcggaaccggcaccaatgcctgctacatggaggagctccggaatgtggcgggcgtgcctggggactcaggccgcatgtgcatcaacatggagtggggcgcctttggggacgatggctctctggccatgctcagcacccgctttgatgcaagtgtggaccaggcgtccatcaaccccggcaagcagaggtttgaaaagatgatcagcggcatgtacctgggggagatcgtccgccacatccttttacatttaaccagccttggcgttctcttccggggccagcagatccagcgccttcagaccagggacatcttcaagaccaagttcctctctgagatcgaaagtgacagcctggccctgcggcaggtccgagccatcctagaggatctggggctacccctgacctcagatgacgccctgatggtgctagaggtgtgccaggctgtgtcccagagggctgcccagctctgtggggcgggtgtagctgccgtggtggagaagatccgggggaaccggggcctggaagagctggcagtgtctgtgggggtggatggaacgctctacaagctgcacccgcgcttctccagcctggtggcggccacagtgcgggagctggcccctcgctgtgtggtcacgttcctgcagtcagaggatgggtccggcaaaggtgcggccctggtcaccgctgttgcctgccgccttgcgcagttgactcgtgtctgaggaaacctccaggctgaggaggtctccgccgcagccttgctggagccgggtcggggtctgcctgtttcccagccaggcccagccacccaggactcctgggacatcccatgtgtgacccctctgcggccatttggccttgctccctggctttccctgagagaagtagcactcaggttagcaatatatatatataatttatttacaaaaaaaaaaaaa

SEQ ID NO: 44 - protein, 357 aa, Homo sapiens
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSLSDDGALGPVLTTFDHTLDHESLNPGAQRFEKMIGGLYLGELVRLVLAHLARCGVLFGGCTSPALLSQGSILLEHVAEMEE

SEQ ID NO: 45 - DNA, 1096 bp, Homo sapiens
  CDS (2)..(1096)
caccaagcttcccaccatggactccattgggtcttcagggttgcggcagggggaagaaaccctgagttgctctgaggagggcttgcccgggccctcagacagctcagagctggtgcaggagtgcctgcagcagttcaaggtgacaagggcacagctacagcagatccaagccagcctcttgggttccatggagcaggcgctgaggggacaggccagccctgcccctgcggtccggatgctgcctacatacgtggggtccaccccacatggcactgagcaaggagacttcgtggtgctggagctgggggccacaggggcctcactgcgtgttttgtgggtgactctaactggcattgaggggcatagggtggagcccagaagccaggagtttgtgatcccccaagaggtgatgctgggtgctggccagcagctctttgactttgctgcccactgcctgtctgagttcctggatgcgcagcctgtgaacaaacagggtctgcagcttggcttcagcttctctttcccttgtcaccagacgggcttggacaggagcaccctcatttcctggaccaaaggttttaggtgcagtggtgtggaaggccaggatgtggtccagctgctgagagatgccattcggaggcagggggcctacaacatcgacgtggttgctgtggtgaacgacacagtgggcaccatgatgggctgtgagccgggggtcaggccgtgtgaggttgggctagttgtagacacgggcaccaacgcgtgttacatggaggaggcacggcatgtggcagtgctggacgaagaccggggccgcgtctgcgtcagcgtcgagtggggctccttcagcgatgatggggcgctgggaccagtgctgaccaccttcgaccataccctggaccatgagtccctgaatcctggtgctcagaggtttgagaagatgatcggaggcctgtacctgggtgagctggtgcggctggtgctggctcacttggcccggtgtggggtcctctttggtggctgcacctcccctgccctgctgagccaaggcagcatcctcctggaacacgtggctgagatggaggaggtcgacggc

SEQ ID NO: 46 - protein, 365 aa, Homo sapiens
TKLPTMDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSFSDDGALGPVLTTFDHTLDHESLNPGAQRFEKMIGGLYLGELVRLVLAHLARCGVLFGGCTSPALLSQGSILLEHVAEMEEVDG

SEQ ID NO: 47 - DNA, 1096 bp, Homo sapiens; identical to SEQ ID NO: 45
  CDS (17)..(1087)

SEQ ID NO: 48 - protein, 357 aa, Homo sapiens
MDSIGSSGLRQGEETLSCSEEGLPGPSDSSELVQECLQQFKVTRAQLQQIQASLLGSMEQALRGQASPAPAVRMLPTYVGSTPHGTEQGDFVVLELGATGASLRVLWVTLTGIEGHRVEPRSQEFVIPQEVMLGAGQQLFDFAAHCLSEFLDAQPVNKQGLQLGFSFSFPCHQTGLDRSTLISWTKGFRCSGVEGQDVVQLLRDAIRRQGAYNIDVVAVVNDTVGTMMGCEPGVRPCEVGLVVDTGTNACYMEEARHVAVLDEDRGRVCVSVEWGSFSDDGALGPVLTTFDHTLDHESLNPGAQRFEKMIGGLYLGELVRLVLAHLARCGVLFGGCTSPALLSQGSILLEHVAEMEE

SEQ ID NO: 49 - DNA, 1602 bp, Homo sapiens
  CDS (1)..(1599)
atggcttcgaccaccacctgcaccaggttcacggacgagtatcagcttttcgaggagcttggaaagggggcattctcagtggtgagaagatgtatgaaaattcctactggacaaggatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaagataaacaacaaagccaacgtggtaaccagccccaaagaaaatattcctaccccagcgctggagccccaaactactgtaatccacaaccctgatggaaacaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatctga

SEQ ID NO: 50 - protein, 533 aa, Homo sapiens
MASTTTCTRFTDEYQLFEELGKGAFSVVRRCMKIPTGQGYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKINNKANVVTSPKENIPTPALEPQTTVIHNPDGNKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIKPPCIPNGKENFSGGTSLWQNI

SEQ ID NO: 51 - DNA, 1519 bp, Homo sapiens
  CDS (2)..(1519)
caccggatccaccatggcttcgaccaccacctgcaccaggttcacggacgagtatcagcttttcgaggagcttggaaagggggcattctcagtggtgagaagatgtatgaaaattcctactggacaagaatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatcctcgagggc

SEQ ID NO: 52 - protein, 506 aa, Homo sapiens
TGSTMASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIKPPCIPNGKENFSGGTSLWQNILEG

SEQ ID NO: 53 - DNA, 1561 bp, Homo sapiens
  CDS (2)..(1561)
caccggatccaccatggcttcgaccaccacctgcaccaggttcacggacgagtatcagcttttcgaggagcttggaaagggggcattctcagtggtgagaagatgtatgaaaattcctactggacaagaatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagcagccaagagtttgttgaagaaaccagatggagtaaaggagccccaaactactgtaatccacaaccctgatggaaacaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatcctcgagggc

SEQ ID NO: 54 - protein, 520 aa, Homo sapiens
TGSTMASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAAKSLLKKPDGVKEPQTTVIHNPDGNKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIKPPCIPNGKENFSGGTSLWQNILEG

SEQ ID NO: 55 - DNA, 1558 bp, Homo sapiens
  CDS (2)..(1558)
caccggatccaccatggcttcgaccaccacctgcaccaggttcacggacgagtatcagcttttcgaggagcttggaaagggggcattctcagtggtgagaagatgtatgaaaattcctactggacaagaatatgctgccaaaattatcaacaccaaaaagctttctgctagggatcatcagaaactagaaagagaagctagaatctgccgtcttttgaagcaccctaatattgtgcgacttcatgatagcatatcagaagagggctttcactacttggtgtttgatttagttactggaggtgaactgtttgaagacatagtggcaagagaatactacagtgaagctgatgccagtcattgtatacagcagattctagaaagtgttaatcattgtcacctaaatggcatagttcacagggacctgaagcctgagaatttgcttttagctagcaaatccaagggagcagctgtgaaattggcagactttggcttagccatagaagttcaaggggaccagcaggcgtggtttggttttgctggcacacctggatatctttctccagaagttttacgtaaagatccttatggaaagccagtggatatgtgggcatgtggtgtcattctctatattctacttgtggggtatccacccttctgggatgaagaccaacacagactctatcagcagatcaaggctggagcttatgattttccatcaccagaatgggacacggtgactcctgaagccaaagacctcatcaataaaatgcttactatcaaccctgccaaacgcatcacagcctcagaggcactgaagcacccatggatctgtcaacgttctactgttgcttccatgatgcacagacaggagactgtagactgcttgaagaaatttaatgctagaagaaaactaaagggtgccatcttgacaactatgctggctacaaggaatttctcagccaagagtttgttgaagaaaccagatggagtaaaggagccccaaactactgtaatccacaaccctgatggaaacaaggagtcaactgagagttcaaatacaacaattgaggatgaagatgtgaaagcacgaaagcaagagattatcaaagtcactgaacaactgatcgaagctatcaacaatggggactttgaagcctacacaaaaatctgtgacccaggccttactgcttttgaacctgaagctttgggtaatttagtggaagggatggattttcaccgattctactttgaaaatgctttgtccaaaagcaataaaccaatccacactattattctaaaccctcatgtacatctggtaggggatgatgccgcctgcatagcatatattaggctcacacagtacatggatggcagtggaatgccaaagacaatgcagtcagaagagactcgtgtgtggcaccgccgggatggaaagtggcagaatgttcattttcatcgctcggggtcaccaacagtacccatcaagccaccctgtattccaaatgggaaagaaaacttctcaggaggcacctctttgtggcaaaacatcctcgagggc

SEQ ID NO: 56 - protein, 519 aa, Homo sapiens
TGSTMASTTTCTRFTDEYQLFEELGKGAFSVVRRCMKIPTGQEYAAKIINTKKLSARDHQKLEREARICRLLKHPNIVRLHDSISEEGFHYLVFDLVTGGELFEDIVAREYYSEADASHCIQQILESVNHCHLNGIVHRDLKPENLLLASKSKGAAVKLADFGLAIEVQGDQQAWFGFAGTPGYLSPEVLRKDPYGKPVDMWACGVILYILLVGYPPFWDEDQHRLYQQIKAGAYDFPSPEWDTVTPEAKDLINKMLTINPAKRITASEALKHPWICQRSTVASMMHRQETVDCLKKFNARRKLKGAILTTMLATRNFSAKSLLKKPDGVKEPQTTVIHNPDGNKESTESSNTTIEDEDVKARKQEIIKVTEQLIEAINNGDFEAYTKICDPGLTAFEPEALGNLVEGMDFHRFYFENALSKSNKPIHTIILNPHVHLVGDDAACIAYIRLTQYMDGSGMPKTMQSEETRVWHRRDGKWQNVHFHRSGSPTVPIKPPCIPNGKENFSGGTSLWQNILEG

SEQ ID NO: 57 - DNA, 1390 bp, Homo sapiens
  CDS (3)..(1376)
caccgaattccaccatggggtgcatgaagtccaagttcctccaggtcggaggcaatacattctcaaaaactgaaaccagcgccagtgcacactgtcctgtgtacgtgccggatcccacatccaccatcaagccggggcctaatagccacaacagcaacacaccaggaatcagggaggcaggctctgaggacatcatcgtggttgccctgtacgattacgaggccattcaccacgaagacctcagcttccagaagggggaccagatggtggtcctagaggaatccggggagtggtggaaggctcgatccctggccacccggaaggagggctacatcccaagcaactatgtcgcccgcgttgactctctggagacagaggagtggtttttcaagggcatcagccggaaggacgcagagcgccaactgctggctcccggcaacatgctgggctccttcatgatccgggatagcgagaccactaaaggaagctactctttgtccgtgcgagactacgaccctcggcagggagataccgtgaaacattacaagatccggaccctggacaacgggggcttctacatatccccccgaagcaccttcagcactctgcaggagctggtggaccactacaagaccacctacaacaagcacaccaaggtggcagtgaagacgatgaagccagggagcatgtcggtggaggccttcctggcagaggccaacgtgatgaaaactctgcagcatgacaagctggtcaaacttcatgcggtggtcaccaaggagcccatctacatcatcacggagttcatggccaaaggaagcttgctggactttctgaaaagtgatgagggcagcaagcagccattgccaaaactcattgacttctcagcccagattgcagaaggcatggccttcatcgagcagaggaactacatccaccgagacctccgagctgccaacatcttggtctctgcatccctggtgtgtaagattgctgactttggcctggcccgggtcattgaggacaacgagtacacggctcgggaaggggccaagttccccatcaagtggacagctcctgaagccatcaactttggctccttcaccatcaagtcagacgtctggtcctttggtatcctgctgatggagatcgtcacctacggccggatcccttacccagggatgtcaaaccctgaagtgatccgagctctggagcgtggataccggatgcctcgcccagagaactgcccagaggagctctacaacatcatgatgcgctgctggaaaaaccgtccggaggagcggccgaccttcgaatacatccagagtgtgctggatgacttctacacggccacagagagccagtaccaacagcagccatgagcggccgctat

SEQ ID NO: 58 - protein, 458 aa, Homo sapiens
PNSTMGCMKSKFLQVGGNTFSKTETSASAHCPVYVPDPTSTIKPGPNSHNSNTPGIREAGSEDIIVVALYDYEAIHHEDLSFQKGDQMVVLEESGEWWKARSLATRKEGYIPSNYVARVDSLETEEWFFKGISRKDAERQLLAPGNMLGSFMIRDSETTKGSYSLSVRDYDPRQGDTVKHYKIRTLDNGGFYISPRSTFSTLQELVDHYKTTYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQYQQQP

SEQ ID NO: 59 - DNA, 1926 bp, Homo sapiens
  CDS (76)..(1590)
gaattcctttctaaaatccaaccattccaggaaatagaaatatcaacttgggggcttcctgagaatgtcagattgatggggtgcatgaagtccaagttcctccaggtcggaggcaatacattctcaaaaactgaaaccagcgccagcccacactgtcctgtgtacgtgccggatcccacatccaccatcaagccggggcctaatagccacaacagcaacacaccaggaatcagggaggcaggctctgaggacatcatcgtggttgccctgtatgattacgaggccattcaccacgaagacctcagcttccagaagggggaccagatggtggtcctagaggaatccggggagtggtggaaggctcgatccctggccacccggaaggagggctacatcccaagcaactatgtcgcccgcgttgactctctggagacagaggagtggtttttcaagggcatcagccggaaggacgcagagcgccaactgctggctcccggcaacatgctgggctccttcatgatccgggatagcgagaccactaaaggaagctactctttgtccgtgcgagactacgaccctcggcagggagataccgtgaaacattacaagatccggaccctggacaacgggggcttctacatatccccccgaagcaccttcagcactctgcaggagctggtggaccactacaagaaggggaacgacgggctctgccagaaactgtcggtgccctgcatgtcttccaagccccagaagccttgggagaaagatgcctgggagatccctcgggaatccctcaagctggagaagaaacttggagctgggcagtttggggaagtctggatggccacctacaacaagcacaccaaggtggcagtgaagacgatgaagccagggagcatgtcggtggaggccttcctggcagaggccaacgtgatgaaaactctgcagcatgacaagctggtcaaacttcatgcggtggtcaccaaggagcccatctacatcatcacggagttcatggccaaaggaagcttgctggactttctgaaaagtgatgagggcagcaagcagccattgccaaaactcattgacttctcagcccagattgcagaaggcatggccttcatcgagcagaggaactacatccaccgagacctccgagctgccaacatcttggtctctgcatccctggtgtgtaagattgctgactttggcctggcccgggtcattgaggacaacgagtacacggctcgggaaggggccaagttccccatcaagtggacagctcctgaagccatcaactttggctccttcaccatcaagtcagacgtctggtcctttggtatcctgctgatggagatcgtcacctacggccggatcccttacccagggatgtcaaaccctgaagtgatccgagctctggagcgtggataccggatgcctcgcccagagaactgcccagaggagctctacaacatcatgatgcgctgctggaaaaaccgtccggaggagcggccgaccttcgaatacatccagagtgtgctggatgacttctacacggccacagagagccagtaccaacagcagccatgatagggaggaccagggcagggcagggggtgcccaggtggtggctgcaaggtggctccagcaccatccgccagggcccacacccccttccttcacccagacacccaccctcgcttcaggccacagtttcctcatctgtccagtgggtaggttggactggaaaatctctttttgactcttgcaatggacaatctgacattctcaggaagcccccaagttgatatttctatttcctggaatggttggattttagttacagctgtgatttggaagggaaactttcaaaatagtgaaatgaatatttaaataaaagatataaatgccaaagtctttacc

SEQ ID NO: 60 - protein, 505 aa, Homo sapiens
MGCMKSKFLQVGGNTFSKTETSASPHCPVYVPDPTSTIKPGPNSHNSNTPGIREAGSEDIIVVALYDYEAIHHEDLSFQKGDQMVVLEESGEWWKARSLATRKEGYIPSNYVARVDSLETEEWFFKGISRKDAERQLLAPGNMLGSFMIRDSETTKGSYSLSVRDYDPRQGDTVKHYKIRTLDNGGFYISPRSTFSTLQELVDHYKKGNDGLCQKLSVPCMSSKPQKPWEKDAWEIPRESLKLEKKLGAGQFGEVWMATYNKHTKVAVKTMKPGSMSVEAFLAEANVMKTLQHDKLVKLHAVVTKEPIYIITEFMAKGSLLDFLKSDEGSKQPLPKLIDFSAQIAEGMAFIEQRNYIHRDLRAANILVSASLVCKIADFGLARVIEDNEYTAREGAKFPIKWTAPEAINFGSFTIKSDVWSFGILLMEIVTYGRIPYPGMSNPEVIRALERGYRMPRPENCPEELYNIMMRCWKNRPEERPTFEYIQSVLDDFYTATESQYQQQP

SEQ ID NO: 61 - DNA, 3109 bp, Homo sapiens
  CDS (75)..(1478)
aggggctgaggaggtactggaaaagaaagaggagcaggagctggaggaagacgtggaggaggagctggaggaggatgaagagaaggagtgggacgcccacaaccctgtgtaaggagctcaagtactccaaggacccgccccagatatccatcatattcatcttcgtgaacgaggccctgtcggtgatcctgcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacagctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagtagagggagggagctggggcactggagcctggcccccaggacatggctgctccccccaacatctggaccagctgccctggcggagagacagcaaggggccggcaggtgctcgatgggccccccagggcttctccagggcagcacagggaccccggatgaagactctgtcccccctcaggcattcagctgcccacaagtttcctgcaccctggaaaagccccccacccttcctctgggaaactgacagctgtcttccacagcctctgatgtggacctggtactgaggagcaagactgtccagttctcctccacatctcccatcccagaatcaggatctgggactggcagggtcccctcctgtgtctcatctcttgcagcagcagctgctgaactccagccatcaacacggtgggaggcagcgggggcttcagccatgtcctagctccccgccctaaaaggaggcagtgaggaccaggcactatttcctccgaggttacttctacccagatgacacctgcctgttcacgccccaaggcagctactgcccctaacccttcccaccagggtagctttgggcactgcagctctggacttttctggcccctcctgagatgacctgatggagctgatgctttctctcctaatccctgggcactaggctcttatcagtgtgcttgggccagctctcctgcctgtgtctagaggaagccagagacagaaataggctaagcctgcagtaggatctcagccacaagggccccgcaggatggagctgggtcaaggaccagggagccctgactcccagaggctgccaccggggagaagcagcggtcctccatccagaacctaagggctgaagcaaaggctgccaggacccttgaagatgcttttggctcacctcatttcaccccacgctctgctggctggcagaggagaaggcagtcgtttcctctctgaagagtatttttttcgattgccctctggttagggtgcacatataaatcagagttaatatatgaacgcgtgtgcatgcacaagtgtgtgtgtgcctgcgtgctgtgcgtggcagggtgtgtgtgtgtgtgtctggctgtgcgttccggagtgtgtgacgatgctgacctagctgtgtggccttgggcttgctgcttcattactcacctggatggggacgagggatgagaagggtgtgggtttggccccatgtcactggccggaaggatgtgtctcagccctgccctgtggggtgcccccgatgggaggctgtcccatctcccagtccccatctctttttccccacactgtccctggccaagccctgcccagagctgaaccctgtagctgcccccttgccctgtgtgggattcgcagtgtctcatttggtgacgtcttactggtgatcatctcctcaccccatctcccaccttgtggaataaatacatgttagcacttcccagagcagcctcctttgtgtcttgatttctccagaactggaggtggggaggggagtgatggagacataggaggagagcttctttggctttgagggtttagtgttacttatttatctatttattcgagatggggtcttgctctgtggcccaggctggagtgcagtggtgcaatcatga

SEQ ID NO: 62 - protein, 468 aa, Homo sapiens
MKRRSGTPTTLCKELKYSKDPPQISIIFIFVNEALSVILRSVHSAVNHTPTHLLKEIILVDDNSDEEELKVPLEEYVHKRYPGLVKVVRNQKREGLIRARIEGWKVATGQVTGFFDAHVEFTAGWAEPVLSRIQENRKRVILPSIDNIKQDNFEVQRYENSAHGYSWELWCMYISPPKDWWDAGDPSLPIRTPAMIGCSFVVNRKFFGEIGLLDPGMDVYGGENIELGIKVWLCGGSMEVLPCSRVAHIERKKKPYNSNIGFYTKRNALRVAEVWMDDYKSHVYIAWNLPLENPGIDIGDVSERRALRKSLKCKNFQWYLDHVYPEMRRYNNTVAYGELRNNKAKDVCLDQGPLENHTAILYPCHGWGPQLARYTKEGFLHLGALGTTTLLPDTRCLVDNSKSRLPQLLDCDKVKSSLYKRWNFIQNGAIMNKGTGRCLEVENRGLAGIDLILRSCTGQRWTIKNSIK

SEQ ID NO: 63 - DNA, 1306 bp, Homo sapiens
  CDS (2)..(1306)
caccagatctcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacagctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagctcgagggc

SEQ ID NO: 64 - protein, 435 aa, Homo sapiens
TRSRSVHSAVNHTPTHLLKEIILVDDNSDEEELKVPLEEYVHKRYPGLVKVVRNQKREGLIRARIEGWKVATGQVTGFFDAHVEFTAGWAEPVLSRIQENRKRVILPSIDNIKQDNFEVQRYENSAHGYSWELWCMYISPPKDWWDAGDPSLPIRTPAMIGCSFVVNRKFFGEIGLLDPGMDVYGGENIELGIKVWLCGGSMEVLPCSRVAHIERKKKPYNSNIGFYTKRNALRVAEVWMDDYKSHVYIAWNLPLENPGIDIGDVSERRALRKSLKCKNFQWYLDHVYPEMRRYNNTVAYGELRNNKAKDVCLDQGPLENHTAILYPCHGWGPQLARYTKEGFLHLGALGTTTLLPDTRCLVDNSKSRLPQLLDCDKVKSSLYKRWNFIQNGAIMNKGTGRCLEVENRGLAGIDLILRSCTGQRWTIKNSIKLEG

SEQ ID NO: 65 - DNA, 3109 bp, Homo sapiens
  CDS (75)..(1478)
aggggctgaggaggtactggaaaagaaagaggagcaggagctggaggaagacgtggaggaggagctggaggaggatgaagagaaggagtgggacgcccacaaccctgtgtaaggagctcaagtactccaaggacccgccccagatatccatcatattcatcttcgtgaacgaggccctgtcggtgatcctgcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacggctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagtagagggagggagctggggcactggagcctggcccccaggacatggctgctccccccaacatctggaccagctgccctggcggagagacagcaaggggccggcaggtgctcgatgggccccccagggcttctccagggcagcacagggaccccggatgaagactctgtcccccctcaggcattcagctgcccacaagtttcctgcaccctggaaaagccccccacccttcctctgggaaactgacagctgtcttccacagcctctgatgtggacctggtactgaggagcaagactgtccagttctcctccacatctcccatcccagaatcaggatctgggactggcagggtcccctcctgtgtctcatctcttgcagcagcagctgctgaactccagccatcaacacggtgggaggcagcgggggcttcagccatgtcctagctccccgccctaaaaggaggcagtgaggaccaggcactatttcctccgaggttacttctacccagatgacacctgcctgttcacgccccaaggcagctactgcccctaacccttcccaccagggtagctttgggcactgcagctctggacttttctggcccctcctgagatgacctgatggagctgatgctttctctcctaatccctgggcactaggctcttatcagtgtgcttgggccagctctcctgcctgtgtctagaggaagccagagacagaaataggctaagcctgcagtaggatctcagccacaagggccccgcaggatggagctgggtcaaggaccagggagccctgactcccagaggctgccaccggggagaagcagcggtcctccatccagaacctaagggctgaagcaaaggctgccaggacccttgaagatgcttttggctcacctcatttcaccccacgctctgctggctggcagaggagaaggcagtcgtttcctctctgaagagtatttttttcgattgccctctggttagggtgcacatataaatcagagttaatatatgaacgcgtgtgcatgcacaagtgtgtgtgtgcctgcgtgctgtgcgtggcagggtgtgtgtgtgtgtgtctggctgtgcgttccggagtgtgtgacgatgctgacctagctgtgtggccttgggcttgctgcttcattactcacctggatggggacgagggatgagaagggtgtgggtttggccccatgtcactggccggaaggatgtgtctcagccctgccctgtggggtgcccccgatgggaggctgtcccatctcccagtccccatctctttttccccacactgtccctggccaagccctgcccagagctgaaccctgtagctgcccccttgccctgtgtgggattcgcagtgtctcatttggtgacgtcttactggtgatcatctcctcaccccatctcccaccttgtggaataaatacatgttagcacttcccagagcagcctcctttgtgtcttgatttctccagaactggaggtggggaggggagtgatggagacataggaggagagcttctttggctttgagggtttagtgttacttatttatctatttattcgagatggggtcttgctctgtggcccaggctggagtgcagtggtgcaatcatga

SEQ ID NO: 66 - protein, 468 aa, Homo sapiens
MKRRSGTPTTLCKELKYSKDPPQISIIFIFVNEALSVILRSVHSAVNHTPTHLLKEIILVDDNSDEEELKVPLEEYVHKRYPGLVKVVRNQKREGLIRARIEGWKVATGQVTGFFDAHVEFTAGWAEPVLSRIQENRKRVILPSIDNIKQDNFEVQRYENSAHGYGWELWCMYISPPKDWWDAGDPSLPIRTPAMIGCSFVVNRKFFGEIGLLDPGMDVYGGENIELGIKVWLCGGSMEVLPCSRVAHIERKKKPYNSNIGFYTKRNALRVAEVWMDDYKSHVYIAWNLPLENPGIDIGDVSERRALRKSLKCKNFQWYLDHVYPEMRRYNNTVAYGELRNNKAKDVCLDQGPLENHTAILYPCHGWGPQLARYTKEGFLHLGALGTTTLLPDTRCLVDNSKSRLPQLLDCDKVKSSLYKRWNFIQNGAIMNKGTGRCLEVENRGLAGIDLILRSCTGQRWTIKNSIK

SEQ ID NO: 67 - DNA, 3109 bp, Homo sapiens
  CDS (75)..(1478)
aggggctgaggaggtactggaaaagaaagaggagcaggagctggaggaagacgtggaggaggagctggaggaggatgaagagaaggagtgggacgcccacaaccctgtgtaaggagctcaagtactccaaggacccgccccagatatccatcatattcatcttcgtgaacgaggccctgtcggtgatcctgcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacagctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagtagagggagggagctggggcactggagcctggcccccaggacatggctcctccccccaacatctggaccagctgccctggcggagagacagcaaggggccggcaggtgctcgatgggccccccagggcttctccagggcagcacagggaccccggatgaagactctgtcccccctcaggcattcagctgcccacaagtttcctgcaccctggaaaagccccccacccttcctctgggaaactgacagctgtcttccacagcctctgatgtggacctggtactgaggagcaagactgtccagttctcctccacatctcccatcccagaatcaggatctgggactggcagggtcccctcctgtgtctcatctcttgcagcagcagctgctgaactccagccatcaacacggtgggaggcagcgggggcttcagccatgtcctagctccccgccctaaaaggaggcagtgaggaccaggcactatttcctccgaggttacttctacccagatgacacctgcctgttcacgccccaaggcagctactgcccctaacccttcccaccagggtagctttgggcactgcagctctggacttttctggcccctcctgagatgacctgatggagctgatgctttctctcctaatccctgggcactaggctcttatcagtgtgcttgggccagctctcctgcctgtgtctagaggaagccagagacagaaataggctaagcctgcagtaggatctcagccacaagggccccgcaggatggagctgggtcaaggaccagggagccctgactcccagaggctgccaccggggagaagcagcggtcctccatccagaacctaagggctgaagcaaaggctgccaggacccttgaagatgcttttggctcacctcatttcaccccacgctctgctggctggcagaggagaaggcagtcgtttcctctctgaagagtatttttttcgattgccctctggttagggtgcacatataaatcagagttaatatatgaacgcgtgtgcatgcacaagtgtgtgtgtgcctgcgtgctgtgcgtggcagggtgtgtgtgtgtgtgtctggctgtgcgttccggagtgtgtgacgatgctgacctagctgtgtggccttgggcttgctgcttcattactcacctggatggggacgagggatgagaagggtgtgggtttggccccatgtcactggccggaaggatgtgtctcagccctgccctgtggggtgcccccgatgggaggctgtcccatctcccagtccccatctctttttccccacactgtccctggccaagccctgcccagagctgaaccctgtagctgcccccttgccctgtgtgggattcgcagtgtctcatttggtgacgtcttactggtgatcatctcctcaccccatctcccaccttgtggaataaatacatgttagcacttcccagagcagcctcctttgtgtcttgatttctccagaactggaggtggggaggggagtgatggagacataggaggagagcttctttggctttgagggtttagtgttacttatttatctatttattcgagatggggtcttgctctgtggcccaggctggagtgcagtggtgcaatcatga

SEQ ID NO: 68 - protein, 468 aa, Homo sapiens; identical to SEQ ID NO: 62

SEQ ID NO: 69 - DNA, 3109 bp, Homo sapiens
  CDS (75)..(1478)
aggggctgaggaggtactggaaaagaaagaggagcaggagctggaggaagacgtggaggaggagctggaggaggatgaagagaaggagtgggacgcccacaaccctgtgtaaggagctcaagtactccaaggacccgccccagatatccatcatattcatcttcgtgaacgaggccctgtcggtgatcctgcggtccgtgcacagtgccgtcaatcacacgcccacacacctgctgaaggaaatcattctggtggatgacaacagcgacgaagaggagctgaaggtccccctagaggagtatgtccacaaacgctaccccgggctggtgaaggtggtaagaaatcagaagagggaaggcctgatccgcgctcgcattgagggctggaaggtggctaccgggcaggtcactggcttctttgatgcccacgtggaattcaccgctggctgggctgagccggttctatcccgcatccaggaaaaccggaagcgtgtgatcctcccctccattgacaacatcaaacaggacaactttgaggtgcagcggtacgagaactcggcccacgggtacagctgggagctgtggtgcatgtacatcagccccccaaaagactggtgggacgccggagacccttctctccccatcaggaccccagccatgataggctgctcgttcgtggtcaacaggaagttcttcggtgaaattggtcttctggatcctggcatggatgtatacggaggagaaaatattgaactgggaatcaaggtatggctctgtgggggcagcatggaggtccttccttgctcacgggtggcccacattgagcggaagaagaagccatataatagcaacattggcttctacaccaagaggaatgctcttcgcgttgctgaggtctggatggacgattacaagtctcatgtgtacatagcgtggaacctgccgctggagaatccgggaattgacatcggtgatgtctccgaaagaagagcattaaggaaaagtttaaagtgtaagaatttccagtggtacctggaccatgtttacccagaaatgagaagatacaataataccgttgcttacggggagcttcgcaacaacaaggcaaaagacgtctgcttggaccaggggccgctggagaaccacacagcaatattgtatccgtgccatggctggggaccacagcttgcccgctacaccaaggaaggcttcctgcacttgggtgccctggggaccaccacactcctccctgacacccgctgcctggtggacaactccaagagtcggctgccccagctcctggactgcgacaaggtcaagagcagcctgtacaagcgctggaacttcatccagaatggagccatcatgaacaagggcacgggacgctgcctggaggtggagaaccggggcctggctggcatcgacctcatcctccgcagctgcacaggtcagaggtggaccattaagaactccatcaagtagagggagggagctggggcactggagcctggcccccaggacatggctgctccccccaacatctggaccagctgccctggcggagagacagcaaggggccggcaggtgctcgatgggccccccagggcttctccagggcagcacagggaccccggatgaagactctgtcccccctcaggcattcagctgcccacaagtttcctgcaccctggaaaagccccccacccttcctctgggaaactgacagctgtcttccacagcctctgatgtggacctggtactgaggagcaagactgtccagttctcctccacatctcccatcccagaatcaggatctgggactggcagggtcccctcctgtgtctcatctcttgcagcagcagctgctgaactccagccatcaacacggtgggaggcagcgggggcttcagccatgtcctagctccccgccctaaaaggaggcagtgaggaccaggcactatttcctccgaggttacttctacccagatgacacctgcctgttcacgccccaaggcagctactgcccctaacccttcccaccagggtagctttgggcactgcagctctggacttttctggcccctcctgagatgacctgatggagctgatgctttctctcctaatccctgggcactaggctcttatcagtgtgcttgggccagctctcctgcctgtgtctagaggaagccagagacagaaataggctaagcctgcagtaggatctcagccacaagggccccgcaggatggagctgggtcaaggaccagggagccctgactcccagaggctgccaccggggagaagcagcggtcctccatccagaacctaagggctgaagcaaaggctgccaggacccttgaagatgcttttggctcacctcatttcaccccacgctctgctggctggcagaggagaaggcagtcgtttcctctctgaagagtatttttttcgattgccctctggttagggtgcacatataaatcagagttaatatatgaacgcgtgtgcatgcacaagtgtgtgtgtgcctgcgtgctgtgcgtggcagggtgtgtgtgtgtgtgtctggctgtgcgttccggagtgtgtgacgatgctgacctagctgtgcggccttgggcttgctgcttcattactcacctggatggggacgagggatgagaagggtgtgggtttggccccatgtcactggccggaaggatgtgtctcagccctgccctgtggggtgcccccgatgggaggctgtcccatctcccagtccccatctctttttccccacactgtccctggccaagccctgcccagagctgaaccctgtagctgcccccttgccctgtgtgggattcgcagtgtctcatttggtgacgtcttactggtgatcatctcctcaccccatctcccaccttgtggaataaatacatgttagcacttcccagagcagcctcctttgtgtcttgatttctccagaactggaggtggggaggggagtgatggagacataggaggagagcttctttggctttgagggtttagtgttacttatttatctatttattcgagatggggtcttgctctgtggcccaggctggagtgcagtggtgcaatcatga

SEQ ID NO: 70 - protein, 468 aa, Homo sapiens; identical to SEQ ID NO: 62

SEQ ID NO: 71 - DNA, 1066 bp, Homo sapiens
  CDS (2)..(1057)
caccggatccatgaaggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaagtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtggggtgaatttaaggtgtcagagagatacctcaccatggatgacttgacaacagccctggaggggaacagagtgagagagatgtttggctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatccgtcgacggc

SEQ ID NO: 72 - protein, 352 aa, Homo sapiens
TGSMKDCSNGCSAECTGEGGSKEVVGTFKAKDLIVTPATILKEKPDPNNLVFGTVFTDHMLTVEWSSEFGWEKPHIKPLQNLSLHPGSSALHYAVEVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWGEFKVSERYLTMDDLTTALEGNRVREMFGSGTACVVCPVSDILYKGETIHIPTMENGPKLASRILSKLTDIQYGREESDWTIVLS

SEQ ID NO: 73 - DNA, 2043 bp, Homo sapiens
  CDS (528)..(1685)
agtagggaggtgggcaggagccagtgatgacggaatggcaatcacatttgacctctgatctgtttatttcctcctccttgacgtctccatataaatgttacacgggcatccccacactcggatacgcacccacagtggctgattcgggggtaaccgtgtcatttgcttgcaacactggcacctctgccctgcaccccgggagtgagcagtgagtgaggctcgggtctgggcgctggctccgaatcttcgggctgggagagactccaccatctgggggcggcctgggggagcagccttagtgtcttcctgctgatgcaatccgctaggtcgcgagtctccgccgcgagagggccggtctgcaatccagcccgccacgtgtactcgccgccgcctcgggcactgccccaggtcttgctgcagccgggaccgcgctctgcagccgcagacccggtccacacggccaggggctacgacccttgggatctgccctccgctcagctcgagcttccctcgtggccgacggaacaatgaaggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaattatttgaaggattgaaggcatttcgaggagtagataataaaattcgactgtttcagccaaacctcaacatggatagaatgtatcgctctgctgtgagggcaactctgccggtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtggggtgaatttaaggtgtcagagagatacctcaccatggatgacttgtcaacagccctggaggggaacagagtgagagagatgtttggctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatcctgaatggaaaatagaggatacaatggaaaatagaggataccaactgtatgctactgggacagactgttgcatttgaattgtgatagatttctttggctacctgtgcataatgtagtttgtagtatcaatgtgttacaagagtgattgtttcttcatgccagagaaaatgaattgcaatcatcaaatggtgtttcataacttggtagtagtaacttaccttaccttacctagaaaaatattaatgtaagccatataacatgggattttcctcaatgattttagtgcctccttttgtacttcactcagatactaaatagtagtttattctttaatataagttacattctgctcctcaacc

SEQ ID NO: 74 - protein, 386 aa, Homo sapiens [sequence omitted]

SEQ ID NO: 75 - DNA, 1177 bp, Homo sapiens
  CDS (11)..(1168)
caccggatccatgaaggattgcagtaacggatgctccgcagagtgtaccggagaaggaggatcaaaagaggtggtggggacttttaaggctaaagacctaatagtcacaccagctaccattttaaaggaaaaaccagaccccaataatctggtttttggaactgtgttcacggatcatatgctgacggtggagtggtcctcagagtttggatgggagaaacctcatatcaagcctcttcagaacctgtcattgcaccctggctcatcagctttgcactatgcagtggaattatttgaaggattgaaggcatttcgaggagtagataataaaattcgactgtttcagccaaacctcaacatggatagaatgtatcgctctgctgtgagggcaactctgccggtatttgacaaagaagagctcttagagtgtattcaacagcttgtgaaattggatcaagaatgggtcccatattcaacatctgctagtctgtatattcgtcctacattcattggaactgagccttctcttggagtcaagaagcctaccaaagccctgctctttgtactcttgagcccagtgggaccttatttttcaagtggaacctttaatccagtgtccctgtgggccaatcccaagtatgtaagagcctggaaaggtggaactggggactgcaagatgggagggaattacggctcatctctttttgcccaatgtgaagcagtagataatgggtgtcagcaggtcctgtggctctatggagaggaccatcagatcactgaagtgggaactatgaatctttttctttactggataaatgaagatggagaagaagaactggcaactcctccactagatggcatcattcttccaggagtgacaaggcggtgcattctggacctggcacatcagtggggtgaatttaaggtgtcagagagatacctcaccatggatgacttgacaacagccctggaggggaacagagtgagagagatgtttggctctggtacagcctgtgttgtttgcccagtttctgatatactgtacaaaggcgagacaatacacattccaactatggagaatggtcctaagctggcaagccgcatcttgagcaaattaactgatatccagtatggaagagaagagagcgactggacaattgtgctatccgtcgacggc

SEQ ID NO: 76 - protein, 386 aa, Homo sapiens
MKDCSNGCSAECTGEGGSKEVVGTFKAKDLIVTPATILKEKPDPNNLVFGTVFTDHMLTVEWSSEFGWEKPHIKPLQNLSLHPGSSALHYAVELFEGLKAFRGVDNKIRLFQPNLNMDRMYRSAVRATLPVFDKEELLECIQQLVKLDQEWVPYSTSASLYIRPTFIGTEPSLGVKKPTKALLFVLLSPVGPYFSSGTFNPVSLWANPKYVRAWKGGTGDCKMGGNYGSSLFAQCEAVDNGCQQVLWLYGEDHQITEVGTMNLFLYWINEDGEEELATPPLDGIILPGVTRRCILDLAHQWGEFKVSERYLTMDDLTTALEGNRVREMFGSGTACVVCPVSDILYKGETIHIPTMENGPKLASRILSKLTDIQYGREESDWTIVLS

SEQ ID NO: 77 - DNA, 1057 bp, Homo sapiens
  CDS (11)..(1048)
caccagatctatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcacaagcttggc

SEQ ID NO: 78 - protein, 346 aa, Homo sapiens
MYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 79 - DNA, 961 bp, Homo sapiens
  CDS (2)..(961)
cacctcgcgaaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcacgtcgacggc

SEQ ID NO: 80 - protein, 320 aa, Homo sapiens
TSRNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWHVDG

SEQ ID NO: 81 - DNA, 1050 bp, Homo sapiens
  CDS (6)..(1043)
tcgccatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactgaacaa

SEQ ID NO: 82 - protein, 346 aa, Homo sapiens; identical to SEQ ID NO: 78

SEQ ID NO: 83 - DNA, 1104 bp, Homo sapiens
  CDS (60)..(1097)
gtgccattgtggggactccctgggctgctctgcacccggacacttgctctgtccccgccatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcgggcaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactgaacaa

SEQ ID NO: 84 - protein, 346 aa, Homo sapiens
MYNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAAGRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 85 - DNA, 1104 bp, Homo sapiens
  CDS (60)..(1097)
gtgccattgtggggactccctgggctgctctgcacccggacacttgctctgtccccgccatgtacaacgggtcgtgctgccgcatcgagggggacaccatctcccaggtgatgccgccgctgctcattgtggcctttgtgctgggcgcactagacaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcgggcaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcactgaacaa

SEQ ID NO: 86 - protein, 346 aa, Homo sapiens
MYNGSCCRIEGDTISQVMPPLLIVAFVLGALDNGVALCGFCFHMKTWKPSTVYLFNLAVADFLLMICLPFRTDYYLRRRHWAFGDIPCRVGLFTLAMNRAGSIVFLTVVAAGRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 87 - DNA, 961 bp, Homo sapiens
  CDS (11)..(952)
caccagatctaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcacaagcttggc

SEQ ID NO: 88 - protein, 314 aa, Homo sapiens
NGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 89 - DNA, 1060 bp, Homo sapiens
  CDS (14)..(1051)
cacctcgcgaaccatgtacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcacgtcgacggc

SEQ ID NO: 90 - protein, 346 aa, Homo sapiens; identical to SEQ ID NO: 78

SEQ ID NO: 91 - DNA, 961 bp, Homo sapiens; identical to SEQ ID NO: 79
  CDS (2)..(961)

SEQ ID NO: 92 - protein, 320 aa, Homo sapiens; identical to SEQ ID NO: 80

SEQ ID NO: 93 - DNA, 1044 bp, Homo sapiens
  CDS (1)..(1035)
tacaacgggtcgtgctgccgcatcgagggggacaccatctcccaggtgatgccgccgctgctcattgtggcctttgtgctgggcgcactaggcaatggggtcgccctgtgtggtttctgcttccacatgaagacctggaagcccagcactgtttaccttttcaatttggccgtggctgatttcctccttatgatctgcctgccttttcggacagactattacctcagacgtagacactgggcttttggggacattccctgccgagtggggctcttcacgttggccatgaacagggccgggagcatcgtgttccttacggtggtggctgcggacaggtatttcaaagtggtccacccccaccacgcggtgaacactatctccacccgggtggcggctggcatcgtctgcaccctgtgggccctggtcatcctgggaacagtgtatcttttgctggagaaccatctctgcgtgcaagagacggccgtctcctgtgagagcttcatcatggagtcggccaatggctggcatgacatcatgttccagctggagttctttatgcccctcggcatcatcttattttgctccttcaagattgtttggagcctgaggcggaggcagcagctggccagacaggctcggatgaagaaggcgacccggttcatcatggtggtggcaattgtgttcatcacatgctacctgcccagcgtgtctgctagactctatttcctctggacggtgccctcgagtgcctgcgatccctctgtccatggggccctgcacataaccctcagcttcacctacatgaacagcatgctggatcccctggtgtattatttttcaagcccctcctttcccaaattctacaacaagctcaaaatctgcagtctgaaacccaagcagccaggacactcaaaaacacaaaggccggaagagatgccaatttcgaacctcggtcgcaggagttgcatcagtgtggcaaatagtttccaaagccagtctgatgggcaatgggatccccacattgttgagtggcacgaattcggc

SEQ ID NO: 94 - protein, 345 aa, Homo sapiens
YNGSCCRIEGDTISQVMPPLLIVAFVLGALGNGVALCGFCFHMKTWKPSTVYLFNLAVADFLLMICLPFRTDYYLRRRHWAFGDIPCRVGLFTLAMNRAGSIVFLTVVAADRYFKVVHPHHAVNTISTRVAAGIVCTLWALVILGTVYLLLENHLCVQETAVSCESFIMESANGWHDIMFQLEFFMPLGIILFCSFKIVWSLRRRQQLARQARMKKATRFIMVVAIVFITCYLPSVSARLYFLWTVPSSACDPSVHGALHITLSFTYMNSMLDPLVYYFSSPSFPKFYNKLKICSLKPKQPGHSKTQRPEEMPISNLGRRSCISVANSFQSQSDGQWDPHIVEWH

SEQ ID NO: 95 - DNA, 1254 bp, Homo sapiens
  CDS (7)..(999)
ctaaagatgaaacggctggtttgtgtgctcttggtgtgctcctctgcagtggcacagttgcataaagatcctaccctggatcaccactggcatctctggaagaaaacctatggcaaacaatacaaggaaaagaatgaagaagcagtacgacgtctcatctgggaaaagaatctaaagtttgtgatgcttcacaacctggagcattcaatgggaatgcactcatacgatctgggcatgaaccacctgggagacatgaccagtgaagaagtgatgtctttgatgagttccctgagagttcccagccagtggcagagaaatatcacatataagtcaaaccctaatcggatattgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaatagaggcccagtgtctgttggtgtagatgcgcgtcatccttccttcttcctctacagaagtggtgtctactatgaaccatcctgtactcagaatgtgaatcatggtgtacttgtggttggctatggtgatcttaatgggaaagaatactggcttgtgaaaaacagctggggtcacaactttggtgaagaaggatatattcggatggcaagaaataaaggaaatcattgtgggattgctagctttccctcttacccagaaatctagaggatctctcctttttataacaaatcaagaaatatgaagcactttctcttaacttaatttttcctgctgtatccagaagaaataattgtgtcatgattaatgtgtatttactgtactaattagaaaatatagtttgaggccgggcacggtggctcacgcctgtaatcccagtacttgggaggccaaggcaggcatatcaacttgaggccaggagttaaagagcagcctggctaactgtgaaacccctctact

SEQ ID NO: 96 - protein, 331 aa, Homo sapiens
MKRLVCVLLVCSSAVAQLHKDPTLDHHWHLWKKTYGKQYKEKNEEAVRRLIWEKNLKFVMLHNLEHSMGMHSYDLGMNHLGDMTSEEVMSLMSSLRVPSQWQRNITYKSNPNRILPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANRGPVSVGVDARHPSFFLYRSGVYYEPSCTQNVNHGVLVVGYGDLNGKEYWLVKNSWGHNFGEEGYIRMARNKGNHCGIASFPSYPEI

SEQ ID NO: 97 - DNA, 667 bp, Homo sapiens
  CDS (2)..(667)
caccaagcttttgcctgattctgtggactggagagagaaagggtgtgttactgaagtgaaatatcaaggttcttgtggtgcttgctgggctttcagtgctgtgggggccctggaagcacagctgaagctgaaaacaggaaagctggtgtctctcagtgcccagaacctggtggattgctcaactgaaaaatatggaaacaaaggctgcaatggtggcttcatgacaacggctttccagtacatcattgataacaagggcatcgactcagacgcttcctatccctacaaagccatggatcagaaatgtcaatatgactcaaaatatcgtgctgccacatgttcaaagtacactgaacttccttatggcagagaagatgtcctgaaagaagctgtggccaatagaggcccagtgtctgttggtgtagatgcgcgtcatccttccttcttcctctacagaagtggtgtctactatgaaccatcctgtactcagaatgtgaatcatggtgtacttgtggttggctatggtgatcttaatgggaaagaatactggcttgtgaaaaacagctggggtcacaactttggtgaagaaggatatattcggatggcaagaaataaaggaaatcattgtgggattgctagctttccctcttacccagaagtcgacggc

SEQ ID NO: 98 - protein, 222 aa, Homo sapiens
TKLLPDSVDWREKGCVTEVKYQGSCGACWAFSAVGALEAQLKLKTGKLVSLSAQNLVDCSTEKYGNKGCNGGFMTTAFQYIIDNKGIDSDASYPYKAMDQKCQYDSKYRAATCSKYTELPYGREDVLKEAVANRGPVSVGVDARHPSFFLYRSGVYYEPSCTQNVNHGVLVVGYGDLNGKEYWLVKNSWGHNFGEEGYIRMARNKGNHCGIASFPSYPEVDG

SEQ ID NO: 99 - DNA, 1222 bp, Homo sapiens
  CDS (67)..(1155)
attgcgggcggcggcgttcggagtcgccgggagctgccaggctgtccgcgccgccgctgcggggccatgatccggaacgggcgcggggctgcaggcggcgcagagcagccgggcccggggggcaggcgcgccgtgagggtgtggtgcgatggctgctatgacatggtgcattacggccactccaaccagctgcgccaggcacgggccatgggtgactacctcatcgtaggcgtgcacaccgatgaggagatcgccaagcacaaggggcccccggtgttcactcaggaggagagatacaagatggtgcaggccatcaaatgggtggacgaggtggtgccagcggctccctacgtcactacactagagaccctggacaaatacaactgtgacttctgtgttcacggcaatgacatcaccctgactgtagatggccgggacacctatgaggaagtaaagcaggctgggaggtacagagaatgcaagcgcacgcaaggggtgtccaccacagacctcgtgggccgcatgctgctggtaaccaaagcccatcacagcagccaggagatgtcctctgagtaccgggagtatgcagacagttttggcaagtgccctggtgggcggaacccctggaccggggtatcccagttcctgcagacatctcagaagatcatccagtttgcttctgggaaggagccccagccaggggagacagtcatctatgtggctggtgccttcgacctgttccacatcgggcatgtggacttcctggagaaggtgcacaggctggcagagaggccctacatcatcgcgggcttacactttgaccagtacgtgtcagaagtggtgattggagccccgtacgcggtcacagcagagctcctaagtcacttcaaggtggacctggtgtgtcacggcaagacaggaattatccctgacagggatggctccgacccataccaggagcccaagagaaggggcatcttccgtcagattgacagtggcagcaacctcaccacagacctcatcgtccagcggatcatcaccaacaggttggagtatgaggcgcgaaaccagaagaaggaagccaaggagctggccttcctggaggctgccaggcagcaggcggcacagcccctgggggagcgcgatggtgacttctaacctggcagaggccctggccggccctccccctgctctgcttctgcgccttctgcgtttggacata

SEQ ID NO: 100 - protein, 363 aa, Homo sapiens
MIRNGRGAAGGAEQPGPGGRRAVRVWCDGCYDMVHYGHSNQLRQARAMGDYLIVGVHTDEEIAKHKGPPVFTQEERYKMVQAIKWVDEVVPAAPYVTTLETLDKYNCDFCVHGNDITLTVDGRDTYEEVKQAGRYRECKRTQGVSTTDLVGRMLLVTKAHHSSQEMSSEYREYADSFGKCPGGRNPWTGVSQFLQTSQKIIQFASGKEPQPGETVIYVAGAFDLFHIGHVDFLEKVHRLAERPYIIAGLHFDQYVSEVVIGAPYAVTAELLSHFKVDLVCHGKTGIIPDRDGSDPYQEPKRRGIFRQIDSGSNLTTDLIVQRIITNRLEYEARNQKKEAKELAFLEAARQQAAQPLGERDGDF

SEQ ID NO: 101 - DNA, 1243 bp, Homo sapiens
  CDS (2)..(1243)
caccggatccaccatgatccggaacgggcgcggggctgcaggcggcgcagagcagccgggcccggggggcaggcgcgccgtgagggtgtggtgcgatggctgctatgacatggtgcattacggccactccaaccagctgcgccaggcacgggccatgggtgactacctcatcgtaggcgtgcacaccgatgaggagatcgccaagcacaaggggcccccggtgttcactcaggaggagagatacaagatggtgcaggccatcaaatgggtggacgaggtggtgccagcggctccctacgtcactacactagagaccctggacaaatacaactgtgacttctgtgttcacggcaatgacatcaccctgactgtagatggccgggacacctatgaggaagtaaagcaggctgggaggtacagagaatgcaagcgcacgcaaggggtgtccaccacagacctcgtgggccgcatgctgctggtaaccaaagcccatcacagcagccaggagatgtcctctgagtaccgggagtatgcagacagttttggcaagccccctcacccgatacccgccggggacatactttcctcagaaggctgctcccagtgccctggtgggcggaacccctggaccggggtatcccagttcctgcagacatctcagaagatcatccagtttgcttctgggaaggagccccagccaggggagacagtcatctatgtggctggtgccttcgacctgttccacatcgggcatgtggacttcctggagaaggtgcacaggctggcagagaggccctacatcatcgcgggcttacactttgaccaggaggtcaatcactacaaggggaagaactaccccatcatgaatctgcatgaacggactctgagcgtgctggcctgccggtacgtgtcagaagtggtgattggagccccgtacgcggtcacagcagagctcctaagtcacttcaaggtggacctggtgtgtcacggcaagacagaaattatccctgacagggatggctccgacccataccaggagcccaagagaaggggcatcttccgtcagattgacagtggcagcaacctcaccacagacctcatcgtccagcggatcatcaccaacaggttggagtatgaggcgcgaaaccagaagaaggaagccaaggagctggccttcctggaggctgccaggcagcaggcggcacagcccctgggggagcgcgatggtgacttcctcgagggc

SEQ ID NO: 102 - protein, 414 aa, Homo sapiens
TGSTMIRNGRGAAGGAEQPGPGGRRAVRVWCDGCYDMVHYGHSNQLRQARAMGDYLIVGVHTDEEIAKHKGPPVFTQEERYKMVQAIKWVDEVVPAAPYVTTLETLDKYNCDFCVHGNDITLTVDGRDTYEEVKQAGRYRECKRTQGVSTTDLVGRMLLVTKAHHSSQEMSSEYREYADSFGKPPHPIPAGDILSSEGCSQCPGGRNPWTGVSQFLQTSQKIIQFASGKEPQPGETVIYVAGAFDLFHIGHVDFLEKVHRLAERPYIIAGLHFDQEVNHYKGKNYPIMNLHERTLSVLACRYVSEVVIGAPYAVTAELLSHFKVDLVCHGKTEIIPDRDGSDPYQEPKRRGIFRQIDSGSNLTTDLIVQRIITNRLEYEARNQKKEAKELAFLEAARQQAAQPLGERDGDFLEG

SEQ ID NO: 103 - DNA, 1237 bp, Homo sapiens
  CDS (14)..(1234)
caccggatccaccatgatccggaacgggcgcggggctgcaggcggcgcagagcagccgggcccggggggcaggcgcgccgtgagggtgtggtgcgatggctgctatgacatggtgcattacggccactccaaccagctgcgccaggcacgggccatgggtgactacctcatcgtaggcgtgcacaccgatgaggagatcgccaagcacaaggggcccccggtgttcactcaggaggagagatacaagatggtgcaggccatcaaatgggtggacgaggtggtgccagcggctccctacgtcactacactagagaccctggacaaatacaactgtgacttctgtgttcacggcaatgacatcaccctgactgtagatggccgggacacctatgaggaagtaaagcaggctgggaggtacagagaatgcaagcgcacgcaaggggtgtccaccacagacctcgtgggccgcatgctgctggtaaccaaagcccatcacagcagccaggagatgtcctctgagtaccgggagtatgcagacagttttggcaagccccctcacccgatacccgccggggacatactttcctcagaaggctgctcccagtgccctggtgggcggaacccctggaccggggtatcccagttcctgcagacatctcagaagatcatccagtttgcttctgggaaggagccccagccaggggagacagtcatctatgtggctggtgccttcgacctgttccacatcgggcatgtggacttcctggagaaggtgcacaggctggcagagaggccctacatcatcgcgggcttacactttgaccaggaggtcaatcactacaaggggaagaactaccccatcatgaatctgcatgaacggactctgagcgtgctggcctgccggtacgtgtcagaagtggtgattggagccccgtacgcggtcacagcagagctcctaagtcacttcaaggtggacctggtgtgtcacggcaagacagaaattatccctgacagggatggctccgacccataccaggagcccaagagaaggggcatcttccgtcagattgacagtggcagcaacctcaccacagacctcatcgtccagcggatcatcaccaacaggttggagtatgaggcgcgaaaccagaagaaggaagccaaggagctggccttcctggaggctgccaggcagcaggcggcacagcccctgggggagcgcgatggtgacttctaa

SEQ ID NO: 104 - protein, 407 aa, Homo sapiens
MIRNGRGAAGGAEQPGPGGRRAVRVWCDGCYDMVHYGHSNQLRQARAMGDYLIVGVHTDEEIAKHKGPPVFTQEERYKMVQAIKWVDEVVPAAPYVTTLETLDKYNCDFCVHGNDITLTVDGRDTYEEVKQAGRYRECKRTQGVSTTDLVGRMLLVTKAHHSSQEMSSEYREYADSFGKPPHPIPAGDILSSEGCSQCPGGRNPWTGVSQFLQTSQKIIQFASGKEPQPGETVIYVAGAFDLFHIGHVDFLEKVHRLAERPYIIAGLHFDQEVNHYKGKNYPIMNLHERTLSVLACRYVSEVVIGAPYAVTAELLSHFKVDLVCHGKTEIIPDRDGSDPYQEPKRRGIFRQIDSGSNLTTDLIVQRIITNRLEYEARNQKKEAKELAFLEAARQQAAQPLGERDGDF

SEQ ID NO: 105 - DNA, 1856 bp, Homo sapiens
  CDS (67)..(1233)
attgcgggcggcggcgttcggagtcgccgggagctgccaggctgtccgcgccgccgctgcggggccatgatccggaacgggcgcggggctgcaggcggcgcagagcagccgggcccggggggcaggcgcgccgtgagggtgtggtgcgatggctgctatgacatggtgcattacggccactccaaccagctgcgccaggcacgggccatgggtgactacctcatcgtaggcgtgcacaccgatgaggagatcgccaagcacaaggggcccccggtgttcactcaggaggagagatacaagatggtgcaggccatcaaatgggtggacgaggtggtgccagcggctccctacgtcactacactagagaccctggacaaatacaactgtgacttctgtgttcacggcaatgacatcaccctgactgtagatggccgggacacctatgaggaagtaaagcaggctgggaggtacagagaatgcaagcgcacgcaaggggtgtccaccacagacctcgtgggccgcatgctgctggtaaccaaagcccatcacagcagccaggagatgtcctctgagtaccgggagtatgcagacagttttggcaagtgccctggtgggcggaacccctggaccggggtatcccagttcctgcagacatctcagaagatcatccagtttgcttctgggaaggagccccagccaggggagacagtcatctatgtggctggtgccttcgacctgttccacatcgggcatgtggacttcctggagaaggtgcacaggctggcagagaggccctacatcatcgcgggcttacactttgaccaggaggtcaatcactacaaggggaagaactaccccatcatgaatctgcatgaacggactctgagcgtgctggcctgccggtacgtgtcagaagtggtgattggagccccgtacgcggtcacagcagagctcctaagtcacttcaaggtggacctggtgtgtcacggcaagacagaaattatccctgacagggatggctccgacccataccaggagcccaagagaaggggcatcttccgtcagattgacagtggcagcaacctcaccacagacctcatcgtccagcggatcatcaccaacaggttggagtatgaggcgcgaaaccagaagaaggaagccaaggagctggccttcctggaggctgccaggcagcaggcggcacagcccctgggggagcgcgatggtgacttctaacctggcagaggccctggccggccctccccctgctctgcttctgcgccttctgcgtttggacataggactctgcagggccgccctctctaactggcctggctctggaagggctggtgaggactctgcctccttgcctgcctacaaggtgcctggtttgcagcaggctctccgctctttccagcaaagctgctcagagagggtgtccagcacagtggagaggccggaagtgagacgggcagacggcacctgcagcctgaaacgcaccgctcctgcgtgcgcccccacctggtccccggatgcccccaccacctggacagaggccacactgactgcccacccagctgtggcgggaggtgcagagcagggggctttagggagcagtgactgcggtcacccctttagttctctgggtgtagaccacaccacctcccactgggcaccccccaacacggtgtcctgccacccagcgcctggctccaggaaaacacgcttgccttccttcccggcagcttcgccactctccttatggactctgttctgtttgtacatggctgacggaaatctctttggtacaaccgaataaagcctggtggcagtgctgcgcggggctcccagccaat

SEQ ID NO: 106 - protein, 389 aa, Homo sapiens
MIRNGRGAAGGAEQPGPGGRRAVRVWCDGCYDMVHYGHSNQLRQARAMGDYLIVGVHTDEEIAKHKGPPVFTQEERYKMVQAIKWVDEVVPAAPYVTTLETLDKYNCDFCVHGNDITLTVDGRDTYEEVKQAGRYRECKRTQGVSTTDLVGRMLLVTKAHHSSQEMSSEYREYADSFGKCPGGRNPWTGVSQFLQTSQKIIQFASGKEPQPGETVIYVAGAFDLFHIGHVDFLEKVHRLAERPYIIAGLHFDQEVNHYKGKNYPIMNLHERTLSVLACRYVSEVVIGAPYAVTAELLSHFKVDLVCHGKTEIIPDRDGSDPYQEPKRRGIFRQIDSGSNLTTDLIVQRIITNRLEYEARNQKKEAKELAFLEAARQQAAQPLGERDGDF

SEQ ID NO: 107 - DNA, 1908 bp, Homo sapiens
  CDS (24)..(1718)
gcagactcagttcctggagaaagatggcgacagccgagaagcagaaacacgacgggcgggtgaagatcggccactacattctgggtgacacgctgggggtcggcaccttcggcaaagtgaaggttggcaaacatgaattgactgggcataaagtagctgtgaagatactcaatcgacagaagattcggagccttgatgtggtaggaaaaatccgcagagaaattcagaacctcaagcttttcaggcatcctcatataattaaactgtaccaggtcatcagtacaccatctgatattttcatggtgatggaatatgtctcaggaggagagctatttgattatatctgtaagaatggaaggaaatctgatgtacctggagtagtaaaaacaggctccacgaaggagctggatgaaaaagaaagtcggcgtctgttccaacagatcctttctggtgtggattattgtcacaggcatatggtggtccatagagatttgaaacctgaaaatgtcctgcttgatgcacacatgaatgcaaagatagctgattttggtctttcaaacatgatgtcagatggtgaatttttaagaacaagttgtggctcacccaactatgctgcaccagaagtaatttcaggaagattgtatgcaggcccagaggtagatatatggagcagtggggttattctctatgctttattatgtggaacccttccatttgatgatgaccatgtgccaactctttttaagaagatatgtgatgggatcttctatacccctcaatatttaaatccttctgtgattagccttttgaaacatatgctgcaggtggatcccatgaagagggcctcaatcaaagatatcagggaacatgaatggtttaaacaggaccttccaaaatatctctttcctgaggatccatcatatagttcaaccatgattgatgatgaagccttaaaagaagtatgtgaaaagtttgagtgctcagaagaggaagttctcagctgtctttacaacagaaatcaccaggatcctttggcagttgcctaccatctcataatagataacaggagaataatgaatgaagccaaagatttctatttggcgacaagcccacctgattcttttcttgatgatcatcacctgactcggccccatcctgaaagagtaccattcttggttgctgaaacaccaagggcacgccatacccttgatgaattaaatccacagaaatccaaacaccaaggtgtaaggaaagcaaaatggcatttaggaattagaagtcaaagtcgaccaaatgatattatggcagaagtatgtagagcaatcaaacaattggattatgaatggaaggttgtaaacccatattatttgcgtgtacgaaggaagaatcctgtgacaagcacttactccaaaatgagtctacagttataccaagtggatagtagaacttatctactggatttccgtagtattgatgatgaaattacagaagccaaatcagggactgctactccacagagatcgggatcagttagcaactatcgatcttgccaaaggagtgattcagatgctgaggctcaaggaaaatcctcagaagtttctcttacctcatctgtgacctcacttgactcttctcctgttgacctaactccaagacctggaagtcacacaatagaattttttgagatgtgtgcaaatctaattaaaattcttgcacaataaacagaaaactttgcttatttcttttgcagcaataagcatgcataataagtcacagccaaatgcttccatttgtaatcaagttatacataattataaccgagggctggcgttttggaatcgaatttcgacagggattggaacatgatttatagttaaaagcctaatatcgagaaatgaattaagatca

SEQ ID NO: 108 - protein, 565 aa, Homo sapiens
MATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRKSDVPGVVKTGSTKELDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRASIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 109 - DNA, 1863 bp, Homo sapiens
  CDS (24)..(1673)
gcagactcagttcctggagaaagatggcgacagccgagaagcagaaacacgacgggcgggtgaagatcggccactacattctgggtgacacgctgggggtcggcaccttcggcaaagtgaaggttggcaaacatgaattgactgggcataaagtagctgtgaagatactcaatcgacagaagattcggagccttgatgtggtaggaaaaatccgcagagaaattcagaacctcaagcttttcaggcatcctcatataattaaactgtaccaggtcatcagtacaccatctgatattttcatggtgatggaatatgtctcaggaggagagctatttgattatatctgtaagaatggaaggctggatgaaaaagaaagtcggcgtctgttccaacagatcctttctggtgtggattattgtcacaggcatatggtggtccatagagatttgaaacctgaaaatgtcctgcttgatgcacacatgaatgcaaagatagctgattttggtctttcaaacatgatgtcagatggtgaatttttaagaacaagttgtggctcacccaactatgctgcaccagaagtaatttcaggaagattgtatgcaggcccagaggtagatatatggagcagtggggttattctctatgctttattatgtggaacccttccatttgatgatgaccatgtgccaactctttttaagaagatatgtgatgggatcttctatacccctcaatatttaaatccttctgtgattagccttttgaaacatatgctgcaggtggatcccatgaagagggcctcaatcaaagatatcagggaacatgaatggtttaaacaggaccttccaaaatatctctttcctgaggatccatcatatagttcaaccatgattgatgatgaagccttaaaagaagtatgtgaaaagtttgagtgctcagaagaggaagttctcagctgtctttacaacagaaatcaccaggatcctttggcagttgcctaccatctcataatagataacaggagaataatgaatgaagccaaagatttctatttggcgacaagcccacctgattcttttcttgatgatcatcacctgactcggccccatcctgaaagagtaccattcttggttgctgaaacaccaagggcacgccatacccttgatgaattaaatccacagaaatccaaacaccaaggtgtaaggaaagcaaaatggcatttaggaattagaagtcaaagtcgaccaaatgatattatggcagaagtatgtagagcaatcaaacaattggattatgaatggaaggttgtaaacccatattatttgcgtgtacgaaggaagaatcctgtgacaagcacttactccaaaatgagtctacagttataccaagtggatagtagaacttatctactggatttccgtagtattgatgatgaaattacagaagccaaatcagggactgctactccacagagatcgggatcagttagcaactatcgatcttgccaaaggagtgattcagatgctgaggctcaaggaaaatcctcagaagtttctcttacctcatctgtgacctcacttgactcttctcctgttgacctaactccaagacctggaagtcacacaatagaattttttgagatgtgtgcaaatctaattaaaattcttgcacaataaacagaaaactttgcttatttcttttgcagcaataagcatgcataataagtcacagccaaatgcttccatttgtaatcaagttatacataattataaccgagggctggcgttttggaatcgaatttcgacagggattggaacatgatttatagttaaaagcctaatatcgagaaatgaattaagatca

SEQ ID NO: 110 - protein, 550 aa, Homo sapiens
MATAEKQKHDGRVKIGHYILGDTLGVGTFGKVKVGKHELTGHKVAVKILNRQKIRSLDVVGKIRREIQNLKLFRHPHIIKLYQVISTPSDIFMVMEYVSGGELFDYICKNGRLDEKESRRLFQQILSGVDYCHRHMVVHRDLKPENVLLDAHMNAKIADFGLSNMMSDGEFLRTSCGSPNYAAPEVISGRLYAGPEVDIWSSGVILYALLCGTLPFDDDHVPTLFKKICDGIFYTPQYLNPSVISLLKHMLQVDPMKRASIKDIREHEWFKQDLPKYLFPEDPSYSSTMIDDEALKEVCEKFECSEEEVLSCLYNRNHQDPLAVAYHLIIDNRRIMNEAKDFYLATSPPDSFLDDHHLTRPHPERVPFLVAETPRARHTLDELNPQKSKHQGVRKAKWHLGIRSQSRPNDIMAEVCRAIKQLDYEWKVVNPYYLRVRRKNPVTSTYSKMSLQLYQVDSRTYLLDFRSIDDEITEAKSGTATPQRSGSVSNYRSCQRSDSDAEAQGKSSEVSLTSSVTSLDSSPVDLTPRPGSHTIEFFEMCANLIKILAQ

SEQ ID NO: 111 - DNA, 1863 bp, Homo sapiens; identical to SEQ ID NO: 109
  CDS (24)..(1673)

SEQ ID NO: 112 - protein, 550 aa, Homo sapiens; identical to SEQ ID NO: 110

SEQ ID NO: 113 - DNA, 22 bp, Artificial Sequence
  Description of Artifical Sequence  Primer/ Probe
aaagttcaatgccaggagaaag

SEQ ID NO: 114 - DNA, 23 bp, Artificial Sequence
  Description of Artifical Sequence  Primer/ Probe
taagggagccatcctcaccacca

SEQ ID NO: 115 - DNA, 22 bp, Artificial Sequence
  Description of Artifical Sequence  Primer/ Probe
aaactcttggctgagaaattcc

SEQ ID NO: 116 - DNA, 22 bp, Artificial Sequence
  Description of Artifical Sequence  Primer/ Probe
tccatacagatcaccaactgtg

SEQ ID NO: 117 - DNA, 25 bp, Artificial Sequence
  Description of Artifical Sequence  Primer/ Probe
tcgtccgaactcttccccttgcttc

SEQ ID NO: 118 - DNA, 21 bp, Artificial Sequence
  Description of Artifical Sequence  Primer/ Probe
agacattatggaacgtggaga

SEQ ID NO: 119 - DNA, 22 bp, Artificial Sequence
  Description of Artifical Sequence  Primer/ Probe
cttcagcttctctttcccttgt

SEQ ID NO: 120 - DNA, 26 bp, Artificial Sequence
  Description of Artifical Sequence  Primer/ Probe
tcttggacaggagcaccctcatttcc

SEQ ID NO: 121 - DNA, 22 bp, Artificial Sequence
  Description of Artifical Sequence  Primer/ Probe
acaccactgcacctaaaacctt

SEQ ID NO: 122 - DNA, 22 bp, Artificial Sequence
  Description of Artifical Sequence  Primer/ Probe
tagaatctgccgtcttttgaag

SEQ ID NO: 123 - DNA, 26 bp, Artificial Sequence
  Description of Artifical Sequence  Primer/ Probe
tcaccctaatattgtgcgacttcatg

SEQ ID NO: 124 - DNA, 22 bp, Artificial Sequence
  Description of Artifical Sequence  Primer/ Probe
ccaagtagtgaaagccctcttc

SEQ ID NO: 125 - DNA, 17 bp, Artificial Sequence
  Description of Artifical Sequence  Primer/ Probe
aaggcctccaccgacat

SEQ ID NO: 126 - DNA, 25 bp, Artificial Sequence
  Description of Artifical Sequence  Primer/ Probe
tatcgtcttcactgccaccttggtg

SEQ ID NO: 127 - DNA, 24 bp, Artificial Sequence
  Description of Artifical Sequence  Primer/ Probe
gaccactacaagaccacctacaac

SEQ ID NO: 128 - DNA, 22 bp, Artificial Sequence
  Description of Artifical Sequence  Primer/ Probe
gcggaagaagaagccatataat

SEQ ID NO: 129 - DNA, 26 bp, Artificial Sequence
  Description of Artifical Sequence  Primer/ Probe
ttacaccaagaggaatgctcttcgcg

SEQ ID NO: 130 - DNA, 22 bp, Artificial Sequence
  Description of Artifical Sequence  Primer/ Probe
gagacttgtaatcgtccatcca

SEQ ID NO: 131 - DNA, 20 bp, Artificial Sequence
  Description of Artifical Sequence  Primer/ Probe
cctgtgttgtttgcccagtt

SEQ ID NO: 132 - DNA, 26 bp, Artificial Sequence
  Description of Artifical Sequence  Primer/ Probe
taaggcgagacaatacacattccaac

SEQ ID NO: 133 - DNA, 20 bp, Artificial Sequence
  Description of Artifical Sequence  Primer/ Probe
cttgccagcttaggaccatt

SEQ ID NO: 134 - DNA, 25 bp, Artificial Sequence
  Description of Artifical Sequence  Primer/ Probe
agctcttctttgtcaaatacttcca

SEQ ID NO: 135 - DNA, 26 bp, Artificial Sequence
  Description of Artifical Sequence  Primer/ Probe
taccctggctcatcagctttgcacta

SEQ ID NO: 136 - DNA, 24 bp, Artificial Sequence
  Description of Artifical Sequence  Primer/ Probe
agaaacctcatatcaagcctcttc

SEQ ID NO: 137 - DNA, 22 bp, Artificial Sequence
  Description of Artifical Sequence  Primer/ Probe
catcttccgtcagattgacagt

SEQ ID NO: 138 - DNA, 26 bp, Artificial Sequence
  Description of Artifical Sequence  Primer/ Probe
tagcaacctcaccacagacctcatcg

SEQ ID NO: 139 - DNA, 22 bp, Artificial Sequence
  Description of Artifical Sequence  Primer/ Probe
atactccaacctgttggtgatg

SEQ ID NO: 140 - DNA, 22 bp, Artificial Sequence
  Description of Artifical Sequence  Primer/ Probe
aacagaaatcaccaggatcctt

SEQ ID NO: 141 - DNA, 26 bp, Artificial Sequence
  Description of Artifical Sequence  Primer/ Probe
ttggcagttgcctaccatctcataat

SEQ ID NO: 142 - DNA, 22 bp, Artificial Sequence
  Description of Artifical Sequence  Primer/ Probe
tgtcgccaaatagaaatctttg

SEQ ID NO: 143 - DNA, 24 bp, Artificial Sequence
  Description of Artifical Sequence  Primer/ Probe
accatctgatattttcatggtgat

SEQ ID NO: 144 - DNA, 30 bp, Artificial Sequence
  Description of Artifical Sequence  Primer/ Probe
ttcaaatagctctcctcctgagacatattc

SEQ ID NO: 145 - DNA, 27 bp, Artificial Sequence
  Description of Artifical Sequence  Primer/ Probe
gtacatcagatttccttccattcttac

SEQ ID NO: 146 - DNA, 30 bp, Artificial Sequence
  Description of Artifical Sequence  Primer/ Probe
tcttacccagaaatctagaggatctctcct

(19) United States
(12) Patent Application Publication
(10) Pub. No.: US 2005/0037957 A1
(43) Pub. Date: Feb. 17, 2005

(54) Novel proteins and nucleic acids encoding same

(76) Inventors: David Anderson, Plantsville, CT (US); Constance Berghs, New Haven, CT (US); Elina Catterton, Milford, CT (US); Shlomit Edinger, New Haven, CT (US); Linda Gorman, Branford, CT (US); Xiaojia (Sasha) Guo, Branford, CT (US); John Herrmann, Guilford, CT (US); Ramesh Kekuda, Norwalk, CT (US); Li Li, Branford, CT (US); Daniel Rieger, Branford, CT (US); Mei Zhong, Branford, CT (US)

Correspondence Address: MINTZ, LEVIN, COHN, FERRIS, GLOVSKY;AND POPEO, P.C., ONE FINANCIAL CENTER, BOSTON, MA, 02111, US

(21) Appl. No.: 10/635,398

(22) Filed: Aug. 6, 2003

Related U.S. Application Data
(63) Continuation-in-part of application No. 10/160,619, filed on Jun. 3, 2002.
(60) Provisional application No. 60/295,661, filed on Jun. 4, 2001.
(60) Provisional application No. 60/359,122, filed on Feb. 21, 2002.
(60) Provisional application No. 60/296,404, filed on Jun. 6, 2001.
(60) Provisional application No. 60/359,035, filed on Feb. 22, 2002.
(60) Provisional application No. 60/299,949, filed on Jun. 21, 2001.
(60) Provisional application No. 60/295,607, filed on Jun. 4, 2001.
(60) Provisional application No. 60/359,964, filed on Feb. 27, 2002.
(60) Provisional application No. 60/296,418, filed on Jun. 6, 2001.
(60) Provisional application No. 60/341,562, filed on Dec. 14, 2001.
(60) Provisional application No. 60/308,890, filed on Jul. 31, 2001.
(60) Provisional application No. 60/296,575, filed on Jun. 7, 2001.
(60) Provisional application No. 60/337,477, filed on Dec. 3, 2001.
(60) Provisional application No. 60/322,297, filed on Sep. 14, 2001.
(60) Provisional application No. 60/297,414, filed on Jun. 11, 2001.
(60) Provisional application No. 60/297,573, filed on Jun. 12, 2001.
(60) Provisional application No. 60/358,978, filed on Feb. 22, 2002.
(60) Provisional application No. 60/297,567, filed on Jun. 12, 2001.
(60) Provisional application No. 60/359,121, filed on Feb. 22, 2002.
(60) Provisional application No. 60/298,285, filed on Jun. 14, 2001.
(60) Provisional application No. 60/298,528, filed on Jun. 15, 2001.
(60) Provisional application No. 60/299,133, filed on Jun. 18, 2001.
(60) Provisional application No. 60/324,669, filed on Sep. 25, 2001.
(60) Provisional application No. 60/358,656, filed on Feb. 21, 2002.
(60) Provisional application No. 60/299,230, filed on Jun. 19, 2001.
(60) Provisional application No. 60/299,949, filed on Jun. 21, 2001.
(60) Provisional application No. 60/300,177, filed on Jun. 22, 2001.
(60) Provisional application No. 60/359,034, filed on Feb. 22, 2002.
(60) Provisional application No. 60/300,883, filed on Jun. 26, 2001.
(60) Provisional application No. 60/301,530, filed on Jun. 28, 2001.
(60) Provisional application No. 60/301,550, filed on Jun. 28, 2001.
(60) Provisional application No. 60/363,676, filed on Mar. 12, 2002.
(60) Provisional application No. 60/371,346, filed on Apr. 10, 2002.
(60) Provisional application No. 60/302,951, filed on Jul. 3, 2001.
(60) Provisional application No. 60/363,430, filed on Mar. 12, 2002.
(60) Provisional application No. 60/360,858, filed on Mar. 1, 2002.
(60) Provisional application No. 60/379,444, filed on May 10, 2002.
(60) Provisional application No. 60/311,285, filed on Aug. 9, 2001.
(60) Provisional application No. 60/299,949, filed on Jun. 21, 2001.
(60) Provisional application No. 60/300,290, filed on Jun. 22, 2001.
(60) Provisional application No. 60/327,892, filed on Oct. 9, 2001.
(60) Provisional application No. 60/327,345, filed on Oct. 5, 2001.

(51) Int. Cl. A61K 38/17; C07K 14/47; C07H 21/04

(52) U.S. Cl. 514/12; 530/350; 536/23.5; 435/69.1; 435/320.1; 435/325

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application is a continuation-in-part of U.S. Ser. No. 10/160,619, filed Jun. 3, 2002, which claims the benefit of U.S. Ser. No. 60/295,661, filed Jun. 4, 2001; U.S. Ser. No. 60/359,122, filed Feb. 21, 2002; U.S. Ser. No. 60/296,404, filed Jun. 6, 2001; U.S. Ser. No. 60/359,035, filed Feb. 22, 2002; U.S. Ser. No. 60/299,949, filed Jun. 21, 2002; U.S. Ser. No. 60/295,607, filed Jun. 4, 2001; U.S. Ser. No. 60/359,964, filed Feb. 27, 2002; U.S. Ser. No. 60/296,418, filed Jun. 6, 2001; U.S. Ser. No. 60/341,562, filed Dec. 14, 2001; U.S. Ser. No. 60/308,890, filed Jul. 31, 2001; U.S. Ser. No. 60/296,575, filed Jun. 7, 2001; U.S. Ser. No. 60/337,477, filed Dec. 3, 2001; U.S. Ser. No. 60/322,297, filed Sep. 14, 2001; U.S. Ser. No. 60/297,414, filed Jun. 11, 2001; U.S. Ser. No. 60/297,573, filed Jun. 12, 2001; U.S. Ser. No. 60/358,978, filed Feb. 22, 2002; U.S. Ser. No. 60/297,567, filed Jun. 12, 2001; U.S. Ser. No. 60/359,121, filed Feb. 22, 2002; U.S. Ser. No. 60/298,285, filed Jun. 14, 2001; U.S. Ser. No. 60/298,528, filed Jun. 15, 2001; U.S. Ser. No. 60/299,133, filed Jun. 18, 2001; U.S. Ser. No. 60/324,669, filed Sep. 25, 2001; U.S. Ser. No. 60/358,656, filed Feb. 21, 2002; U.S. Ser. No. 60/299,230, filed Jun. 19, 2001; U.S. Ser. No. 60/299,949, filed Jun. 21, 2001; U.S. Ser. No. 60/300,177, filed Jun. 22, 2001; U.S. Ser. No. 60/359,034, filed Feb. 22, 2002; U.S. Ser. No. 60/300,883, filed Jun. 26, 2001; U.S. Ser. No. 60/301,530, filed Jun. 28, 2001; U.S. Ser. No. 60/301,550, filed Jun. 28, 2001; U.S. Ser. No. 60/363,676, filed Mar. 12, 2002; U.S. Ser. No. 60/371,346, filed Apr. 10, 2002; U.S. Ser. No. 60/302,951, filed Jul. 3, 2001; U.S. Ser. No. 60/363,430, filed Mar. 12, 2002; U.S. Ser. No. 60/360,858, filed Mar. 1, 2002; and U.S. Ser. No. 60/379,444, filed May 10, 2002; of U.S. Ser. No. 10/177,809, filed Jun. 21, 2002; U.S. Ser. No. 60/311,285, filed August 9. 2001; U.S. Ser. No. 60/299,949, filed Jun. 21, 2001; U.S. Ser. No. 60/300,290, filed Jun. 22, 2001; U.S. Ser. No. 60/327,892, filed Oct. 9, 2001; and U.S. Ser. No. 60/327,345, filed Oct. 5, 2001; of U.S. Ser. No. 10/210,130, filed Aug. 1, 2002, which claims the benefit of U.S. Ser. No. 60/309,501 filed Aug. 2, 2001; U.S. Ser. No. 60/316,508 filed Aug. 31, 2001; U.S. Ser. No. 60/354,655 filed Feb. 5, 2002; U.S. Ser. No. 60/310,291 filed Aug. 3, 2001; U.S. Ser. No. 60/383,887 filed May 29, 2002; U.S. Ser. No. 60/310,951 filed Aug. 8, 2001; U.S. Ser. No. 60/323,936 filed Sep. 21, 2001; U.S. Ser. No. 60/381,039 filed May 16, 2002; U.S. Ser. No. 60/311,292 filed Aug. 9, 2001; U.S. Ser. No. 60/311,979 filed Aug. 13, 2001; U.S. Ser. No. 60/312,203 filed Aug. 14, 2001; U.S. Ser. No. 60/361,764 filed Mar. 5, 2002; U.S. Ser. No. 60/313,201 filed Aug. 17, 2001; U.S. Ser. No. 60/338,078 filed Dec. 3, 2001; U.S. Ser. No. 60/380,971 filed May 15, 2001; U.S. Ser. No. 60/313,156 filed Aug. 17, 2001; U.S. Ser. No. 60/313,702 filed Aug. 20, 2001; U.S. Ser. No. 60/380,980 filed May 15, 2002; U.S. Ser. No. 60/313,643 filed Aug. 20, 2001; U.S. Ser. No. 60/383,761 filed May 28, 2002; U.S. Ser. No. 60/322,716 filed Sep. 17, 2001; U.S. Ser. No. 60/314,031 filed Aug. 21, 2001; U.S. Ser. No. 60/314,466 filed Aug. 23, 2001; U.S. Ser. No. 60/315,403 filed Aug. 28, 2001; U.S. Ser. No. 60/315,853 filed Aug. 29, 2001; and U.S. Ser. No. 60/373,825 filed Apr. 19, 2002; of U.S. Ser. No. 10/211,689 filed Aug. 1, 2002, which claims the benefit of U.S. Ser. No. 60/311,751 filed Aug. 10, 2001; of U.S. Ser. No. 09/823,172, filed Mar. 29, 2001 which claims the benefit of U.S. Ser. No. 60/193,664, filed Mar. 31, 2000; U.S. Ser. No. 60/194,614, Apr. 5, 2000; U.S. Ser. No. 60/195,063, Apr. 6, 2000; U.S. Ser. No. 60/195,066, Apr. 6, 2000; U.S. Ser. No. 60/195,067, Apr. 6, 2000; U.S. Ser. No. 60/195,068, Apr. 6, 2000; U.S. Ser. No. 60/196,069, Apr. 6, 2000; U.S. Ser. No. 60/195,070, Apr. 6, 2000; U.S. Ser. No. 60/195,510, Apr. 6, 2000; U.S. Ser. No. 60/219,855, Jul. 21, 2000; U.S. Ser. No. 60/221,284, Jul. 27, 2000; U.S. Ser. No. 60/221,325, Jul. 28, 2000; U.S. Ser. No. 60/224,588, Aug. 11, 2000; U.S. Ser. No. 60/239,613, Oct. 11, 2000; U.S. Ser. No. 60/262,508, Jan. 18, 2001; U.S. Ser. No. 60/263,604, Jan. 23, 2001; U.S. Ser. No. 60/263,433, Jan. 23, 2001; and U.S. Ser. No. 60/265,161, Jan. 30, 2001; and of U.S. Ser. No. 10/210,172, filed Aug. 1, 2002, which claims the benefit of U.S. Ser. No. 60/193,664, filed Mar. 31, 2000; U.S. Ser. No. 60/239,613, filed Oct. 11, 2000; U.S. Ser. No. 60/263,604, filed Jan. 23, 2001; U.S. Ser. No. 60/309,501, filed Aug. 2, 2001; U.S. Ser. No. 60/310,291, filed Aug. 3, 2001; U.S. Ser. No. 60/310,544, filed Aug. 7, 2001; U.S. Ser. No. 60/310,951, filed Aug. 8, 2001; U.S. Ser. No. 60/311,292, filed Aug. 9, 2001; U.S. Ser. No. 60/311,979, filed Aug. 13, 2001; U.S. Ser. No. 60/312,892, filed Aug. 16, 2001; U.S. Ser. No. 60/313,201, filed Aug. 17, 2001; U.S. Ser. No. 60/313,415, filed Aug. 17, 2001; U.S. Ser. No. 60/313,702, filed Aug. 20, 2001; U.S. Ser. No. 60/313,643, filed Aug. 20, 2001; U.S. Ser. No. 60/314,031, filed Aug. 21, 2001; U.S. Ser. No. 60/314,466, filed Aug. 23, 2001; U.S. Ser. No. 60/315,403, filed Aug. 28, 2001; U.S. Ser. No. 60/315,853, filed Aug. 29, 2001; U.S. Ser. No. 60/322,716, filed Sep. 17, 2001; U.S. Ser. No. 60/323,994, filed Sep. 21, 2001; U.S. Ser. No. 60/340,233, filed Dec. 14, 2001; U.S. Ser. No. 60/365,478, filed Mar. 19, 2002; U.S. Ser. No. 60/373,814, filed Apr. 19, 2002; U.S. Ser. No. 60/373,825, filed Apr. 19, 2002; U.S. Ser. No. 60/373,989, filed Apr. 19, 2002; and U.S. Ser. No. 60/374,632, filed Apr. 23, 2002; U.S. Ser. No. 60/354,591, filed Feb. 5, 2002; and U.S. Ser. No. 60/386,971; and this application claims priority to the following provisional patent applications: U.S. Ser. No. 60/403,732, filed Aug. 15, 2002; U.S. Ser. No. 60/406,392, filed Aug. 27, 2002; U.S. Ser. No. 60/401,597, filed Aug. 7, 2002; U.S. Ser. No. 60/404,829, filed Aug. 20, 2002; U.S. Ser. No. 60/403,574, filed Aug. 14, 2002; U.S. Ser. No. 60/402,248, filed Aug. 9, 2002; U.S. Ser. No. 60/403,485, filed Aug. 13, 2002; and, U.S. Ser. No. 60/402,815, filed Aug. 12, 2002, each of which is incorporated by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 56, or polypeptide sequences, which represents the group consisting of SEQ ID NO: 2n, wherein n is an integer between 1 and 56.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 56, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a is naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
SEQUENCES AND CORRESPONDING SEQ ID NUMBERS

| | | SEQ ID NO | SEQ ID NO | |
|---|---|---|---|---|
| NOVX | Internal | (nucleic | (amino | |
| Assignment | Identification | acid) | acid) | Homology |
| NOV1a | CG101025-01 | 1 | 2 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1b | CG101025-07 | 3 | 4 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1c | 278987803 | 5 | 6 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1d | 278987807 | 7 | 8 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1e | 278987811 | 9 | 10 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1f | 278987831 | 11 | 12 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1g | CG101025-02 | 13 | 14 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1h | CG101025-03 | 15 | 16 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1i | CG101025-04 | 17 | 18 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1j | CG101025-05 | 19 | 20 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1k | CG101025-06 | 21 | 22 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV1l | SNP13379569 | 23 | 24 | Calcium/calmodulin-dependent protein kinase |
| | | | | type II beta chain (EC 2.7.1.123) (CaM-kinase |
| | | | | II beta chain) (CaM kinase II beta subunit) |
| | | | | (CaMK-II beta subunit) - Homo sapiens |
| NOV2a | CG101826-02 | 25 | 26 | Adenylate kinase 5 - Homo sapiens |
| NOV2b | 308782075 | 27 | 28 | Adenylate kinase 5 - Homo sapiens |
| NOV2c | 308782087 | 29 | 30 | Adenylate kinase 5 - Homo sapiens |
| NOV2d | 309326609 | 31 | 32 | Adenylate kinase 5 - Homo sapiens |
| NOV2e | 309326618 | 33 | 34 | Adenylate kinase 5 - Homo sapiens |
| NOV2f | CG101826-01 | 35 | 36 | Adenylate kinase 5 - Homo sapiens |
| NOV2g | CG101826-03 | 37 | 38 | Adenylate kinase 5 - Homo sapiens |
| NOV2h | CG101826-04 | 39 | 40 | Adenylate kinase 5 - Homo sapiens |
| NOV2i | SNP13376052 | 41 | 42 | Adenylate kinase 5 - Homo sapiens |
| NOV3a | CG105201-01 | 43 | 44 | Hexokinase type III (EC 2.7.1.1) (HK III) - |
| | | | | Homo sapiens |
| NOV3b | 277575154 | 45 | 46 | Hexokinase type III (EC 2.7.1.1) (HK III) - |
| | | | | Homo sapiens |
| NOV3c | CG105201-02 | 47 | 48 | Hexokinase type III (EC 2.7.1.1) (HK III) - |
| | | | | Homo sapiens |
| NOV4a | CG106773-01 | 49 | 50 | Calcium/calmodulin-dependent protein kinase |
| | | | | type II delta chain (EC 2.7.1.123) |
| | | | | (CaM-kinase II delta chain) (CaM kinase II |
| | | | | delta subunit) (CaMK-II delta subunit) - |
| | | | | Rattus norvegicus |
| NOV4b | 278908476 | 51 | 52 | Calcium/calmodulin-dependent protein kinase |
| | | | | type II delta chain (EC 2.7.1.123) |
| | | | | (CaM-kinase II delta chain) (CaM kinase II |
| | | | | delta subunit) (CaMK-II delta subunit) - |
| | | | | Rattus norvegicus |
| NOV4c | 278908492 | 53 | 54 | Calcium/calmodulin-dependent protein kinase |
| | | | | type II delta chain (EC 2.7.1.123) |
| | | | | (CaM-kinase II delta chain) (CaM kinase II |
| | | | | delta subunit) (CaMK-II delta subunit) - |
| | | | | Rattus norvegicus |
| NOV4d | 278908496 | 55 | 56 | Calcium/calmodulin-dependent protein kinase |
| | | | | type II delta chain (EC 2.7.1.123) |
| | | | | (CaM-kinase II delta chain) (CaM kinase II |
| | | | | delta subunit) (CaMK-II delta subunit) - |
| | | | | Rattus norvegicus |
| NOV5a | CG119621-02 | 57 | 58 | protein-tyrosine kinase (EC 2.7.1.112) hck |
| NOV5b | CG119621-01 | 59 | 60 | protein-tyrosine kinase (EC 2.7.1.112) hck |
| NOV6a | CG124553-01 | 61 | 62 | Putative polypeptide |
| | | | | N-acetylgalactosaminyltransferase - Homo |
| | | | | sapiens |
| NOV6b | 277206285 | 63 | 64 | Putative polypeptide |
| | | | | N-acetylgalactosaminyltransferase - Homo |
| | | | | sapiens |
| NOV6c | SNP13379778 | 65 | 66 | Putative polypeptide |
| | | | | N-acetylgalactosaminyltransferase - Homo |
| | | | | sapiens |
| NOV6d | SNP13379831 | 67 | 68 | Putative polypeptide |
| | | | | N-acetylgalactosaminyltransferase - Homo |
| | | | | sapiens |
| NOV6e | SNP13379833 | 69 | 70 | Putative polypeptide |
| | | | | N-acetylgalactosaminyltransferase - Homo |
| | | | | sapiens |
| NOV7a | CG187738-02 | 71 | 72 | Branched-chain amino acid aminotransferase, |
| | | | | cytosolic |
| NOV7b | CG187738-01 | 73 | 74 | Branched-chain amino acid aminotransferase, |
| | | | | cytosolic |
| NOV7c | CG187738-03 | 75 | 76 | Branched-chain amino acid aminotransferase, |
| | | | | cytosolic |
| NOV8a | CG55676-04 | 77 | 78 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8b | 304967299 | 79 | 80 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8c | CG55676-01 | 81 | 82 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8d | CG55676-02 | 83 | 84 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8e | CG55676-03 | 85 | 86 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8f | CG55676-05 | 87 | 88 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8g | CG55676-06 | 89 | 90 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8h | CG55676-07 | 91 | 92 | Putative chemokine receptor (G |
| | | | | protein-coupled receptor) (Putative G-protein |
| | | | | coupled receptor) (Seven transmembrane helix |
| | | | | receptor) - Homo sapiens |
| NOV8i | CG55676-08 | 93 | 94 | Putative chemokine receptor (G |
| | | | | protein-coupled receptor) (Putative G-protein |
| | | | | coupled receptor) (Seven transmembrane helix |
| | | | | receptor) - Homo sapiens |
| NOV9a | CG57042-01 | 95 | 96 | Cathepsin S precursor (EC 3.4.22.27) - Homo |
| | | | | sapiens |
| NOV9b | 275620530 | 97 | 98 | Cathepsin S precursor (EC 3.4.22.27) - Homo |
| | | | | sapiens |
| NOV10a | CG57589-01 | 99 | 100 | Ethanolamine-phosphate cytidylyltransferase |
| | | | | (EC 2.7.7.14) (Phosphorylethanolamine |
| | | | | transferase) (CTP: phosphoethanolamine |
| | | | | cytidylyltransferase) - Homo sapiens |
| NOV10b | 283866181 | 101 | 102 | Ethanolamine-phosphate cytidylyltransferase |
| | | | | (EC 2.7.7.14) (Phosphorylethanolamine |
| | | | | transferase) (CTP: phosphoethanolamine |
| | | | | cytidylyltransferase) - Homo sapiens |
| NOV10c | CG57589-03 | 103 | 104 | Ethanolamine-phosphate cytidylyltransferase |
| | | | | (EC 2.7.7.14) (Phosphorylethanolamine |
| | | | | transferase) (CTP: phosphoethanolamine |
| | | | | cytidylyltransferase) - Homo sapiens |
| NOV10d | CG57589-02 | 105 | 106 | Ethanolamine-phosphate cytidylyltransferase |
| | | | | (EC 2.7.7.14) (Phosphorylethanolamine |
| | | | | transferase) (CTP: phosphoethanolamine |
| | | | | cytidylyltransferase) - Homo sapiens |
| NOV11a | CG91149-03 | 107 | 108 | PRKAA1 protein - Homo sapiens |
| NOV11b | CG91149-01 | 109 | 110 | PRKAA1 protein - Homo sapiens |
| NOV11c | CG91149-02 | 111 | 112 | PRKAA1 protein - Homo sapiens |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to, e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g., detection of a variety of cancers. SNP analysis for each NOVX, if applicable, is presented in Example D.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 56; (b) a variant of a mature form of the amino acid sequence selected from the group consisting of SEQ ID NO: 2n, wherein n is an integer between 1 and 56, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 56; (d) a variant of the amino acid sequence selected from the group consisting of SEQ ID NO:2n, wherein n is an integer between 1 and 56 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 56; (b) a variant of a mature form of the amino acid sequence selected from the group consisting of SEQ ID NO: 2n, wherein n is an integer between 1 and 56 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 56; (d) a variant of the amino acid sequence selected from the group consisting of SEQ ID NO: 2n, wherein n is an integer between 1 and 56, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 56 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 56; (b) a nucleotide sequence wherein one or more nucleotides in the nucleotide sequence selected from the group consisting of SEQ ID NO: 2n−1, wherein n is an integer between 1 and 56 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 56; and (d) a nucleic acid fragment wherein one or more nucleotides in the nucleotide sequence selected from the group consisting of SEQ ID NO: 2n−1, wherein n is an integer between 1 and 56 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO:2n−1, wherein n is an integer between 1 and 56, or a complement of this nucleotide sequence, can be isolated using standard molecular biology techniques and the sequence information provided herein. Using all or a portion of the nucleic acid sequence of SEQ ID NO:2n−1, wherein n is an integer between 1 and 56, as a hybridization probe, NOVX molecules can be isolated using standard hybridization and cloning techniques (e.g., as described in Sambrook, et al., (eds.), MOLECULAR CLONING: A LABORATORY MANUAL 2nd Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO:2n−1, wherein n is an integer between 1 and 56, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO:2n−1, wherein n is an integer between 1 and 56, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO:2n−1, wherein n is an integer between 1 and 56, is one that is sufficiently complementary to the nucleotide sequence of SEQ ID NO:2n−1, wherein n is an integer between 1 and 56, that it can hydrogen bond with few or no mismatches to the nucleotide sequence shown in SEQ ID NO:2n−1, wherein n is an integer between 1 and 56, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence.

Fragments may be derived from any contiguous portion of a nucleic acid or amino acid sequence of choice. A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g., they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g., Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO:2n−1, wherein n is an integer between 1 and 56, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g., from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO:2n−1, wherein n is an integer between 1 and 56; or an anti-sense strand nucleotide sequence of SEQ ID NO:2n−1, wherein n is an integer between 1 and 56; or of a naturally occurring mutant of SEQ ID NO:2n−1, wherein n is an integer between 1 and 56.

Probes based on the human NOVX nucleotide sequences can be used to detect transcripts or genomic sequences encoding the same or homologous proteins. In various embodiments, the probe has a detectable label attached, e.g., the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO:2n−1, wherein n is an integer between 1 and 56, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO:2n−1, wherein n is an integer between 1 and 56, due to degeneracy of the genetic code and thus encode the same NOVX proteins as that encoded by the nucleotide sequences of SEQ ID NO:2n−1, wherein n is an integer between 1 and 56. In another embodiment, an isolated nucleic acid molecule of the invention has a nucleotide sequence encoding a protein having an amino acid sequence of SEQ ID NO:2n, wherein n is an integer between 1 and 56.

In addition to the human NOVX nucleotide sequences of SEQ ID NO:2n−1, wherein n is an integer between 1 and 56, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO:2n−1, wherein n is an integer between 1 and 56, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO:2n−1, wherein n is an integer between 1 and 56. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO:2n−1, wherein n is an integer between 1 and 56, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO:2n−1, wherein n is an integer between 1 and 56, or fragments, analogs or derivatives thereof, under conditions of moderate stringency is provided. A non-limiting example of moderate stringency hybridization conditions are hybridization in 6×SSC, 5× Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Krieger, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NO:2n−1, wherein n is an integer between 1 and 56, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO:2n−1, wherein n is an integer between 1 and 56, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO:2n, wherein n is an integer between 1 and 56. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are not particularly amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO:2n−1, wherein n is an integer between 1 and 56, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO:2n, wherein n is an integer between 1 and 56. Preferably, the protein encoded by the nucleic acid molecule is at least about 60% homologous to SEQ ID NO:2n, wherein n is an integer between 1 and 56; more preferably at least about 70% homologous to SEQ ID NO:2n, wherein n is an integer between 1 and 56; still more preferably at least about 80% homologous to SEQ ID NO:2n, wherein n is an integer between 1 and 56; even more preferably at least about 90% homologous to SEQ ID NO:2n, wherein n is an integer between 1 and 56; and most preferably at least about 95% homologous to SEQ ID NO:2n, wherein n is an integer between 1 and 56.

An isolated nucleic acid molecule encoding a NOVX protein homologous to the protein of SEQ ID NO:2n, wherein n is an integer between 1 and 56, can be created by introducing one or more nucleotide substitutions, additions or deletions into the nucleotide sequence of SEQ ID NO:2n−1, wherein n is an integer between 1 and 56, such that one or more amino acid substitutions, additions or deletions are introduced into the encoded protein.

Mutations can be introduced any one of SEQ ID NO:2n−1, wherein n is an integer between 1 and 56, by standard techniques, such as site-directed mutagenesis and PCR-mediated mutagenesis. Preferably, conservative amino acid substitutions are made at one or more non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g. aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO:2n−1, wherein n is an integer between 1 and 56, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, UFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thymidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where is long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (NI 9)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g., inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g., commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the mRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynucleotide expression is observed in the subject sample relative to the control sample, using the assays described. This NOVX gene knockdown approach provides a rapid method for determination of a NOVX minus (NOVX) phenotype in the treated subject sample. The NOVX⁻ phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2X-proteinase-K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37° C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in-vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1 and 56, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO:2n, wherein n is an integer between 1 and 56, or antisense nucleic acids complementary to a NOVX nucleic acid sequence of SEQ ID NO:2n−1, wherein n is an integer between 1 and 56, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences that flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N-6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO:2n−1, wherein n is an integer between 1 and 56). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performed using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996. supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)-amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Natl. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization-triggered cleavage agents (see, e.g., Krol, et al., 1988. BioTechniques 6:958-976) or intercalating agents (see, e.g., Zon, 1988. Pharm. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO:2n, wherein n is an integer between 1 and 56. The invention also includes a mutant or variant protein any of whose residues may be changed from the corresponding residues shown in any one of SEQ ID NO:2n, wherein n is an integer between 1 and 56, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), is more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO:2n, wherein n is an integer between 1 and 56) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO:2n, wherein n is an integer between 1 and 56. In other embodiments, the NOVX protein is substantially homologous to SEQ ID NO:2n, wherein n is an integer between 1 and 56, and retains the functional activity of the protein of SEQ ID NO:2n, wherein n is an integer between 1 and 56, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO:2n, wherein n is an integer between 1 and 56, and retains the functional activity of the NOVX proteins of SEQ ID NO:2n, wherein n is an integer between 1 and 56.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO:2n−1, wherein n is an integer between 1 and 56.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO:2n, wherein n is an integer between 1 and 56, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand/NOVX interaction may be useful therapeutically for both the treatment of proliferative and differentiative disorders, as well as modulating (e.g., promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g. for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S1 nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen-binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO:2n, wherein n is an integer between 1 and 56, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three-dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen that is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, MONOCLONAL ANTIBODIES: PRINCIPLES AND PRACTICE, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, 1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals. For example, mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers that are recovered from recombinant cell culture. The preferred interface comprises at least a part of the CH3 region of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies can be prepared as full length antibodies or antibody fragments (e.g., F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol is complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets. Various techniques for making and isolating bispecific antibody fragments directly from recombinant cell culture have also been described. For example, bispecific antibodies have been produced using leucine zippers. Kostelny et al., J. Immunol. 148(5): 1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker that is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g., CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, P-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand that may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Theory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, useful expression vectors in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, is thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saccharomyces cerivisae include pYepSec1 (Baldari, et al., 1987. EMBO J. 6: 229-234), pMFa (Kudjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banedji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of anti sense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing a NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO:2n−1, wherein n is an integer between 1 and 56, can be introduced as a transgene into the genome of a non-human animal. Alternatively, a non-human homologue of the human NOVX gene, such as a mouse NOVX gene, can be isolated based on hybridization to the human NOVX cDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO:2n−1, wherein n is an integer between 1 and 56), but more preferably, is a non-human homologue of a human NOVX gene. For example, a mouse homologue of human NOVX gene of SEQ ID NO:2n−1, wherein n is an integer between 1 and 56, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage Pl. For a description of the cre/loxP recombinase system, see, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser that contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds that bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12:145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37:2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g., a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e., intracellular Ca²⁺, diacylglycerol, IP₃, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g., stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chemicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO:2n−1, wherein n is an integer between 1 and 56, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO:2n−1, wherein n is an integer between 1 and 56, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence or absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO:2n−1, wherein n is an integer between 1 and 56, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qu Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S1 nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A

### Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | | |
|---|---|---|
| NOV1a, | SEQ ID NO: 1 | 2239 bp |
| CG101025-01 | ORF Start: | ORF Stop: |
| DNA Sequence | ATG at 25 | TGA at 2077 |

TCGCCGAGCCCGTCCGCCGCCGCCATGGCCACCACGGTGACCTGCACCCG
CTTCACCGACGAGTACCAGCTCTACGAGGATATTGGCAAGGGGGCTTTCT
CTGTGGTCCGACGCTGTGTCAAGCTCTGCACCGGCCATGAGTATGCAGCC
AAGATCATCAACACCAAGAAGCTGTCAGCCAGAGATCACCAGAAGCTGGA
GAGAGAGGCTCGGATCTGCCGCCTTCTGAAGCATTCCAACATCGTGCGTC
TCCACGACAGCATCTCCGAGGAGGGCTTCCACTACCTGGTCTTCGATCTG
GTCACTGGTGGGGAGCTCTTTGAAGACATTGTGGCGAGAGAGTACTACAG
CGAGGCTGATGCCAGTCACTGTATCCAGCAGATCCTGGAGGCCGTTCTCC
ATTGTCACCAAATGGGGGTCGTCCACAGAGACCTCAAGCCGGAGAACCTG
CTTCTGGCCAGCAAGTGCAAAGGGGCTGCAGTGAAGCTGGCAGACTTCGG
CCTAGCTATCGAGGTGCAGGGGGACCAGCAGGCATGGTTTGGTTTCGCTG
GCACACCAGGCTACCTGTCCCCTGAGGTCCTTCGCAAAGAGGCGTATGGC
AAGCCTGTGGACATCTGGGCATGTGGGGTGATCCTGTACATCCTGCTCGT
GGGCTACCCACCCTTCTGGGACGAGGACCAGCACAAGCTGTACCAGCAGA
TCAAGGCTGGTGCCTATGACTTCCCGTCCCCTGAGTGGGACACCGTCACT
CCTGAAGCCAAAAACCTCATCAACCAGATGCTGACCATCAACCCTGCCAA
GCGCATCACAGCCCATGAGGCCCTGAAGCACCCGTGGGTCTGCCAACGCT
CCACGGTAGCATCCATGATGCACAGACAGGAGACTGTGGAGTGTCTGAAA
AAGTTCAATGCCAGGAGAAAGCTCAAGGGAGCCATCCTCACCACCATGCT
GGCCACACGGAATTTCTCAGCCAAGAGTTTACTCAACAAGAAAGCAGATG
GAGTCAAGCCCCAGACGAATAGCACCAAAAACAGTGCAGCCGCCACCAGC
CCCAAAGGGACGCTTCCTCCTGCCGCCCTGGAGCCTCAAACCACCGTCAT
CCATAACCCAGTGGACGGGATTAAGGAGTCTTCTGACAGTGCCAATACCA
CCATAGAGGATGAAGACGCTAAAGCCCCCAGGGTCCCCGACATCCTGAGC
TCAGTGAGGAGGGGCTCGGGAGCCCCAGAAGCCGAGGGGCCCCTGCCCTG
CCCATCTCCGGCTCCCTTTAGCCCCCTGCCAGCCCCATCCCCCAGGATCT
CTGACATCCTGAACTCTGTGAGAAGGGGTTCAGGAACCCCAGAAGCCGAG
GGCCCCCTCTCAGCGGGGCCCCCGCCCTGCCTGTCTCCGGCTCTCCTAGG
CCCCCTGTCCTCCCCGTCCCCCAGGATCTCTGACATCCTGAACTCTGTGA
GGAGGGGCTCAGGGACCCCAGAAGCCGAGGGCCCCTCGCCAGTGGGGCCC
CCGCCCTGCCCATCTCCGACTATCCCTGGCCCCCTGCCCACCCCATGGAT
GGATGACATCCCAGGGCTGCTGCCACCCCCACCTGTGGGGAGACACCAGA
CTGGGGGTGGTGTGGAGATACTCTTAGAGAAGAGGCTGCTGGGCCACGGG
CTCGGCATGGCAGGGCAGTGGCTAGCCCGGAAGCAGGAGATCATTAAGAC
CACGGAGCAGCTCATCGAGGCCGTCAACAACGGTGACTTTGAGGCCTACG
CGAAAATCTGTGACCCAGGGCTGACCTCGTTTGAGCCTGAAGCACTGGGC
AACCTGGTTGAAGGGATGGACTTCCACAGATTCTACTTCGAGAACCTGCT
GGCCAAGAACAGCAAGCCGATCCACACGACCATCCTGAACCCACACGTGC
ACGTCATTGGAGAGGATGCCGCCTGCATCGCTTACATCCGGCTCACGCAG
TACATTGACGGGCAGGGCCGGCCCCGCACCAGCCAGTCTGAGGAGACCCG
CGTGTGGCACCGCCGCGACGGCAAGTGGCAGAATGTGCACTTCCACTGCT
CGGGCGCGCCTGTGGCCCCGCTGCAGTGAAGAGCTGCGCCCTGGTTTCGC
CGGACAGAGTTGGTGTTTGGAGCCCGACTGCCCTCGGGCACACGGCCTGC
CTGTCGCATGTTTGTGTCTGCCTCGTTCCCTCCCCTGGTGCCTGTGTCTG
CAGAAAAACAAGACCAGATGTGATTTGTTAAAAAAAAAA

| | | | |
|---|---|---|---|
| NOV1a, CG101025-01 | | | MW at |
| Protein Sequence | SEQ ID NO: 2 | 684 aa | 74807.5 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIM
QMLTINPAKRITAHEALKHPWVCQRSTVASMMHRQETVECLKKFNARRKL
KGAILTTMLATRNFSAKSLLNKKADGVKPQTNSTKNSAAATSPKGTLPPA
ALEPQTTVIHNPVDGIKESSDSANTTIEDEDAKAPRVPDILSSVRRGSGA
PEAEGPLPCPSPAPFSPLPAPSPRISDILNSVRRGSGTPEAEGPLSAGPP
PCLSPALLGPLSSPSPRISDILNSVRRGSGTPEAEGPSPVGPPPCPSPTI
PGPLPTPWMDDIPGLLPPPPVGRHQTGGGVEILLEKRLLGHGLGMAGQWL
ARKQEIIKTTEQLIEAVNNGDFEAYAKICDPGLTSFEPEALGNLVEGMDF
HRFYFENLLAKNSKPIHTTILNPHVHVIGEDAACIAYIRLTQYIDGQGRP
RTSQSEETRVWHRRDGKWQNVHFHCSGAPVAPLQ

| | | |
|---|---|---|
| NOV1b, | SEQ ID NO: 3 | 2014 bp |
| CG101025-07 | ORF Start: | ORF Stop: |
| DNA Sequence | ATG at 14 | TGA at 2012 |

CACCGGATCCACCATGGCCACCACGGTGACCTGCACCCGCTTCACCGACG
AGTACCAGCTCTACGAGGATATTGGCAAGGGGGCTTTCTCTGTGGTCCGA
CGCTGTGTCAAGCTCTGCACCGGCCATGAGTATGCAGCCAAGATCATCAA
CACCAAGAAGCTGTCAGCCAGAGATCACCAGAAGCTGGAGAGAGAGGCTC
GGATCTGCCGCCTTCTGAAGCATTCCAACATCGTGCGTCTCCACGACAGC
ATCTCCGAGGAGGGCTTCCACTACCTGGTCTTCGATCTGGTCACTGGTGG
GGAGCTCTTTGAAGACATTGTGGCGAGAGAGTACTACAGCGAGGCTGATG
CCAGTCACTGTATCCAGCAGATCCTGGAGGCCGTTCTCCATTGTCACCAA
ATGGGGGTCGTCCACAGAGACCTCAAGCCGGAGAACCTGCTTCTGGCCAG
CAAGTGCAAAGGGGCTGCAGTGAAGCTGGCAGACTTCGGCCTAGCTATCG
AGGTGCAGGGGGACCAGCAGGCATGGTTTGGTTTCGCTGGCACACCAGGC
TACCTGTCCCCTGAGGTCCTTCGCAAAGAGGCGTATGGCAAGCCTGTGGA
CATCTGGGCATGTGGGGTGATCCTGTACATCCTGCTCGTGGGCTACCCAC
CCTTCTGGGACGAGGACCAGCACAAGCTGTACCAGCAGATCAAGGCTGGT
GCCTATGACTTCCCGTCCCCTGAGTGGGACACCGTCACTCCTGAAGCCAA
AAACCTCATCAACCAGATGCTGACCATCAACCCTGCCAAGCGCATCACAG
CCCATGAGGCCCTGAAGCACCCGTGGGTCTGCCAACGCTCCACGGTAGCA
TCCATGATGCACAGACAGGAGACTGTGGAGTGTCTGAAAAAGTTCAATGC
CAGGAGAAAGCTCAAGGGAGCCATCCTCACCACCATGCTGGCCACACGGA
ATTTCTCAGTGGGCAGACAGACCACCGCTCCGGCCACAATGTCCACCGCG
GCCTCCGGCACCACCATGGGGCTGGTGGAACAAGCCAAGAGTTTACTCAA
CAAGAAAGCAGATGGAGTCAAGCCCCAGACGAATAGCACCAAAAACAGTG
CAGCCGCCACCAGCCCCAAAGGGACGCTTCCTCCTGCCGCCCTGGAGCCT
CAAACCACCGTCATCCATAACCCAGTGGACGGGATTAAGGAGTCTTCTGA
CAGTGCCAATACCACCATAGAGGATGAAGACGCTAAAGCCCCCAGGGTCC
CCGACATCCTGAGCTCAGTGAGGAGGGGCTCGGGAGCCCCAGAAGCCGAG
GGGCCCCTGCCCTGCCCATCTCCGGCTCCCTTTAGCCCCCTGCCAGCCCC
ATCCCCCAGGATCTCTGACATCCTGAACTCTGTGAGAAGGGGTTCAGGAA
CCCCAGAAGCCGAGGGCCCCCTCTCAGCGGGGCCCCCGCCCTGCCTGTCT
CCGGCTCTCCTAGGCCCCCTGTCCTCCCCGTCCCCCAGGATCTCTGACAT
CCTGAACTCTGTGAGGAGGGGCTCAGGGACCCCAGAAGCCGAGGGCCCCT
CGCCAGTGGGGCCCCCGCCCTGCCCATCTCCGACTATCCCTGGCCCCCTG
CCCACCCCATCCCGGAAGCAGGAGATCATTAAGACCACGGAGCAGCTCAT
CGAGGCCGTCAACAACGGTGACTTTGAGGCCTACGCGAAAATCTGTGACC
CAGGGCTGACCTCGTTTGAGCCTGAAGCACTGGGCAACCTGGTTGAAGGG
ATGGACTTCCACAGATTCTACTTCGAGAACCTGCTGGCCAAGAACAGCAA
GCCAATCCACACGACCATCCTGAACCCACACGTGCACGTCATTGGAGAGG
ATGCCGCCTGCATCGCTTACATCCGGCTCACGCAGTACATTGACGGGCAG
GGCCGGCCCCGCACCAGCCAGTCTGAGGAGACCCGCGTGTGGCACCGCCG
CGACGGCAAGTGGCAGAACGTGCACTTCCACTGCTCGGGCGCGCCTGTGG
CCCCGCTGCAGTGA

| | | | |
|---|---|---|---|
| NOV1b, CG101025-07 | | | MW at |
| Protein Sequence | SEQ ID NO: 4 | 666 aa | 72676.9 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSVGRQTTAPATMSTAASGTTMGLVEQAKSLLNKKAD
GVKPQTNSTKNSAAATSPKGTLPPAALEPQTTVIHNPVDGIKESSDSANT
TIEDEDAKAPRVPDILSSVRRGSGAPEAEGPLPCPSPAPFSPLPAPSPRI
SDILNSVRRGSGTPEAEGPLSAGPPPCLSPALLGPLSSPSPRISDILNSV
RRGSGTPEAEGPSPVGPPPCPSPTIPGPLPTPSRKQEIIKTTEQLIEAVN
NGDFEAYAKICDPGLTSFEPEALGNLVEGMDFHRFYFENLLAKNSKPIHT
TILNPHVHVIGEDAACIAYIRLTQYIDGQGRPRTSQSEETRVWHRRDGKW
QNVHFHCSGAPVAPLQ

| | | |
|---|---|---|
| NOV1c, | SEQ ID NO: 5 | 1998 bp |
| 278987803 | ORF Start: | ORF Stop: |
| DNA Sequence | at 1 | end of sequence |

ATGGCCACCACGGTGACCTGCACCCGCTTCACCGACGAGTACCAGCTCTA
CGAGGATATTGGCAAGGGGGCTTTCTCTGTGGTCCGACGCTGTGTCAAGC
TCTGCACCGGCCATGAGTATGCAGCCAAGATCATCAACACCAAGAAGCTG
TCAGCCAGAGATCACCAGAAGCTGGAGAGAGAGGCTCGGATCTGCCGCCT
TCTGAAGCATTCCAACATCGTGCGTCTCCACGACAGCATCTCCGAGGAGG
GCTTCCACTACCTGGTCTTCGATCTGGTCACTGGTGGGGAGCTCTTTGAA
GACATTGTGGCGAGAGAGTACTACAGCGAGGCTGATGCCAGTCACTGTAT
CCAGCAGATCCTGGAGGCCGTTCTCCATTGTCACCAAATGGGGGTCGTCC
ACAGAGACCTCAAGCCGGAGAACCTGCTTCTGGCCAGCAAGTGCAAAGGG
GCTGCAGTGAAGCTGGCAGACTTCGGCCTAGCTATCGAGGTGCAGGGGGA
CCAGCAGGCATGGTTTGGTTTCGCTGGCACACCAGGCTACCTGTCCCCTG
AGGTCCTTCGCAAAGAGGCGTATGGCAAGCCTGTGGACATCTGGGCATGT
GGGGTGATCCTGTACATCCTGCTCGTGGGCTACCCACCCTTCTGGGACGA
GGACCAGCACAAGCTGTACCAGCAGATCAAGGCTGGTGCCTATGACTTCC
CGTCCCCTGAGTGGGACACCGTCACTCCTGAAGCCAAAAACCTCATCAAC
CAGATGCTGACCATCAACCCTGCCAAGCGCATCACAGCCCATGAGGCCCT
GAAGCACCCGTGGGTCTGCCAACGCTCCACGGTAGCATCCATGATGCACA
GACAGGAGACTGTGGAGTGTCTGAAAAAGTTCAATGCCAGGAGAAAGCTC
AAGGGAGCCATCCTCACCACCATGCTGGCCACACGGAATTTCTCAGTGGG
CAGACAGACCACCGCTCCGGCCACAATGTCCACCGCGGCCTCCGGCACCA
CCATGGGGCTGGTGGAACAAGCCAAGAGTTTACTCAACAAGAAAGCAGAT
GGAGTCAAGCCCCAGACGAATAGCACCAAAAACAGTGCAGCCGCCACCAG
CCCCAAAGGGACGCTTCCTCCTGCCGCCCTGGAGCCTCAAACCACCGTCA
TCCATAACCCAGTGGACGGGATTAAGGAGTCTTCTGACAGTGCCAATACC
ACCATAGAGGATGAAGACGCTAAAGCCCCCAGGGTCCCCGACATCCTGAG
CTCAGTGAGGAGGGGCTCGGGAGCCCCAGAAGCCGAGGGGCCCCTGCCCT
GCCCATCTCCGGCTCCCTTTAGCCCCCTGCCAGCCCCATCCCCCAGGATC
TCTGACATCCTGAACTCTGTGAGAAGGGGTTCAGGAACCCCAGAAGCCGA
GGGCCCCCTCTCAGCGGGGCCCCCGCCCTGCCTGTCTCCGGCTCTCCTAG
GCCCCCTGTCCTCCCCGTCCCCCAGGATCTCTGACATCCTGAACTCTGTG
AGGAGGGGCTCAGGGACCCCAGAAGCCGAGGGCCCCTCGCCAGTGGGGCC
CCCGCCCTGCCCATCTCCGACTATCCCTGGCCCCCTGCCCACCCCATCCC
GGAAGCAGGAGATCATTAAGACCACGGAGCAGCTCATCGAGGCCGTCAAC
AACGGTGACTTTGAGGCCTACGCGAAAATCTGTGACCCAGGGCTGACCTC
GTTTGAGCCTGAAGCACTGGGCAACCTGGTTGAAGGGATGGACTTCCACA
GATTCTACTTCGAGAACCTGCTGGCCAAGAACAGCAAGCCAATCCACACG
ACCATCCTGAACCCACACGTGCACGTCATTGGAGAGGATGCCGCCTGCAT
CGCTTACATCCGGCTCACGCAGTACATTGACGGGCAGGGCCGGCCCCGCA
CCAGCCAGTCTGAGGAGACCCGCGTGTGGCACCGCCGCGACGGCAAGTGG
CAGAACGTGCACTTCCACTGCTCGGGCGCGCCTGTGGCCCCGCTGCAG

| | | | |
|---|---|---|---|
| NOV1c, 278987803 | | | MW at |
| Protein Sequence | SEQ ID NO: 6 | 666 aa | 73322.5 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSVGRQTTAPATMSTAASGTTMGLVEQAKSLLNKKAD
GVKPQTNSTKNSAAATSPKGTLPPAALEPQTTVIHNPVDGIKESSDSANT
TIEDEDAKAPRVPDILSSVRRGSGAPEAEGPLPCPSPAPFSPLPAPSPRI
SDILNSVRRGSGTPEAEGPLSAGPPPCLSPALLGPLSSPSPRISDILNSV
RRGSGTPEAEGPSPVGPPPCPSPTIPGPLPTPSRKQEIIKTTEQLIEAVN
NGDFEAYAKICDPGLTSFEPEALGNLVEGMDFNRFYFENLLAKNSKPIHT
TILMPHVHVIGEDAACIAYIRLTQYIDGQGRPRTSQSEETRVWHRRDGKW
QNVHFHCSGAPVAPLQ

| | | |
|---|---|---|
| NOV1d, | SEQ ID NO: 7 | 1809 bp |
| 278987807 | ORF Start: | ORF Stop: |
| DNA Sequence | at 1 | end of sequence |

ATGGCCACCACGGTGACCTGCACCCGCTTCACCGACGAGTACCAGCTCTA
CGAGGATATTGGCAAGGGGGCTTTCTCTGTGGTCCGACGCTGTGTCAAGC
TCTGCACCGGCCATGAGTATGCAGCCAAGATCATCAACACCAAGAAGCTG
TCAGCCAGAGATCACCAGAAGCTGGAGAGAGAGGCTCGGATCTGCCGCCT
TCTGAAGCATTCCAACATCGTGCGTCTCCACGACAGCATCTCCGAGGAGG
GCTTCCACTACCTGGTCTTCGATCTGGTCACTGGTGGGGAGCTCTTTGAA
GACATTGTGGCGAGAGAGTACTACAGCGAGGCTGATGCCAGTCACTGTAT
CCAGCAGATCCTGGAGGCCGTTCTCCATTGTCACCAAATGGGGGTCGTCC
ACAGAGACCTCAAGCCGGAGAACCTGCTTCTGGCCAGCAAGTGCAAAGGG
GCTGCAGTGAAGCTGGCAGACTTCGGCCTAGCTATCGAGGTGCAGGGGGA
CCAGCAGGCATGGTTTGGTTTCGCTGGCACACCAGGCTACCTGTCCCCTG
AGGTCCTTCGCAAAGAGGCGTATGGCAAGCCTGTGGACATCTGGGCATGT
GGGGTGATCCTGTACATCCTGCTCGTGGGCTACCCACCCTTCTGGGACGA
GGACCAGCACAAGCTGTACCAGCAGATCAAGGCTGGTGCCTATGACTTCC
CGTCCCCTGAGTGGGACACCGTCACTCCTGAAGCCAAAAACCTCATCAAC
CAGATGCTGACCATCAACCCTGCCAAGCGCATCACAGCCCATGAGGCCCT
GAAGCACCCGTGGGTCTGCCAACGCTCCACGGTAGCATCCATGATGCACA
GACAGGAGACTGTGGAGTGTCTGAAAAAGTTCAATGCCAGGAGAAAGCTC
AAGGGAGCCATCCTCACCACCATGCTGGCCACACGGAATTTCTCAGCCAA
GAGTTTACTCAACAAGAAAGCAGATGGAGTCAAGCCCCAGACGAATAGCA
CCAAAAACAGTGCAGCCGCCACCAGCCCCAAAGGGACGCTTCCTCCTGCC
GCCCTGGAGCCTCAAACCACCGTCATCCATAACCCAGTGGACGGGATTAA
GGAGTCTTCTGACAGTGCCAATACCACCATAGAGGATGAAGACGCTAAAG
CCCCCAGGATCTCTGACATCCTGAACTCTGTGAGAAGGGGTTCAGGAACC
CCAGAAGCCGAGGGCCCCCTCTCAGCGGGGCCCCCGCCCTGCCTGTCTCC
GGCTCTCCTAGGCCCCCTGTCCTCCCCGTCCCCCAGGATCTCTGACATCC
TGAACTCTGTGAGGAGGGGCTCAGGGACCCCAGAAGCCGAGGGCCCCTCG
CCAGTGGGGCCCCCGCCCTGCCCATCTCCGACTATCCCTGGCCCCCTGCC
CACCCCATCCCGGAAGCAGGAGATCATTAAGACCACGGAGCAGCTCATCG
AGGCCGTCAACAACGGTGACTTTGAGGCCTACGCGAAAATCTGTGACCCA
GGGCTGACCTCGTTTGAGCCTGAAGCACTGGGCAACCTGGTTGAAGGGAT
GGACTTCCACAGATTCTACTTCGAGAACCTGCTGGCCAAGAACAGCAAGC
CGATCCACACGACCATCCTGAACCCACACGTGCACGTCATTGGAGAGGAT
GCCGCCTGCATCGCTTACATCCGGCTCACGCAGTACATTGACGGGCAGGG
CCGGCCCCGCACCAGCCAGTCTGAGGAGACCCGCGTGTGGCACCGCCGCG
ACGGCAAGTGGCAGAACGTGCACTTCCACTGCTCGGGCGCGCCTGTGGCC
CCGCTGCAG

| | | | |
|---|---|---|---|
| NOV1d, 278987807 | | | MW at |
| Protein Sequence | SEQ ID NO: 8 | 603 aa | 67084.6 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSAKSLLNKKADGVKPQTNSTKNSAAATSPKGTLPPA
ALEPQTTVIHNPVDGIKESSDSANTTIEDEDAKAPRISDILNSVRRGSGT
PEAEGPLSAGPPPCLSPALLGPLSSPSPRISDILNSVRRGSGTPEAEGPS
PVGPPPCPSPTIPGPLPTPSRKQEIIKTTEQLIEAVNNGDFEAYAKICDP
GLTSFEPEALGNLVEGMDFHRFYFENLLAKNSKPIHTTILNPHVHVIGED
AACIAYIRLTQYIDGQGRPRTSQSEETRVWHRRDGKWQNVHFHCSGAPVA
PLQ

| | | |
|---|---|---|
| NOV1e, | SEQ ID NO: 9 | 1923 bp |
| 278987811 | ORF Start: | ORF Stop: |
| DNA Sequence | at 1 | end of sequence |

ATGGCCACCACGGTGACCTGCACCCGCTTCACCGACGAGTACCAGCTCTA
CGAGGATATTGGCAAGGGGGCTTTCTCTGTGGTCCGACGCTGTGTCAAGC
TCTGCACCGGCCATGAGTATGCAGCCAAGATCATCAACACCAAGAAGCTG
TCAGCCAGAGATCACCAGAAGCTGGAGAGAGAGGCTCGGATCTGCCGCCT
TCTGAAGCATTCCAACATCGTGCGTCTCCACGACAGCATCTCCGAGGAGG
GCTTCCACTACCTGGTCTTCGATCTGGTCACTGGTGGGGAGCTTTTTGAA
GACATTGTGGCGAGAGAGTACTACAGCGAGGCTGATGCCAGTCACTGTAT
CCAGCAGATCCTGGAGGCCGTTCTCCATTGTCACCAAATGGGGGTCGTCC
ACAGAGACCTCAAGCCGGAGAACCTGCTTCTGGCCAGCAAGTGCAAAGGG
GCTGCAGTGAAGCTGGCAGACTTCGGCCTAGCTATCGAGGTGCAGGGGGA
CCAGCAGGCATGGTTTGGTTTCGCTGGCACACCAGGCTACCTGTCCCCTG
AGGTCCTTCGCAAAGAGGCGTACGGCAAGCCCGTGGACATCTGGGCATGT
GGGGTGATCCTGTACATCCTGCTCGTGGGCTACCCACCCTTCTGGGACGA
GGACCAGCACAAGCTGTACCAGCAGATCAAGGCTGGTGCCTATGACTTCC
CGTCCCCTGAGTGGGACACCGTCACTCCTGAAGCCAAAAACCTCATCAAC
CAGATGCTGACCATCAACCCTGCCAAGCGCATCACAGCCCATGAGGCCCT
GAAGCACCCGTGGGTCTGCCAACGCTCCACGGTAGCATCCATGATGCACA
GACAGGAGACTGTGGAGTGTCTGAAAAAGTTCAATGCCAGGAGAAAGCTC
AAGGGAGCCATCCTCACCACCATGCTGGCCACACGGAATTTCTCAGCCAA
GAGTTTACTCAACAAGAAAGCAGATGGAGTCAAGCCCCAGACGAATAGCA
CCAAAAACAGTGCAGCCGCCACCAGCCCCAAAGGGACGCTTCCTCCTGCC
GCCCTGGAGCCTCAAACCACCGTCATCCATAACCCAGTGGACGGGATTAA
GGAGTCTTCTGACAGTGCCAATACCACCATAGAGGATGAAGACGCTAAAG
CCCCCAGGGTCCCCGACATCCTGAGCTCAGTGAGGAGGGGCTCGGGAGCC
CCAGAAGCCGAGGGGCCCCTGCCCTGCCCATCTCCGGCTCCCTTTAGCCC
CCTGCCAGCCCCATCCCCCAGGATCTCTGACATCCTGAACTCTGTGAGAA
GGGGTTCAGGAACCCCAGAAGCCGAGGGCCCCCTCTCAGCGGGGCCCCCG
CCCTGCCTGTCTCCGGCTCTCCTAGGCCCCCTGTCCTCCCCGTCCCCCAG
GATCTCTGACATCCTGAACTCTGTGAGGAGGGGCTCAGGGACCCCAGAAG
CCGAGGGCCCCTCGCCAGTGGGGCCCCCGCCCTGCCCATCTCCGACTATC
CCTGGCCCCCTGCCCACCCCATCCCGGAAGCAGGAGATCATTAAGACCAC
GGAGCAGCTCATCGAGGCCGTCAACAACGGTGACTTTGAGGCCTACGCGA
AAATCTGTGACCCAGGGCTGACCTCGTTTGAGCCTGAAGCACTGGGCAAC
CTGGTTGAAGGGATGGACTTCCACAGATTCTACTTCGAGAACCTGCTGGC
CAAGAACAGCAAGCCAATCCACACGACCATCCTGAACCCACACGTGCACG
TCATTGGAGAGGATGCCGCCTGCATCGCTTACATCCGGCTCACGCAGTAC
ATTGACGGGCAGGGCCGGCCCCGCACCAGCCAGTCTGAGGAGACCCGCGT
GTGGCACCGCCGCGACGGCAAGTGGCAGAATGTGCACTTCCACTGCTCGG
GCGCGCCTGTGGCCCCGCTGCAG

| | | | |
|---|---|---|---|
| NOV1e, 278987811 | | | MW at |
| Protein Sequence | SEQ ID NO: 10 | 641 aa | 70873.8 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEAIKHPWVCQRSTVASMMHRQETVECLKKFNARRKL
KGAILTTMLATRNFSAKSLLNKKADGVKPQTNSTKNSAAATSPKGTLPPA
ALEPQTTVIHNPVDGIKESSDSANTTIEDEDAKAPRVPDILSSVRRGSGA
PEAEGPLPCPSPAPFSPLPAPSPRISDILNSVRRGSGTPEAEGPLSAGPP
PCLSPALLGPLSSPSPRISDILNSVRRGSGTPEAEGPSPVGPPPCPSPTI
PGPLPTPSRKQEIIKTTEQLIEAVNNGDFEAYAKICDPGLTSFEPEALGN
LVEGMDFHRFYFENLLAKNSKPIHTTILNPHVHVIGEDAACIAYIRLTQY
IDGQGRPRTSQSEETRVWHRRDGKWQNVHFNCSGAPVAPLQ

| | | |
|---|---|---|
| NOV1f | , SEQ ID NO: 11 | 840 bp |
| 278987831 | ORF Start: | ORF Stop: |
| DNA Sequence | at 1 | end of sequence |

ATGGCCACCACGGTGACCTGCACCCGCTTCACCGACGAGTACCAGCTCT
ACGAGGATATTGGCAAGGGGGCTTTCTCTGTGGTCCGACGCTGTGTCAA
GCTCTGCACCGGCCATGAGTATGCAGCCAAGATCATCAACACCAAGAAG
CTGTCAGCCAGAGATCACCAGAAGCTGGAGAGAGAGGCTCGGATCTGCC
GCCTTCTGAAGCATTCCAACATCGTGCGTCTCCACGACAGCATCTCCGA
GGAGGGCTTCCACTACCTGGTCTTCGATCTGGTCACTGGTGGGGAGCTC
TTTGAAGACATTGTGGCGAGAGAGTACTACAGCGAGGCTGATGCCAGTC
ACTGTATCCAGCAGATCCTGGAGGCCGTTCTCCATTGTCACCAAATGGG
GGTCGTCCACAGAGACCTCAAGCCGGAGAACCTGCTTCTGGCCAGCAAG
TGCAAAGGGGCTGCAGTGAAGCTGGCAGACTTCGGCCTAGCTATCGAGG
TGCAGGGGGACCAGCAGGCATGGTTTGGTTTCGCTGGCACACCAGGCTA
CCTGTCCCCTGAGGTCCTTCGCAAAGAGGCGTATGGCAAGCCTGTGGAC
ATCTGGGCATGTGGGGTGATCCTGTACATCCTGCTCGTGGGCTACCCAC
CCTTCTGGGACGAGGACCAGCACAAGCTGTACCAGCAGATCAAGGCTGG
TGCCTATGACTTCCCGTCCCCTGAGTGGGACACCGTCACTCCTGAAGCC
AAAAACCTCATCAACCAGATGCTGACCATCAACCCTGCCAAGCGCATCA
CAGCCCATGAGGCCCTGAAGCACCCGTGGGTCTGCCAACGCTCCACGGT
AGCATCC

| | | | |
|---|---|---|---|
| NOV1f, 278987831 | | | MW at |
| Protein Sequence | SEQ ID NO: 12 | 280 aa | 32296.6 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVAS

| | | |
|---|---|---|
| NOV1g, | SEQ ID NO: 13 | 1824 bp |
| CG101025-02 | ORF Start: | ORF Stop: |
| DNA Sequence | ATG at 433 | TGA at 1672 |

GCGGCCCGCGTCGACCGAGCGCACGCCGAGCCCGTCCGCCGCCGCCATGG
CCACCACGGTGACCTGCACCCGCTTCACCGACGAGTACCAGCTCTACGAG
GATATTGGCAAGGGGGCTTTCTCTGTGGTCCGACGCTGTGTCAAGCTCTG
CACCGGCCATGAGTATGCAGCCAAGATCATCAACACCAAGAAGCTGTCAG
CCAGAGATCACCAGAAGCTGGAGAGAGAGGCTCGGATCTGCCGCCTTGTG
AAGCATTCCAACATCGTGCGTCTCCACGACAGCATCTCCGAGGAGGGCTT
CCACTACCTGGTCTTCGATCTGGTCACTGGTGGGGAGCTCTTTGAAGACA
TTGTGGCGAGAGAGTACTACAGCGAGGCTGATGCCAGTCACTGTATCCAG
CAGATCCTGAGGCCGTTCTCCATTGTCACCAAATGGGGGTCGTCCACAGA
GACCTCAAGCCGGAGAACCTGCTTCTGGCCAGCAAGTGCAAAGGGGCTGC
AGTGAAGCTGGCAGACTTCGGCCTAGCTATCGAGGTGCAGGGGGACCAGC
AGGCATGGTTTGGTTTCGCTGGCACACCAGGCTACCTGTCCCCTGAGGTC
CTTCGCAAAGAGGCGTACGGCAAGCCCGTGGACATCTGGGCATGTGGGGT
GATCCTGTACATCCTGCTCGTGGGCTACCCACCCTTCTGGGACGAGGACC
AGCACAAGCTGTACCAGCAGATCAAGGCTGGTGCCTATGACTTCCCGTCC
CCTGAGTGGGACACCGTCACTCCTGAAGCCAAAAACCTCATCAACCAGAT
GCTGACCATCAACCCTGCCAAGCGCATCACAGCCCATGAGGCCCTGAAGC
ACCCGTGGGTCTGCCAACGCTCCACGGTAGCATCCATGATGCACAGACAG
GAGACTGTGGAGTGTCTGAAAAAGTTCAATGCCAGGAGAAAGCTCAAGGG
AGCCATCCTCACCACCATGCTGGCCACACGGAATTTCTCAGTGGGCAGAC
AGACCACCGCTCCGGCCACAATGTCCACCGCGGCCTCCGGCACCACCATG
GGGCTGGTGGAACAAGCCAAGAGTTTACTCAACAAGAAAGCAGATGGAGT
CAAGCCCCAGACGAATAGTACCAAAAACAGTGCAGCCGCCACCAGCCCCA
AAGGGACGCTTCCTCCTGCCGCCCTGGAGCCTCAAACCACCGTCATCCAT
AACCCAGTGGACGGGATTAAGGAGTCTTCTGACAGTGCCAATACCACCAT
AGAGGATGAAGACGCTAAAGCCCGGAAGCAGGAGATCATTAAGACCACGG
AGCAGCTCATCGAGGCCGTCAACAACGGTGACTTTGAGGCCTACGCGAAA
ATCTGTGACCCAGGGCTGACCTCGTTTGAGCCTGAAGCACTGGGCAACCT
GGTTGAAGGGATGGACTTCCACAGATTCTACTTCGAGAACCTGCTGGCCA
AGAACAGCAAGCCGATCCACACGACCATCCTGAACCCACACGTGCACGTC
ATTGGAGAGGATGCCGCCTGCATCGCTTACATCCGGCTCACGCAGTACAT
TGACGGGCAGGGCCGGCCCCGCACCAGCCAGTCTGAGGAGACCCGCGTGT
GGCACCGCCGCGACGGCAAGTGGCAGAACGTGCACTTCCACTGCTCGGGC
GCGCCTGTGGCCCCGCTGCAGTGAAGAGCTGCGCCCTGGTTTCGCCGGAC
AGAGTTGGTGTTTGGAGCCCGACTGCCCTCGGGCACACGGCCTGCCTGTC
GCATGTTTGTGTCTGCCTCGTTCCCTCCCCTGGTTCCTGTGTCTGCAGAA
AAACAAGACCAGATGTGATTTGTT

| | | | |
|---|---|---|---|
| NOV1g, CG101025-02 | | | MW at |
| Protein Sequence | SEQ ID NO: 14 | 413 aa | 45557.4 kD |

MGVVHRDLKPENLLLASKCKGAAVKLADFGLAIEVQGDQQAWFGFAGTPG
YLSPEVLRKEAYGKPVDIWACGVILYILLVGYPPFWDEDQHKLYQQIKAG
AYDFPSPEWDTVTPEAKNLINQMLTINPAKRITAHEALKHPWVCQRSTVA
SMMHRQETVECLKKFNARRKLKGAILTTMLATRNFSVGRQTTAPATMSTA
ASGTTMGLVEQAKSLLNKKADGVKPQTNSTKNSAAATSPKGTLPPAALEP
QTTVIHNPVDGIKESSDSANTTIEDEDAKARKQEIIKTTEQLIEAVNNGD
FEAYAKICDPGLTSFEPEALGMLVEGMDFHRFYFENLLAKNSKPIHTTIL
NPHVHVIGEDAACIAYIRLTQYIDGQGRPRTSQSEETRVWHRRDGKWQNV
HFHCSGAPVAPLQ

| | | |
|---|---|---|
| NOV1b, | SEQ ID NO: 15 | 1676 bp |
| CG101025-03 | ORF Start: | ORF Stop: |
| DNA Sequence | ATG at 25 | TGA at 1534 |

TCGCCGAGCCCGTCCGCCGCCGCCATGGCCACCACGGTGACCTGCACCCG
CTTCACCGACGAGTACCAGCTCTACGAGGATATTGGCAAGGGGGCTTTCT
CTGTGGTCCGACGCTGTGTCAAGCTCTGCACCGGCCATGAGTATGCAGCC
AAGATCATCAACACCAAGAAGCTGTCAGCCAGAGATCACCAGAAGCTGGA
GAGAGAGGCTCGGATCTGCCGCCTTCTGAAGCATTCCAACATCGTGCGTC
TCCACGACAGCATCTCCGAGGAGGGCTTCCACTACCTGGTCTTCGATCTG
GTCACTGGTGGGGAGCTCTTTGAAGACATTGTGGCGAGAGAGTACTACAG
CGAGGCTGATGCCAGTCACTGTATCCAGCAGATCCTGGAGGCCGTTCTCC
ATTGTCACCAAATGGGGGTCGTCCACAGAGACCTCAAGCCGGAGAACCTG
CTTCTGGCCAGCAAGTGCAAAGGGGCTGCAGTGAAGCTGGCAGACTTCGG
CCTAGCTATCGAGGTGCAGGGGGACCAGCAGGCATGGTTTGGTTTCGCTG
GCACACCAGGCTACCTGTCCCCTGAGGTCCTTCGCAAAGAGGCGTACGGC
AAGCCCGTGGACATCTGGGCATGTGGGGTGATCCTGTACATCCTGCTCGT
GGGCTACCCACCCTTCTGGGACGAGGACCAGCACAAGCTGTACCAGCAGA
TCAAGGCTGGTGCCTATGACTTCCCGTCCCCTGAGTGGGACACCGTCACT
CCTGAAGCCAAAAACCTCATCAACCAGATGCTGACCATCAACCCTGCCAA
GCGCATCACAGCCCATGAGGCCCTGAAGCACCCGTGGGTCTGCCAACGCT
CCACGGTAGCATCCATGATGCACAGACAGGAGACTGTGGAGTGTCTGAAA
AAGTTCAATGCCAGGAGAAAGCTCAAGGGAGCCATCCTCACCACCATGCT
GGCCACACGGAATTTCTCAGCAGCCAAGAGTTTACTCAACAAGAAAGCAG
ATGGAGTCAAGCCCCATACGAATAGCACCAAAAACAGTGCAGCCGCCACC
AGCCCCAAAGGGACGCTTCCTCCTGCCGCCCTGGAGTCTTCTGACAGTGC
CAATACCACCATAGAGGATGAAGACGCTAAAGCCCGGAAGCAGGAGATCA
TTAAGACCACGGAGCAGCTCATCGAGGCCGTCAACAACGGTGACTTTGAG
GCCTACGCGAAAATCTGTGACCCAGGGCTGACCTCGTTTGAGCCTGAAGC
ACTGGGCAACCTGGTTGAAGGGATGGACTTCCACAGATTCTACTTCGAGA
ACCTGCTGGCCAAGAACAGCAAGCCGATCCACACGACCATCCTGAACCCA
CACGTGCACGTCATTGGAGAGGATGCCGCCTGCATCGCTTACATCCGGCT
CACGCAGTACATTGACGGGCAGGGCCGGCCCCGCACCAGCCAGTCTGAGG
AGACCCGCGTGTGGCACCGCCGCGACGGCAAGTGGCAGAACGTGCACTTC
CACTGCTCGGGCGCGCCTGTGGCCCCGCTGCAGTGAAGAGCTGCGCCCTG
GTTTCGCCGGACAGAGTTGGTGTTTGGAGCCCGACTGCCCTCGGGCACAC
GGCCTGCCTGTCGCATGTTTGTGTCTGCCTCGTTCCCTCCCCTGGTGCCT
GTGTCTGCAGAAAAACAAGCCCGACT

| | | | |
|---|---|---|---|
| NOV1h, CG101025-03 | | | MW at |
| Protein Sequence | SEQ ID NO: 16 | 503 aa | 56387.7 kD |

MATTVTCTRFTDEYQLYEDIGKGAFSVVRRCVKLCTGHEYAAKIINTKKL
SARDNQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSAAKSLLNKKADGVKPHTNSTKNSAAATSPKGTLPP
AALESSDSANTTIEDEDAKARKQEIIKTTEQLIEAVNNGDFEAYAKICDP
GLTSFEPEALGNLVEGMDFHRFYFENLLAKNSKPIHTTILNPHVHVIGED
AACIAYIRLTQYIDGQGRPRTSQSEETRVWHRRDGKWQNVHFHCSGAPVA
PLQ

| | | |
|---|---|---|
| NOV1i, | SEQ ID NO: 17 | 2251 bp |
| CG101025-04 | ORF Start: | ORF Stop: |
| DNA Sequence | ATG at 1 | TGA at 2090 |

ATGGCCACCACGGTGACCTGCACCCGCTTCACCGACGAGTACCAGCTCTA
CGAGGATATTGGCAAGGGGGCTTTCTCTGTGGTCCGACGCTGTGTCAAGC
TCTGCACCGGCCATGAGTATGCAGCCAAGATCATCAACACCAAGAAGCTG
TCAGCCAGAGATCACCAGAAGCTGGAGAGAGAGGCTCGGATCTGCCGCCT
TCTGAAGCATTCCAACATCGTGCGTCTCCACGACAGCATCTCCGAGGAGG
GCTTCCACTACCTGGTCTTCGATCTGGTCACTGGTGGGGAGCTCTTTGAA
GACATTGTGGCGAGAGAGTACTACAGCGAGGCTGATGCCAGGGCCACTCG
CACTAACCCACCTGCTGTTTGCCACAGTCACTGTATCCAGCAGATCCTGG
AGGCCGTTCTCCATTGTCACCAAATGGGGGTCGTCCACAGAGACCTCAAG
CCGGAGAACCTGCTTCTGGCCAGCAAGTGCAAAGGGGCTGCAGTGAAGCT
GGCAGACTTCGGCCTAGCTATCGAGGTGCAGGGGGACCAGCAGGCATGGT
TTGGTTTCGCTGGCACACCAGGCTACCTGTCCCCTGAGGTCCTTCGCAAA
GAGGCGTATGGCAAGCCTGTGGACATCTGGGCATGTGGGGTGATCCTGTA
CATCCTGCTCGTGGGCTACCCACCCTTCTGGGACGAGGACCAGCACAAGC
TGTACCAGCAGATCAAGGCTGGTGCCTATGACTTCCCGTCCCCTGAGTGG
GACACCGTCACTCCTGAAGCCAAAAACCTCATCAACCAGATGCTGACCAT
CAACCCTGCCAAGCGCATCACAGCCCATGAGGCCCTGAAGCACCCGTGGG
TCTGCCAACGCTCCACGGTAGCATCCATGATGCACAGACAGGAGACTGTG
GAGTGTCTGAAAAAGTTCAATGCCAGGAGAAAGCTCAAGGGAGCCATCCT
CACCACCATGCTGGCCACACGGAATTTCTCAGCCAAGAGTTTACTCAACA
AGAAAGCAGATGGAGTCAAGCCCCAGACGAATAGCACCAAAAACAGTGCA
GCCGCCACCAGCCCCAAAGGGACGCTTCCTCCTGCCGCCCTGGAGCCTCA
AACCACCGTCATCCATAACCCAGTGGACGGGATTAAGGAGTCTTCTGACA
GTGCCAATACCACCATAGAGGATGAAGACGCTAAAGCCCCCAGGGTCCCC
GACATCCTGAGCTCAGTGAGGAGGGGCTCGGGAGCCCCAGAAGCCGAGGG
GCCCCTGCCCTGCCCATCTCCGGCTCCCTTTAGCCCCCTGCCAGCCCCAT
CCCCCAGGATCTCTGACATCCTGAACTCTGTGAGAAGGGGTTCAGGAACC
CCAGAAGCCGAGGGCCCCCTCTCAGCGGGGCCCCCGCCCTGCCTGTCTCC
GGCTCTCCTAGGCCCCCTGTCCTCCCCGTCCCCCAGGATCTCTGACATCC
TGAACTCTGTGAGGAGGGGCTCAGGGACCCCAGAAGCCGAGGGCCCCTCG
CCAGTGGGGCCCCCGCCCTGCCCATCTCCGACTATCCCTGGCCCCCTGCC
CACCCCATGGATGGATGACATCCCAGGGCTGCTGCCACCCCCACCTGTGG
GGAGACACCAGACTGGGGGTGGTGTGGAGATACTCTTAGAGAAGAGGCTG
CTGGGCCACGGGCTCGGCATGGCAGGGCAGTGGCTAGCCCGGAAGCAGGA
GATCATTAAGACCACGGAGCAGCTCATCGAGGCCGTCAACAACGGTGACT
TTGAGGCCTACGCGAAAATCTGTGACCCAGGGCTGACCTCGTTTGAGCCT
GAAGCACTGGGCAACCTGGTTGAAGGGATGGACTTCCACAGATTCTACTT
CGAGAACCTGCTGGCCAAGAACAGCAAGCCGATCCACACGACCATCCTGA
ACCCACACGTGCACGTCATTGGAGAGGATGCCGCCTGCATCGCTTACATC
CGGCTCACGCAGTACATTGACGGGCAGGGCCGGCCCCGCACCAGCCAGTC
TGAGGAGACCCGCGTGTGGCACCGCCGCGACGGCAAGTGGCAGAATGTGC
ACTTCCACTGCTCGGGCGCGCCTGTGGCCCCGCTGCAGTGAAGAGCTGCG
CCCTGGTTTCGCCGGACAGAGTTGGTGTTTGGAGCCCGACTGCCCTCGGG
CACACGGCCTGCCTGTCGCATGTTTGTGTCTGCCTCGTTCCCTCCCCTGG
TGCCTGTGTCTGCAGAAAAACAAGACCAGATGTGATTTGTTAAAAAAAAA
A

| | | | |
|---|---|---|---|
| NOV1i, CG101025-04 | | | MW at |
| Protein Sequence | SEQ ID NO: 18 | 696 aa | 76112.0 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADARATRTNPPAVCHSHCIQQILEAVLHCHQMGVVHRDLK
PENLLLASKCKGAAVKLADFGLAIEVQGDQQAWFGFAGTPGYLSPEVLRK
EAYGKPVDIWACGVILYILLVGYPPFWDEDQHKLYQQIKAGAYDFPSPEW
DTVTPEAKNLINQMLTINPAKRITAHEALKHPWVCQRSTVASMMHRQETV
ECLKKFNARRKLKGAILTTMLATRNFSAKSLLNKKADGVKPQTNSTKNSA
AATSPKGTLPPAALEPQTTVIHNPVDGIKESSDSANTTIEDEDAKAPRVP
DILSSVRRGSGAPEAEGPLPCPSPAPFSPLPAPSPRISDILNSVRRGSGT
PEAEGPLSAGPPPCLSPALLGPLSSPSPRISDILNSVRRGSGTPEAEGPS
PVGPPPCPSPTIPGPLPTPWMDDIPGLLPPPPVGRHQTGGGVEILLEKRL
LGHGLGMAGQWLARKQEIIKTTEQLIEAVNNGDFEAYAKICDPGLTSFEP
EALGNLVEGMDFHRFYFENLLAKNSKPIHTTILNPHVHVIGEDAACIAYI
RLTQYIDGQGRPRTSQSEETRVWHRRDGKWQNVHFHCSGAPVAPLQ

| | | |
|---|---|---|
| NOV1j, | SEQ ID NO: 19 | 1727 bp |
| CG101025-05 | ORF Start: | ORF Stop: |
| DNA Sequence | ATG at 4 | TGA at 1630 |

GCCATGGCCACCACGGTGACCTGCACCCGCTTCACCGACGAGTACCAGCT
ATACGAGGATATTGGCAAGGGGGCTTTCTCTGTGGTCCGACGCTGTGTCA
AGCTCTGCACCGGCCATGAGTATGCAGCCAAGATCATCAACACCAAGAAG
CTGTCAGCCAGAGATCACCAGAAGCTGGAGAGAGAGGCTCGGATCTGCCG
CCTTCTGAAGCATTCCAACATCGTGCGTCTCCACGACAGCATCTCCGAGG
AGGGCTTCCACTACCTGGTCTTCGATCTGGTCACTGGTGGGGAGCTCTTT
GAAGACATTGTGGCGAGAGAGTACTACAGCGAGGCTGATGCCAGTCACTG
TATCCAGCAGATCCTGGAGGCCGTTCTCCATTGTCACCAAATGGGGGTCG
TCCACAGAGACCTCAAGCCGGAGAACCTGCTTCTGGCCAGCAAGTGCAAA
GGGGCTGCAGTGAAGCTGGCAGACTTCGGCCTAGCTATCGAGGTGCAGGG
GGACCAGCAGGCATGGTTTGGTTTCGCTGGCACACCAGGCTACCTGTCCC
CTGAGGTCCTTCGCAAAGAGGCGTATGGCAAGCCTGTGGACATCTGGGCA
TGTGGGGTGATCCTGTACATCCTGCTCGTGGGCTACCCACCCTTCTGGGA
CGAGGACCAGCACAAGCTGTACCAGCAGATCAAGGCTGGTGCCTATGACT
TCCCGTCCCCTGAGTGGGACACCGTCACTCCTGAAGCCAAAAACCTCATC
AACCAGATGCTGACCATCAACCCTGCCAAGCGCATCACAGCCCATGAGGC
CCTGAAGCACCCGTGGGTCTGCCAACGCTCCACGGTAGCATCCATGATGC
ACAGACAGGAGACTGTGGAGTGTCTGAAAAAGTTCAATGCCAGGAGAAAG
CTCAAGGGAGCCATCCTCACCACCATGCTGGCCACACGGAATTTCTCAGT
GGGCAGACAGACCACCGCTCCGGCCACAATGTCCACCGCGGCCTCCGGCA
CCACCATGGGGCTGGTGGAACAAGCCAAGAGTTTACTCAACAAGAAAGCA
GATGGAGTCAAGCCCCAGACGAATAGCACCAAAAACAGTGCAGCCGCCAC
CAGCCCCAAAGGGACGCTTCCTCCTGCCGCCCTGGAGCCTCAAACCACCG
TCATCCATAACCCAGTGGACGGGATTAAGGAGTCTTCTGACAGTGCCAAT
ACCACCATAGAGGATGAAGACGCTAAAGCCCGGAAGCAGGAGATCATTAA
GACCACGGAGCAGCTCATCGAGGCCGTCAACAACGGTGACTTTGAGGCCT
ACGCGAAAATCTGTGACCCAGGGCTGACCTCGTTTGAGCCTGAAGCACTG
GGCAACCTGGTTGAAGGGATGGACTTCCACAGATTCTACTTCGAGAACCT
GCTGGCCAAGAACAGCAAGCCAATCCACACGACCATCCTGAACCCACACG
TGCACGTCATTGGAGAGGATGTCGCCTGCACCGCTTACATCCGGCTCACG
CAGTACATTGACGGGCAGGGCCGGCCCCGCACCAGCCAGTCTGAGGAGAC
CCGCGTGTGGCACCGCCGCGACGGCAAGTGGCAGAACGTGCACTTCCACT
GCTCGGGCGCGCCTGTGGCCCCGCTGCAGTGAAGAGCTGCGCCCTGGTTT
CGCCGGACAGAGTTGGTGTTTGGAGCCCGACTGCCCTCGGGCACACGGCC
TGCCTGTCGCATGTTTGTGTCTGCCTC

| | | | |
|---|---|---|---|
| NOV1j, CG101025-05 | | | MW at |
| Protein Sequence | SEQ ID NO: 20 | 542 aa | 60402.2 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSVGRQTTAPATMSTAASGTTMGLVEQAKSLLNKKAD
GVKPQTNSTKNSAAATSPKGTLPPAALEPQTTVIHNPVDGIKESSDSANT
TIEDEDAKARKQEIIKTTEQLIEAVNNGDFEAYAKICDPGLTSFEPEALG
NLVEGMDFHRFYFENLLAKNSKPIHTTILNPHVHVIGEDVACTAYIRLTQ
YIDGQGRPRTSQSEETRVWHRRDGKWQNVHFHCSGAPVAPLQ

| | | |
|---|---|---|
| NOV1k, | SEQ ID NO: 21 | 1689 bp |
| CG101025-06 | ORF Start: | ORF Stop: |
| DNA Sequence | ATG at 2 | TGA at 1547 |

CATGGCCACCACGGTGACCTGCACCCGCTTCACCGACGAGTACCAGCTCT
ACGAGGATATTGGCAAGGGGGCTTTCTCTGTGGTCCGACGCTGTGTCAAG
CTCTGCACCGGCCATGAGTATGCAGCCAAGATCATCAACACCAAGAAGCT
GTCAGCCAGAGATCACCAGAAGCTGGAGAGAGAGGCTCGGATCTGCCGCC
TTCTGAAGCATTCCAACATCGTGCGTCTCCACGACAGCATCTCCGAGGAG
GGCTTCCACTACCTGGTCTTCGATCTGGTCACTGGTGGGGAGCTCTTTGA
AGACATTGTGGCGAGAGAGTACTACAGCGAGGCTGATGCCAGGGCCACTC
GCACTAACCCACCTGCTGTTTGCCACAGTCACTGTATCCAGCAGATCCTG
GAGGCCGTTCTCCATTGTCACCAAATGGGGGTCGTCCACAGAGACCTCAA
GCCGGAGAACCTGCTTCTGGCCAGCAAGTGCAAAGGGGCTGCAGTGAAGC
TGGCAGACTTCGGCCTAGCTATCGAGGTGCAGGGGGACCAGCAGGCATGG
TTTGGTTTCGCTGGCACACCAGGCTACCTGTCCCCTGAGGTCCTTCGCAA
AGAGGCGTACGGCAAGCCCGTGGACATCTGGGCATGTGGGGTGATCCTGT
ACATCCTGCTCGTGGGCTACCCACCCTTCTGGGACGAGGACCAGCACAAG
CTGTACCAGCAGATCAAGGCTGGTGCCTATGACTTCCCGTCCCCTGAGTG
GGACACCGTCACTCCTGAAGCCAAAAACCTCATCAACCAGATGCTGACCA
TCAACCCTGCCAAGCGCATCACAGCCCATGAGGCCCTGAAGCACCCGTGG
GTCTGCCAACGCTCCACGGTAGCATCCATGATGCACAGACAGGAGACTGT
GGAGTGTCTGAAAAAGTTCAATGCCAGGAGAAAGCTCAAGGGAGCCATCC
TCACCACCATGCTGGCCACACGGAATTTCTCAGCAGCCAAGAGTTTACTC
AACAAGAAAGCAGATGGAGTCAAGCCCCATACGAATAGCACCAAAAACAG
TGCAGCCGCCACCAGCCCCAAAGGGACGCTTCCTCCTGCCGCCCTGGAGT
CTTCTGACAGTGCCAATACCACCATAGAGGATGAAGACGCTAAAGCCCGG
AAGCAGGAGATCATTAAGACCACGGAGCAGCTCATCGAGGCCGTCAACAA
CGGTGACTTTGAGGCCTACGCGAAAATCTGTGACCCAGGGCTGACCTCGT
TTGAGCCTGAAGCACTGGGCAACCTGGTTGAAGGGATGGACTTCCACAGA
TTCTACTTCGAGAACCTGCTGGCCAAGAACAGCAAGCCGATCCACACGAC
CATCCTGAACCCACACGTGCACGTCATTGGAGAGGATGCCGCCTGCATCG
CTTACATCCGGCTCACGCAGTACATTGACGGGCAGGGCCGGCCCCGCACC
AGCCAGTCTGAGGAGACCCGCGTGTGGCACCGCCGCGACGGCAAGTGGCA
GAACGTGCACTTCCACTGCTCGGGCGCGCCTGTGGCCCCGCTGCAGTGAA
GAGCTGCGCCCTGGTTTCGCCGGACAGAGTTGGTGTTTGGAGCCCGACTG
CCCTCGGGCACACGGCCTGCCTGTCGCATGTTTGTGTCTGCCTCGTTCCC
TCCCCTGGTGCCTGTGTCTGCAGAAAAACAAGCCCGACT

| | | | |
|---|---|---|---|
| NOV1k, CG101025-06 | | | MW at |
| Protein Sequence | SEQ ID NO: 22 | 515 aa | 57692.2 kD |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADARATRTNPPAVCHSHCIQQILEAVLHCHQMGVVHRDLK
PENLLLASKCKGAAVKLADFGLAIEVQGDQQAWFGFAGTPGYLSPEVLRK
EAYGKPVDIWACGVILYILLVGYPPFWDEDQHKLYQQIKAGAYDFPSPEW
DTVTPEAKNLINQMLTINPAKRITAHEALKHPWVCQRSTVASMMHRQETV
ECLKKFNARRKLKGAILTTMLATRNFSAAKSLLNKKADGVKPHTNSTKNS
AAATSPKGTLPPAALESSDSANTTIEDEDAKARKQEIIKTTEQLIEAVNN
GDFEAYAKICDPGLTSFEPEALGNLVEGMDFHRFYFENLLAKNSKPIHTT
ILNPHVHVIGEDAACIAYIRLTQYIDGQGRPRTSQSEETRVWHRRDGKWQ
NVHFHCSGAPVAPLQ

| | | |
|---|---|---|
| NOV1l, | SEQ ID NO: 23 | 2239 bp |
| SNP13379569 of | ORF Start: | ORF Stop: |
| CG101025-01, | ATG at 25 | TGA at 2077 |
| DNA Sequence | SNP Pos: 1818 | SNP Change: G to A |

TCGCCGAGCCCGTCCGCCGCCGCCATGGCCACCACGGTGACCTGCACCCG
CTTCACCGACGAGTACCAGCTCTACGAGGATATTGGCAAGGGGGCTTTCT
CTGTGGTCCGACGCTGTGTCAAGCTCTGCACCGGCCATGAGTATGCAGCC
AAGATCATCAACACCAAGAAGCTGTCAGCCAGAGATCACCAGAAGCTGGA
GAGAGAGGCTCGGATCTGCCGCCTTCTGAAGCATTCCAACATCGTGCGTC
TCCACGACAGCATCTCCGAGGAGGGCTTCCACTACCTGGTCTTCGATCTG
GTCACTGGTGGGGAGCTCTTTGAAGACATTGTGGCGAGAGAGTACTACAG
CGAGGCTGATGCCAGTCACTGTATCCAGCAGATCCTGGAGGCCGTTCTCC
ATTGTCACCAAATGGGGGTCGTCCACAGAGACCTCAAGCCGGAGAACCTG
CTTCTGGCCAGCAAGTGCAAAGGGGCTGCAGTGAAGCTGGCAGACTTCGG
CCTAGCTATCGAGGTGCAGGGGGACCAGCAGGCATGGTTTGGTTTCGCTG
GCACACCAGGCTACCTGTCCCCTGAGGTCCTTCGCAAAGAGGCGTATGGC
AAGCCTGTGGACATCTGGGCATGTGGGGTGATCCTGTACATCCTGCTCGT
GGGCTACCCACCCTTCTGGGACGAGGACCAGCACAAGCTGTACCAGCAGA
TCAAGGCTGGTGCCTATGACTTCCCGTCCCCTGAGTGGGACACCGTCACT
CCTGAAGCCAAAAACCTCATCAACCAGATGCTGACCATCAACCCTGCCAA
GCGCATCACAGCCCATGAGGCCCTGAAGCACCCGTGGGTCTGCCAACGCT
CCACGGTAGCATCCATGATGCACAGACAGGAGACTGTGGAGTGTCTGAAA
AAGTTCAATGCCAGGAGAAAGCTCAAGGGAGCCATCCTCACCACCATGCT
GGCCACACGGAATTTCTCAGCCAAGAGTTTACTCAACAAGAAAGCAGATG
GAGTCAAGCCCCAGACGAATAGCACCAAAAACAGTGCAGCCGCCACCAGC
CCCAAAGGGACGCTTCCTCCTGCCGCCCTGGAGCCTCAAACCACCGTCAT
CCATAACCCAGTGGACGGGATTAAGGAGTCTTCTGACAGTGCCAATACCA
CCATAGAGGATGAAGACGCTAAAGCCCCCAGGGTCCCCGACATCCTGAGC
TCAGTGAGGAGGGGCTCGGGAGCCCCAGAAGCCGAGGGGCCCCTGCCCTG
CCCATCTCCGGCTCCCTTTAGCCCCCTGCCAGCCCCATCCCCCAGGATCT
CTGACATCCTGAACTCTGTGAGAAGGGGTTCAGGAACCCCAGAAGCCGAG
GGCCCCCTCTCAGCGGGGCCCCCGCCCTGCCTGTCTCCGGCTCTCCTAGG
CCCCCTGTCCTCCCCGTCCCCCAGGATCTCTGACATCCTGAACTCTGTGA
GGAGGGGCTCAGGGACCCCAGAAGCCGAGGGCCCCTCGCCAGTGGGGCCC
CCGCCCTGCCCATCTCCGACTATCCCTGGCCCCCTGCCCACCCCATGGAT
GGATGACATCCCAGGGCTGCTGCCACCCCCACCTGTGGGGAGACACCAGA
CTGGGGGTGGTGTGGAGATACTCTTAGAGAAGAGGCTGCTGGGCCACGGG
CTCGGCATGGCAGGGCAGTGGCTAGCCCGGAAGCAGGAGATCATTAAGAC
CACGGAGCAGCTCATCGAGGCCGTCAACAACGGTGACTTTGAGGCCTACG
CGAAAATCTGTGACCCAGGGCTGACCTCGTTTGAGCCTGAAGCACTGGGC
AACCTGGTTGAAGGGATAGACTTCCACAGATTCTACTTCGAGAACCTGCT
GGCCAAGAACAGCAAGCCGATCCACACGACCATCCTGAACCCACACGTGC
ACGTCATTGGAGAGGATGCCGCCTGCATCGCTTACATCCGGCTCACGCAG
TACATTGACGGGCAGGGCCGGCCCCGCACCAGCCAGTCTGAGGAGACCCG
CGTGTGGCACCGCCGCGACGGCAAGTGGCAGAATGTGCACTTCCACTGCT
CGGGCGCGCCTGTGGCCCCGCTGCAGTGAAGAGCTGCGCCCTGGTTTCGC
CGGACAGAGTTGGTGTTTGGAGCCCGACTGCCCTCGGGCACACGGCCTGC
CTGTCGCATGTTTGTGTCTGCCTCGTTCCCTCCCCTGGTGCCTGTGTCTG
CAGAAAAACAAGACCAGATGTGATTTGTTAAAAAAAAAA

| | | | |
|---|---|---|---|
| | | | MW at |
| NOV1l, SNP13379569 | | | 74789.5 kD |
| of CG101025-01, | SEQ ID NO: 24 | | SNP Change: |
| Protein Sequence | SNP Pos: 598 | 684 aa | Met to Ile |

MATTVTCTRFTDEYQLYEDIGKGAFSVVRRCVKLCTGHEYAAKIINTKKL
SARDHQKLEREARICRLLKHSNIVRLHDSISEEGFHYLVFDLVTGGELFE
DIVAREYYSEADASHCIQQILEAVLHCHQMGVVHRDLKPENLLLASKCKG
AAVKLADFGLAIEVQGDQQAWFGFAGTPGYLSPEVLRKEAYGKPVDIWAC
GVILYILLVGYPPFWDEDQHKLYQQIKAGAYDFPSPEWDTVTPEAKNLIN
QMLTINPAKRITAHEALKHPWVCQRSTVASMMHRQETVECLKKFNARRKL
KGAILTTMLATRNFSAKSLLNKKADGVKPQTNSTKNSAAATSPKGTLPPA
ALEPQTTVIHNPVDGIKESSDSANTTIEDEDAKAPRVPDILSSVRRGSGA
PEAEGPLPCPSPAPFSPLPAPSPRISDILNSVRRGSGTPEAEGPLSAGPP
PCLSPALLGPLSSPSPRISDILNSVRRGSGTPEAEGPSPVGPPPCPSPTI
PGPLPTPWMDDIPGLLPPPPVGRHQTGGGVEILLEKRLLGHGLGMAGQWL
ARKQEIIKTTEQLIEAVNNGDFEAYAKICDPGLTSFEPEALGNLVEGIDF
HRFYFENLLAKNSKPIHTTILNPHVHVIGEDAACIAYIRLTQYIDGQGRP
RTSQSEETRVWHRRDGKWQNVHFHCSGAPVAPLQ

A ClustalW comparison of the above protein sequences yields the following sequence alignment shown in Table 1B.

TABLE 1B
Comparison of the NOV1 protein sequences.

| | |
|---|---|
| NOV1a | ----MATTVTCTRFTDEYQLYEDIGKGAFSVVRRCVKLCTGHEYAAKIINTKKLSARDHQ |
| NOV1b | ----------------MATTVTCTRFTDEYQLYEDIGKGAFSVVRRCVKLCTGHEYAAKI |
| NOV1c | ------------TGSTMATTVTCTRFTDEYQLYEDIGKGAFSVVRRCVKLCTGHEYAAKI |
| NOV1d | TGSTMATTVTCTRFTDEYQLYEDIGKGAFSVVRRCVKLCTGHEYAAKIINTKKLSARDHQ |
| NOV1e | TGSTMATTVTCTRFTDEYQLYEDIGKGAFSVVRRCVKLCTGHEYAAKIINTKKLSARDHQ |
| NOV1f | TGSTMATTVTCTRFTDEYQLYEDIGKGAFSVVRRCVKLCTGHEYAAKIINTKKLSARDHQ |
| NOV1g | ------------------------------------------------------------ |
| NOV1h | ----MATTVTCTRFTDEYQLYEDIGKGAFSVVRRCVKLCTGHEYAAKIINTKKLSARDHQ |
| NOV1i | ----MATTVTCTRFTDEYQLYEDIGKGAFSVVRRCVKLCTGHEYAAKIINTKKLSARDHQ |
| NOV1j | ----------------MATTVTCTRFTDEYQLYEDIGKGAFSVVRRCVKLCTGHEYAAKI |
| NOV1k | ----MATTVTCTRFTDEYQLYEDIGKGAFSVVRRCVKLCTGHEYAAKIINTKKLSARDHQ |
| NOV1a | KLEREARICRLLKHSNIVRLHDSISEEGFHYLVFDLVTGGELFEDIVAREYYSEADA--- |
| NOV1b | INTKKLSARDHQKLEREARICRLLKHSNIVRLHDSISEEGFHYLVFDLVTGGELFEDIVA |
| NOV1c | INTKKLSARDHQKLEREARICRLLKHSNIVRLHDSISEEGFHYLVFDLVTGGELFEDIVA |
| NOV1d | KLEREARICRLLKHSNIVRLHDSISEEGFHYLVFDLVTGGELFEDIVAREYYSEADA--- |
| NOV1e | KLEREARICRLLKHSNIVRLHDSISEEGFHYLVFDLVTGGELFEDIVAREYYSEADA--- |
| NOV1f | KLEREARICRLLKHSNIVRLHDSISEEGFHYLVFDLVTGGELFEDIVAREYYSEADA--- |
| NOV1g | ------------------------------------------------------------ |
| NOV1h | KLEREARICRLLKHSNIVRLHDSISEEGFHYLVFDLVTGGELFEDIVAREYYSEADA--- |
| NOV1i | KLEREARICRLLKHSNIVRLHDSISEEGFHYLVFDLVTGGELFEDIVAREYYSEADARAT |
| NOV1j | INTKKLSARDHQKLEREARICRLLKHSNIVRLHDSISEEGFHYLVFDLVTGGELFEDIVA |
| NOV1k | KLEREARICRLLKHSNIVRLHDSISEEGFHYLVFDLVTGGELFEDIVAREYYSEADARAT |
| NOV1a | ---------SHCIQQILEAVLHCHQMGVVHRDLKPENLLLASKCKGAAVKLADFGLAIEV |
| NOV1b | REYYSEADASHCIQQILEAVLHCHQMGVVHRDLKPENLLLASKCKGAAVKLADFGLAIEV |
| NOV1c | REYYSEADASHCIQQILEAVLHCHQMGVVHRDLKPENLLLASKCKGAAVKLADFGLAIEV |
| NOV1d | ---------SHCIQQILEAVLHCHQMGVVHRDLKPENLLLASKCKGAAVKLADFGLAIEV |
| NOV1e | ---------SHCIQQILEAVLHCHQMGVVHRDLKPENLLLASKCKGAAVKLADFGLAIEV |
| NOV1f | ---------SHCIQQILEAVLHCHQMGVVHRDLKPENLLLASKCKGAAVKLADFGLAIEV |
| NOV1g | -------------------------MGVVHRDLKPENLLLASKCKGAAVKLADFGLAIEV |
| NOV1h | ---------SHCIQQILEAVLHCHQMGVVHRDLKPENLLLASKCKGAAVKLADFGLAIEV |
| NOV1i | RTNPPAVCHSHCIQQILEAVLHCHQMGVVHRDLKPENLLLASKCKGAAVKLADFGLAIEV |
| NOV1j | REYYSEADASHCIQQILEAVLHCHQMGVVHRDLKPENLLLASKCKGAAVKLADFGLAIEV |
| NOV1k | RTNPPAVCHSHCIQQILEAVLHCHQMGVVHRDLKPENLLLASKCKGAAVKLADFGLAIEV |
| NOV1a | QGDQQAWFGFAGTPGYLSPEVLRKEAYGKPVDIWACGVILYILLVGYPPFWDEDQHKLYQ |
| NOV1b | QGDQQAWFGFAGTPGYLSPEVLRKEAYGKPVDIWACGVILYILLVGYPPFWDEDQHKLYQ |
| NOV1c | QGDQQAWFGFAGTPGYLSPEVLRKEAYGKPVDIWACGVILYILLVGYPPFWDEDQHKLYQ |
| NOV1d | QGDQQAWFGFAGTPGYLSPEVLRKEAYGKPVDIWACGVILYILLVGYPPFWDEDQHKLYQ |
| NOV1e | QGDQQAWFGFAGTPGYLSPEVLRKEAYGKPVDIWACGVILYILLVGYPPFWDEDQHKLYQ |
| NOV1f | QGDQQAWFGFAGTPGYLSPEVLRKEAYGKPVDIWACGVILYILLVGYPPFWDEDQHKLYQ |
| NOV1g | QGDQQAWFGFAGTPGYLSPEVLRKEAYGKPVDIWACGVILYILLVGYPPFWDEDQHKLYQ |
| NOV1h | QGDQQAWFGFAGTPGYLSPEVLRKEAYGKPVDIWACGVILYILLVGYPPFWDEDQHKLYQ |
| NOV1i | QGDQQAWFGFAGTPGYLSPEVLRKEAYGKPVDIWACGVILYILLVGYPPFWDEDQHKLYQ |
| NOV1j | QGDQQAWFGFAGTPGYLSPEVLRKEAYGKPVDIWACGVILYILLVGYPPFWDEDQHKLYQ |
| NOV1k | QGDQQAWFGFAGTPGYLSPEVLRKEAYGKPVDIWACGVILYILLVGYPPFWDEDQHKLYQ |
| NOV1a | QIKAGAYDFPSPEWDTVTPEAKNLINQMLTINPAKRITAHEALKMPWVCQRSTVASMMHR |
| NOV1b | QIKAGAYDFPSPEWDTVTPEAKNLINQMLTINPAKRITAHEALKHPWVCQRSTVASMMHR |
| NOV1c | QIKAGAYDFPSPEWDTVTPEAKNLINQMLTINPAKRITAHEALKHPWVCQRSTVASMMHR |
| NOV1d | QIKAGAYDFPSPEWDTVTPEAKNLINQMLTINPAKRITAHEALKHPWVCQRSTVASMMHR |
| NOV1e | QIKAGAYDFPSPEWDTVTPEAKNLINQMLTINPAKRITAHEALKHPWVCQRSTVASMMHR |
| NOV1f | QIKAGAYDFPSPEWDTVTPEAKNLINQMLTINPAKRITAHEALKHPWVCQRSTVASLEG- |
| NOV1g | QIKAGAYDFPSPEWDTVTPEAKNLINQMLTINPAKRITAHEALKHPWVCQRSTVASMMHR |
| NOV1h | QIKAGAYDFPSPEWDTVTPEAKNLINQMLTINPAKRITAHEALKHPWVCQRSTVASMMHR |
| NOV1i | QIKAGAYDFPSPEWDTVTPEAKNLINQMLTINPAKRITAHEALKHPWVCQRSTVASMMHR |
| NOV1j | QIKAGAYDFPSPEWDTVTPEAKNLINQMLTINPAKRITAHEALKHPWVCQRSTVASMMHR |
| NOV1k | QIKAGAYDFPSPEWDTVTPEAKNLINQMLTINPAKRITAHEALKHPWVCQRSTVASMMHR |
| NOV1a | QETVECLKKFNARRKLKGAILTTMLATRNFS-------------------------AKSL |
| NOV1b | QETVECLKKFNARRKLKGAILTTMLATRNFSVGRQTTAPATMSTAASGTTMGLVEQAKSL |
| NOV1c | QETVECLKKFNARRKLKGAILTTMLATRNFSVGRQTTAPATMSTAASGTTMGLVEQAKSL |
| NOV1d | QETVECLKKFNARRKLKGAILTTMLATRNFS-------------------------AKSL |
| NOV1e | QETVECLKKFNARRKLKGAILTTMLATRNFS-------------------------AKSL |
| NOV1f | ------------------------------------------------------------ |
| NOV1g | QETVECLKKFNARRKLKGAILTTMLATRNFSVGRQTTAPATMSTAASGTTMGLVEQAKSL |
| NOV1h | QETVECLKKFNARRKLKGAILTTMLATRNFSA------------------------AKSL |
| NOV1i | QETVECLKKFNARRKLKGAILTTMLATRNFS-------------------------AKSL |
| NOV1j | QETVECLKKFNARRKLKGAILTTMLATRNFSVGRQTTAPATMSTAASGTTMGLVEQAKSL |
| NOV1k | QETVECLKKFNARRKLKGAILTTMLATRNFSA------------------------AKSL |
| NOV1a | LNKKADGVKPQTNSTKNSAAATSPKGTLPPAALEPQTTVIHNPVDGIKESSDSANTTIED |
| NOV1b | LNKKADGVKPQTNSTKNSAAATSPKGTLPPAALEPQTTVIHNPVDGIKESSDSANTTIED |
| NOV1c | LNKKADGVKPQTNSTKNSAAATSPKGTLPPAALEPQTTVIHNPVDGIKESSDSANTTIED |
| NOV1d | LNKKADGVKPQTNSTKNSAAATSPKGTLPPAALEPQTTVIHNPVDGIKESSDSANTTIED |
| NOV1e | LNKKADGVKPQTNSTKNSAAATSPKGTLPPAALEPQTTVIHNPVDGIKESSDSANTTIED |
| NOV1f | ------------------------------------------------------------ |
| NOV1g | LNKKADGVKPQTNSTKNSAAATSPKGTLPPAALEPQTTVIHNPVDGIKESSDSANTTIED |
| NOV1h | LNKKADGVKPHTNSTKNSAAATSPKGTLPPAALE---------------SSDSANTTIED |
| NOV1i | LNKKADGVKPQTNSTKNSAAATSPKGTLPPAALEPQTTVIHNPVDGIKESSDSANTTIED |
| NOV1j | LNKKADGVKPQTNSTKNSAAATSPKGTLPPAALEPQTTVIHNPVDGIKESSDSANTTIED |
| NOV1k | LNKKADGVKPHTNSTKNSAAATSPKGTLPPAALE---------------SSDSANTTIED |
| NOV1a | EDAKAPRVPDILSSVRRGSGAPEAEGPLPCPSPAPFSPLPAPSPRISDILNSVRRGSGTP |
| NOV1b | EDAKAPRVPDILSSVRRGSGAPEAEGPLPCPSPAPFSPLPAPSPRISDILNSVRRGSGTP |
| NOV1c | EDAKAPRVPDILSSVRRGSGAPEAEGPLPCPSPAPFSPLPAPSPRISDILNSVRRGSGTP |
| NOV1d | EDAKAPRISDILNSVRRGSG--------------------------------------TP |
| NOV1e | EDAKAPRVPDILSSVRRGSGAPEAEGPLPCPSPAPFSPLPAPSPRISDILNSVRRGSGTP |
| NOV1f | ------------------------------------------------------------ |
| NOV1g | EDAKAR------------------------------------------------------ |
| NOV1h | EDAKAR------------------------------------------------------ |
| NOV1i | EDAKAPRVPDILSSVRRGSGAPEAEGPLPCPSPAPFSPLPAPSPRISDILNSVRRGSGTP |
| NOV1j | EDAKAR------------------------------------------------------ |
| NOV1k | EDAKAR------------------------------------------------------ |
| NOV1a | EAEGPLSAGPPPCLSPALLGPLSSPSPRISDILNSVRRGSGTPEAEGPSPVGPPPCPSPT |
| NOV1b | EAEGPLSAGPPPCLSPALLGPLSSPSPRISDILNSVRRGSGTPEAEGPSPVGPPPCPSPT |
| NOV1c | EAEGPLSAGPPPCLSPALLGPLSSPSPRISDILNSVRRGSGTPEAEGPSPVGPPPCPSPT |
| NOV1d | EAEGPLSAGPPPCLSPALLGPLSSPSPRISDILNSVRRGSGTPEAEGPSPVGPPPCPSPT |
| NOV1e | EAEGPLSAGPPPCLSPALLGPLSSPSPRISDILNSVRRGSGTPEAEGPSPVGPPPCPSPT |
| NOV1f | ------------------------------------------------------------ |
| NOV1g | ------------------------------------------------------------ |
| NOV1h | ------------------------------------------------------------ |
| NOV1i | EAEGPLSAGPPPCLSPALLGPLSSPSPRISDILNSVRRGSGTPEAEGPSPVGPPPCPSPT |
| NOV1j | ------------------------------------------------------------ |
| NOV1k | ------------------------------------------------------------ |
| NOV1a | IPGPLPTPWMDDIPGLLPPPPVGRHQTGGGVEILLEKRLLGHGLGMAGQWLARKQEIIKT |
| NOV1b | IPGPLPTPSR-------------------------------------------KQEIIKT |
| NOV1c | IPGPLPTPSR-------------------------------------------KQEIIKT |
| NOV1d | IPGPLPTP-------------------------------------------SRKQEIIKT |
| NOV1e | IPGPLPTP-------------------------------------------SRKQEIIKT |
| NOV1f | ------------------------------------------------------------ |
| NOV1g | -----------------------------------------------------KQEIIKT |
| NOV1h | -----------------------------------------------------KQEIIKT |
| NOV1i | IPGPLPTPWMDDIPGLLPPPPVGRHQTGGGVEILLEKRLLGHGLGMAGQWLARKQEIIKT |
| NOV1j | -----------------------------------------------------KQEIIKT |
| NOV1k | -----------------------------------------------------KQEIIKT |
| NOV1a | TEQLIEAVNNGDFEAYAKICDPGLTSFEPEALGNLVEGMDFHRFYFENLLAKNSKPIHTT |
| NOV1b | TEQLIEAVNNGDFEAYAKICDPGLTSFEPEALGNLVEGMDFHRFYFENLLAKNSKPIHTT |
| NOV1c | TEQLIEAVNNGDFEAYAKICDPGLTSFEPEALGNLVEGMDFNRFYFENLLAKNSKPIHTT |
| NOV1d | TEQLIEAVNNGDFEAYAKICDPGLTSFEPEALGNLVEGMDFHRFYFENLLAKNSKPIHTT |
| NOV1e | TEQLIEAVNNGDFEAYAKICDPGLTSFEPEALGNLVEGMDFHRFYFENLLAKNSKPIHTT |
| NOV1f | ------------------------------------------------------------ |
| NOV1g | TEQLIEAVNNGDFEAYAKICDPGLTSFEPEALGNLVEGMDFHRFYFENLLAKNSKPIHTT |
| NOV1h | TEQLIEAVNNGDFEAYAKICDPGLTSFEPEALGNLVEGMDFHRFYFENLLAKNSKPIHTT |
| NOV1i | TEQLIEAVNNGDFEAYAKICDPGLTSFEPEALGNLVEGMDFHRFYFENLLAKNSKPIHTT |
| NOV1j | TEQLIEAVNNGDFEAYAKICDPGLTSFEPEALGNLVEGMDFHRFYFENLLAKNSKPIHTT |
| NOV1k | TEQLIEAVNNGDFEAYAKICDPGLTSFEPEALGNLVEGMDFHRFYFENLLAKNSKPIHTT |
| NOV1a | ILNPHVHVIGEDAACIAYIRLTQYIDGQGRPRTSQSEETRVWHRRDGKWQNVHFHCSGAP |
| NOV1b | ILNPHVHVIGEDAACIAYIRLTQYIDGQGRPRTSQSEETRVWHRRDGKWQNVHFHCSGAP |
| NOV1c | ILNPHVHVIGEDAACIAYIRLTQYIDGQGRPRTSQSEETRVWHRRDGKWQNVHFHCSGAP |
| NOV1d | ILNPHVHVIGEDAACIAYIRLTQYIDGQGRPRTSQSEETRVWHRRDGKWQNVHFHCSGAP |
| NOV1e | ILNPHVHVIGEDAACIAYIRLTQYIDGQGRPRTSQSEETRVWHRRDGKWQNVHFHCSGAP |
| NOV1f | ------------------------------------------------------------ |
| NOV1g | ILNPHVHVIGEDAACIAYIRLTQYIDGQGRPRTSQSEETRVWHRRDGKWQNVHFHCSGAP |
| NOV1h | ILNPHVHVIGEDAACIAYIRLTQYIDGQGRPRTSQSEETRVWHRRDGKWQNVHFHCSGAP |
| NOV1i | ILNPHVHVIGEDAACIAYIRLTQYIDGQGRPRTSQSEETRVWHRRDGKWQNVHFHCSGAP |
| NOV1j | ILNPHVHVIGEDVACTAYIRLTQYIDGQGRPRTSQSEETRVWHRRDGKWQNVHFHCSGAP |
| NOV1k | ILNPHVHVIGEDAACIAYIRLTQYIDGQGRPRTSQSEETRVWHRRDGKWQNVHFHCSGAP |
| NOV1a | VAPLQ--- |
| NOV1b | VAPLQ--- |
| NOV1c | VAPLQLEG |
| NOV1d | VAPLQLEG |
| NOV1e | VAPLQLEG |
| NOV1f | -------- |
| NOV1g | VAPLQ--- |
| NOV1h | VAPLQ--- |
| NOV1i | VAPLQ--- |
| NOV1j | VAPLQ--- |
| NOV1k | VAPLQ--- |
| NOV1a | (SEQ ID NO: 2) |
| NOV1b | (SEQ ID NO: 4) |
| NOV1c | (SEQ ID NO: 6) |
| NOV1d | (SEQ ID NO: 8) |
| NOV1e | (SEQ ID NO: 10) |
| NOV1f | (SEQ ID NO: 12) |
| NOV1g | (SEQ ID NO: 14) |
| NOV1h | (SEQ ID NO: 16) |
| NOV1i | (SEQ ID NO: 18) |
| NOV1j | (SEQ ID NO: 20) |
| NOV1k | (SEQ ID NO: 22) |

Further analysis of the NOV1a protein yielded the following properties shown in Table 1C.

TABLE 1C
Protein Sequence Properties NOV1a

| SignalP | No Known Signal Sequence Predicted | | | |
|---|---|---|---|---|
| analysis: | | | | |
| PSORT II | PSG: a new signal peptide prediction method | | | |
| analysis: | N-region: length 9; pos. chg 1; neg. chg 0 | | | |
| | H-region: length 2; peak value −7.95 | | | |
| | PSG score: −12.35 | | | |
| | GvH: von Heijne's method for signal seq. recognition | | | |
| | GvH score (threshold: −2.1): −8.96 | | | |
| | possible cleavage site: between 37 and 38 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −3.77 | Transmembrane 195-211 |
| | PERIPHERAL | Likelihood = | 3.45 (at 88) | |
| | ALOM score: −3.77 (number of TMSs: 1) | | | |
| | MTOP: Prediction of membrane topology (Hartmann et al.) | | | |
| | Center position for calculation: 202 | | | |
| | Charge difference: −0.5 C(−0.5)-N(0.0) | | | |
| | N >= C: N-terminal side will be inside | | | |
| | >>> membrane topology: type 2 (cytoplasmic tail 1 to 195) | | | |
| | MITDISC: discrimination of mitochondrial targeting seq | | | |
| | R content: | 1 | Hyd Moment (75): | 1.90 |
| | Hyd Moment (95): | 1.72 | G content: | 0 |
| | D/E content: | 2 | S/T content: | 5 |

```
Score:−5.72
Gavel: prediction of cleavage sites for mitochondrial preseq
R-2 motif at 19 TRF|TD
NUCDISC: discrimination of nuclear localization signals
pat4: none
pat7: none
bipartite: none
content of basic residues: 10.7%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 67
LL at 142
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
34.8%: nuclear
30.4%: mitochondrial
  8.7%: cytoplasmic
  4.3%: Golgi
  4.3%: plasma membrane
  4.3%: vesicles of secretory system
  4.3%: extracellular, including cell wall
  4.3%: endoplasmic reticulum
  4.3%: peroxisomal
>> prediction for CG101025-01 is nuc (k = 23)
```

A search of the NOV1a protein against the Geneseq database, a proprietary database that contains sequences published in patents and patent publication, yielded several homologous proteins shown in Table 1D.

TABLE 1D
Geneseq Results for NOV1a

| | | NOV1a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY68791 | Amino acid sequence of a human | 1 . . . 684 | 638/684 (93%) | 0.0 |
| | phosphorylation effector PHSP-23 - | 1 . . . 641 | 640/684 (93%) | |
| | Homo sapiens, 641 aa. | | | |
| | [WO200006728-A2, 10 FEB. 2000] | | | |
| ABP70158 | Amino acid sequence of oestrogen | 1 . . . 417 | 392/447 (87%) | 0.0 |
| | receptor alpha cofactor CF19 - Homo | 1 . . . 446 | 398/447 (88%) | |
| | sapiens, 542 aa. [WO200270699-A2, | | | |
| | 12 SEP. 2002] | | | |
| AAE30198 | Human kinase protein - Homo sapiens, | 1 . . . 384 | 384/409 (93%) | 0.0 |
| | 516 aa. [WO200279431-A2, | 1 . . . 409 | 384/409 (93%) | |
| | 10 OCT. 2002] | | | |
| AAY68786 | Amino acid sequence of a human | 1 . . . 417 | 376/423 (88%) | 0.0 |
| | phosphorylation effector PHSP-18 - | 1 . . . 407 | 382/423 (89%) | |
| | Homo sapiens, 503 aa. | | | |
| | [WO200006728-A2, 10 FEB. 2000] | | | |
| AAM25814 | Human protein sequence SEQ ID | 1 . . . 383 | 330/385 (85%) | 0.0 |
| | NO: 1329 - Homo sapiens, 525 aa. | 8 . . . 391 | 352/385 (90%) | |
| | [WO200153455-A2, 26 JUL. 2001] | | | |

In a BLAST search of public sequence databases, the NOV1a protein was found to have homology to the proteins shown in the BLASTP data in Table 1E.

TABLE 1E
Public BLASTP Results for NOV1a

| | | NOV1a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q13554 | Calcium/calmodulin-dependent protein | 1 . . . 684 | 628/709 (88%) | 0.0 |
| | kinase type II beta chain (EC 2.7.1.123) | 1 . . . 664 | 630/709 (88%) | |
| | (CaM-kinase II beta chain) (CaM kinase | | | |
| | II beta subunit) (CaMK-II beta subunit) - | | | |
| | Homo sapiens (Human), 664 aa. | | | |
| Q63094 | Calcium/calmodulin-dependent protein | 1 . . . 684 | 574/685 (83%) | 0.0 |
| | kinase II, beta 3 isoform - Rattus | 1 . . . 589 | 579/685 (83%) | |
| | norvegicus (Rat), 589 aa. | | | |
| P08413 | Calcium/calmodulin-dependent protein | 1 . . . 417 | 388/447 (86%) | 0.0 |
| | kinase type II beta chain (EC 2.7.1.123) | 1 . . . 446 | 396/447 (87%) | |
| | (CaM-kinase II beta chain) (CaM kinase | | | |
| | II beta subunit) (CaMK-II beta subunit) - | | | |
| | Rattus norvegicus (Rat), 542 aa. | | | |
| P28652 | Calcium/calmodulin-dependent protein | 1 . . . 417 | 387/447 (86%) | 0.0 |
| | kinase type II beta chain (EC 2.7.1.123) | 1 . . . 446 | 396/447 (88%) | |
| | (CaM-kinase II beta chain) (CaM kinase | | | |
| | II beta subunit) (CaMK-II beta subunit) - | | | |
| | Mus musculus (Mouse), 542 aa. | | | |
| Q8BL41 | Calcium/calmodulin-dependent protein | 1 . . . 417 | 385/447 (86%) | 0.0 |
| | kinase type II beta chain - Mus musculus | 1 . . . 446 | 394/447 (88%) | |
| | (Mouse), 542 aa. | | | |

PFam analysis predicts that the NOV1a protein contains the domains shown in the Table 1F.

TABLE 1F
Domain Analysis of NOV1a

| | | Identities/ | |
|---|---|---|---|
| Pfam | NOV1a Match | Similarities for | Expect |
| Domain | Region | the Matched Region | Value |
| pkinase | 14 . . . 272 | 95/300 (32%) | 1.9e−89 |
| | | 209/300 (70%) | |

### Example 2

The NOV2 clone was analyzed, and the nucleotide and encoded polypeptide sequences are shown in Table 2A.

TABLE 2A
NOV2 Sequence Analysis

| | | |
|---|---|---|
| NOV2a, | SEQ ID NO: 25 | 3309 bp |
| CG101826-02 | ORF Start: | ORF Stop: |
| DNA Sequence | ATG at 295 | TGA at 1984 |

GCGGCCGCGCTGCCTGGCAGCCCGGGAAGCCGCGGCACAGCTGCTCGGCG
CCTGCAGCTCCGGCTCGGGGGCTGGAACCGAAGCGGGGGCGGCGGGAGCG
CGGAGACCACAGCCCCCGGGGAGAGGCGGAGGGGGTCCCTGGCCTGGGCG
GAGAGGCTGAGCTGAGTGCGCGTGAGAAAGAGGGCTGCACCGCTGCTCGG
CGCGGACTCTGCCAGCCCCAGCTTCAGCCCCGGCTCAGGTCGCCGCAGCC
CGGGAGCCTCCCCGCTTGCGCCCCAAGGCACGCGCGGCACAGCCATGAAC
ACCAACGATGCCAAGGAGTATCTGGCCCGGAGGGAAATCCCTCAGCTTTT
TGAGAGCCTTTTGAATGGACTGATGTGTTCTAAGCCCGAAGATCCAGTAG
AATACTTGGAAAGTTGTTTACAAAAAGTAAAGGAACTGGGTGGCTGTGAC
AAGGTGAAATGGGATACATTTGTAAGCCAGGAAAAGAAGACCTTACCTCC
ACTAAATGGAGGACAGTCACGGAGATCCTTTCTAAGAAATGTAATGCCTG
AAAACTCAAACTTTCCATATCGGCGGTATGACCGGCTCCCTCCAATCCAT
CAATTCTCCATAGAAAGTGACACGGATCTCTCTGAGACTGCAGAGTTGAT
TGAGGAGTATGAGGTTTTTGATCCTACCAGACCTCGACCAAAAATCATTC
TTGTTATAGGTGGTCCAGGAAGTGGAAAGGGTACTCAGAGTTTGAAAATT
GCAGAACGATATGGATTCCAATACATTTCTGTGGGAGAATTATTAAGAAA
GAAGATCCACAGTACCAGCAGCAATAGGAAATGGAGTCTTATTGCCAAGA
TAATTACAACTGGAGAATTGGCCCCACAGGAAACAACAATTACAGAGATA
AAACAAAAATTGATGCAAATACCTGATGAAGAGGGCATTGTTATTGATGG
ATTTCCAAGAGATGTTGCCCAGGCTCTATCTTTTGAGGACCAAATCTGTA
CCCCCGATTTTGTGGTATTCCTGGCTTGTGCTAATCAGAGACTCAAAGAA
AGATTACTGAAGCGTGCAGAACAGCAGGGCCGACCAGACGACAATGTAAA
AGCTACCCAAAGGAGACTAATGAACTTCAAGCAGAATGCTGCTCCATTGG
TTAAATACTTCCAGGAAAAGGGGCTCATCATGACATTTGATGCCGACCGC
GATGAGGATGAGGTGTTCTATGACATCAGCATGGCAGTTGACAACAAGTT
ATTTCCAAACAAAGAGGCTGCAGCAGGTTCAAGTGACCTTGATCCTTCGA
TGATATTGGACACTGGAGAGATCATTGATACAGGATCTGATTATGAAGAT
CAGGGTGATGACCAGTTAAATGTATTTGGAGAGGACACTATGGGAGGTTT
CATGGAAGATTTGAGAAAGTGTAAAATTATTTTCATAATTGGTGGTCCTG
GCTCTGGCAAAGGCACACAGTGTGAAAAGCTGGTGGAAAAATATGGATTT
ACACATCTCTCAACTGGCGAGCTCCTGCGTGAGGAACTGGCATCAGAATC
TGAAAGAAGCAAATTGATCAGAGACATTATGGAACGTGGAGACCTGGTGC
CCTCAGGCATCGTTTTGGAGCTCCTGAAGGAGGCCATGGTGGCCAGCCTC
GGGGACACCAGGGGCTTCCTGATTGACGGCTATCCTCGGGAGGTGAAGCA
AGGGGAAGAGTTCGGACGCAGGATTGGAGACCCACAGTTGGTGATCTGTA
TGGACTGCTCGGCAGACACCATGACCAACCGCCTTCTCCAAAGGAGCCGG
AGCAGCCTGCCTGTGGACGACACCACCAAGACCATCGCCAAGCGCCTAGA
AGCCTACTACCGAGCGTCCATCCCCGTGATCGCCTACTACGAGACAAAAA
CACAGCTACACAAGATAAATGCAGAGGGAACACCAGAGGACGTTTTTCTT
CAACTCTGCACAGCTATTGACTCTATTATTTTCTGAAGGCAAAAATGCAT
GTTTGTTAGAATGGAAACAGAAAAACATTAAAAAGTTCATTCCTTAACAC
AATGTTTCAAGTTAAACCTTTTGTGTCACCGCCCCCCACCAACCACCACC
TCCTAAATCCTGACAGCACTGTTTGCTTCCCAGCTAGACCTGTGTGAGAG
GTGTCTGGAAATCATGCATGGTGTATTTGGGACTATATCAACCTATTCTC
CACACTTCAGACAACTGTCTGCACTCACGGCACGCACACTTTGTATCATG
CAGGCCACACTCAGAGCTAGTCAGTACATGAACAGTGGTGCGGTGCCAGT
CTGTGTCCGTTGTGATCACAGGCCTTGCTAGACCCTGATCATCTGGTTCT
CCTCTCATTAAGCATCCCTAACCCCCAGTCACACCTTCCTCTTACATACT
GTTCCCCAATGGAGGCCCCTGGCATAGGGGACAGCCCTGGGCATCTTCCT
TTGGTGTCTGGCTGTTTTGTCAACTCTCATCCACTGGTGGCTCAGAGCCA
TAAGGTGGGTTGATTACACAATGCCTTGTACATGATATAGAGGCATCAAG
CAAGTAAAATTTGACAGAAATTTTAAAATATGAAGATGTATAGCTTTCCC
AAGATGATGGTAAAACCCAGGTTAGTCATCAGTAACCTTCTCTATTATTA
TTATTTTTTAGAAACTTGGAATACTGTCATTATGGCTAAGAGAACAAATC
TGATAAATTGTGTAACCTAGTCTCTTCTCTACATGGTGATGCATTTCAGC
AATTATAAATTAATATAAATGACCAAAAGTAACTTAAAAGCATGAGATAT
TTGCTATTTCATTCATTGGGCACATATCAAATTATAATTTTGATTTTAAA
TGGTCACCCATGTATTTGTTGCCAAGCAAGTAAAAAAATACCCTAACAAA
CCTGATGTGGGTGGGAGGGGCATGTCAGTAAGTGGTGTGTTCAATGTGTT
TGTTTCATATGGGCCCTTTCCAGGAGTTTGCAAACCTTGTCATACCCATA
TGCAAAACTGTGTTTCCTTGCATTAAACCAGTGAAGTTTGGGTTCTCTTT
TGTGCTATCAATCAGTTGTAAAATCAGAGCTTCTTATATATTCTACTGGA
ATAACTGCATCTTCCACTCAGTCACTACAAAAAAGCATAGTTTCAGTTTG
CATGAATTTTTTTTTTTTTCTTCAATGGTTGTGCAGATAAGGATCCATTT
CTGGGATAGAATTGTATTTTTTAAGTCATTTTTTTTTCTTGAAATGGATA
TGTACAAATAAAATTAAATGGAAGACAGGAAAAAAAAAAAAAAAAAAAAA
AAAAAAAAA

| | | | |
|---|---|---|---|
| NOV2a, CG101826-02 | | | MW at |
| Protein Sequence | SEQ ID NO: 26 | 563 aa | 63479.4 kD |

MNTNDAKEYLARREIPQLFESLLNGLMCSKPEDPVEYLESCLQKVKELGG
CDKVKWDTFVSQEKKTLPPLNGGQSRRSFLRNVMPENSNFPYRRYDRLPP
IHQFSIESDTDLSETAELIEEYEVFDPTRPRPKIILVIGGPGSGKGTQSL
KIAERYGFQYISVGELLRKKIHSTSSNRKWSLIAKIITTGELAPQETTIT
EIKQKLMQIPDEEGIVIDGFPRDVAQALSFEDQICTPDFVVFLACANQRL
KERLLKRAEQQGRPDDNVKATQRRLMNFKQNAAPLVKYFQEKGLIMTFDA
DRDEDEVFYDISMAVDNKLFPNKEAAAGSSDLDPSMILDTGEIIDTGSDY
EDQGDDQLNVFGEDTMGGFMEDLRKCKIIFIIGGPGSGKGTQCEKLVEKY
GFTHLSTGELLREELASESERSKLIRDIMERGDLVPSGIVLELLKEANVA
SLGDTRGFLIDGYPREVKQGEEFGRRIGDPQLVICMDCSADTMTNRLLQR
SRSSLPVDDTTKTIAKRLEAYYRASIPVIAYYETKTQLHKINAEGTPEDV
FLQLCTAIDSIIF

| | | |
|---|---|---|
| NOV2b, | SEQ ID NO: 27 | 490 bp |
| 308782075 | ORF Start: | ORF Stop: |
| DNA Sequence | at 2 | end of sequence |

CACCAGATCTATAATTGGTGGTCCTGGCTCTGGCAAAGGCACACAGTGTG
AAAAGCTGGTGGAAAAATATGGATTTACACATCTCTCAACTGGCGAGCTC
CTGCGTGAGGAACTGGCATCAGAATCTGAAAGAAGCAAATTGATCAGAGA
CATTATGGAACGTGGAGACCTGGTGCCCTCAGGCATCGTTTTGGAGCTCC
TGAAGGAGGCCATGGTGGCCAGCCTCGGGGACACCAGGGGCTTCCTGATT
GACGGCTATCCTCGGGAGGTGAAGCAAGGGGAAGAGTTCGGACGCAGGAT
TGGAGACCCACAGTTGGTGATCTGTATGGACTGCTCGGCAGACACCATGA
CCAACCGCCTTCTCCAAAGGAGCCGGAGCAGCCTGCCTGTGGACGACACC
ACCAAGACCATCGCCAAGCGCCTAGAAGCCTACTACCGAGCGTCCATCCC
CGTGATCGCCTACTACGAGACAAAAACACAGGTCGACGGC

| | | | |
|---|---|---|---|
| NOV2b, 308782075 | | | MW at |
| Protein Sequence | SEQ ID NO: 28 | 163 aa | 18062.4 kD |

TRSIIGGPGSGKGTQCEKLVEKYGFTHLSTGELLREELASESERSKLIRD
IMERGDLVPSGIVLELLKEAMVASLGDTRGFLIDGYPREVKQGEEFGRRI
GDPQLVICMDCSADTMTNRLLQRSRSSLPVDDTTKTIAKRLEAYYRASIP
VIAYYETKTQVDG

| | | |
|---|---|---|
| NOV2c, | SEQ ID NO: 29 | 493 bp |
| 308782087 | ORF Start: | ORF Stop: |
| DNA Sequence | at 2 | end of sequence |

CACCAGATCTGTTATAGGTGGTCCAGGAAGTGGAAAGGGTACTCAGAGTT
TGAAAATTGCAGAACGATATGGATTCCAATACATTTCTGTGGGAGAATTA
TTAAGAAAGAAGATCCACAGTACCAGCAGCAATAGGAAATGGAGTCTTAT
TGCCAAGATAATTACAACTGGAGAATTGGCCCCACAGGAAACAACAATTA
CAGAGATAAAACAAAAATTGATGCAAATACCTGATGAAGAGGGCATTGTT
ATTGATGGATTTCCAAGAGATGTTGCCCAGGCTCTATCTTTTGAGGACCA
AATCTGTACCCCCGATTTGGTGGTATTCCTGGCTTGTGCTAATCAGAGAC
TCAAAGAAAGATTACTGAAGCGTGCAGAACAGCAGGGCCGACCAGACGAC
AATGTAAAAGCTACCCAAAGGAGACTAATGAACTTCAAGCAGAATGCTGC
TCCATTGGTTAAATACTTCCAGGAAAAGGGGCTCGTCGACGGC

| | | | |
|---|---|---|---|
| NOV2c, 308782087 | | | MW at |
| Protein Sequence | SEQ ID NO: 30 | 164 aa | 18330.9 kD |

TRSVIGGPGSGKGTQSLKIAERYGFQYISVGELLRKKIHSTSSNRKWSLI
AKIITTGELAPQETTITEIKQKLMQIPDEEGIVIDGFPRDVAQALSFEDQ
ICTPDLVVFLACANQRLKERLLKRAEQQGRPDDNVKATQRRLMNFKQNAA
PLVKYFQEKGLVDG

| | | |
|---|---|---|
| NOV2d, | SEQ ID NO: 31 | 1714 bp |
| 309326609 | ORF Start: | ORF Stop: |
| DNA Sequence | at 2 | end of sequence |

CACCAGATCTCCCACCATGAACACCAACGATGCCAAGGAGTATCTGGCCC
GGAGGGAAATCCCTCAGCTTTTTGAGAGCCTTTTGAATGGACTGATGTGT
TCTAAGCCCGAAGATCCAGTAGAATACTTGGAAAGTTGTTTACAAAAAGT
AAAGGAACTGGGTGGCTGTGACAAGGTGAAATGGGATACATTTGTAAGCC
AGGAAAAGAAGACCTTACCTCCACTAAATGGAGGACAGTCACGGAGATCC
TTTCTAAGAAATGTAATGCCTGAAAACTCAAACTTTCCATATCGGCGGTA
TGACCGGCTCCCTCCAATCCATCAATTCTCCATAGAAAGTGACACGGATC
TCTCTGAGACTGCAGAGTTGATTGAGGAGTATGAGGTTTTTGATCCTACC
AGACCTCGACCAAAAATCATTCTTGTTATAGGTGGTCCAGGAAGTGGAAA
GGGTACTCAGAGTTTGAAAATTGCAGAACGATATGGATTCCAATACATTT
CTGTGGGAGAATTATTAAGAAAGAAGATCCACAGTACCAGCAGCAATAGG
AAATGGAGTCTTATTGCCAAGATAATTACAACTGGAGAATTGGCCCCACA
GGAAACAACAATTACAGAGATAAAACAAAAATTGATGCAAATACCTGATG
AAGAGGGCATTGTTATTGATGGATTTCCAAGAGATGTTGCCCAGGCTCTA
TCTTTTGAGGACCAAATCTGTACCCCCGATTTGGTGGTATTCCTGGCTTG
TGCTAATCAGAGACTCAAAGAAAGATTACTGAAGCGTGCAGAACAGCAGG
GCCGACCAGACGACAATGTAAAAGCTACCCAAAGGAGACTAATGAACTTC
AAGCAGAATGCTGCTCCATTGGTTAAATACTTCCAGGAAAAGGGGCTCAT
CATGACATTTGATGCCGACCGCGATGAGGATGAGGTGTTCTATGACATCA
GCATGGCAGTTGACAACAAGTTATTTCCAAACAAAGAGGCTGCAGCAGGT
TCAAGTGACCTTGATCCTTCGATGATATTGGACACTGGAGAGATCATTGA
TACAGGATCTGATTATGAAGATCAGGGTGATGACCAGTTAAATGTATTTG
GAGAGGACACTATGGGAGGTTTCATGGAAGATTTGAGAAAGTGTAAAATT
ATTTTCATAATTGGTGGTCCTGGCTCTGGCAAAGGCACACAGTGTGAAAA
GCTGGTGGAAAAATATGGATTTACACATCTCTCAACTGGCGAGCTCCTGC
GTGAGGAACTGGCATCAGAATCTGAAAGAAGCAAATTGATCAGAGACATT
ATGGAACGTGGAGACCTGGTGCCCTCAGGCATCGTTTTGGAGCTCCTGAA
GGAGGCCATGGTGGCCAGCCTCGGGGACACCAGGGGCTTCCTGATTGACG
GCTATCCTCGGGAGGTGAAGCAAGGGGAAGAGTTCGGACGCAGGATTGGA
GACCCACAGTTGGTGATCTGTATGGACTGCTCGGCAGACACCATGACCAA
CCGCCTTCTCCAAAGGAGCCGGAGCAGCCTGCCTGTGGACGACACCACCA
AGACCATCGCCAAGCGCCTAGAAGCCTACTACCGAGCGTCCATCCCCGTG
ATCGCCTACTACGAGACAAAAACACAGCTACACAAGATAAATGCAGAGGG
AACACCAGAGGACGTTTTTCTTCAACTCT

| | | | |
|---|---|---|---|
| NOV2d, 309326609 | | | MW at |
| Protein Sequence | SEQ ID NO: 32 | 571 aa | 64259.2 kD |

TRSPTMNTNDAKEYLARREIPQLFESLLNGLMCSKPEDPVEYLESCLQKV
KELGGCDKVKWDTFVSQEKKTLPPLNGGQSRRSFLRNVMPENSNFPYRRY
DRLPPIHQFSIESDTDLSETAELIEEYEVFDPTRPRPKIILVIGGPGSGK
GTQSLKIAERYGFQYISVGELLRKKIHSTSSNRKWSLIAKIITTGELAPQ
ETTITEIKQKLMQIPDEEGIVIDGFPRDVAQALSFEDQICTPDLVVFLAC
ANQRLKERLLKRAEQQGRPDDNVKATQRRLMNFKQNAAPLVKYFQEKGLI
MTFDADRDEDEVFYDISMAVDNKLFPNKEAAAGSSDLDPSMILDTGEIID
TGSDYEDQGDDQLNVFGEDTMGGFMEDLRKCKIIFIIGGPGSGKGTQCEK
LVEKYGFTHLSTGELLREELASESERSKLIRDIMERGDLVPSGIVLELLK
EAMVASLGDTRGFLIDGYPREVKQGEEFGRRIGDPQLVICMDCSADTMTN
RLLQRSRSSLPVDDTTKTIAKRLEAYYRASIPVIAYYETKTQLHKINAEG
TPEDVFLQLCTAIDSIIFVDG

| | | |
|---|---|---|
| NOV2e, | SEQ ID NO: 33 | 1642 bp |
| 309326618 | ORF Start: | ORF Stop: |
| DNA Sequence | at 2 | end of sequence |

CACCAGATCTCCCACCATGAACACCAACGATGCCAAGGAGTATCTGGCCC
GGAGGGAAATCCCTCAGCTTTTTGAGAGCCTTTTGAATGGACTGATGTGT
TCTAAGCCCGAAGATCCAGTAGAATACTTGGAAAGTTGTTTACAAAAAGT
AAAGGAACTGGGTGGCTGTGACAAGGTGAAATGGGATACATTTGTAAGCC
AGGAAAAGAAGACCTTACCTCCACTAAATGGAGGACAGTCACGGAGATCC
TTTCTAAGAAATGAAAGTGACACGGATCTCTCTGAGACTGCAGAGTTGAT
TGAGGAGTATGAGGTTTTTGATCCTACCAGACCTCGACCAAAAATCATTC
TTGTTATAGGTGGTCCAGGAAGTGGAAAGGGTACTCAGAGTTTGAAAATT
GCAGAACGATATGGATTCCAATACATTTCTGTGGGAGAATTATTAAGAAA
GAAGATCCACAGTACCAGCAGCAATAGGAAATGGAGTCTTATTGCCAAGA
TAATTACAACTGGAGAATTGGCCCCACAGGAAACAACAATTACAGAGATA
AAACAAAAATTGATGCAAATACCTGATGAAGAGGGCATTGTTATTGATGG
ATTTCCAAGAGATGTTGCCCAGGCTCTATCTTTTGAGGACCAAATCTGTA
CCCCCGATTTGGTGGTATTCCTGGCTTGTGCTAATCAGAGACTCAAAGAA
AGATTACTGAAGCGTGCAGAACAGCAGGGCCGACCAGACGACAATGTAAA
AGCTACCCAAAGGAGACTAATGAACTTCAAGCAGAATGCTGCTCCATTGG
TTAAATACTTCCAGGAAAAGGGGCTCATCATGACATTTGATGCCGACCGC
GATGAGGATGAGGTGTTCTATGACATCAGCATGGCAGTTGACAACAAGTT
ATTTCCAAACAAAGAGGCTGCAGCAGGTTCAAGTGACCTTGATCCTTCGA
TGATATTGGACACTGGAGAGATCATTGATACAGGATCTGATTATGAAGAT
CAGGGTGATGACCAGTTAAATGTATTTGGAGAGGACACTATGGGAGGTTT
CATGGAAGATTTGAGAAAGTGTAAAATTATTTTCATAATTGGTGGTCCTG
GCTCTGGCAAAGGCACACAGTGTGAAAAGCTGGTGGAAAAATATGGATTT
ACACATCTCTCAACTGGCGAGCTCCTGCGTGAGGAACTGGCATCAGAATC
TGAAAGAAGCAAATTGATCAGAGACATTATGGAACGTGGAGACCTGGTGC
CCTCAGGCATCGTTTTGGAGCTCCTGAAGGAGGCCATGGTGGCCAGCCTC
GGGGACACCAGGGGCTTCCTGATTGACGGCTATCCTCGGGAGGTGAAGCA
AGGGGAAGAGTTCGGACGCAGGATTGGAGACCCACAGTTGGTGATCTGTA
TGGACTGCTCGGCAGACACCATGACCAACCGCCTTCTCCAAAGGAGCCGG
AGCAGCCTGCCTGTGGACGACACCACCAAGACCATCGCCAAGCGCCTAGA
AGCCTACTACCGAGCGTCCATCCCCGTGATCGCCTACTACGAGACAAAAA
CACAGCTACACAAGATAAATGCAGAGGGAACACCAGAGGACGTTTTTCTT
CAACTCTGCACAGCTATTGACTCTATTATTTTCGTCGACGGC

| | | | |
|---|---|---|---|
| NOV2e, 309326618 | | | MW at |
| Protein Sequence | SEQ ID NO: 34 | 547 aa | 61299.9 kD |

TRSPTMNTNDAKEYLARREIPQLFESLLNGLMCSKPEDPVEYLESCLQKV
KELGGCDKVKWDTFVSQEKKTLPPLNGGQSRRSFLRNESDTDLSETAELI
EEYEVFDPTRPRPKIILVIGGPGSGKGTQSLKIAERYGFQYISVGELLRK
KIHSTSSNRKWSLIAKIITTGELAPQETTITEIKQKLMQIPDEEGIVIDG
FPRDVAQALSFEDQICTPDLVVFLACANQRLKERLLKRAEQQGRPDDNVK
ATQRRLMNFKQNAAPLVKYFQEKGLIMTFDADRDEDEVFYDISMAVDNKL
FPNKEAAAGSSDLDPSMILDTGEIIDTGSDYEDQGDDQLNVFGEDTMGGF
MEDLRKCKIIFIIGGPGSGKGTQCEKLVEKYGFTHLSTGELLREELASES
ERSKLIRDIMERGDLVPSGIVLELLKEAMVASLGDTRGFLIDGYPREVKQ
GEEFGRRIGDPQLVICMDCSADTMTNRLLQRSRSSLPVDDTTKTIAKRLE
AYYRASIPVIAYYETKTQLHKINAEGTPEDVFLQLCTAIDSIIFVDG

| | | |
|---|---|---|
| NOV2f, | SEQ ID NO: 35 | 1842 bp |
| CG101826-01 | ORF Start: | ORF Stop: |
| DNA Sequence | ATG at 375 | TGA at 1626 |

GCGGCCGCGCTGCCTGGCAGCCCGGGAAGCCGCGGCACAGCTGCTCGGCG
CCTGCAGCTCCGGCTCGGGGGCTGGAACCGAAGCGGGGGCGGCGGGAGCG
CGGAGACCACAGCCCCCGGGGAGAGGCGGAGGGGGTCCCTGGCCTGGGCG
GAGAGGCTGAGCTGAGTGCGCGTGAGAAAGAGGGCTGCACCGCTGCTCGG
CGCGGACTCTGCCAGCCCCAGCTTCAGCCCCGGCTCAGGTCGCCGCAGCC
CGGGAGCCTCCCCGCTTGCGCCCCAAGGCACGCGCGGCACAGCCATGAAC
ACCAACGATGCCAAGGAGTATCTGGCCCGGAGGGAAATCCCTCAGTTATA
TTTGAGAGCCTTTTGAATGGACTGATGTGTTCTAAGCCCGAAGATCCAGT
AGAATACTTGGAAAGTTGTTTACAAAAAGTAAAGGAACTGGGTGGCTGTG
ACAAGGTGAAATGGGATACATTTGTAAGCCAGGAAAAGAAGACCTTACCT
CCACTAAATGGAGGACAGTCACGGAGATCCTTTCTAAGAAATGTAATGCC
TGAAAACTCAAACTTTCCATATCGGCGGTATGACCGGCTCCCTCCAATCC
ATCAATTCTCCATAGAAAGTGACACGGATCTCTCTGAGACTGCGGAGTTG
ATTGAGGAGTATGAGGTTTTTGATCCTACCAGACCTCGACCAAAAATCAT
TCTTGTTATAGGTGGTCCAGGAAGTGGAAAGGGTACTCAGAGTTTGAAAA
TTGCAGAACGATATGGATTCCAATACATTTCTGTGGGAGAATTATTAAGA
AAGAAGATCCACAGTACCAGCAGCAATAGGAAATGGAGTCTTATTGCCAA
GATAATTACAACTGGAGAATTGGCCCCACAGGAAACAACAATTACAGAGA
TAAAACAAAAATTGATGCAAATACCTGATGAAGAGGGCATTGTTATTGAT
GGATTTCCAAGAGATGTTGCCCAGGCTCTATCTTTTGAGGACCAGATCTG
TACCCCCGATTTGGTGGTATTCCTGGCTTGTGCTAATCAGAGACTCAAAG
AAAGATTACTGAAGCGTGCAGAACAGCAGGGCCGACCAGACGACAATGTA
AAAGCTACCCAAAGGAGACTAATGAACTTCAAGCAGAATGCTGCTCCATT
GGTTAAATACTTCCAGGAAAAGGGGCTCATCATGACATTTGATGCCGACC
GCGATGAGGATGAGGTGTTCTATGACATCAGCATGGCAGTTGACAACAAG
TTATTTCCAAACAAAGAGGCTGCAGCAGGTTCAAGTGACCTTGATCCTTC
GATGATATTGGACACTGGAGAGATCATTGATACAGGATCTGATTATGAAG
ATCAGGGTGATGACCAGTTAAATGTATTTGGAGAGGACACTATGGGAGGT
TTCATGGAAGATTTGAGAAAGTGTAAAATTATTTTCATAATTGGTGGTCC
TGGCTCTGGCAAAGGCACACAGTGTGAAAAGCTGGTGGAAAAATATGGAT
TTACACATCTCTCAACTGGCGAGCTCCTGCGTGAGGAACTGGCATCAGAA
TCTGAAAGAAGCAAATTGATCAGAGACATTATGGAACGTGGAGACCTGGT
GCCCTCAGGCATCGTTTGGAGCTCCTGAAGGAGGCATGGTGGCAGCTCGG
GGACACCAGGGGCTTCTGATGACGGTTATCTCGGGAGGGGAAGCAGGGGA
AGAGTCGGACGGCAGATGGAGACCAACAGTGGGACTGGATGGACTGCCCG
AAACACCAGACAACGCTTCCAAAGGACGGAGATTGCTGGGAGAACACAAA
AACAGGAACGCAAGCAAACGAAGGAACCAGGGAGCCCAGAAA

| | | | |
|---|---|---|---|
| NOV2f, CG101826-01 | | | MW at |
| Protein Sequence | SEQ ID NO: 36 | 417 aa | 46958.7 kD |

MCSKPEDPVEYLESCLQKVKELGGCDKVKWDTFVSQEKKTLPPLNGGQSR
RSFLRNVMPENSNFPYRRYDRLPPIHQFSIESDTDLSETAELIEEYEVFD
PTRPRPKIILVIGGPGSGKGTQSLKIAERYGFQYISVGELLRKKIHSTSS
NRKWSLIAKIITTGELAPQETTITEIKQKLMQIPDEEGIVIDGFPRDVAQ
ALSFEDQICTPDLVVFLACANQRLKERLLKRAEQQGRPDDNVKATQRRLM
NFKQNAAPLVKYFQEKGLIMTFDADRDEDEVFYDISMAVDNKLFPNKEAA
AGSSDLDPSMILDTGEIIDTGSDYEDQGDDQLNVFGEDTMGGFMEDLRKC
KIIFIIGGPGSGKGTQCEKLVEKYGFTHLSTGELLREELASESERSKLIR
DIMERGDLVPSGIVWSS

| | | |
|---|---|---|
| NOV2g, | SEQ ID NO: 37 | 1714 bp |
| CG101826-03 | ORF Start: | ORF Stop: |
| DNA Sequence | ATG at 17 | at 1706 |

CACCAGATCTCCCACCATGAACACCAACGATGCCAAGGAGTATCTGGCCC
GGAGGGAAATCCCTCAGCTTTTTGAGAGCCTTTTGAATGGACTGATGTGT
TCTAAGCCCGAAGATCCAGTAGAATACTTGGAAAGTTGTTTACAAAAAGT
AAAGGAACTGGGTGGCTGTGACAAGGTGAAATGGGATACATTTGTAAGCC
AGGAAAAGAAGACCTTACCTCCACTAAATGGAGGACAGTCACGGAGATCC
TTTCTAAGAAATGTAATGCCTGAAAACTCAAACTTTCCATATCGGCGGTA
TGACCGGCTCCCTCCAATCCATCAATTCTCCATAGAAAGTGACACGGATC
TCTCTGAGACTGCAGAGTTGATTGAGGAGTATGAGGTTTTTGATCCTACC
AGACCTCGACCAAAAATCATTCTTGTTATAGGTGGTCCAGGAAGTGGAAA
GGGTACTCAGAGTTTGAAAATTGCAGAACGATATGGATTCCAATACATTT
CTGTGGGAGAATTATTAAGAAAGAAGATCCACAGTACCAGCAGCAATAGG
AAATGGAGTCTTATTGCCAAGATAATTACAACTGGAGAATTGGCCCCACA
GGAAACAACAATTACAGAGATAAAACAAAAATTGATGCAAATACCTGATG
AAGAGGGCATTGTTATTGATGGATTTCCAAGAGATGTTGCCCAGGCTCTA
TCTTTTGAGGACCAAATCTGTACCCCCGATTTGGTGGTATTCCTGGCTTG
TGCTAATCAGAGACTCAAAGAAAGATTACTGAAGCGTGCAGAACAGCAGG
GCCGACCAGACGACAATGTAAAAGCTACCCAAAGGAGACTAATGAACTTC
AAGCAGAATGCTGCTCCATTGGTTAAATACTTCCAGGAAAAGGGGCTCAT
CATGACATTTGATGCCGACCGCGATGAGGATGAGGTGTTCTATGACATCA
GCATGGCAGTTGACAACAAGTTATTTCCAAACAAAGAGGCTGCAGCAGGT
TCAAGTGACCTTGATCCTTCGATGATATTGGACACTGGAGAGATCATTGA
TACAGGATCTGATTATGAAGATCAGGGTGATGACCAGTTAAATGTATTTG
GAGAGGACACTATGGGAGGTTTCATGGAAGATTTGAGAAAGTGTAAAATT
ATTTTCATAATTGGTGGTCCTGGCTCTGGCAAAGGCACACAGTGTGAAAA
GCTGGTGGAAAAATATGGATTTACACATCTCTCAACTGGCGAGCTCCTGC
GTGAGGAACTGGCATCAGAATCTGAAAGAAGCAAATTGATCAGAGACATT
ATGGAACGTGGAGACCTGGTGCCCTCAGGCATCGTTTTGGAGCTCCTGAA
GGAGGCCATGGTGGCCAGCCTCGGGGACACCAGGGGCTTCCTGATTGACG
GCTATCCTCGGGAGGTGAAGCAAGGGGAAGAGTTCGGACGCAGGATTGGA
GACCCACAGTTGGTGATCTGTATGGACTGCTCGGCAGACACCATGACCAA
CCGCCTTCTCCAAAGGAGCCGGAGCAGCCTGCCTGTGGACGACACCACCA
AGACCATCGCCAAGCGCCTAGAAGCCTACTACCGAGCGTCCATCCCCGTG
ATCGCCTACTACGAGACAAAAACACAGCTACACAAGATAAATGCAGAGGG
AACACCAGAGGACGTTTTTCTTCAACTCTGCACAGCTATTGACTCTATTA
TTTTCGTCGACGGC

| | | | |
|---|---|---|---|
| NOV2g, CG101826-03 | | | MW at |
| Protein Sequence | SEQ ID NO: 38 | 563 aa | 63445.4 kD |

MNTNDAKEYLARREIPQLFESLLNGLMCSKPEDPVEYLESCLQKVKELGG
CDKVKWDTFVSQEKKTLPPLNGGQSRRSFLRNVMPENSNFPYRRYDRLPP
IHQFSIESDTDLSETAELIEEYEVFDPTRPRPKIILVIGGPGSGKGTQSL
KIAERYGFQYISVGELLRKKIHSTSSNRKWSLIAKIITTGELAPQETTIT
EIKQKLMQIPDEEGIVIDGFPRDVAQALSFEDQICTPDLVVFLACANQRL
KERLLKRAEQQGRPDDNVKATQRRLMNFKQNAAPLVKYFQEKGLIMTFDA
DRDEDEVFYDISMAVDNKLFPNKEAAAGSSDLDPSMILDTGEIIDTGSDY
EDQGDDQLNVFGEDTMGGFMEDLRKCKIIFIIGGPGSGKGTQCEKLVEKY
GFTHLSTGELLREELASESERSKLIRDIMERGDLVPSGIVLELLKEAMVA
SLGDTRGFLIDGYPREVKQGEEFGRRIGDPQLVICMDCSADTMTNRLLQR
SRSSLPVDDTTKTIAKRLEAYYRASIPVIAYYETKTQLHKINAEGTPEDV
FLQLCTAIDSIIF

| | | |
|---|---|---|
| NOV2h, | SEQ ID NO: 39 | 1714 bp |
| CG101826-04 | ORF Start: | ORF Stop: |
| DNA Sequence | ATG at 14 | TAG at 1703 |

CACCGGATCCACCATGAACACCAACGATGCCAAGGAGTATCTGGCCCGGA
GGGAAATCCCTCAGCTTTTTGAGAGCCTTTTGAATGGACTGATGTGTTCT
AAGCCCGAAGATCCAGTAGAATACTTGGAAAGTTGTTTACAAAAAGTAAA
GGAACTGGGTGGCTGTGACAAGGTGAAATGGGATACATTTGTAAGCCAGG
AAAAGAAGACCTTACCTCCACTAAATGGAGGACAGTCACGGAGATCCTTT
CTAAGAAATGTAATGCCTGAAAACTCAAACTTTCCATATCGGCGGTATGA
CCGGCTCCCTCCAATCCATCAATTCTCCATAGAAAGTGACACGGATCTCT
CTGAGACTGCAGAGTTGATTGAGGAGTATGAGGTTTTTGATCCTACCAGA
CCTCGACCAAAAATCATTCTTGTTATAGGTGGTCCAGGAAGTGGAAAGGG
TACTCAGAGTTTGAAAATTGCAGAACGATATGGATTCCAATACATTTCTG
TGGGAGAATTATTAAGAAAGAAGATCCACAGTACCAGCAGCAATAGGAAA
TGGAGTCTTATTGCCAAGATAATTACAACTGGAGAATTGGCCCCACAGGA
AACAACAATTACAGAGATAAAACAAAAATTGATGCAAATACCTGATGAAG
AGGGCATTGTTATTGATGGATTTCCAAGAGATGTTGCCCAGGCTCTATCT
TTTGAGGACCAAATCTGTACCCCCGATTTGGTGGTATTCCTGGCTTGTGC
TAATCAGAGACTCAAAGAAAGATTACTGAAGCGTGCAGAACAGCAGGGCC
GACCAGACGACAATGTAAAAGCTACCCAAAGGAGACTAATGAACTTCAAG
CAGAATGCTGCTCCATTGGTTAAATACTTCCAGGAAAAGGGGCTCATCAT
GACATTTGATGCCGACCGCGATGAGGATGAGGTGTTCTATGACATCAGCA
TGGCAGTTGACAACAAGTTATTTCCAAACAAAGAGGCTGCAGCAGGTTCA
AGTGACCTTGATCCTTCGATGATATTGGACACTGGAGAGATCATTGATAC
AGGATCTGATTATGAAGATCAGGGTGATGACCAGTTAAATGTATTTGGAG
AGGACACTATGGGAGGTTTCATGGAAGATTTGAGAAAGTGTAAAATTATT
TTCATAATTGGTGGTCCTGGCTCTGGCAAAGGCACACAGTGTGAAAAGCT
GGTGGAAAAATATGGATTTACACATCTCTCAACTGGCGAGCTCCTGCGTG
AGGAACTGGCATCAGAATCTGAAAGAAGCAAATTGATCAGAGACATTATG
GAACGTGGAGACCTGGTGCCCTCAGGCATCGTTTTGGAGCTCCTGAAGGA
GGCCATGGTGGCCAGCCTCGGGGACACCAGGGGCTTCCTGATTGACGGCT
ATCCTCGGGAGGTGAAGCAAGGGGAAGAGTTCGGACGCAGGATTGGAGAC
CCACAGTTGGTGATCTGTATGGACTGCTCGGCAGACACCATGACCAACCG
CCTTCTCCAAAGGAGCCGGAGCAGCCTGCCTGTGGACGACACCACCAAGA
CCATCGCCAAGCGCCTAGAAGCCTACTACCGAGCGTCCATCCCCGTGATC
GCCTACTACGAGACAAAAACACAGCTACACAAGATAAATGCAGAGGGAAC
ACCAGAGGACGTTTTTCTTCAACTCTGCACAGCTATTGACTCTATTATTT
TCTAGCTCGAGGGC

| | | | |
|---|---|---|---|
| NOV2h, CG101826-04 | | | MW at |
| Protein Sequence | SEQ ID NO: 40 | 563 aa | 63445.4 kD |

MNTNDAKEYLARREIPQLFESLLNGLMCSKPEDPVEYLESCLQKVKELGG
CDKVKWDTFVSQEKKTLPPLNGGQSRRSFLRNVMPENSNFPYRRYDRLPP
IHQFSIESDTDLSETAELIEEYEVFDPTRPRPKIILVIGGPGSGKGTQSL
KIAERYGFQYISVGELLRKKIHSTSSNRKWSLIAKIITTGELAPQETTIT
EIKQKLMQIPDEEGIVIDGFPRDVAQALSFEDQICTPDLVVFLACANQRL
KERLLKRAEQQGRPDDNVKATQRRLMNFKQNAAPLVKYFQEKGLIMTFDA
DRDEDEVFYDISMAVDNKLFPNKEAAAGSSDLDPSMILDTGEIIDTGSDY
EDQGDDQLNVFGEDTMGGFMEDLRKCKIIFIIGGPGSGKGTQCEKLVEKY
GFTHLSTGELLREELASESERSKLIRDIMERGDLVPSGIVLELLKEAMVA
SLGDTRGFLIDGYPREVKQGEEFGRRIGDPQLVICMDCSADTMTNRLLQR
SRSSLPVDDTTKTIAKRLEAYYRASIPVIAYYETKTQLHKINAEGTPEDV
FLQLCTAIDSIIF

| | | |
|---|---|---|
| | SEQ ID NO: 41 | 13309 bp |
| NOV2i, SNP13376052 | ORF Start: | ORF Stop: |
| of CG101826-02, | ATG at 295 | TGA at 1984 |
| DNA Sequence | SNP Pos: 642 | SNP Change: A to G |

GCGGCCGCGCTGCCTGGCAGCCCGGGAAGCCGCGGCACAGCTGCTCGGCG
CCTGCAGCTCCGGCTCGGGGGCTGGAACCGAAGCGGGGGCGGCGGGAGCG
CGGAGACCACAGCCCCCGGGGAGAGGCGGAGGGGGTCCCTGGCCTGGGCG
GAGAGGCTGAGCTGAGTGCGCGTGAGAAAGAGGGCTGCACCGCTGCTCGG
CGCGGACTCTGCCAGCCCCAGCTTCAGCCCCGGCTCAGGTCGCCGCAGCC
CGGGAGCCTCCCCGCTTGCGCCCCAAGGCACGCGCGGCACAGCCATGAAC
ACCAACGATGCCAAGGAGTATCTGGCCCGGAGGGAAATCCCTCAGCTTTT
TGAGAGCCTTTTGAATGGACTGATGTGTTCTAAGCCCGAAGATCCAGTAG
AATACTTGGAAAGTTGTTTACAAAAAGTAAAGGAACTGGGTGGCTGTGAC
AAGGTGAAATGGGATACATTTGTAAGCCAGGAAAAGAAGACCTTACCTCC
ACTAAATGGAGGACAGTCACGGAGATCCTTTCTAAGAAATGTAATGCCTG
AAAACTCAAACTTTCCATATCGGCGGTATGACCGGCTCCCTCCAATCCAT
CAATTCTCCATAGAAAGTGACACGGATCTCTCTGAGACTGCGGAGTTGAT
TGAGGAGTATGAGGTTTTTGATCCTACCAGACCTCGACCAAAAATCATTC
TTGTTATAGGTGGTCCAGGAAGTGGAAAGGGTACTCAGAGTTTGAAAATT
GCAGAACGATATGGATTCCAATACATTTCTGTGGGAGAATTATTAAGAAA
GAAGATCCACAGTACCAGCAGCAATAGGAAATGGAGTCTTATTGCCAAGA
TAATTACAACTGGAGAATTGGCCCCACAGGAAACAACAATTACAGAGATA
AAACAAAAATTGATGCAAATACCTGATGAAGAGGGCATTGTTATTGATGG
ATTTCCAAGAGATGTTGCCCAGGCTCTATCTTTTGAGGACCAAATCTGTA
CCCCCGATTTTGTGGTATTCCTGGCTTGTGCTAATCAGAGACTCAAAGAA
AGATTACTGAAGCGTGCAGAACAGCAGGGCCGACCAGACGACAATGTAAA
AGCTACCCAAAGGAGACTAATGAACTTCAAGCAGAATGCTGCTCCATTGG
TTAAATACTTCCAGGAAAAGGGGCTCATCATGACATTTGATGCCGACCGC
GATGAGGATGAGGTGTTCTATGACATCAGCATGGCAGTTGACAACAAGTT
ATTTCCAAACAAAGAGGCTGCAGCAGGTTCAAGTGACCTTGATCCTTCGA
TGATATTGGACACTGGAGAGATCATTGATACAGGATCTGATTATGAAGAT
CAGGGTGATGACCAGTTAAATGTATTTGGAGAGGACACTATGGGAGGTTT
CATGGAAGATTTGAGAAAGTGTAAAATTATTTTCATAATTGGTGGTCCTG
GCTCTGGCAAAGGCACACAGTGTGAAAAGCTGGTGGAAAAATATGGATTT
ACACATCTCTCAACTGGCGAGCTCCTGCGTGAGGAACTGGCATCAGAATC
TGAAAGAAGCAAATTGATCAGAGACATTATGGAACGTGGAGACCTGGTGC
CCTCAGGCATCGTTTTGGAGCTCCTGAAGGAGGCCATGGTGGCCAGCCTC
GGGGACACCAGGGGCTTCCTGATTGACGGCTATCCTCGGGAGGTGAAGCA
AGGGGAAGAGTTCGGACGCAGGATTGGAGACCCACAGTTGGTGATCTGTA
TGGACTGCTCGGCAGACACCATGACCAACCGCCTTCTCCAAAGGAGCCGG
AGCAGCCTGCCTGTGGACGACACCACCAAGACCATCGCCAAGCGCCTAGA
AGCCTACTACCGAGCGTCCATCCCCGTGATCGCCTACTACGAGACAAAAA
CACAGCTACACAAGATAAATGCAGAGGGAACACCAGAGGACGTTTTTCTT
CAACTCTGCACAGCTATTGACTCTATTATTTTCTGAAGGCAAAAATGCAT
GTTTGTTAGAATGGAAACAGAAAAACATTAAAAAGTTCATTCCTTAACAC
AATGTTTCAAGTTAAACCTTTTGTGTCACCGCCCCCCACCAACCACCACC
TCCTAAATCCTGACAGCACTGTTTGCTTCCCAGCTAGACCTGTGTGAGAG
GTGTCTGGAAATCATGCATGGTGTATTTGGGACTATATCAACCTATTCTC
CACACTTCAGACAACTGTCTGCACTCACGGCACGCACACTTTGTATCATG
CAGGCCACACTCAGAGCTAGTCAGTACATGAACAGTGGTGCGGTGCCAGT
CTGTGTCCGTTGTGATCACAGGCCTTGCTAGACCCTGATCATCTGGTTCT
CCTCTCATTAAGCATCCCTAACCCCCAGTCACACCTTCCTCTTACATACT
GTTCCCCAATGGAGGCCCCTGGCATAGGGGACAGCCCTGGGCATCTTCCT
TTGGTGTCTGGCTGTTTTGTCAACTCTCATCCACTGGTGGCTCAGAGCCA
TAAGGTGGGTTGATTACACAATGCCTTGTACATGATATAGAGGCATCAAG
CAAGTAAAATTTGACAGAAATTTTAAAATATGAAGATGTATAGCTTTCCC
AAGATGATGGTAAAACCCAGGTTAGTCATCAGTAACCTTCTCTATTATTA
TTATTTTTTAGAAACTTGGAATACTGTCATTATGGCTAAGAGAACAAATC
TGATAAATTGTGTAACCTAGTCTCTTCTCTACATGGTGATGCATTTCAGC
AATTATAAATTAATATAAATGACCAAAAGTAACTTAAAAGCATGAGATAT
TTGCTATTTCATTCATTGGGCACATATCAAATTATAATTTTGATTTTAAA
TGGTCACCCATGTATTTGTTGCCAAGCAAGTAAAAAAATACCCTAACAAA
CCTGATGTGGGTGGGAGGGGCATGTCAGTAAGTGGTGTGTTCAATGTGTT
TGTTTCATATGGGCCCTTTCCAGGAGTTTGCAAACCTTGTCATACCCATA
TGCAAAACTGTGTTTCCTTGCATTAAACCAGTGAAGTTTGGGTTCTCTTT
TGTGCTATCAATCAGTTGTAAAATCAGAGCTTCTTATATATTCTACTGGA
ATAACTGCATCTTCCACTCAGTCACTACAAAAAAGCATAGTTTCAGTTTG
CATGAATTTTTTTTTTTTTCTTCAATGGTTGTGCAGATAAGGATCCATTT
CTGGGATAGAATTGTATTTTTTAAGTCATTTTTTTTTCTTGAAATGGATA
TGTACAAATAAAATTAAATGGAAGACAGGAAAAAAAAAAAAAAAAAAAAA
AAAAAAAAA

| | | | |
|---|---|---|---|
| | | | MW at |
| NOV2i, SNP13376052 | | | 63479.4 kD |
| of CG101826-02, | SEQ ID NO: 42 | | SNP Change: |
| Protein Sequence | SNP Pos: 116 | 563 aa | Ala to Ala |

MNTNDAKEYLARREIPQLFESLLNGLMCSKPEDPVEYLESCLQKVKELGG
CDKVKWDTFVSQEKKTLPPLNGGQSRRSFLRNVMPENSNFPYRRYDRLPP
IHQFSIESDTDLSETAELIEEYEVFDPTRPRPKIILVIGGPGSGKGTQSL
KIAERYGFQYISVGELLRKKIHSTSSNRKWSLIAKIITTGELAPQETTIT
EIKQKLMQIPDEEGIVIDGFPRDVAQALSFEDQICTPDFVVFLACANQRL
KERLLKRAEQQGRPDDNVKATQRRLMNFKQNAAPLVKYFQEKGLIMTFDA
DRDEDEVFYDISMAVDNKLFPNKEAAAGSSDLDPSMILDTGEIIDTGSDY
EDQGDDQLNVFGEDTMGGFMEDLRKCKIIFIIGGPGSGKGTQCEKLVEKY
GFTHLSTGELLREELASESERSKLIRDIMERGDLVPSGIVLELLKEAMVA
SLGDTRGFLIDGYPREVKQGEEFGRRIGDPQLVICMDCSADTMTNRLLQR
SRSSLPVDDTTKTIAKRLEAYYRASIPVIAYYETKTQLHKINAEGTPEDV
FLQLCTAIDSIIF

A ClustalW comparison of the above protein sequences yields the following sequence alignment shown in Table 2B.

TABLE 2B
Comparison of the NOV2 protein sequences.

| | |
|---|---|
| NOV2a | -----MNTNDAKEYLARREIPQLFESLLNGLMCSKPEDPVEYLESCLQKVKELGGCDKVK |
| NOV2b | ------------------------------------------------------------ |
| NOV2c | ------------------------------------------------------------ |
| NOV2d | TRSPTMNTNDAKEYLARREIPQLFESLLNGLMCSKPEDPVEYLESCLQKVKELGGCDKVK |
| NOV2e | TRSPTMNTNDAKEYLARREIPQLFESLLNGLMCSKPEDPVEYLESCLQKVKELGGCDKVK |
| NOV2f | -------------------------------MCSKPEDPVEYLESCLQKVKELGGCDKVK |
| NOV2g | -----MNTNDAKEYLARREIPQLFESLLNGLMCSKPEDPVEYLESCLQKVKELGGCDKVK |
| NOV2h | -----MNTNDAKEYLARREIPQLFESLLNGLMCSKPEDPVEYLESCLQKVKELGGCDKVK |
| NOV2a | WDTFVSQEKKTLPPLNGGQSRRSFLRNVMPENSNFPYRRYDRLPPIHQFSIESDTDLSET |
| NOV2b | ------------------------------------------------------------ |
| NOV2c | ------------------------------------------------------------ |
| NOV2d | WDTFVSQEKKTLPPLNGGQSRRSFLRNVMPENSNFPYRRYDRLPPIHQFSIESDTDLSET |
| NOV2e | WDTFVSQEKKTLPPLNGGQSRRSFLRNES------------------------DTDLSET |
| NOV2f | WDTFVSQEKKTLPPLNGGQSRRSFLRNVMPENSNFPYRRYDRLPPIHQFSIESDTDLSET |
| NOV2g | WDTFVSQEKKTLPPLNGGQSRRSFLRNVMPENSNFPYRRYDRLPPIHQFSIESDTDLSET |
| NOV2h | WDTFVSQEKKTLPPLNGGQSRRSFLRNVMPENSNFPYRRYDRLPPIHQFSIESDTDLSET |
| NOV2a | AELIEEYEVFDPTRPRPKIILVIGGPGSGKGTQSLKIAERYGFQYISVGELLRKKIHSTS |
| NOV2b | ------------------TRSIIGGPGSGKGTQCEKLVEKYGFTHLSTGELLREELASES |
| NOV2c | ------------------TRSVIGGPGSGKGTQSLKIAERYGFQYISVGELLRKKIHSTS |
| NOV2d | AELIEEYEVFDPTRPRPKIILVIGGPGSGKGTQSLKIAERYGFQYISVGELLRKKIHSTS |
| NOV2e | AELIEEYEVFDPTRPRPKIILVIGGPGSGKGTQSLKIAERYGFQYISVGELLRKKIHSTS |
| NOV2f | AELIEEYEVFDPTRPRPKIILVIGGPGSGKGTQSLKIAERYGFQYISVGELLRKKIHSTS |
| NOV2g | AELIEEYEVFDPTRPRPKIILVIGGPGSGKGTQSLKIAERYGFQYISVGELLRKKIHSTS |
| NOV2h | AELIEEYEVFDPTRPRPKIILVIGGPGSGKGTQSLKIAERYGFQYISVGELLRKKIHSTS |
| NOV2a | SNRKWSLIAKIITTGELAPQETTITEIKQKLMQIP-DEEGIVIDGFPRDVAQALSFEDQI |
| NOV2b | ERS--KLIRDIMERGDLVPSGIVLELLKEAMVASLGDTRGFLIDGYPREVKQGEEFGRRI |
| NOV2c | SNRKWSLIAKIITTGELAPQETTITEIKQKLMQIP-DEEGIVIDGFPRDVAQALSFEDQI |
| NOV2d | SNRKWSLIAKIITTGELAPQETTITEIKQKLMQIP-DEEGIVIDGFPRDVAQALSFEDQI |
| NOV2e | SNRKWSLIAKIITTGELAPQETTITEIKQKLMQIP-DEEGIVIDGFPRDVAQALSFEDQI |
| NOV2f | SNRKWSLIAKIITTGELAPQETTITEIKQKLMQIP-DEEGIVIDGFPRDVAQALSFEDQI |
| NOV2g | SNRKWSLIAKIITTGELAPQETTITEIKQKLMQIP-DEEGIVIDGFPRDVAQALSFEDQI |
| NOV2h | SNRKWSLIAKIITTGELAPQETTITEIKQKLMQIP-DEEGIVIDGFPRDVAQALSFEDQI |
| NOV2a | CTPDFVVFLACANQRLKERLLKRAEQQGRPDDNVKATQRRLMNFKQNAAPLVKYFQEKGL |
| NOV2b | GDPQLVICMDCSADTMTNRLLQRSRSSLPVDDTTKTIAKRLEAYYRASIPVIAYYETKTQ |
| NOV2c | CTPDLVVFLACANQRLKERLLKRAEQQGRPDDNVKATQRRLMNFKQNAAPLVKYFQEKGL |
| NOV2d | CTPDLVVFLACANQRLKERLLKRAEQQGRPDDNVKATQRRLMNFKQNAAPLVKYFQEKGL |
| NOV2e | CTPDLVVFLACANQRLKERLLKRAEQQGRPDDNVKATQRRLMNFKQNAAPLVKYFQEKGL |
| NOV2f | CTPDLVVFLACANQRLKERLLKRAEQQGRPDDNVKATQRRLMNFKQNAAPLVKYFQEKGL |
| NOV2g | CTPDLVVFLACANQRLKERLLKRAEQQGRPDDNVKATQRRLMNFKQNAAPLVKYFQEKGL |
| NOV2h | CTPDLVVFLACANQRLKERLLKRAEQQGRPDDNVKATQRRLMNFKQNAAPLVKYFQEKGL |
| NOV2a | IMTFDADRDEDEVFYDISMAVDNKLFPNKEAAAGSSDLDPSMILDTGEIIDTGSDYEDQG |
| NOV2b | VDG--------------------------------------------------------- |
| NOV2c | VDG--------------------------------------------------------- |
| NOV2d | IMTFDADRDEDEVFYDISMAVDNKLFPNKEAAAGSSDLDPSMILDTGEIIDTGSDYEDQG |
| NOV2e | IMTFDADRDEDEVFYDISMAVDNKLFPNKEAAAGSSDLDPSMILDTGEIIDTGSDYEDQG |
| NOV2f | IMTFDADRDEDEVFYDISMAVDNKLFPNKEAAAGSSDLDPSMILDTGEIIDTGSDYEDQG |
| NOV2g | IMTFDADRDEDEVFYDISMAVDNKLFPNKEAAAGSSDLDPSMILDTGEIIDTGSDYEDQG |
| NOV2h | IMTFDADRDEDEVFYDISMAVDNKLFPNKEAAAGSSDLDPSMILDTGEIIDTGSDYEDQG |
| NOV2a | DDQLNVFGEDTMGGFMEDLRKCKIIFIIGGPGSGKGTQCEKLVEKYGFTHLSTGELLREE |
| NOV2b | ------------------------------------------------------------ |
| NOV2c | ------------------------------------------------------------ |
| NOV2d | DDQLNVFGEDTMGGFMEDLRKCKIIFIIGGPGSGKGTQCEKLVEKYGFTHLSTGELLREE |
| NOV2e | DDQLNVFGEDTMGGFMEDLRKCKIIFIIGGPGSGKGTQCEKLVEKYGFTHLSTGELLREE |
| NOV2f | DDQLNVFGEDTMGGFMEDLRKCKIIFIIGGPGSGKGTQCEKLVEKYGFTHLSTGELLREE |
| NOV2g | DDQLNVFGEDTMGGFMEDLRKCKIIFIIGGPGSGKGTQCEKLVEKYGFTHLSTGELLREE |
| NOV2h | DDQLNVFGEDTMGGFMEDLRKCKIIFIIGGPGSGKGTQCEKLVEKYGFTHLSTGELLREE |
| NOV2a | LASESERSKLIRDIMERGDLVPSGIVLELLKEAMVASLGDTRGFLIDGYPREVKQGEEFG |
| NOV2b | ------------------------------------------------------------ |
| NOV2c | ------------------------------------------------------------ |
| NOV2d | LASESERSKLIRDIMERGDLVPSGIVLELLKEAMVASLGDTRGFLIDGYPREVKQGEEFG |
| NOV2e | LASESERSKLIRDIMERGDLVPSGIVLELLKEAMVASLGDTRGFLIDGYPREVKQGEEFG |
| NOV2f | LASESERSKLIRDIMERGDLVPSGIVWSS------------------------------- |
| NOV2g | LASESERSKLIRDIMERGDLVPSGIVLELLKEAMVASLGDTRGFLIDGYPREVKQGEEFG |
| NOV2h | LASESERSKLIRDIMERGDLVPSGIVLELLKEAMVASLGDTRGFLIDGYPREVKQGEEFG |
| NOV2a | RRIGDPQLVICMDCSADTMTNRLLQRSRSSLPVDDTTKTIAKRLEAYYRASIPVIAYYET |
| NOV2b | ------------------------------------------------------------ |
| NOV2c | ------------------------------------------------------------ |
| NOV2d | RRIGDPQLVICMDCSADTMTNRLLQRSRSSLPVDDTTKTIAKRLEAYYRASIPVIAYYET |
| NOV2e | RRIGDPQLVICMDCSADTMTNRLLQRSRSSLPVDDTTKTIAKRLEAYYRASIPVIAYYET |
| NOV2f | ------------------------------------------------------------ |
| NOV2g | RRIGDPQLVICMDCSADTMTNRLLQRSRSSLPVDDTTKTIAKRLEAYYRASIPVIAYYET |
| NOV2h | RRIGDPQLVICMDCSADTMTNRLLQRSRSSLPVDDTTKTIAKRLEAYYRASIPVIAYYET |
| NOV2a | KTQLHKINAEGTPEDVFLQLCTAIDSIIF--- |
| NOV2b | -------------------------------- |
| NOV2c | -------------------------------- |
| NOV2d | KTQLHKINAEGTPEDVFLQLCTAIDSIIFVDG |
| NOV2e | KTQLHKINAEGTPEDVFLQLCTAIDSIIFVDG |
| NOV2f | -------------------------------- |
| NOV2g | KTQLHKINAEGTPEDVFLQLCTAIDSIIF--- |
| NOV2h | KTQLHKINAEGTPEDVFLQLCTAIDSIIF--- |
| NOV2a | (SEQ ID NO: 26) |
| NOV2b | (SEQ ID NO: 28) |
| NOV2c | (SEQ ID NO: 30) |
| NOV2d | (SEQ ID NO: 32) |
| NOV2e | (SEQ ID NO: 34) |
| NOV2f | (SEQ ID NO: 36) |
| NOV2g | (SEQ ID NO: 38) |
| NOV2h | (SEQ ID NO: 40) |

Further analysis of the NOV2a protein yielded the following properties shown in Table 2C.

TABLE 2C
Protein Sequence Properties NOV2a

| SignalP | No Known Signal Sequence Predicted | | | |
|---|---|---|---|---|
| analysis: | | | | |
| PSORT II | PSG: a new signal peptide prediction method | | | |
| analysis: | N-region: length 8; pos. chg 1; neg. chg 2 | | | |
| | H-region: length 3; peak value 0.00 | | | |
| | PSG score: −4.40 | | | |
| | GvH: von Heijne's method for signal seq. recognition | | | |
| | GvH score (threshold: −2.1): −8.05 | | | |
| | possible cleavage site: between 28 and 29 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 1.48 (at 434) | |
| | ALOM score: 1.48 (number of TMSs: 0) | | | |
| | MITDISC: discrimination of mitochondrial targeting seq | | | |
| | R content: | 0 | Hyd Moment (75): | 5.18 |
| | Hyd Moment (95): | 5.42 | G content: | 0 |
| | D/E content: | 2 | S/T content: | 1 |

```
Score: −6.95
Gavel: prediction of cleavage sites for mitochondrial preseq
cleavage site motif not found
NUCDISC: discrimination of nuclear localization signals
pat4: none
pat7: none
bipartite: none
content of basic residues: 12.8%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
43.5%: cytoplasmic
26.1%: nuclear
21.7%: mitochondrial
  4.3%: Golgi
  4.3%: endoplasmic reticulum
>> prediction for CG101826-02 is cyt (k = 23)
```

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU17301 | Novel signal transduction pathway | 178 . . . 563 | 385/386 (99%) | 0.0 |
| | protein, Seq ID 866 - Homo sapiens, | 1 . . . 386 | 385/386 (99%) | |
| | 386 aa. [WO200154733-A1, | | | |
| | 02 AUG. 2001] | | | |
| AAE11776 | Human kinase (PKIN)-10 protein - | 207 . . . 563 | 356/357 (99%) | 0.0 |
| | Homo sapiens, 357 aa. | 1 . . . 357 | 356/357 (99%) | |
| | [WO200181555-A2, 01 NOV. 2001] | | | |
| AAE32029 | Human kinase and phosphatase | 84 . . . 375 | 291/292 (99%) | e−167 |
| | (KPP)-10 - Homo sapiens, 293 aa. | 1 . . . 292 | 291/292 (99%) | |
| | [WO200283709-A2, 24 OCT. 2002] | | | |
| AAU30543 | Novel human secreted protein #1034 - | 279 . . . 563 | 270/286 (94%) | e−153 |
| | Homo sapiens, 297 aa. | 3 . . . 288 | 277/286 (96%) | |
| | [WO200179449-A2, 25 OCT. 2001] | | | |
| AAU17300 | Novel signal transduction pathway | 321 . . . 561 | 241/241 (100%) | e−136 |
| | protein, Seq ID 865 - Homo sapiens, | 4 . . . 244 | 241/241 (100%) | |
| | 245 aa. [WO200154733-A1, | | | |
| | 02 AUG. 2001] | | | |

In a BLAST search of public sequence databases, the NOV2a protein was found to have homology to the proteins shown in the BLASTP data in Table 2E.

TABLE 2E
Public BLASTP Results for NOV2a

| | | | Identities/ | |
|---|---|---|---|---|
| | | NOV2a | Similarities | |
| Protein | | Residues/ | for the | |
| Accession | | Match | Matched | Expect |
| Number | Protein/Organism/Length | Residues | Portion | Value |
| Q8N464 | Adenylate kinase 5 - Homo sapiens | 1 . . . 561 | 558/561 (99%) | 0.0 |
| | (Human), 562 aa. | 1 . . . 561 | 559/561 (99%) | |
| Q86YS0 | Adenylate kinase 6 - Homo sapiens | 27 . . . 561 | 531/535 (99%) | 0.0 |
| | (Human), 536 aa. | 1 . . . 535 | 531/535 (99%) | |
| Q95KE9 | Hypothetical 49.0 kDa protein - | 27 . . . 430 | 398/404 (98%) | 0.0 |
| | Macaca fascicularis (Crab eating | 1 . . . 404 | 401/404 (98%) | |
| | macaque) (Cynomolgus monkey), 432 aa. | | | |
| Q8N291 | Hypothetical protein FLJ33648 - | 1 . . . 233 | 209/233 (89%) | e−113 |
| | Homo sapiens (Human), 241 aa. | 1 . . . 209 | 209/233 (89%) | |
| Q9Y6K8 | Adenylate kinase isoenzyme 5 (EC | 366 . . . 563 | 198/198 (100%) | e−109 |
| | 2.7.4.3) (ATP-AMP | 1 . . . 198 | 198/198 (100%) | |
| | transphosphorylase) - Homo sapiens | | | |
| | (Human), 198 aa. | | | |

PFam analysis predicts that the NOV2a protein contains the domains shown in the Table 2F.

TABLE 2F
Domain Analysis of NOV2a

| | NOV2a | Identities/ | |
|---|---|---|---|
| | Match | Similarities for | Expect |
| Pfam Domain | Region | the Matched Region | Value |
| Dpy-30 | 5 . . . 47 | 15/43 (35%) | 0.0016 |
| | | 32/43 (74%) | |
| ADK | 137 . . . 294 | 140/159 (88%) | 2.8e−39 |
| | | 149/159 (94%) | |
| Thymidylate_kin | 137 . . . 311 | 40/203 (20%) | 0.3 |
| | | 108/203 (53%) | |
| ADK | 381 . . . 537 | 145/160 (91%) | 2.1e−64 |
| | | 151/160(94%) | |

### Example 3

The NOV3 clone was analyzed, and the nucleotide and encoded polypeptide sequences are shown in Table 3A.

TABLE 3A
NOV3 Sequence Analysis

| | | |
|---|---|---|
| NOV3a, | SEQ ID NO: 43 | 3205 bp |
| CG105201-01 | ORF Start: | ORF Stop: |
| DNA Sequence | ATG at 75 | TGA at 1146 |

GACAAGAGCTCAGACCTGAGGAGAGTGACTAGCTTCTCTGTGTCCCAGGT
GGCCACCTTCCACTGTGGAAGCTCATGGACTCCATTGGGTCTTCAGGGTT
GCGGCAGGGGGAAGAAACCCTGAGTTGCTCTGAGGAGGGCTTGCCCGGGC
CCTCAGACAGCTCAGAGCTGGTGCAGGAGTGCCTGCAGCAGTTCAAGGTG
ACAAGGGCACAGCTACAGCAGATCCAAGCCAGCCTCTTGGGTTCCATGGA
GCAGGCGCTGAGGGGACAGGCCAGCCCTGCCCCTGCGGTCCGGATGCTGC
CTACATACGTGGGGTCCACCCCACATGGCACTGAGCAAGGAGACTTCGTG
GTGCTGGAGCTGGGGGCCACAGGGGCCTCACTGCGTGTTTTGTGGGTGAC
TCTAACTGGCATTGAGGGGCATAGGGTGGAGCCCAGAAGCCAGGAGTTTG
TGATCCCCCAAGAGGTGATGCTGGGTGCTGGCCAGCAGCTCTTTGACTTT
GCTGCCCACTGCCTGTCTGAGTTCCTGGATGCGCAGCCTGTGAACAAACA
GGGTCTGCAGCTTGGCTTCAGCTTCTCTTTCCCTTGTCACCAGACGGGCT
TGGACAGGAGCACCCTCATTTCCTGGACCAAAGGTTTTAGGTGCAGTGGT
GTGGAAGGCCAGGATGTGGTCCAGCTGCTGAGAGATGCCATTCGGAGGCA
GGGGGCCTACAACATCGACGTGGTTGCTGTGGTGAACGACACAGTGGGCA
CCATGATGGGCTGTGAGCCGGGGGTCAGGCCGTGTGAGGTTGGGCTAGTT
GTAGACACGGGCACCAACGCGTGTTACATGGAGGAGGCACGGCATGTGGC
AGTGCTGGACGAAGACCGGGGCCGCGTCTGCGTCAGCGTCGAGTGGGGCT
CCTTAAGCGATGATGGGGCGCTGGGACCAGTGCTGACCACCTTCGACCAT
ACCCTGGACCATGAGTCCCTGAATCCTGGTGCTCAGAGGTTTGAGAAGAT
GATCGGAGGCCTGTACCTGGGTGAGCTGGTGCGGCTGGTGCTGGCTCACT
TGGCCCGGTGTGGGGTCCTCTTTGGTGGCTGCACCTCCCCTGCCCTGCTG
AGCCAAGGCAGCATCCTCCTGGAACACGTGGCTGAGATGGAGGAGTGAGT
CGGGGAGATGGTGGTTTAGTGGGGGATTCTTGGCTTGGAGGAAGGGGATG
ATACTCTGTTCCCAAGGTAGCCATGGGGCTTTAGTGGGATGGGGAGCTTC
TGGGCTGAGCCCCAAACCACTTCCCTTTCCCCTCCAGCCCCTCTACTGGG
GCAGCCCGTGTCCATGCTATCCTGCAGGACTTGGGCCTGAGCCCTGGGGC
TTCGGATGTTGAGCTTGTGCAGCACGTCTGTGCGGCCGTGTGCACGCGGG
CTGCCCAGCTCTGTGCTGCCGCCCTGGCCGCTGTTCTCTCCTGCCTCCAG
CACAGCCGGGAGCAACAAACACTCCAGGTTGCTGTGGCCACCGGAGGCCG
AGTGTGTGAGCGGCACCCCAGGTTCTGCAGCGTCCTGCAGGGGACAGTGA
TGCTCCTGGCCCCGGAATGCGATGTCTCCTTAATCCCCTCTGTGGATGGT
GGTGGCCGGGGAGTGGCGATGGTGACTGCTGTGGCTGCCCGTCTGGCTGC
CCACCGGCGCCTGCTGGAGGAGACCCTGGCCCCATTCCGGTTGAACCATG
ATCAACTGGCTGCGGTTCAGGCACAGATGCGGAAGGCCATGGCCAAGGGG
CTCCGAGGGGAGGCCTCCTCCCTTCGCATGCTGCCCACTTTCGTCCGGGC
CACCCCTGACGGCAGCGAGCGAGGGGATTTCCTGGCCCTGGACCTCGGGG
GCACGAACTTCCGTGTCCTCCTGGTACGTGTGACCACAGGCGTGCAGATC
ACCAGCGAGATCTACTCCATTCCCGAGACTGTGGCCCAGGGTTCTGGGCA
GCAGCTCTTTGACCACATCGTGGACTGCATCGTGGACTTCCAGCAGAAGC
AGGGCCTGAGCGGGCAGAGCCTCCCACTGGGTTTTACCTTCTCCTTCCCA
TGTAGGCAGCTTGGCCTAGACCAGGGCATCCTCCTGAACTGGACCAAGGG
TTTCAAGGCATCAGACTGCGAGGGCCAAGATGTCCTGAGTCTGTTGCGGG
AAGCCATCACTCGCAGACAGGCAGTGGAGCTGAATGTGGTTGCCATTGTC
AATGACACGGTGGGGACCATGATGTCCTGTGGCTATGAGGACCCCCGTTG
CGAGATAGGCCTCATTGTCGGAACCGGCACCAATGCCTGCTACATGGAGG
AGCTCCGGAATGTGGCGGGCGTGCCTGGGGACTCAGGCCGCATGTGCATC
AACATGGAGTGGGGCGCCTTTGGGGACGATGGCTCTCTGGCCATGCTCAG
CACCCGCTTTGATGCAAGTGTGGACCAGGCGTCCATCAACCCCGGCAAGC
AGAGGTTTGAAAAGATGATCAGCGGCATGTACCTGGGGGAGATCGTCCGC
CACATCCTTTTACATTTAACCAGCCTTGGCGTTCTCTTCCGGGGCCAGCA
GATCCAGCGCCTTCAGACCAGGGACATCTTCAAGACCAAGTTCCTCTCTG
AGATCGAAAGTGACAGCCTGGCCCTGCGGCAGGTCCGAGCCATCCTAGAG
GATCTGGGGCTACCCCTGACCTCAGATGACGCCCTGATGGTGCTAGAGGT
GTGCCAGGCTGTGTCCCAGAGGGCTGCCCAGCTCTGTGGGGCGGGTGTAG
CTGCCGTGGTGGAGAAGATCCGGGGGAACCGGGGCCTGGAAGAGCTGGCA
GTGTCTGTGGGGGTGGATGGAACGCTCTACAAGCTGCACCCGCGCTTCTC
CAGCCTGGTGGCGGCCACAGTGCGGGAGCTGGCCCCTCGCTGTGTGGTCA
CGTTCCTGCAGTCAGAGGATGGGTCCGGCAAAGGTGCGGCCCTGGTCACC
GCTGTTGCCTGCCGCCTTGCGCAGTTGACTCGTGTCTGAGGAAACCTCCA
GGCTGAGGAGGTCTCCGCCGCAGCCTTGCTGGAGCCGGGTCGGGGTCTGC
CTGTTTCCCAGCCAGGCCCAGCCACCCAGGACTCCTGGGACATCCCATGT
GTGACCCCTCTGCGGCCATTTGGCCTTGCTCCCTGGCTTTCCCTGAGAGA
AGTAGCACTCAGGTTAGCATATATATATATAATTTATTTACAAAAAAAAA
AAAAAA

| | | | |
|---|---|---|---|
| NOV3a, CG105201-01 | | | MW at |
| Protein Sequence | SEQ ID NO: 44 | 357 aa | 38361.1 kD |

MDSIGSSGLRQGEETLSCSEEGLPGPSDSSELVQECLQQFKVTRAQLQQI
QASLLGSMEQALRGQASPAPAVRMLPTYVGSTPHGTEQGDFVVLELGATG
ASLRVLWVTLTGIEGHRVEPRSQEFVIPQEVMLGAGQQLFDFAAHCLSEF
LDAQPVNKQGLQLGFSFSFPCHQTGLDRSTLISWTKGFRCSGVEGQDVVQ
LLRDAIRRQGAYNIDVVAVVNDTVGTMMGCEPGVRPCEVGLVVDTGTNAC
YNEEARHVAVLDEDRGRVCVSVEWGSLSDDGALGPVLTTFDHTLDHESLN
PGAQRFEKMIGGLYLGELVRLVLAHLARCGVLFGGCTSPALLSQGSILLE
HVAEMEE

| | | |
|---|---|---|
| NOV3b, | SEQ ID NO: 45 | 1096 bp |
| 277575154 | ORF Start: | ORF Stop: |
| DNA Sequence | at 2 | end of sequence |

CACCAAGCTTCCCACCATGGACTCCATTGGGTCTTCAGGGTTGCGGCAGG
GGGAAGAAACCCTGAGTTGCTCTGAGGAGGGCTTGCCCGGGCCCTCAGAC
AGCTCAGAGCTGGTGCAGGAGTGCCTGCAGCAGTTCAAGGTGACAAGGGC
ACAGCTACAGCAGATCCAAGCCAGCCTCTTGGGTTCCATGGAGCAGGCGC
TGAGGGGACAGGCCAGCCCTGCCCCTGCGGTCCGGATGCTGCCTACATAC
GTGGGGTCCACCCCACATGGCACTGAGCAAGGAGACTTCGTGGTGCTGGA
GCTGGGGGCCACAGGGGCCTCACTGCGTGTTTTGTGGGTGACTCTAACTG
GCATTGAGGGGCATAGGGTGGAGCCCAGAAGCCAGGAGTTTGTGATCCCC
CAAGAGGTGATGCTGGGTGCTGGCCAGCAGCTCTTTGACTTTGCTGCCCA
CTGCCTGTCTGAGTTCCTGGATGCGCAGCCTGTGAACAAACAGGGTCTGC
AGCTTGGCTTCAGCTTCTCTTTCCCTTGTCACCAGACGGGCTTGGACAGG
AGCACCCTCATTTCCTGGACCAAAGGTTTTAGGTGCAGTGGTGTGGAAGG
CCAGGATGTGGTCCAGCTGCTGAGAGATGCCATTCGGAGGCAGGGGGCCT
ACAACATCGACGTGGTTGCTGTGGTGAACGACACAGTGGGCACCATGATG
GGCTGTGAGCCGGGGGTCAGGCCGTGTGAGGTTGGGCTAGTTGTAGACAC
GGGCACCAACGCGTGTTACATGGAGGAGGCACGGCATGTGGCAGTGCTGG
ACGAAGACCGGGGCCGCGTCTGCGTCAGCGTCGAGTGGGGCTCCTTCAGC
GATGATGGGGCGCTGGGACCAGTGCTGACCACCTTCGACCATACCCTGGA
CCATGAGTCCCTGAATCCTGGTGCTCAGAGGTTTGAGAAGATGATCGGAG
GCCTGTACCTGGGTGAGCTGGTGCGGCTGGTGCTGGCTCACTTGGCCCGG
TGTGGGGTCCTCTTTGGTGGCTGCACCTCCCCTGCCCTGCTGAGCCAAGG
CAGCATCCTCCTGGAACACGTGGCTGAGATGGAGGAGGTCGACGGC

| | | | |
|---|---|---|---|
| NOV3b, 277575154 | | | MW at |
| Protein Sequence | SEQ ID NO: 46 | 365 aa | 39207.1 kD |

TKLPTMDSIGSSGLRQGEETLSCSEEGLPGPSDSSELVQECLQQFKVTRA
QLQQIQASLLGSMEQALRGQASPAPAVRMLPTYVGSTPHGTEQGDFVVLE
LGATGASLRVLWVTLTGIEGHRVEPRSQEFVIPQEVMLGAGQQLFDFAAH
CLSEFLDAQPVNKQGLQLGFSFSFPCHQTGLDRSTLISWTKGFRCSGVEG
QDVVQLLRDAIRRQGAYNIDVVAVVNDTVGTMMGCEPGVRPCEVGLVVDT
GTNACYMEEARHVAVLDEDRGRVCVSVEWGSFSDDGALGPVLTTFDHTLD
HESLNPGAQRFEKMIGGLYLGELVRLVLAHLARCGVLFGGCTSPALLSQG
SILLEHVAEMEEVDG

| | | |
|---|---|---|
| NOV3c, | SEQ ID NO: 47 | 1096 bp |
| CG105201-02 | ORF Start: | ORF Stop: |
| DNA Sequence | ATG at 17 | at 1088 |

CACCAAGCTTCCCACCATGGACTCCATTGGGTCTTCAGGGTTGCGGCAGG
GGGAAGAAACCCTGAGTTGCTCTGAGGAGGGCTTGCCCGGGCCCTCAGAC
AGCTCAGAGCTGGTGCAGGAGTGCCTGCAGCAGTTCAAGGTGACAAGGGC
ACAGCTACAGCAGATCCAAGCCAGCCTCTTGGGTTCCATGGAGCAGGCGC
TGAGGGGACAGGCCAGCCCTGCCCCTGCGGTCCGGATGCTGCCTACATAC
GTGGGGTCCACCCCACATGGCACTGAGCAAGGAGACTTCGTGGTGCTGGA
GCTGGGGGCCACAGGGGCCTCACTGCGTGTTTTGTGGGTGACTCTAACTG
GCATTGAGGGGCATAGGGTGGAGCCCAGAAGCCAGGAGTTTGTGATCCCC
CAAGAGGTGATGCTGGGTGCTGGCCAGCAGCTCTTTGACTTTGCTGCCCA
CTGCCTGTCTGAGTTCCTGGATGCGCAGCCTGTGAACAAACAGGGTCTGC
AGCTTGGCTTCAGCTTCTCTTTCCCTTGTCACCAGACGGGCTTGGACAGG
AGCACCCTCATTTCCTGGACCAAAGGTTTTAGGTGCAGTGGTGTGGAAGG
CCAGGATGTGGTCCAGCTGCTGAGAGATGCCATTCGGAGGCAGGGGGCCT
ACAACATCGACGTGGTTGCTGTGGTGAACGACACAGTGGGCACCATGATG
GGCTGTGAGCCGGGGGTCAGGCCGTGTGAGGTTGGGCTAGTTGTAGACAC
GGGCACCAACGCGTGTTACATGGAGGAGGCACGGCATGTGGCAGTGCTGG
ACGAAGACCGGGGCCGCGTCTGCGTCAGCGTCGAGTGGGGCTCCTTCAGC
GATGATGGGGCGCTGGGACCAGTGCTGACCACCTTCGACCATACCCTGGA
CCATGAGTCCCTGAATCCTGGTGCTCAGAGGTTTGAGAAGATGATCGGAG
GCCTGTACCTGGGTGAGCTGGTGCGGCTGGTGCTGGCTCACTTGGCCCGG
TGTGGGGTCCTCTTTGGTGGCTGCACCTCCCCTGCCCTGCTGAGCCAAGG
CAGCATCCTCCTGGAACACGTGGCTGAGATGGAGGAGGTCGACGGC

| | | | |
|---|---|---|---|
| NOV3c, CG105201-02 | | | MW at |
| Protein Sequence | SEQ ID NO: 48 | 357 aa | 38395.1 kD |

MDSIGSSGLRQGEETLSCSEEGLPGPSDSSELVQECLQQFKVTRAQLQQI
QASLLGSMEQALRGQASPAPAVRMLPTYVGSTPHGTEQGDFVVLELGATG
ASLRVLWVTLTGIEGHRVEPRSQEFVIPQEVMLGAGQQLFDFAAHCLSEF
LDAQPVNKQGLQLGFSFSFPCHQTGLDRSTLISWTKGFRCSGVEGQDVVQ
LLRDAIRRQGAYNIDVVAVVNDTVGTMMGCEPGVRPCEVGLVVDTGTNAC
YMEEARHVAVLDEDRGRVCVSVEWGSFSDDGALGPVLTTFDHTLDHESLN
PGAQRFEKMIGGLYLGELVRLVLAHLARCGVLFGGCTSPALLSQGSILLE
HVAEMEE

A ClustalW comparison of the above protein sequences yields the following sequence alignment shown in Table 3B.

TABLE 3B
Comparison of the NOV3 protein sequences.

| | |
|---|---|
| NOV3a | -----MDSIGSSGLRQGEETLSCSEEGLPGPSDSSELVQECLQQFKVTRAQLQQIQASLL |
| NOV3b | TKLPTMDSIGSSGLRQGEETLSCSEEGLPGPSDSSELVQECLQQFKVTRAQLQQIQASLL |
| NOV3c | -----MDSIGSSGLRQGEETLSCSEEGLPGPSDSSELVQECLQQFKVTRAQLQQIQASLL |
| NOV3a | GSMEQALRGQASPAPAVRMLPTYVGSTPHGTEQGDFVVLELGATGASLRVLWVTLTGIEG |
| NOV3b | GSMEQALRGQASPAPAVRMLPTYVGSTPHGTEQGDFVVLELGATGASLRVLWVTLTGIEG |
| NOV3c | GSMEQALRGQASPAPAVRMLPTYVGSTPHGTEQGDFVVLELGATGASLRVLWVTLTGIEG |
| NOV3a | HRVEPRSQEFVIPQEVMLGAGQQLFDFAAHCLSEFLDAQPVNKQGLQLGFSFSFPCHQTG |
| NOV3b | HRVEPRSQEFVIPQEVMLGAGQQLFDFAAHCLSEFLDAQPVNKQGLQLGFSFSFPCHQTG |
| NOV3c | HRVEPRSQEFVIPQEVMLGAGQQLFDFAAHCLSEFLDAQPVNKQGLQLGFSFSFPCHQTG |
| NOV3a | LDRSTLISWTKGFRCSGVEGQDVVQLLRDAIRRQGAYNIDVVAVVNDTVGTMMGCEPGVR |
| NOV3b | LDRSTLISWTKGFRCSGVEGQDVVQLLRDAIRRQGAYNIDVVAVVNDTVGTMMGCEPGVR |
| NOV3c | LDRSTLISWTKGFRCSGVEGQDVVQLLRDAIRRQGAYNIDVVAVVNDTVGTMMGCEPGVR |
| NOV3a | PCEVGLVVDTGTNACYMEEARHVAVLDEDRGRVCVSVEWGSLSDDGALGPVLTTFDHTLD |
| NOV3b | PCEVGLVVDTGTNACYMEEARHVAVLDEDRGRVCVSVEWGSFSDDGALGPVLTTFDHTLD |
| NOV3c | PCEVGLVVDTGTNACYMEEARHVAVLDEDRGRVCVSVEWGSFSDDGALGPVLTTFDHTLD |
| NOV3a | HESLNPGAQRFEKMIGGLYLGELVRLVLAHLARCGVLFGGCTSPALLSQGSILLEHVAEM |
| NOV3b | HESLNPGAQRFEKMIGGLYLGELVRLVLAHLARCGVLFGGCTSPALLSQGSILLEHVAEM |
| NOV3c | HESLNPGAQRFEKMIGGLYLGELVRLVLAHLARCGVLFGGCTSPALLSQGSILLEHVAEM |
| NOV3a | EE--- |
| NOV3b | EEVDG |
| NOV3c | EE--- |
| NOV3a | (SEQ ID NO: 44) |
| NOV3b | (SEQ ID NO: 46) |
| NOV3c | (SEQ ID NO: 48) |

Further analysis of the NOV3a protein yielded the following properties shown in Table 3C.

TABLE 3C
Protein Sequence Properties NOV3a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 1; neg. chg 1 | | | |
| | H-region: length 2; peak value −6.75 | | | |
| | PSG score: −11.15 | | | |
| | GvH: von Heijne's method for signal seq. recognition | | | |
| | GvH score (threshold: −2.1): −8.20 | | | |
| | possible cleavage site: between 29 and 30 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL | Likelihood = | 1.32 (at 92) | |
| | ALOM score: −0.32 (number of TMSs: 0) | | | |
| | MITDISC: discrimination of mitochondrial targeting seq | | | |
| | R content: | 1 | Hyd Moment (75): | 5.63 |
| | Hyd Moment(95): | 7.08 | G content: | 3 |
| | D/E content: | 2 | S/T content: | 3 |

```
Score: −6.61
Gavel: prediction of cleavage sites for mitochondrial preseq
cleavage site motif not found
NUCDISC: discrimination of nuclear localization signals
pat4: none
pat7: none
bipartite: none
content of basic residues: 6.4%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
39.1%: cytoplasmic
30.4%: mitochondrial
21.7%: nuclear
  4.3%: vacuolar
  4.3%: vesicles of secretory system
>> prediction for CG105201-01 is cyt (k = 23)
```

A search of the NOV3a protein against the Geneseq database, a proprietary database that contains sequences published in patents and patent publication, yielded several homologous proteins shown in Table 3D.

TABLE 3D
Geneseq Results for NOV3a

| | | | Identities/ | |
|---|---|---|---|---|
| | | NOV3a | Similarities | |
| | | Residues/ | for the | |
| Geneseq | Protein/Organism/Length | Match | Matched | Expect |
| Identifier | [Patent #, Date] | Residues | Region | Value |
| ABP70634 | Amino acid sequence of human | 1 . . . 357 | 357/357 (100%) | 0.0 |
| | NOV1 polypeptide - Homo sapiens, | 1 . . . 357 | 357/357 (100%) | |
| | 357 aa. [WO2003000918-A2, | | | |
| | 03 JAN. 2003] | | | |
| AAW37430 | Rat hexokinase III - Rattus sp, 924 | 1 . . . 357 | 295/357 (82%) | e−169 |
| | aa. [WO9726357-A1, 24 JUL. 1997] | 1 . . . 357 | 315/357 (87%) | |
| AAW37443 | Rat hexokinase III - Rattus sp, 924 | 1 . . . 357 | 295/357 (82%) | e−169 |
| | aa. [WO9726322-A2, 24 JUL. 1997] | 1 . . . 357 | 315/357 (87%) | |
| ABP65143 | Hypoxia-regulated protein #17 - | 28 . . . 356 | 164/329 (49%) | 2e−92 |
| | Homo sapiens, 917 aa. | 17 . . . 343 | 226/329 (67%) | |
| | [WO200246465-A2, 13 JUN. 2002] | | | |
| AAW37429 | Rat hexokinase II - Rattus sp, 917 aa. | 33 . . . 357 | 165/325 (50%) | 9e−92 |
| | [WO9726357-A1, 24 JUL. 1997] | 22 . . . 344 | 225/325 (68%) | |

In a BLAST search of public sequence databases, the NOV3a protein was found to have homology to the proteins shown in the BLASTP data in Table 3E.

TABLE 3E
Public BLASTP Results for NOV3a

| | | | Identities/ | |
|---|---|---|---|---|
| | | NOV3a | Similarities | |
| Protein | | Residues/ | for the | |
| Accession | | Match | Matched | Expect |
| Number | Protein/Organism/Length | Residues | Portion | Value |
| P52790 | Hexokinase type III (EC 2.7.1.1) (HK | 1 . . . 357 | 356/357 (99%) | 0.0 |
| | III) - Homo sapiens (Human), 923 aa. | 1 . . . 357 | 357/357 (99%) | |
| Q8N1E7 | Hexokinase 3 (White cell) - Homo | 1 . . . 357 | 355/357 (99%) | 0.0 |
| | sapiens (Human), 923 aa. | 1 . . . 357 | 356/357 (99%) | |
| P27926 | Hexokinase type III (EC 2.7.1.1) (HK | 1 . . . 357 | 295/357 (82%) | e−169 |
| | III) - Rattus norvegicus (Rat), 924 aa. | 1 . . . 357 | 315/357 (87%) | |
| Q8WU87 | Hexokinase 2 - Homo sapiens | 28 . . . 356 | 164/329 (49%) | 6e−92 |
| | (Human), 917 aa. | 17 . . . 343 | 226/329 (67%) | |
| P52789 | Hexokinase, type II (EC 2.7.1.1) (HK | 28 . . . 356 | 164/329 (49%) | 6e−92 |
| | II) (Muscle form hexokinase) - Homo | 17 . . . 343 | 226/329 (67%) | |
| | sapiens (Human), 917 aa. | | | |

PFam analysis predicts that the NOV3a protein contains the domains shown in the Table 3F.

TABLE 3F
Domain Analysis of NOV3a

| | | Identities/ | |
|---|---|---|---|
| | | Similarities | |
| | NOV3a | for the | |
| Pfam Domain | Match Region | Matched Region | Expect Value |
| hexokinase | 27 . . . 234 | 102/235 (43%) | 3.6e−122 |
| | | 200/235 (85%) | |
| hexokinase2 | 236 . . . 357 | 61/259 (24%) | 2.2e−16 |
| | | 119/259 (46%) | |

### Example 4

The NOV4 clone was analyzed, and the nucleotide and encoded polypeptide sequences are shown in Table 4A.

TABLE 4A
NOV4 Sequence Analysis

| | | |
|---|---|---|
| NOV4a, | SEQ ID NO: 49 | 1602 bp |
| CG106773-01 | ORF Start: | ORF Stop: |
| DNA Sequence | ATG at 1 | TGA at 1600 |

ATGGCTTCGACCACCACCTGCACCAGGTTCACGGACGAGTATCAGCTTTT
CGAGGAGCTTGGAAAGGGGGCATTCTCAGTGGTGAGAAGATGTATGAAAA
TTCCTACTGGACAAGGATATGCTGCCAAAATTATCAACACCAAAAAGCTT
TCTGCTAGGGATCATCAGAAACTAGAAAGAGAAGCTAGAATCTGCCGTCT
TTTGAAGCACCCTAATATTGTGCGACTTCATGATAGCATATCAGAAGAGG
GCTTTCACTACTTGGTGTTTGATTTAGTTACTGGAGGTGAACTGTTTGAA
GACATAGTGGCAAGAGAATACTACAGTGAAGCTGATGCCAGTCATTGTAT
ACAGCAGATTCTAGAAAGTGTTAATCATTGTCACCTAAATGGCATAGTTC
ACAGGGACCTGAAGCCTGAGAATTTGCTTTTAGCTAGCAAATCCAAGGGA
GCAGCTGTGAAATTGGCAGACTTTGGCTTAGCCATAGAAGTTCAAGGGGA
CCAGCAGGCGTGGTTTGGTTTTGCTGGCACACCTGGATATCTTTCTCCAG
AAGTTTTACGTAAAGATCCTTATGGAAAGCCAGTGGATATGTGGGCATGT
GGTGTCATTCTCTATATTCTACTTGTGGGGTATCCACCCTTCTGGGATGA
AGACCAACACAGACTCTATCAGCAGATCAAGGCTGGAGCTTATGATTTTC
CATCACCAGAATGGGACACGGTGACTCCTGAAGCCAAAGACCTCATCAAT
AAAATGCTTACTATCAACCCTGCCAAACGCATCACAGCCTCAGAGGCACT
GAAGCACCCATGGATCTGTCAACGTTCTACTGTTGCTTCCATGATGCACA
GACAGGAGACTGTAGACTGCTTGAAGAAATTTAATGCTAGAAGAAAACTA
AAGGGTGCCATCTTGACAACTATGCTGGCTACAAGGAATTTCTCAGCAGC
CAAGAGTTTGTTGAAGAAACCAGATGGAGTAAAGATAAACAACAAAGCCA
ACGTGGTAACCAGCCCCAAAGAAAATATTCCTACCCCAGCGCTGGAGCCC
CAAACTACTGTAATCCACAACCCTGATGGAAACAAGGAGTCAACTGAGAG
TTCAAATACAACAATTGAGGATGAAGATGTGAAAGCACGAAAGCAAGAGA
TTATCAAAGTCACTGAACAACTGATCGAAGCTATCAACAATGGGGACTTT
GAAGCCTACACAAAAATCTGTGACCCAGGCCTTACTGCTTTTGAACCTGA
AGCTTTGGGTAATTTAGTGGAAGGGATGGATTTTCACCGATTCTACTTTG
AAAATGCTTTGTCCAAAAGCAATAAACCAATCCACACTATTATTCTAAAC
CCTCATGTACATCTGGTAGGGGATGATGCCGCCTGCATAGCATATATTAG
GCTCACACAGTACATGGATGGCAGTGGAATGCCAAAGACAATGCAGTCAG
AAGAGACTCGTGTGTGGCACCGCCGGGATGGAAAGTGGCAGAATGTTCAT
TTTCATCGCTCGGGGTCACCAACAGTACCCATCAAGCCACCCTGTATTCC
AAATGGGAAAGAAAACTTCTCAGGAGGCACCTCTTTGTGGCAAAACATCT
GA

| | | | |
|---|---|---|---|
| NOV4a, CG106773-01 | | | MW at |
| Protein Sequence | SEQ ID NO: 50 | 533 aa | 59930.8 kD |

MASTTTCTRFTDEYQLFEELGKGAFSVVRRCMKIPTGQGYAAKIINTKKL
SARDHQKLEREARICRLLKHPNIVRLHDSISEEGFHYLVFDLVTGGELFE
DIVAREYYSEADASHCIQQILESVNHCHLNGIVHRDLKPENLLLASKSKG
AAVKLADFGLAIEVQGDQQAWFGFAGTPGYLSPEVLRKDPYGKPVDMWAC
GVILYILLVGYPPFWDEDQHRLYQQIKAGAYDFPSPEWDTVTPEAKDLIN
KMLTINPAKRITASEALKHPWICQRSTVASMMHRQETVDCLKKFNARRKL
KGAILTTMLATRNFSAAKSLLKKPDGVKINNKANVVTSPKENIPTPALEP
QTTVIHNPDGNKESTESSNTTIEDEDVKARKQEIIKVTEQLIEAINNGDF
EAYTKICDPGLTAFEPEALGNLVEGMDFHRFYFENALSKSNKPIHTIILN
PHVHLVGDDAACIAYIRLTQYMDGSGMPKTMQSEETRVWHRRDGKWQNVH
FHRSGSPTVPIKPPCIPNGKENFSGGTSLWQNI

| | | |
|---|---|---|
| NOV4b, | SEQ ID NO: 51 | 1519 bp |
| 278908476 | ORF Start: | ORF Stop: |
| DNA Sequence | at 2 | end of sequence |

CACCGGATCCACCATGGCTTCGACCACCACCTGCACCAGGTTCACGGACG
AGTATCAGCTTTTCGAGGAGCTTGGAAAGGGGGCATTCTCAGTGGTGAGA
AGATGTATGAAAATTCCTACTGGACAAGAATATGCTGCCAAAATTATCAA
CACCAAAAAGCTTTCTGCTAGGGATCATCAGAAACTAGAAAGAGAAGCTA
GAATCTGCCGTCTTTTGAAGCACCCTAATATTGTGCGACTTCATGATAGC
ATATCAGAAGAGGGCTTTCACTACTTGGTGTTTGATTTAGTTACTGGAGG
TGAACTGTTTGAAGACATAGTGGCAAGAGAATACTACAGTGAAGCTGATG
CCAGTCATTGTATACAGCAGATTCTAGAAAGTGTTAATCATTGTCACCTA
AATGGCATAGTTCACAGGGACCTGAAGCCTGAGAATTTGCTTTTAGCTAG
CAAATCCAAGGGAGCAGCTGTGAAATTGGCAGACTTTGGCTTAGCCATAG
AAGTTCAAGGGGACCAGCAGGCGTGGTTTGGTTTTGCTGGCACACCTGGA
TATCTTTCTCCAGAAGTTTTACGTAAAGATCCTTATGGAAAGCCAGTGGA
TATGTGGGCATGTGGTGTCATTCTCTATATTCTACTTGTGGGGTATCCAC
CCTTCTGGGATGAAGACCAACACAGACTCTATCAGCAGATCAAGGCTGGA
GCTTATGATTTTCCATCACCAGAATGGGACACGGTGACTCCTGAAGCCAA
AGACCTCATCAATAAAATGCTTACTATCAACCCTGCCAAACGCATCACAG
CCTCAGAGGCACTGAAGCACCCATGGATCTGTCAACGTTCTACTGTTGCT
TCCATGATGCACAGACAGGAGACTGTAGACTGCTTGAAGAAATTTAATGC
TAGAAGAAAACTAAAGGGTGCCATCTTGACAACTATGCTGGCTACAAGGA
ATTTCTCAGCAGCCAAGAGTTTGTTGAAGAAACCAGATGGAGTAAAGGAG
TCAACTGAGAGTTCAAATACAACAATTGAGGATGAAGATGTGAAAGCACG
AAAGCAAGAGATTATCAAAGTCACTGAACAACTGATCGAAGCTATCAACA
ATGGGGACTTTGAAGCCTACACAAAAATCTGTGACCCAGGCCTTACTGCT
TTTGAACCTGAAGCTTTGGGTAATTTAGTGGAAGGGATGGATTTTCACCG
ATTCTACTTTGAAAATGCTTTGTCCAAAAGCAATAAACCAATCCACACTA
TTATTCTAAACCCTCATGTACATCTGGTAGGGGATGATGCCGCCTGCATA
GCATATATTAGGCTCACACAGTACATGGATGGCAGTGGAATGCCAAAGAC
AATGCAGTCAGAAGAGACTCGTGTGTGGCACCGCCGGGATGGAAAGTGGC
AGAATGTTCATTTTCATCGCTCGGGGTCACCAACAGTACCCATCAAGCCA
CCCTGTATTCCAAATGGGAAAGAAAACTTCTCAGGAGGCACCTCTTTGTG
GCAAAACATCCTCGAGGGC

| | | | |
|---|---|---|---|
| NOV4b, 278908476 | | | MW at |
| Protein Sequence | SEQ ID NO: 52 | 506 aa | 57014.5 kD |

TGSTMASTTTCTRFTDEYQLFEELGKGAFSVVRRCMKIPTGQEYAAKIIN
TKKLSARDHQKLEREARICRLLKHPNIVRLHDSISEEGFHYLVFDLVTGG
ELFEDIVAREYYSEADASHCIQQILESVNHCHLNGIVHRDLKPENLLLAS
KSKGAAVKLADFGLAIEVQGDQQAWFGFAGTPGYLSPEVLRKDPYGKPVD
MWACGVILYILLVGYPPFWDEDQHRLYQQIKAGAYDFPSPEWDTVTPEAK
DLINKMLTINPAKRITASEALKHPWICQRSTVASMMHRQETVDCLKKFNA
RRKLKGAILTTMLATRNFSAAKSLLKKPDGVKESTESSNTTIEDEDVKAR
KQEIIKVTEQLIEAINNGDFEAYTKICDPGLTAFEPEALGNLVEGMDFHR
FYFENALSKSNKPIHTIILNPHVHLVGDDAACIAYIRLTQYMDGSGMPKT
MQSEETRVWHRRDGKWQNVHFHRSGSPTVPIKPPCIPNGKENFSGGTSLW
QNILEG

| | | |
|---|---|---|
| NOV4c, | SEQ ID NO: 53 | 1561 bp |
| 278908492 | ORF Start: | ORF Stop: |
| DNA Sequence | at 2 | end of sequence |

CACCGGATCCACCATGGCTTCGACCACCACCTGCACCAGGTTCACGGACG
AGTATCAGCTTTTCGAGGAGCTTGGAAAGGGGGCATTCTCAGTGGTGAGA
AGATGTATGAAAATTCCTACTGGACAAGAATATGCTGCCAAAATTATCAA
CACCAAAAAGCTTTCTGCTAGGGATCATCAGAAACTAGAAAGAGAAGCTA
GAATCTGCCGTCTTTTGAAGCACCCTAATATTGTGCGACTTCATGATAGC
ATATCAGAAGAGGGCTTTCACTACTTGGTGTTTGATTTAGTTACTGGAGG
TGAACTGTTTGAAGACATAGTGGCAAGAGAATACTACAGTGAAGCTGATG
CCAGTCATTGTATACAGCAGATTCTAGAAAGTGTTAATCATTGTCACCTA
AATGGCATAGTTCACAGGGACCTGAAGCCTGAGAATTTGCTTTTAGCTAG
CAAATCCAAGGGAGCAGCTGTGAAATTGGCAGACTTTGGCTTAGCCATAG
AAGTTCAAGGGGACCAGCAGGCGTGGTTTGGTTTTGCTGGCACACCTGGA
TATCTTTCTCCAGAAGTTTTACGTAAAGATCCTTATGGAAAGCCAGTGGA
TATGTGGGCATGTGGTGTCATTCTCTATATTCTACTTGTGGGGTATCCAC
CCTTCTGGGATGAAGACCAACACAGACTCTATCAGCAGATCAAGGCTGGA
GCTTATGATTTTCCATCACCAGAATGGGACACGGTGACTCCTGAAGCCAA
AGACCTCATCAATAAAATGCTTACTATCAACCCTGCCAAACGCATCACAG
CCTCAGAGGCACTGAAGCACCCATGGATCTGTCAACGTTCTACTGTTGCT
TCCATGATGCACAGACAGGAGACTGTAGACTGCTTGAAGAAATTTAATGC
TAGAAGAAAACTAAAGGGTGCCATCTTGACAACTATGCTGGCTACAAGGA
ATTTCTCAGCAGCCAAGAGTTTGTTGAAGAAACCAGATGGAGTAAAGGAG
CCCCAAACTACTGTAATCCACAACCCTGATGGAAACAAGGAGTCAACTGA
GAGTTCAAATACAACAATTGAGGATGAAGATGTGAAAGCACGAAAGCAAG
AGATTATCAAAGTCACTGAACAACTGATCGAAGCTATCAACAATGGGGAC
TTTGAAGCCTACACAAAAATCTGTGACCCAGGCCTTACTGCTTTTGAACC
TGAAGCTTTGGGTAATTTAGTGGAAGGGATGGATTTTCACCGATTCTACT
TTGAAAATGCTTTGTCCAAAAGCAATAAACCAATCCACACTATTATTCTA
AACCCTCATGTACATCTGGTAGGGGATGATGCCGCCTGCATAGCATATAT
TAGGCTCACACAGTACATGGATGGCAGTGGAATGCCAAAGACAATGCAGT
CAGAAGAGACTCGTGTGTGGCACCGCCGGGATGGAAAGTGGCAGAATGTT
CATTTTCATCGCTCGGGGTCACCAACAGTACCCATCAAGCCACCCTGTAT
TCCAAATGGGAAAGAAAACTTCTCAGGAGGCACCTCTTTGTGGCAAAACA
TCCTCGAGGGC

| | | | |
|---|---|---|---|
| NOV4c, 278908492 | | | MW at |
| Protein Sequence | SEQ ID NO: 54 | 520 aa | 58546.1 kD |

TGSTMASTTTCTRFTDEYQLFEELGKGAFSVVRRCMKIPTGQEYAAKIIN
TKKLSARDHQKLEREARICRLLKHPNIVRLHDSISEEGFHYLVFDLVTGG
ELFEDIVAREYYSEADASHCIQQILESVNHCHLNGIVHRDLKPENLLLAS
KSKGAAVKLADFGLAIEVQGDQQAWFGFAGTPGYLSPEVLRKDPYGKPVD
MWACGVILYILLVGYPPFWDEDQHRLYQQIKAGAYDFPSPEWDTVTPEAK
DLINKMLTINPAKRITASEALKNPWICQRSTVASMMHRQETVDCLKKFNA
RRKLKGAILTTMLATRNFSAAKSLLKKPDGVKEPQTTVIHNPDGNKESTE
SSNTTIEDEDVKARKQEIIKVTEQLIEAINNGDFEAYTKICDPGLTAFEP
EALGNLVEGMDFHRFYFENALSKSNKPIHTIILNPHVHLVGDDAACIAYI
RLTQYMDGSGMPKTMQSEETRVWHRRDGKWQNVHFHRSGSPTVPIKPPCI
PNGKENFSGGTSLWQNILEG

| | | |
|---|---|---|
| NOV4d, | SEQ ID NO: 55 | 1558 bp |
| 278908496 | ORF Start: | ORF Stop: |
| DNA Sequence | at 2 | end of sequence |

CACCGGATCCACCATGGCTTCGACCACCACCTGCACCAGGTTCACGGACG
AGTATCAGCTTTTCGAGGAGCTTGGAAAGGGGGCATTCTCAGTGGTGAGA
AGATGTATGAAAATTCCTACTGGACAAGAATATGCTGCCAAAATTATCAA
CACCAAAAAGCTTTCTGCTAGGGATCATCAGAAACTAGAAAGAGAAGCTA
GAATCTGCCGTCTTTTGAAGCACCCTAATATTGTGCGACTTCATGATAGC
ATATCAGAAGAGGGCTTTCACTACTTGGTGTTTGATTTAGTTACTGGAGG
TGAACTGTTTGAAGACATAGTGGCAAGAGAATACTACAGTGAAGCTGATG
CCAGTCATTGTATACAGCAGATTCTAGAAAGTGTTAATCATTGTCACCTA
AATGGCATAGTTCACAGGGACCTGAAGCCTGAGAATTTGCTTTTAGCTAG
CAAATCCAAGGGAGCAGCTGTGAAATTGGCAGACTTTGGCTTAGCCATAG
AAGTTCAAGGGGACCAGCAGGCGTGGTTTGGTTTTGCTGGCACACCTGGA
TATCTTTCTCCAGAAGTTTTACGTAAAGATCCTTATGGAAAGCCAGTGGA
TATGTGGGCATGTGGTGTCATTCTCTATATTCTACTTGTGGGGTATCCAC
CCTTCTGGGATGAAGACCAACACAGACTCTATCAGCAGATCAAGGCTGGA
GCTTATGATTTTCCATCACCAGAATGGGACACGGTGACTCCTGAAGCCAA
AGACCTCATCAATAAAATGCTTACTATCAACCCTGCCAAACGCATCACAG
CCTCAGAGGCACTGAAGCACCCATGGATCTGTCAACGTTCTACTGTTGCT
TCCATGATGCACAGACAGGAGACTGTAGACTGCTTGAAGAAATTTAATGC
TAGAAGAAAACTAAAGGGTGCCATCTTGACAACTATGCTGGCTACAAGGA
ATTTCTCAGCCAAGAGTTTGTTGAAGAAACCAGATGGAGTAAAGGAGCCC
CAAACTACTGTAATCCACAACCCTGATGGAAACAAGGAGTCAACTGAGAG
TTCAAATACAACAATTGAGGATGAAGATGTGAAAGCACGAAAGCAAGAGA
TTATCAAAGTCACTGAACAACTGATCGAAGCTATCAACAATGGGGACTTT
GAAGCCTACACAAAAATCTGTGACCCAGGCCTTACTGCTTTTGAACCTGA
AGCTTTGGGTAATTTAGTGGAAGGGATGGATTTTCACCGATTCTACTTTG
AAAATGCTTTGTCCAAAAGCAATAAACCAATCCACACTATTATTCTAAAC
CCTCATGTACATCTGGTAGGGGATGATGCCGCCTGCATAGCATATATTAG
GCTCACACAGTACATGGATGGCAGTGGAATGCCAAAGACAATGCAGTCAG
AAGAGACTCGTGTGTGGCACCGCCGGGATGGAAAGTGGCAGAATGTTCAT
TTTCATCGCTCGGGGTCACCAACAGTACCCATCAAGCCACCCTGTATTCC
AAATGGGAAAGAAAACTTCTCAGGAGGCACCTCTTTGTGGCAAAACATCC
TCGAGGGC

| | | | |
|---|---|---|---|
| NOV4d, 278908496 | | | MW at |
| Protein Sequence | SEQ ID NO: 56 | 519 aa | 58475.0 kD |

TGSTMASTTTCTRFTDEYQLFEELGKGAFSVVRRCMKIPTGQEYAAKIIN
TKKLSARDHQKLEREARICRLLKHPNIVRLHDSISEEGFHYLVFDLVTGG
ELFEDIVAREYYSEADASHCIQQILESVNHCHLNGIVHRDLKPENLLLAS
KSKGAAVKLADFGLAIEVQGDQQAWFGFAGTPGYLSPEVLRKDPYGKPVD
MWACGVILYILLVGYPPFWDEDQHRLYQQIKAGAYDFPSPEWDTVTPEAK
DLINKMLTINPAKRITASEALKHPWICQRSTVASMMHRQETVDCLKKFNA
RRKLKGAILTTMLATRNFSAKSLLKKPDGVKEPQTTVIHNPDGNKESTES
SNTTIEDEDVKARKQEIIKVTEQLIEAINNGDFEAYTKICDPGLTAFEPE
ALGNLVEGMDFHRFYFENALSKSNKPINTIILNPHVHLVGDDAACIAYIR
LTQYMDGSGMPKTMQSEETRVWHRRDGKWQNVHFHRSGSPTVPIKPPCIP
NGKENFSGGTSLWQNILEG

A ClustalW comparison of the above protein sequences yields the following sequence alignment shown in Table 4B.

TABLE 4B
Comparison of the NOV4 protein sequences.

| | |
|---|---|
| NOV4a | ----MASTTTCTRFTDEYQLFEELGKGAFSVVRRCMKIPTGQGYAAKIINTKKLSARDHQ |
| NOV4b | TGSTMASTTTCTRFTDEYQLFEELGKGAFSVVRRCMKIPTGQEYAAKIINTKKLSARDHQ |
| NOV4c | TGSTMASTTTCTRFTDEYQLFEELGKGAFSVVRRCMKIPTGQEYAAKIINTKKLSARDHQ |
| NOV4d | TGSTMASTTTCTRFTDEYQLFEELGKGAFSVVRRCMKIPTGQEYAAKIINTKKLSARDHQ |
| NOV4a | KLEREARICRLLKHPNIVRLHDSISEEGFHYLVFDLVTGGELFEDIVAREYYSEADASHC |
| NOV4b | KLEREARICRLLKHPNIVRLHDSISEEGFHYLVFDLVTGGELFEDIVAREYYSEADASHC |
| NOV4c | KLEREARICRLLKHPNIVRLHDSISEEGFHYLVFDLVTGGELFEDIVAREYYSEADASHC |
| NOV4d | KLEREARICRLLKHPNIVRLHDSISEEGFHYLVFDLVTGGELFEDIVAREYYSEADASHC |
| NOV4a | IQQILESVNHCHLNGIVHRDLKPENLLLASKSKGAAVKLADFGLAIEVQGDQQAWFGFAG |
| NOV4b | IQQILESVNHCHLNGIVHRDLKPENLLLASKSKGAAVKLADFGLAIEVQGDQQAWFGFAG |
| NOV4c | IQQILESVNHCHLNGIVHRDLKPENLLLASKSKGAAVKLADFGLAIEVQGDQQAWFGFAG |
| NOV4d | IQQILESVNHCHLNGIVHRDLKPENLLLASKSKGAAVKLADFGLAIEVQGDQQAWFGFAG |
| NOV4a | TPGYLSPEVLRKDPYGKPVDMWACGVILYILLVGYPPFWDEDQHRLYQQIKAGAYDFPSP |
| NOV4b | TPGYLSPEVLRKDPYGKPVDMWACGVILYILLVGYPPFWDEDQHRLYQQIKAGAYDFPSP |
| NOV4c | TPGYLSPEVLRKDPYGKPVDMWACGVILYILLVGYPPFWDEDQHRLYQQIKAGAYDFPSP |
| NOV4d | TPGYLSPEVLRKDPYGKPVDMWACGVILYILLVGYPPFWDEDQHRLYQQIKAGAYDFPSP |
| NOV4a | EWDTVTPEAKDLINKMLTINPAKRITASEALKHPWICQRSTVASMMHRQETVDCLKKFNA |
| NOV4b | EWDTVTPEAKDLINKMLTINPAKRITASEALKHPWICQRSTVASMMHRQETVDCLKKFNA |
| NOV4c | EWDTVTPEAKDLINKMLTINPAKRITASEALKHPWICQRSTVASMMHRQETVDCLKKFNA |
| NOV4d | EWDTVTPEAKDLINKMLTINPAKRITASEALKHPWICQRSTVASMMHRQETVDCLKKFNA |
| NOV4a | RRKLKGAILTTMLATRNFSAAKSLLKKPDGVKINNKANVVTSPKENIPTPALEPQTTVIH |
| NOV4b | RRKLKGAILTTMLATRNFSAAKSLLKKPD------------------------------- |
| NOV4c | RRKLKGAILTTMLATRNFSAAKSLLKKPDGVKE--------------------PQTTVIH |
| NOV4d | RRKLKGAILTTMLATRNFS-AKSLLKKPDGVKE--------------------PQTTVIH |
| NOV4a | NPDGNKESTESSNTTIEDEDVKARKQEIIKVTEQLIEAINNGDFEAYTKICDPGLTAFEP |
| NOV4b | ---GVKESTESSNTTIEDEDVKARKQEIIKVTEQLIEAINNGDFEAYTKICDPGLTAFEP |
| NOV4c | NFDGNKESTESSNTTIEDEDVKARKQEIIKVTEQLIEAINNGDFEAYTKICDPGLTAFEP |
| NOV4d | NPDGNKESTESSNTTIEDEDVKARKQEIIKVTEQLIEAINNGDFEAYTKICDPGLTAFEP |
| NOV4a | EALGNLVEGMDFHRFYFENALSKSNKPIHTIILNPHVHLVGDDAACIAYIRLTQYMDGSG |
| NOV4b | EALGNLVEGMDFHRFYFENALSKSNKPIHTIILNPHVHLVGDDAACIAYIRLTQYMDGSG |
| NOV4c | EALGNLVEGMDFHRFYFENALSKSNKPIHTIILNPHVHLVGDDAACIAYIRLTQYMDGSG |
| NOV4d | EALGNLVEGMDFHRFYFENALSKSNKPIHTIILNPHVHLVGDDAACIAYIRLTQYMDGSG |
| NOV4a | MPKTMQSEETRVWHRRDGKWQNVHFHRSGSPTVPIKPPCIPNGKENFSGGTSLWQNI--- |
| NOV4b | MPKTMQSEETRVWHRRDGKWQNVHFHRSGSPTVPIKPPCIPNGKENFSGGTSLWQNILEG |
| NOV4c | MPKTMQSEETRVWHRRDGKWQNVHFHRSGSPTVPIKPPCIPNGKENFSGGTSLWQNILEG |
| NOV4d | MPKTMQSEETRVWHRRDGKWQNVHFHRSGSPTVPIKPPCIPNGKENFSGGTSLWQNILEG |
| NOV4a | (SEQ ID NO: 50) |
| NOV4b | (SEQ ID NO: 52) |
| NOV4c | (SEQ ID NO: 54) |
| NOV4d | (SEQ ID NO: 56) |

Further analysis of the NOV4a protein yielded the following properties shown in Table 4C.

TABLE 4C
Protein Sequence Properties NOV4a

| SignalP | No Known Signal Sequence Predicted | | | |
|---|---|---|---|---|
| analysis: | | | | |
| PSORT II | PSG: a new signal peptide prediction method | | | |
| analysis: | N-region: length 9; pos. chg 1; neg. chg 0 | | | |
| | H-region: length 2; peak value −5.65 | | | |
| | PSG score: −10.05 | | | |
| | GvH: von Heijne's method for signal seq. recognition | | | |
| | GvH score (threshold: −2.1): −11.76 | | | |
| | possible cleavage site: between 39 and 40 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −2.39 | Transmembrane 195-211 |
| | PERIPHERAL | Likelihood = | 3.45 (at 88) | |
| | ALOM score: −2.39 (number of TMSs: 1) | | | |
| | MTOP: Prediction of membrane topology (Hartmann et al.) | | | |
| | Center position for calculation: 202 | | | |
| | Charge difference: −0.5 C(−0.5)-N(0.0) | | | |
| | N >= C: N-terminal side will be inside | | | |
| | >>> membrane topology: type 2 (cytoplasmic tail 1 to 195) | | | |
| | MITDISC: discrimination of mitochondrial targeting seq | | | |
| | R content: | 1 | Hyd Moment (75): | 2.09 |
| | Hyd Moment(95): | 1.33 | G content: | 0 |
| | D/E content: | 2 | S/T content: | 6 |
| | Score: | −5.44 | | |

```
Gavel: prediction of cleavage sites for mitochondrial preseq
R-2 motif at 19 TRF|TD
NUCDISC: discrimination of nuclear localization signals
pat4: none
pat7: none
bipartite: none
content of basic residues: 12.0%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: too long tail
Dileucine motif in the tail: found
LL at 67
LL at 142
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
30.4%: mitochondrial
26.1%: cytoplasmic
17.4%: Golgi
  8.7%: endoplasmic reticulum
  4.3%: vacuolar
  4.3%: extracellular, including cell wall
  4.3%: nuclear
  4.3%: vesicles of secretory system
>> prediction for CG106773-01 is mit (k = 23)
```

A search of the NOV4a protein against the Geneseq database, a proprietary database that contains sequences published in patents and patent publication, yielded several homologous proteins shown in Table 4D.

TABLE 4D
Geneseq Results for NOV4a

| | | | Identities/ | |
|---|---|---|---|---|
| | | NOV4a | Similarities | |
| | | Residues/ | for the | |
| Geneseq | Protein/Organism/Length | Match | Matched | Expect |
| Identifier | [Patent #, Date] | Residues | Region | Value |
| ABP70158 | Amino acid sequence of oestrogen | 1 . . . 512 | 445/542 (82%) | 0.0 |
| | receptor alpha cofactor CF19 - Homo | 1 . . . 542 | 478/542 (88%) | |
| | sapiens, 542 aa. [WO200270699-A2, | | | |
| | 12 SEP. 2002] | | | |
| AAM25814 | Human protein sequence SEQ ID | 1 . . . 512 | 438/518 (84%) | 0.0 |
| | NO: 1329 - Homo sapiens, 525 aa. | 8 . . . 525 | 475/518 (91%) | |
| | [WO200153455-A2, 26 JUL. 2001] | | | |
| AAM79441 | Human protein SEQ ID NO 3087 - | 1 . . . 512 | 438/518 (84%) | 0.0 |
| | Homo sapiens, 525 aa. | 8 . . . 525 | 475/518 (91%) | |
| | [WO200157190-A2, 09 AUG. 2001] | | | |
| AAM78457 | Human protein SEQ ID NO 1119 - | 1 . . . 512 | 436/518 (84%) | 0.0 |
| | Homo sapiens, 518 aa. | 1 . . . 518 | 472/518 (90%) | |
| | [WO200157190-A2, 09 AUG. 2001] | | | |
| AAY68786 | Amino acid sequence of a human | 1 . . . 512 | 431/517 (83%) | 0.0 |
| | phosphorylation effector PHSP-18 - | 1 . . . 503 | 465/517 (89%) | |
| | Homo sapiens, 503 aa. | | | |
| | [WO200006728-A2, 10 FEB. 2000] | | | |

In a BLAST search of public sequence databases, the NOV4a protein was found to have homology to the proteins shown in the BLASTP data in Table 4E.

TABLE 4E
Public BLASTP Results for NOV4a

| | | | Identities/ | |
|---|---|---|---|---|
| | | NOV4a | Similarities | |
| Protein | | Residues/ | for the | |
| Accession | | Match | Matched | Expect |
| Number | Protein/Organism/Length | Residues | Portion | Value |
| P15791 | Calcium/calmodulin-dependent protein | 1 . . . 533 | 528/533 (99%) | 0.0 |
| | kinase type II delta chain (EC 2.7.1.123) | 1 . . . 533 | 530/533 (99%) | |
| | (CaM-kinase II delta chain) (CaM kinase | | | |
| | II delta subunit) (CaMK-II delta subunit) - | | | |
| | Rattus norvegicus (Rat), 533 aa. | | | |
| Q8CAC5 | Calcium/calmodulin-dependent protein | 1 . . . 511 | 504/511 (98%) | 0.0 |
| | kinase II - Mus musculus (Mouse), 512 | 1 . . . 511 | 506/511 (98%) | |
| | aa. | | | |
| P28652 | Calcium/calmodulin-dependent protein | 1 . . . 512 | 444/542 (81%) | 0.0 |
| | kinase type II beta chain (EC 2.7.1.123) | 1 . . . 542 | 479/542 (87%) | |
| | (CaM-kinase II beta chain) (CaM kinase | | | |
| | II beta subunit) (CaMK-II beta subunit) - | | | |
| | Mus musculus (Mouse), 542 aa. | | | |
| O93560 | Calcium/calmodulin-dependent kinase | 1 . . . 512 | 443/540 (82%) | 0.0 |
| | type II beta subunit - Gallus gallus | 1 . . . 540 | 476/540 (88%) | |
| | (Chicken), 540 aa. | | | |
| P08413 | Calcium/calmodulin-dependent protein | 1 . . . 512 | 442/542 (81%) | 0.0 |
| | kinase type II beta chain (EC 2.7.1.123) | 1 . . . 542 | 478/542 (87%) | |
| | (CaM-kinase II beta chain) (CaM kinase | | | |
| | II beta subunit) (CaMK-II beta subunit) - | | | |
| | Rattus norvegicus (Rat), 542 aa. | | | |

PFam analysis predicts that the NOV4a protein contains the domains shown in the Table 4F.

TABLE 4F
Domain Analysis of NOV4a

| | | Identities/for | |
|---|---|---|---|
| Pfam | NOV4a Match | the Similarities | Expect |
| Domain | Region | Matched Region | Value |
| pkinase | 14 . . . 272 | 101/300 (34%) | 5.3e−93 |
| | | 214/300 (71%) | |

### Example 5

The NOV5 clone was analyzed, and the nucleotide and encoded polypeptide sequences are shown in Table 5A.

TABLE 5A
NOV5 Sequence Analysis

| | | |
|---|---|---|
| NOV5a, | SEQ ID NO: 57 | 1390 bp |
| CG119621-02 | ORF Start: | ORF Stop: |
| DNA Sequence | at 3 | TGA at 1377 |

CACCGAATTCCACCATGGGGTGCATGAAGTCCAAGTTCCTCCAGGTCGGA
GGCAATACATTCTCAAAAACTGAAACCAGCGCCAGTGCACACTGTCCTGT
GTACGTGCCGGATCCCACATCCACCATCAAGCCGGGGCCTAATAGCCACA
ACAGCAACACACCAGGAATCAGGGAGGCAGGCTCTGAGGACATCATCGTG
GTTGCCCTGTACGATTACGAGGCCATTCACCACGAAGACCTCAGCTTCCA
GAAGGGGGACCAGATGGTGGTCCTAGAGGAATCCGGGGAGTGGTGGAAGG
CTCGATCCCTGGCCACCCGGAAGGAGGGCTACATCCCAAGCAACTATGTC
GCCCGCGTTGACTCTCTGGAGACAGAGGAGTGGTTTTTCAAGGGCATCAG
CCGGAAGGACGCAGAGCGCCAACTGCTGGCTCCCGGCAACATGCTGGGCT
CCTTCATGATCCGGGATAGCGAGACCACTAAAGGAAGCTACTCTTTGTCC
GTGCGAGACTACGACCCTCGGCAGGGAGATACCGTGAAACATTACAAGAT
CCGGACCCTGGACAACGGGGGCTTCTACATATCCCCCCGAAGCACCTTCA
GCACTCTGCAGGAGCTGGTGGACCACTACAAGACCACCTACAACAAGCAC
ACCAAGGTGGCAGTGAAGACGATGAAGCCAGGGAGCATGTCGGTGGAGGC
CTTCCTGGCAGAGGCCAACGTGATGAAAACTCTGCAGCATGACAAGCTGG
TCAAACTTCATGCGGTGGTCACCAAGGAGCCCATCTACATCATCACGGAG
TTCATGGCCAAAGGAAGCTTGCTGGACTTTCTGAAAAGTGATGAGGGCAG
CAAGCAGCCATTGCCAAAACTCATTGACTTCTCAGCCCAGATTGCAGAAG
GCATGGCCTTCATCGAGCAGAGGAACTACATCCACCGAGACCTCCGAGCT
GCCAACATCTTGGTCTCTGCATCCCTGGTGTGTAAGATTGCTGACTTTGG
CCTGGCCCGGGTCATTGAGGACAACGAGTACACGGCTCGGGAAGGGGCCA
AGTTCCCCATCAAGTGGACAGCTCCTGAAGCCATCAACTTTGGCTCCTTC
ACCATCAAGTCAGACGTCTGGTCCTTTGGTATCCTGCTGATGGAGATCGT
CACCTACGGCCGGATCCCTTACCCAGGGATGTCAAACCCTGAAGTGATCC
GAGCTCTGGAGCGTGGATACCGGATGCCTCGCCCAGAGAACTGCCCAGAG
GAGCTCTACAACATCATGATGCGCTGCTGGAAAAACCGTCCGGAGGAGCG
GCCGACCTTCGAATACATCCAGAGTGTGCTGGATGACTTCTACACGGCCA
CAGAGAGCCAGTACCAACAGCAGCCATGAGCGGCCGCTAT

| | | | |
|---|---|---|---|
| NOV5a, CG119621-02 | | | MW at |
| Protein Sequence | SEQ ID NO: 58 | 458 aa | 51943.5 kD |

PNSTMGCMKSKFLQVGGNTFSKTETSASAHCPVYVPDPTSTIKPGPNSHN
SNTPGIREAGSEDIIVVALYDYEAIHHEDLSFQKGDQMVVLEESGEWWKA
RSLATRKEGYIPSNYVARVDSLETEEWFFKGISRKDAERQLLAPGNMLGS
FMIRDSETTKGSYSLSVRDYDPRQGDTVKHYKIRTLDNGGFYISPRSTFS
TLQELVDHYKTTYNKHTKVAVKTMKPGSMSVEAFLAEANVMKTLQHDKLV
KLHAVVTKEPIYIITEFMAKGSLLDFLKSDEGSKQPLPKLIDFSAQIAEG
MAFIEQRNYIHRDLHAANILVSASLVCKIADFGLARVIEDNEYTAREGAK
FPIKWTAPEAINFGSFTIKSDVWSFGILLMEIVTYGRIPYPGMSNPEVIR
ALERGYRMPRPENCPEELYNIMMRCWKNRPEERPTFEYIQSVLDDFYTAT
ESQYQQQP

| | | |
|---|---|---|
| NOV5b, | SEQ ID NO: 59 | 1926 bp |
| CG119621-01 | ORF Start: | ORF Stop: |
| DNA Sequence | ATG at 76 | TGA at 1591 |

GAATTCCTTTCTAAAATCCAACCATTCCAGGAAATAGAAATATCAACTTG
GGGGCTTCCTGAGAATGTCAGATTGATGGGGTGCATGAAGTCCAAGTTCC
TCCAGGTCGGAGGCAATACATTCTCAAAAACTGAAACCAGCGCCAGCCCA
CACTGTCCTGTGTACGTGCCGGATCCCACATCCACCATCAAGCCGGGGCC
TAATAGCCACAACAGCAACACACCAGGAATCAGGGAGGCAGGCTCTGAGG
ACATCATCGTGGTTGCCCTGTATGATTACGAGGCCATTCACCACGAAGAC
CTCAGCTTCCAGAAGGGGGACCAGATGGTGGTCCTAGAGGAATCCGGGGA
GTGGTGGAAGGCTCGATCCCTGGCCACCCGGAAGGAGGGCTACATCCCAA
GCAACTATGTCGCCCGCGTTGACTCTCTGGAGACAGAGGAGTGGTTTTTC
AAGGGCATCAGCCGGAAGGACGCAGAGCGCCAACTGCTGGCTCCCGGCAA
CATGCTGGGCTCCTTCATGATCCGGGATAGCGAGACCACTAAAGGAAGCT
ACTCTTTGTCCGTGCGAGACTACGACCCTCGGCAGGGAGATACCGTGAAA
CATTACAAGATCCGGACCCTGGACAACGGGGGCTTCTACATATCCCCCCG
AAGCACCTTCAGCACTCTGCAGGAGCTGGTGGACCACTACAAGAAGGGGA
ACGACGGGCTCTGCCAGAAACTGTCGGTGCCCTGCATGTCTTCCAAGCCC
CAGAAGCCTTGGGAGAAAGATGCCTGGGAGATCCCTCGGGAATCCCTCAA
GCTGGAGAAGAAACTTGGAGCTGGGCAGTTTGGGGAAGTCTGGATGGCCA
CCTACAACAAGCACACCAAGGTGGCAGTGAAGACGATGAAGCCAGGGAGC
ATGTCGGTGGAGGCCTTCCTGGCAGAGGCCAACGTGATGAAAACTCTGCA
GCATGACAAGCTGGTCAAACTTCATGCGGTGGTCACCAAGGAGCCCATCT
ACATCATCACGGAGTTCATGGCCAAAGGAAGCTTGCTGGACTTTCTGAAA
AGTGATGAGGGCAGCAAGCAGCCATTGCCAAAACTCATTGACTTCTCAGC
CCAGATTGCAGAAGGCATGGCCTTCATCGAGCAGAGGAACTACATCCACC
GAGACCTCCGAGCTGCCAACATCTTGGTCTCTGCATCCCTGGTGTGTAAG
ATTGCTGACTTTGGCCTGGCCCGGGTCATTGAGGACAACGAGTACACGGC
TCGGGAAGGGGCCAAGTTCCCCATCAAGTGGACAGCTCCTGAAGCCATCA
ACTTTGGCTCCTTCACCATCAAGTCAGACGTCTGGTCCTTTGGTATCCTG
CTGATGGAGATCGTCACCTACGGCCGGATCCCTTACCCAGGGATGTCAAA
CCCTGAAGTGATCCGAGCTCTGGAGCGTGGATACCGGATGCCTCGCCCAG
AGAACTGCCCAGAGGAGCTCTACAACATCATGATGCGCTGCTGGAAAAAC
CGTCCGGAGGAGCGGCCGACCTTCGAATACATCCAGAGTGTGCTGGATGA
CTTCTACACGGCCACAGAGAGCCAGTACCAACAGCAGCCATGATAGGGAG
GACCAGGGCAGGGCAGGGGGTGCCCAGGTGGTGGCTGCAAGGTGGCTCCA
GCACCATCCGCCAGGGCCCACACCCCCTTCCTTCACCCAGACACCCACCC
TCGCTTCAGGCCACAGTTTCCTCATCTGTCCAGTGGGTAGGTTGGACTGG
AAAATCTCTTTTTGACTCTTGCAATGGACAATCTGACATTCTCAGGAAGC
CCCCAAGTTGATATTTCTATTTCCTGGAATGGTTGGATTTTAGTTACAGC
TGTGATTTGGAAGGGAAACTTTCAAAATAGTGAAATGAATATTTAAATAA
AAGATATAAATGCCAAAGTCTTTACC

| | | | |
|---|---|---|---|
| NOV5b, CG119621-01 | | | MW at |
| Protein Sequence | SEQ ID NO: 60 | 505 aa | 57311.8 kD |

MGCMKSKFLQVGGNTFSKTETSASPHCPVYVPDPTSTIKPGPNSHNSNTP
GIREAGSEDIIVVALYDYEAIHHEDLSFQKGDQMVVLEESGEWWKARSLA
TRKEGYIPSNYVARVDSLETEEWFFKGISRKDAERQLLAPGNMLGSFMIR
DSETTKGSYSLSVRDYDPRQGDTVKHYKIRTLDNGGFYISPRSTFSTLQE
LVDHYKKGNDGLCQKLSVPCMSSKPQKPWEKDAWEIPRESLKLEKKLGAG
QFGEVWMATYNKHTKVAVKTMKPGSMSVEAFLAEANVMKTLQHDKLVKLH
AVVTKEPIYIITEFMAKGSLLDFLKSDEGSKQPLPKLIDFSAQIAEGMAF
IEQRNYIHRDLRAANILVSASLVCKIADFGLARVIEDNEYTAREGAKFPI
KWTAPEAINFGSFTIKSDVWSFGILLMEIVTYGRIPYPGMSNPEVIRALE
RGYRMPRPEMCPEELYNIMMRCWKNRPEERPTFEYIQSVLDDFYTATESQ
YQQQP

A ClustalW comparison of the above protein sequences yields the following sequence alignment shown in Table 5B.

TABLE 5B
Comparison of the NOV5 protein sequences.

| | |
|---|---|
| NOV5a | PNSTMGCMKSKFLQVGGNTFSKTETSASAHCPVYVPDPTSTIKPGPNSHNSNTPGIREAG |
| NOV5b | ----MGCMKSKFLQVGGNTFSKTETSASPHCPVYVPDPTSTIKPGPNSHNSNTPGIREAG |
| NOV5a | SEDIIVVALYDYEAIHHEDLSFQKGDQMVVLEESGEWWKARSLATRKEGYIPSNYVARVD |
| NOV5b | SEDIIVVALYDYEAIHHEDLSFQKGDQMVVLEESGEWWKARSLATRKEGYIPSNYVARVD |
| NOV5a | SLETEEWFFKGISRKDAERQLLAPGNMLGSFMIRDSETTKGSYSLSVRDYDPRQGDTVKH |
| NOV5b | SLETEEWFFKGISRKDAERQLLAPGNMLGSFMIRDSETTKGSYSLSVRDYDPRQGDTVKH |
| NOV5a | YKIRTLDNGGFYISPRSTFSTLQELVDHYKT----------------------------- |
| NOV5b | YKIRTLDNGGFYISPRSTFSTLQELVDHYKKGNDGLCQKLSVPCMSSKPQKPWEKDAWEI |
| NOV5a | ----------------------TYNKHTKVAVKTMKPGSMSVEAFLAEANVMKTLQNDKL |
| NOV5b | PRESLKLEKKLGAGQFGEVWMATYNKHTKVAVKTMKPGSMSVEAFLAEANVMKTLQHDKL |
| NOV5a | VKLHAVVTKEPIYIITEFMAKGSLLDFLKSDEGSKQPLPKLIDFSAQIAEGMAFIEQRNY |
| NOV5b | VKLHAVVTKEPIYIITEFMAKGSLLDFLKSDEGSKQPLPKLIDFSAQIAEGMAFIEQRNY |
| NOV5a | IHRDLRAANILVSASLVCKIADFGLARVIEDNEYTAREGAKFPIKWTAPEAINFGSFTIK |
| NOV5b | IHRDLRAANILVSASLVCKIADFGLARVIEDNEYTAREGAKFPIKWTAPEAINFGSFTIK |
| NOV5a | SDVWSFGILLMEIVTYGRIPYPGMSNPEVIRALERGYRMPRPENCPEELYNIMMRCWKNR |
| NOV5b | SDVWSFGILLMEIVTYGRIPYPGMSNPEVIRALERGYRMPRPENCPEELYNIMMRCWKNR |
| NOV5a | PEERPTFEYIQSVLDDFYTATESQYQQQP |
| NOV5b | PEERPTFEYIQSVLDDFYTATESQYQQQP |
| NOV5a | (SEQ ID NO: 58) |
| NOV5b | (SEQ ID NO: 60) |

Further analysis of the NOV5a protein yielded the following properties shown in Table 5C.

TABLE 5C
Protein Sequence Properties NOV5a

| SignalP | No Known Signal Sequence Predicted | | | |
|---|---|---|---|---|
| analysis: | | | | |
| PSORT II | PSG: a new signal peptide prediction method | | | |
| analysis: | N-region: length 11; pos. chg 2; neg. chg 0 | | | |
| | H-region: length 10; peak value 1.77 | | | |
| | PSG score: −2.63 | | | |
| | GvH: von Heijne's method for signal seq. recognition | | | |
| | GvH score (threshold: −2.1): −11.03 | | | |
| | possible cleavage site: between 41 and 42 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 0 | | | |
| | PERIPHERAL | Likelihood = | 2.76 (at 372) | |
| | ALOM score: −1.70 (number of TMSs: 0) | | | |
| | MITDISC: discrimination of mitochondrial targeting seq | | | |
| | R content: | 0 | Hyd Moment (75): | 3.16 |
| | Hyd Moment (95): | 4.34 | G content: | 3 |
| | D/E content: | 1 | S/T content: | 6 |
| | Score: | −5.23 | | |

```
Gavel: prediction of cleavage sites for mitochondrial preseq
cleavage site motif not found
NUCDISC: discrimination of nuclear localization signals
pat4: none
pat7: none
bipartite: none
content of basic residues: 11.8%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 55.5
COIL: Lupas's algorithm to detect coiled-coil regions
total: 0 residues
Final Results (k = 9/23):
34.8%: cytoplasmic
30.4%: nuclear
26.1%: mitochondrial
  4.3%: Golgi
  4.3%: endoplasmic reticulum
>> prediction for CG119621-02 is cyt (k = 23)
```

A search of the NOV5a protein against the Geneseq database, a proprietary database that contains sequences published in patents and patent publication, yielded several homologous proteins shown in Table 5D.

TABLE 5D
Geneseq Results for NOV5a

| | | | Identities/ | |
|---|---|---|---|---|
| | | NOV5a | Similarities | |
| | | Residues/ | for the | |
| Geneseq | Protein/Organism/Length | Match | Matched | Expect |
| Identifier | [Patent #, Date] | Residues | Region | Value |
| AAB99332 | Human tyrosine kinase Hck protein | 5 . . . 458 | 452/505 (89%) | 0.0 |
| | sequence SEQ ID NO: 11 - Homo | 1 . . . 505 | 452/505 (89%) | |
| | sapiens, 505 aa. [WO200132869-A1, | | | |
| | 10 MAY 2001] | | | |
| AAU08730 | Xenopus laevis src-family kinase | 5 . . . 458 | 311/506 (61%) | e−173 |
| | (SFK) polypeptide - Xenopus laevis, | 1 . . . 496 | 372/506 (73%) | |
| | 496 aa. [US6291651-B1, | | | |
| | 18 SEP. 2001] | | | |
| ABB81188 | Human KIT protein sequence - Homo | 209 . . . 458 | 247/250 (98%) | e−142 |
| | sapiens, 272 aa. [WO200261055-A2, | 23 . . . 272 | 248/250 (98%) | |
| | 08 AUG. 2002] | | | |
| AAY43957 | Human protein kinase #16 - Homo | 209 . . . 450 | 239/242 (98%) | e−137 |
| | sapiens, 259 aa. [US5958784-A, | 18 . . . 259 | 240/242 (98%) | |
| | 28 SEP. 1999] | | | |
| AAY43954 | Human protein kinase #14 - Homo | 213 . . . 450 | 198/239 (82%) | e−114 |
| | sapiens, 260 aa. [US5958784-A, | 22 . . . 260 | 221/239 (91%) | |
| | 28 SEP. 1999] | | | |

In a BLAST search of public sequence databases, the NOV5a protein was found to have homology to the proteins shown in the BLASTP data in Table 5E.

TABLE 5E
Public BLASTP Results for NOV5a

| | | | Identities/ | |
|---|---|---|---|---|
| | | NOV5a | Similarities | |
| Protein | | Residues/ | for the | |
| Accession | | Match | Matched | Expect |
| Number | Protein/Organism/Length | Residues | Portion | Value |
| TVHUHC | protein-tyrosine kinase (EC 2.7.1.112) | 5 . . . 458 | 452/505 (89%) | 0.0 |
| | hck - human, 505 aa. | 1 . . . 505 | 452/505 (89%) | |
| P08631 | Tyrosine-protein kinase HCK (EC | 5 . . . 458 | 452/505 (89%) | 0.0 |
| | 2.7.1.112) (p59-HCK/p60-HCK) | 22 . . . 526 | 452/505 (89%) | |
| | (Hemopoietic cell kinase) - Homo | | | |
| | sapiens (Human), 526 aa. | | | |
| Q95M30 | Tyrosine-protein kinase HCK (EC | 5 . . . 458 | 443/505 (87%) | 0.0 |
| | 2.7.1.112) (p56-HCK) (Hemopoietic cell | 1 . . . 504 | 447/505 (87%) | |
| | kinase) - Macaca fascicularis (Crab | | | |
| | eating macaque) (Cynomolgus monkey), | | | |
| | 504 aa. | | | |
| O13064 | Lyn protein tyrosine kinase - Xenopus | 45 . . . 458 | 314/473 (66%) | 0.0 |
| | laevis (African clawed frog), 488 aa. | 16 . . . 488 | 373/473 (78%) | |
| Q8N5D7 | Hypothetical protein - Homo sapiens | 41 . . . 455 | 271/417 (64%) | e−160 |
| | (Human), 482 aa. | 62 . . . 477 | 338/417 (80%) | |

PFam analysis predicts that the NOV5a protein contains the domains shown in the Table 5F.

TABLE 5F
Domain Analysis of NOV5a

| | | Identities/ | |
|---|---|---|---|
| | | Similarities | |
| Pfam | NOV5a Match | for the | |
| Domain | Region | Matched Region | Expect Value |
| SH3 | 64 . . . 119 | 33/58 (57%) | 1e−25 |
| | | 50/58 (86%) | |
| SH2 | 127 . . . 209 | 48/83 (58%) | 1.6e−42 |
| | | 77/83 (93%) | |
| pkinase | 199 . . . 443 | 80/298 (27%) | 3e−59 |
| | | 190/298 (64%) | |

### Example 6

The NOV6 clone was analyzed, and the nucleotide and encoded polypeptide sequences are shown in Table 6A.

TABLE 6A
NOV6 Sequence Analysis

| | | |
|---|---|---|
| NOV6a, | SEQ ID NO: 61 | 3109 bp |
| CG124553-01 | ORF Start: | ORF Stop: |
| DNA Sequence | ATG at 75 | TAG at 1479 |

AGGGGCTGAGGAGGTACTGGAAAAGAAAGAGGAGCAGGAGCTGGAGGA
GACGTGGAGGAGGAGCTGGAGGAGGATGAAGAGAAGGAGTGGGACGCCC
ACAACCCTGTGTAAGGAGCTCAAGTACTCCAAGGACCCGCCCCAGATATC
CATCATATTCATCTTCGTGAACGAGGCCCTGTCGGTGATCCTGCGGTCCG
TGCACAGTGCCGTCAATCACACGCCCACACACCTGCTGAAGGAAATCATT
CTGGTGGATGACAACAGCGACGAAGAGGAGCTGAAGGTCCCCCTAGAGGA
GTATGTCCACAAACGCTACCCCGGGCTGGTGAAGGTGGTAAGAAATCAGA
AGAGGGAAGGCCTGATCCGCGCTCGCATTGAGGGCTGGAAGGTGGCTACC
GGGCAGGTCACTGGCTTCTTTGATGCCCACGTGGAATTCACCGCTGGCTG
GGCTGAGCCGGTTCTATCCCGCATCCAGGAAAACCGGAAGCGTGTGATCC
TCCCCTCCATTGACAACATCAAACAGGACAACTTTGAGGTGCAGCGGTAC
GAGAACTCGGCCCACGGGTACAGCTGGGAGCTGTGGTGCATGTACATCAG
CCCCCCAAAAGACTGGTGGGACGCCGGAGACCCTTCTCTCCCCATCAGGA
CCCCAGCCATGATAGGCTGCTCGTTCGTGGTCAACAGGAAGTTCTTCGGT
GAAATTGGTCTTCTGGATCCTGGCATGGATGTATACGGAGGAGAAAATAT
TGAACTGGGAATCAAGGTATGGCTCTGTGGGGGCAGCATGGAGGTCCTTC
CTTGCTCACGGGTGGCCCACATTGAGCGGAAGAAGAAGCCATATAATAGC
AACATTGGCTTCTACACCAAGAGGAATGCTCTTCGCGTTGCTGAGGTCTG
GATGGACGATTACAAGTCTCATGTGTACATAGCGTGGAACCTGCCGCTGG
AGAATCCGGGAATTGACATCGGTGATGTCTCCGAAAGAAGAGCATTAAGG
AAAAGTTTAAAGTGTAAGAATTTCCAGTGGTACCTGGACCATGTTTACCC
AGAAATGAGAAGATACAATAATACCGTTGCTTACGGGGAGCTTCGCAACA
ACAAGGCAAAAGACGTCTGCTTGGACCAGGGGCCGCTGGAGAACCACACA
GCAATATTGTATCCGTGCCATGGCTGGGGACCACAGCTTGCCCGCTACAC
CAAGGAAGGCTTCCTGCACTTGGGTGCCCTGGGGACCACCACACTCCTCC
CTGACACCCGCTGCCTGGTGGACAACTCCAAGAGTCGGCTGCCCCAGCTC
CTGGACTGCGACAAGGTCAAGAGCAGCCTGTACAAGCGCTGGAACTTCAT
CCAGAATGGAGCCATCATGAACAAGGGCACGGGACGCTGCCTGGAGGTGG
AGAACCGGGGCCTGGCTGGCATCGACCTCATCCTCCGCAGCTGCACAGGT
CAGAGGTGGACCATTAAGAACTCCATCAAGTAGAGGGAGGGAGCTGGGGC
ACTGGAGCCTGGCCCCCAGGACATGGCTGCTCCCCCCAACATCTGGACCA
GCTGCCCTGGCGGAGAGACAGCAAGGGGCCGGCAGGTGCTCGATGGGCCC
CCCAGGGCTTCTCCAGGGCAGCACAGGGACCCCGGATGAAGACTCTGTCC
CCCCTCAGGCATTCAGCTGCCCACAAGTTTCCTGCACCCTGGAAAAGCCC
CCCACCCTTCCTCTGGGAAACTGACAGCTGTCTTCCACAGCCTCTGATGT
GGACCTGGTACTGAGGAGCAAGACTGTCCAGTTCTCCTCCACATCTCCCA
TCCCAGAATCAGGATCTGGGACTGGCAGGGTCCCCTCCTGTGTCTCATCT
CTTGCAGCAGCAGCTGCTGAACTCCAGCCATCAACACGGTGGGAGGCAGC
GGGGGCTTCAGCCATGTCCTAGCTCCCCGCCCTAAAAGGAGGCAGTGAGG
ACCAGGCACTATTTCCTCCGAGGTTACTTCTACCCAGATGACACCTGCCT
GTTCACGCCCCAAGGCAGCTACTGCCCCTAACCCTTCCCACCAGGGTAGC
TTTGGGCACTGCAGCTCTGGACTTTTCTGGCCCCTCCTGAGATGACCTGA
TGGAGCTGATGCTTTCTCTCCTAATCCCTGGGCACTAGGCTCTTATCAGT
GTGCTTGGGCCAGCTCTCCTGCCTGTGTCTAGAGGAAGCCAGAGACAGAA
ATAGGCTAAGCCTGCAGTAGGATCTCAGCCACAAGGGCCCCGCAGGATGG
AGCTGGGTCAAGGACCAGGGAGCCCTGACTCCCAGAGGCTGCCACCGGGG
AGAAGCAGCGGTCCTCCATCCAGAACCTAAGGGCTGAAGCAAAGGCTGCC
AGGACCCTTGAAGATGCTTTTGGCTCACCTCATTTCACCCCACGCTCTGC
TGGCTGGCAGAGGAGAAGGCAGTCGTTTCCTCTCTGAAGAGTATTTTTTT
CGATTGCCCTCTGGTTAGGGTGCACATATAAATCAGAGTTAATATATGAA
CGCGTGTGCATGCACAAGTGTGTGTGTGCCTGCGTGCTGTGCGTGGCAGG
GTGTGTGTGTGTGTGTCTGGCTGTGCGTTCCGGAGTGTGTGACGATGCTG
ACCTAGCTGTGTGGCCTTGGGCTTGCTGCTTCATTACTCACCTGGATGGG
GACGAGGGATGAGAAGGGTGTGGGTTTGGCCCCATGTCACTGGCCGGAAG
GATGTGTCTCAGCCCTGCCCTGTGGGGTGCCCCCGATGGGAGGCTGTCCC
ATCTCCCAGTCCCCATCTCTTTTTCCCCACACTGTCCCTGGCCAAGCCCT
GCCCAGAGCTGAACCCTGTAGCTGCCCCCTTGCCCTGTGTGGGATTCGCA
GTGTCTCATTTGGTGACGTCTTACTGGTGATCATCTCCTCACCCCATCTC
CCACCTTGTGGAATAAATACATGTTAGCACTTCCCAGAGCAGCCTCCTTT
GTGTCTTGATTTCTCCAGAACTGGAGGTGGGGAGGGGAGTGATGGAGACA
TAGGAGGAGAGCTTCTTTGGCTTTGAGGGTTTAGTGTTACTTATTTATCT
ATTTATTCGAGATGGGGTCTTGCTCTGTGGCCCAGGCTGGAGTGCAGTGG
TGCAATCATGA

| | | | |
|---|---|---|---|
| NOV6a, CG124553-01 | | | MW at |
| Protein Sequence | SEQ ID NO: 62 | 468 aa | 53596.1 kD |

MKRRSGTPTTLCKELKYSKDPPQISIIFIFVNEALSVILRSVHSAVNHTP
THLLKEIILVDDNSDEEELKVPLEEYVHKRYPGLVKVVRNQKREGLIRAR
IEGWKVATGQVTGFFDAHVEFTAGWAEPVLSRIQENRKRVILPSIDNIKQ
DNFEVQRYENSAHGYSWELWCMYISPPKDWWDAGDPSLPIRTPANIGCSF
VVNRKFFGEIGLLDPGMDVYGGENIELGIKVWLCGGSMEVLPCSRVAHIE
RKKKPYNSNIGFYTKRNALRVAEVWMDDYKSHVYIAWNLPLENPGIDIGD
VSERRALRKSLKCKNFQWYLDHVYPEMRRYNNTVAYGELRNNKAKDVCLD
QGPLENHTAILYPCHGWGPQLARYTKEGFLHLGALGTTTLLPDTRCLVDN
SKSRLPQLLDCDKVKSSLYKRWNFIQNGAIMNKGTGRCLEVENRGLAGID
LILRSCTGQRWTIKNSIK

| | | |
|---|---|---|
| NOV6b, | SEQ ID NO: 63 | 1306 bp |
| 277206285 | ORF start: | ORF Stop: |
| DNA Sequence | at 2 | end of sequence |

CACCAGATCTCGGTCCGTGCACAGTGCCGTCAATCACACGCCCACACACC
TGCTGAAGGAAATCATTCTGGTGGATGACAACAGCGACGAAGAGGAGCTG
AAGGTCCCCCTAGAGGAGTATGTCCACAAACGCTACCCCGGGCTGGTGAA
GGTGGTAAGAAATCAGAAGAGGGAAGGCCTGATCCGCGCTCGCATTGAGG
GCTGGAAGGTGGCTACCGGGCAGGTCACTGGCTTCTTTGATGCCCACGTG
GAATTCACCGCTGGCTGGGCTGAGCCGGTTCTATCCCGCATCCAGGAAAA
CCGGAAGCGTGTGATCCTCCCCTCCATTGACAACATCAAACAGGACAACT
TTGAGGTGCAGCGGTACGAGAACTCGGCCCACGGGTACAGCTGGGAGCTG
TGGTGCATGTACATCAGCCCCCCAAAAGACTGGTGGGACGCCGGAGACCC
TTCTCTCCCCATCAGGACCCCAGCCATGATAGGCTGCTCGTTCGTGGTCA
ACAGGAAGTTCTTCGGTGAAATTGGTCTTCTGGATCCTGGCATGGATGTA
TACGGAGGAGAAAATATTGAACTGGGAATCAAGGTATGGCTCTGTGGGGG
CAGCATGGAGGTCCTTCCTTGCTCACGGGTGGCCCACATTGAGCGGAAGA
AGAAGCCATATAATAGCAACATTGGCTTCTACACCAAGAGGAATGCTCTT
CGCGTTGCTGAGGTCTGGATGGACGATTACAAGTCTCATGTGTACATAGC
GTGGAACCTGCCGCTGGAGAATCCGGGAATTGACATCGGTGATGTCTCCG
AAAGAAGAGCATTAAGGAAAAGTTTAAAGTGTAAGAATTTCCAGTGGTAC
CTGGACCATGTTTACCCAGAAATGAGAAGATACAATAATACCGTTGCTTA
CGGGGAGCTTCGCAACAACAAGGCAAAAGACGTCTGCTTGGACCAGGGGC
CGCTGGAGAACCACACAGCAATATTGTATCCGTGCCATGGCTGGGGACCA
CAGCTTGCCCGCTACACCAAGGAAGGCTTCCTGCACTTGGGTGCCCTGGG
GACCACCACACTCCTCCCTGACACCCGCTGCCTGGTGGACAACTCCAAGA
GTCGGCTGCCCCAGCTCCTGGACTGCGACAAGGTCAAGAGCAGCCTGTAC
AAGCGCTGGAACTTCATCCAGAATGGAGCCATCATGAACAAGGGCACGGG
ACGCTGCCTGGAGGTGGAGAACCGGGGCCTGGCTGGCATCGACCTCATCC
TCCGCAGCTGCACAGGTCAGAGGTGGACCATTAAGAACTCCATCAAGCTC
GAGGGC

| | | | |
|---|---|---|---|
| NOV6b, 277206285 | | | MW at |
| Protein Sequence | SEQ ID NO: 64 | 435 aa | 49819.6 kD |

TRSRSVHSAVNHTPTHLLKEIILVDDNSDEEELKVPLEEYVHKRYPGLVK
VVRNQKREGLIRARIEGWKVATGQVTGFFDAHVEFTAGWAEPVLSRIQEN
RKRVILPSIDNIKQDNFEVQRYENSAHGYSWELWCMYISPPKDWWDAGDP
SLPIRTPAMIGCSFVVNRKFFGEIGLLDPGMDVYGGENIELGIKVWLCGG
SMEVLPCSRVAHIERKKKPYNSNIGFYTKRNALRVAEVWMDDYKSHVYIA
WNLPLENPGIDIGDVSERRALRKSLKCKNFQWYLDHVYPEMRRYNNTVAY
GELRNNKAKDVCLDQGPLENHTAILYPCHGWGPQLARYTKEGFLHLGALG
TTTLLPDTRCLVDNSKSRLPQLLDCDKVKSSLYKRWNFIQNGAIMNKGTG
RCLEVENRGLAGIDLILRSCTGQRWTIKNSIKLEG

| | | |
|---|---|---|
| NOV6c, | SEQ ID NO: 65 | 3109 bp |
| SNP13379778 of | ORF Start: | ORF Stop: |
| CG124553-01, | ATG at 75 | TAG at 1479 |
| DNA Sequence | SNP Pos: 570 | SNP Change: A to G |

AGGGGCTGAGGAGGTACTGGAAAAGAAAGAGGAGCAGGAGCTGGAGGAAG
ACGTGGAGGAGGAGCTGGAGGAGGATGAAGAGAAGGAGTGGGACGCCCAC
AACCCTGTGTAAGGAGCTCAAGTACTCCAAGGACCCGCCCCAGATATCCA
TCATATTCATCTTCGTGAACGAGGCCCTGTCGGTGATCCTGCGGTCCGTG
CACAGTGCCGTCAATCACACGCCCACACACCTGCTGAAGGAAATCATTCT
GGTGGATGACAACAGCGACGAAGAGGAGCTGAAGGTCCCCCTAGAGGAGT
ATGTCCACAAACGCTACCCCGGGCTGGTGAAGGTGGTAAGAAATCAGAAG
AGGGAAGGCCTGATCCGCGCTCGCATTGAGGGCTGGAAGGTGGCTACCGG
GCAGGTCACTGGCTTCTTTGATGCCCACGTGGAATTCACCGCTGGCTGGG
CTGAGCCGGTTCTATCCCGCATCCAGGAAAACCGGAAGCGTGTGATCCTC
CCCTCCATTGACAACATCAAACAGGACAACTTTGAGGTGCAGCGGTACGA
GAACTCGGCCCACGGGTACGGCTGGGAGCTGTGGTGCATGTACATCAGCC
CCCCAAAAGACTGGTGGGACGCCGGAGACCCTTCTCTCCCCATCAGGACC
CCAGCCATGATAGGCTGCTCGTTCGTGGTCAACAGGAAGTTCTTCGGTGA
AATTGGTCTTCTGGATCCTGGCATGGATGTATACGGAGGAGAAAATATTG
AACTGGGAATCAAGGTATGGCTCTGTGGGGGCAGCATGGAGGTCCTTCCT
TGCTCACGGGTGGCCCACATTGAGCGGAAGAAGAAGCCATATAATAGCAA
CATTGGCTTCTACACCAAGAGGAATGCTCTTCGCGTTGCTGAGGTCTGGA
TGGACGATTACAAGTCTCATGTGTACATAGCGTGGAACCTGCCGCTGGAG
AATCCGGGAATTGACATCGGTGATGTCTCCGAAAGAAGAGCATTAAGGAA
AAGTTTAAAGTGTAAGAATTTCCAGTGGTACCTGGACCATGTTTACCCAG
AAATGAGAAGATACAATAATACCGTTGCTTACGGGGAGCTTCGCAACAAC
AAGGCAAAAGACGTCTGCTTGGACCAGGGGCCGCTGGAGAACCACACAGC
AATATTGTATCCGTGCCATGGCTGGGGACCACAGCTTGCCCGCTACACCA
AGGAAGGCTTCCTGCACTTGGGTGCCCTGGGGACCACCACACTCCTCCCT
GACACCCGCTGCCTGGTGGACAACTCCAAGAGTCGGCTGCCCCAGCTCCT
GGACTGCGACAAGGTCAAGAGCAGCCTGTACAAGCGCTGGAACTTCATCC
AGAATGGAGCCATCATGAACAAGGGCACGGGACGCTGCCTGGAGGTGGAG
AACCGGGGCCTGGCTGGCATCGACCTCATCCTCCGCAGCTGCACAGGTCA
GAGGTGGACCATTAAGAACTCCATCAAGTAGAGGGAGGGAGCTGGGGCAC
TGGAGCCTGGCCCCCAGGACATGGCTGCTCCCCCCAACATCTGGACCAGC
TGCCCTGGCGGAGAGACAGCAAGGGGCCGGCAGGTGCTCGATGGGCCCCC
CAGGGCTTCTCCAGGGCAGCACAGGGACCCCGGATGAAGACTCTGTCCCC
CCTCAGGCATTCAGCTGCCCACAAGTTTCCTGCACCCTGGAAAAGCCCCC
CACCCTTCCTCTGGGAAACTGACAGCTGTCTTCCACAGCCTCTGATGTGG
ACCTGGTACTGAGGAGCAAGACTGTCCAGTTCTCCTCCACATCTCCCATC
CCAGAATCAGGATCTGGGACTGGCAGGGTCCCCTCCTGTGTCTCATCTCT
TGCAGCAGCAGCTGCTGAACTCCAGCCATCAACACGGTGGGAGGCAGCGG
GGGCTTCAGCCATGTCCTAGCTCCCCGCCCTAAAAGGAGGCAGTGAGGAC
CAGGCACTATTTCCTCCGAGGTTACTTCTACCCAGATGACACCTGCCTGT
TCACGCCCCAAGGCAGCTACTGCCCCTAACCCTTCCCACCAGGGTAGCTT
TGGGCACTGCAGCTCTGGACTTTTCTGGCCCCTCCTGAGATGACCTGATG
GAGCTGATGCTTTCTCTCCTAATCCCTGGGCACTAGGCTCTTATCAGTGT
GCTTGGGCCAGCTCTCCTGCCTGTGTCTAGAGGAAGCCAGAGACAGAAAT
AGGCTAAGCCTGCAGTAGGATCTCAGCCACAAGGGCCCCGCAGGATGGAG
CTGGGTCAAGGACCAGGGAGCCCTGACTCCCAGAGGCTGCCACCGGGGAG
AAGCAGCGGTCCTCCATCCAGAACCTAAGGGCTGAAGCAAAGGCTGCCAG
GACCCTTGAAGATGCTTTTGGCTCACCTCATTTCACCCCACGCTCTGCTG
GCTGGCAGAGGAGAAGGCAGTCGTTTCCTCTCTGAAGAGTATTTTTTTCG
ATTGCCCTCTGGTTAGGGTGCACATATAAATCAGAGTTAATATATGAACG
CGTGTGCATGCACAAGTGTGTGTGTGCCTGCGTGCTGTGCGTGGCAGGGT
GTGTGTGTGTGTGTCTGGCTGTGCGTTCCGGAGTGTGTGACGATGCTGAC
CTAGCTGTGTGGCCTTGGGCTTGCTGCTTCATTACTCACCTGGATGGGGA
CGAGGGATGAGAAGGGTGTGGGTTTGGCCCCATGTCACTGGCCGGAAGGA
TGTGTCTCAGCCCTGCCCTGTGGGGTGCCCCCGATGGGAGGCTGTCCCAT
CTCCCAGTCCCCATCTCTTTTTCCCCACACTGTCCCTGGCCAAGCCCTGC
CCAGAGCTGAACCCTGTAGCTGCCCCCTTGCCCTGTGTGGGATTCGCAGT
GTCTCATTTGGTGACGTCTTACTGGTGATCATCTCCTCACCCCATCTCCC
ACCTTGTGGAATAAATACATGTTAGCACTTCCCAGAGCAGCCTCCTTTGT
GTCTTGATTTCTCCAGAACTGGAGGTGGGGAGGGGAGTGATGGAGACATA
GGAGGAGAGCTTCTTTGGCTTTGAGGGTTTAGTGTTACTTATTTATCTAT
TTATTCGAGATGGGGTCTTGCTCTGTGGCCCAGGCTGGAGTGCAGTGGTG
CAATCATGA

| | | | |
|---|---|---|---|
| NOV6c, | | | MW at |
| SNP13379778 of | | | 53566.1 kD |
| CG124553-01, | SEQ ID NO: 66 | | SNP Change: |
| Protein Sequence | SNP Pos: 166 | 468 aa | Ser to Gly |

MKRRSGTPTTLCKELKYSKDPPQISIIFIFVNEALSVILRSVHSAVNHTP
THLLKEIILVDDNSDEEELKVPLEEYVHKRYPGLVKVVRNQKREGLIRAR
IEGWKVATGQVTGFFDAHVEFTAGWAEPVLSRIQENRKRVILPSIDNIKQ
DNFEVQRYENSAHGYGWELWCMYISPPKDWWDAGDPSLPIRTPAMIGCSF
VVNRKFFGEIGLLDPGMDVYGGENIELGIKVWLCGGSMEVLPCSRVAHIE
RKKKPYNSNIGFYTKRNALRVAEVWMDDYKSHVYIAWNLPLENPGIDIGD
VSERRALRKSLKCKNFQWYLDHVYPEMRRYNNTVAYGELRNNKAKDVCLD
QGPLENHTAILYPCHGWGPQLARYTKEGFLHLGALGTTTLLPDTRCLVDN
SKSRLPQLLDCDKVKSSLYKRWNFIQNGAIMNKGTGRCLEVENRGLAGID
LILRSCTGQRWTIKNSIK

| | | |
|---|---|---|
| NOV6d, | SEQ ID NO: 67 | 3109 bp |
| SNP13379831 of | ORF Start: | ORF Stop: |
| CG124553-01, | ATG at 75 | TAG at 1479 |
| DNA Sequence | SNP Pos: 1527 | SNP Change: G to C |

AGGGGCTGAGGAGGTACTGGAAAAGAAAGAGGAGCAGGAGCTGGAGGAAG
ACGTGGAGGAGGAGCTGGAGGAGGATGAAGAGAAGGAGTGGGACGCCCAC
AACCCTGTGTAAGGAGCTCAAGTACTCCAAGGACCCGCCCCAGATATCCA
TCATATTCATCTTCGTGAACGAGGCCCTGTCGGTGATCCTGCGGTCCGTG
CACAGTGCCGTCAATCACACGCCCACACACCTGCTGAAGGAAATCATTCT
GGTGGATGACAACAGCGACGAAGAGGAGCTGAAGGTCCCCCTAGAGGAGT
ATGTCCACAAACGCTACCCCGGGCTGGTGAAGGTGGTAAGAAATCAGAAG
AGGGAAGGCCTGATCCGCGCTCGCATTGAGGGCTGGAAGGTGGCTACCGG
GCAGGTCACTGGCTTCTTTGATGCCCACGTGGAATTCACCGCTGGCTGGG
CTGAGCCGGTTCTATCCCGCATCCAGGAAAACCGGAAGCGTGTGATCCTC
CCCTCCATTGACAACATCAAACAGGACAACTTTGAGGTGCAGCGGTACGA
GAACTCGGCCCACGGGTACAGCTGGGAGCTGTGGTGCATGTACATCAGCC
CCCCAAAAGACTGGTGGGACGCCGGAGACCCTTCTCTCCCCATCAGGACC
CCAGCCATGATAGGCTGCTCGTTCGTGGTCAACAGGAAGTTCTTCGGTGA
AATTGGTCTTCTGGATCCTGGCATGGATGTATACGGAGGAGAAAATATTG
AACTGGGAATCAAGGTATGGCTCTGTGGGGGCAGCATGGAGGTCCTTCCT
TGCTCACGGGTGGCCCACATTGAGCGGAAGAAGAAGCCATATAATAGCAA
CATTGGCTTCTACACCAAGAGGAATGCTCTTCGCGTTGCTGAGGTCTGGA
TGGACGATTACAAGTCTCATGTGTACATAGCGTGGAACCTGCCGCTGGAG
AATCCGGGAATTGACATCGGTGATGTCTCCGAAAGAAGAGCATTAAGGAA
AAGTTTAAAGTGTAAGAATTTCCAGTGGTACCTGGACCATGTTTACCCAG
AAATGAGAAGATACAATAATACCGTTGCTTACGGGGAGCTTCGCAACAAC
AAGGCAAAAGACGTCTGCTTGGACCAGGGGCCGCTGGAGAACCACACAGC
AATATTGTATCCGTGCCATGGCTGGGGACCACAGCTTGCCCGCTACACCA
AGGAAGGCTTCCTGCACTTGGGTGCCCTGGGGACCACCACACTCCTCCCT
GACACCCGCTGCCTGGTGGACAACTCCAAGAGTCGGCTGCCCCAGCTCCT
GGACTGCGACAAGGTCAAGAGCAGCCTGTACAAGCGCTGGAACTTCATCC
AGAATGGAGCCATCATGAACAAGGGCACGGGACGCTGCCTGGAGGTGGAG
AACCGGGGCCTGGCTGGCATCGACCTCATCCTCCGCAGCTGCACAGGTCA
GAGGTGGACCATTAAGAACTCCATCAAGTAGAGGGAGGGAGCTGGGGCAC
TGGAGCCTGGCCCCCAGGACATGGCTCCTCCCCCCAACATCTGGACCAGC
TGCCCTGGCGGAGAGACAGCAAGGGGCCGGCAGGTGCTCGATGGGCCCCC
CAGGGCTTCTCCAGGGCAGCACAGGGACCCCGGATGAAGACTCTGTCCCC
CCTCAGGCATTCAGCTGCCCACAAGTTTCCTGCACCCTGGAAAAGCCCCC
CACCCTTCCTCTGGGAAACTGACAGCTGTCTTCCACAGCCTCTGATGTGG
ACCTGGTACTGAGGAGCAAGACTGTCCAGTTCTCCTCCACATCTCCCATC
CCAGAATCAGGATCTGGGACTGGCAGGGTCCCCTCCTGTGTCTCATCTCT
TGCAGCAGCAGCTGCTGAACTCCAGCCATCAACACGGTGGGAGGCAGCGG
GGGCTTCAGCCATGTCCTAGCTCCCCGCCCTAAAAGGAGGCAGTGAGGAC
CAGGCACTATTTCCTCCGAGGTTACTTCTACCCAGATGACACCTGCCTGT
TCACGCCCCAAGGCAGCTACTGCCCCTAACCCTTCCCACCAGGGTAGCTT
TGGGCACTGCAGCTCTGGACTTTTCTGGCCCCTCCTGAGATGACCTGATG
GAGCTGATGCTTTCTCTCCTAATCCCTGGGCACTAGGCTCTTATCAGTGT
GCTTGGGCCAGCTCTCCTGCCTGTGTCTAGAGGAAGCCAGAGACAGAAAT
AGGCTAAGCCTGCAGTAGGATCTCAGCCACAAGGGCCCCGCAGGATGGAG
CTGGGTCAAGGACCAGGGAGCCCTGACTCCCAGAGGCTGCCACCGGGGAG
AAGCAGCGGTCCTCCATCCAGAACCTAAGGGCTGAAGCAAAGGCTGCCAG
GACCCTTGAAGATGCTTTTGGCTCACCTCATTTCACCCCACGCTCTGCTG
GCTGGCAGAGGAGAAGGCAGTCGTTTCCTCTCTGAAGAGTATTTTTTTCG
ATTGCCCTCTGGTTAGGGTGCACATATAAATCAGAGTTAATATATGAACG
CGTGTGCATGCACAAGTGTGTGTGTGCCTGCGTGCTGTGCGTGGCAGGGT
GTGTGTGTGTGTGTCTGGCTGTGCGTTCCGGAGTGTGTGACGATGCTGAC
CTAGCTGTGTGGCCTTGGGCTTGCTGCTTCATTACTCACCTGGATGGGGA
CGAGGGATGAGAAGGGTGTGGGTTTGGCCCCATGTCACTGGCCGGAAGGA
TGTGTCTCAGCCCTGCCCTGTGGGGTGCCCCCGATGGGAGGCTGTCCCAT
CTCCCAGTCCCCATCTCTTTTTCCCCACACTGTCCCTGGCCAAGCCCTGC
CCAGAGCTGAACCCTGTAGCTGCCCCCTTGCCCTGTGTGGGATTCGCAGT
GTCTCATTTGGTGACGTCTTACTGGTGATCATCTCCTCACCCCATCTCCC
ACCTTGTGGAATAAATACATGTTAGCACTTCCCAGAGCAGCCTCCTTTGT
GTCTTGATTTCTCCAGAACTGGAGGTGGGGAGGGGAGTGATGGAGACATA
GGAGGAGAGCTTCTTTGGCTTTGAGGGTTTAGTGTTACTTATTTATCTAT
TTATTCGAGATGGGGTCTTGCTCTGTGGCCCAGGCTGGAGTGCAGTGGTG
CAATCATGA

| | | | |
|---|---|---|---|
| NOV6d, | | | MW at |
| SNP13379831 of | | | 53596.1 kD |
| CG124553-01, | | | SNP Change: |
| Protein Sequence | SEQ ID NO: 68 | 468 aa | no change |

MKRRSGTPTTLCKELKYSKDPPQISIIFIFVNEALSVILRSVHSAVNHTP
THLLKEIILVDDNSDEEELKVPLEEYVHKRYPGLVKVVRNQKREGLIRAR
IEGWKVATGQVTGFFDAHVEFTAGWAEPVLSRIQENRKRVILPSIDNIKQ
DNFEVQRYENSAHGYSWELWCMYISPPKDWWDAGDPSLPIRTPAMIGCSF
VVNRKFFGEIGLLDPGMDVYGGENIELGIKVWLCGGSMEVLPCSRVAHIE
RKKKPYNSNIGFYTKRNALRVAEVWMDDYKSHVYIAWNLPLENPGIDIGD
VSERRALRKSLKCKNFQWYLDHVYPEMRRYNNTVAYGELRNNKAKDVCLD
QGPLENHTAILYPCHGWGPQLARYTKEGFLHLGALGTTTLLPDTRCLVDN
SKSRLPQLLDCDKVKSSLYKRWNFIQNGAIMNKGTGRCLEVENRGLAGID
LILRSCTGQRWTIKNSIK

| | | |
|---|---|---|
| NOV6e, | SEQ ID NO: 69 | 3109 bp |
| SNP13379833 of | ORF Start: | ORF Stop: |
| CG124553-01, | ATG at 75 | TAG at 1479 |
| DNA Sequence | SNP Pos: 2610 | SNP Change: T to C |

AGGGGCTGAGGAGGTACTGGAAAAGAAAGAGGAGCAGGAGCTGGAGGAAG
ACGTGGAGGAGGAGCTGGAGGAGGATGAAGAGAAGGAGTGGGACGCCCAC
AACCCTGTGTAAGGAGCTCAAGTACTCCAAGGACCCGCCCCAGATATCCA
TCATATTCATCTTCGTGAACGAGGCCCTGTCGGTGATCCTGCGGTCCGTG
CACAGTGCCGTCAATCACACGCCCACACACCTGCTGAAGGAAATCATTCT
GGTGGATGACAACAGCGACGAAGAGGAGCTGAAGGTCCCCCTAGAGGAGT
ATGTCCACAAACGCTACCCCGGGCTGGTGAAGGTGGTAAGAAATCAGAAG
AGGGAAGGCCTGATCCGCGCTCGCATTGAGGGCTGGAAGGTGGCTACCGG
GCAGGTCACTGGCTTCTTTGATGCCCACGTGGAATTCACCGCTGGCTGGG
CTGAGCCGGTTCTATCCCGCATCCAGGAAAACCGGAAGCGTGTGATCCTC
CCCTCCATTGACAACATCAAACAGGACAACTTTGAGGTGCAGCGGTACGA
GAACTCGGCCCACGGGTACAGCTGGGAGCTGTGGTGCATGTACATCAGCC
CCCCAAAAGACTGGTGGGACGCCGGAGACCCTTCTCTCCCCATCAGGACC
CCAGCCATGATAGGCTGCTCGTTCGTGGTCAACAGGAAGTTCTTCGGTGA
AATTGGTCTTCTGGATCCTGGCATGGATGTATACGGAGGAGAAAATATTG
AACTGGGAATCAAGGTATGGCTCTGTGGGGGCAGCATGGAGGTCCTTCCT
TGCTCACGGGTGGCCCACATTGAGCGGAAGAAGAAGCCATATAATAGCAA
CATTGGCTTCTACACCAAGAGGAATGCTCTTCGCGTTGCTGAGGTCTGGA
TGGACGATTACAAGTCTCATGTGTACATAGCGTGGAACCTGCCGCTGGAG
AATCCGGGAATTGACATCGGTGATGTCTCCGAAAGAAGAGCATTAAGGAA
AAGTTTAAAGTGTAAGAATTTCCAGTGGTACCTGGACCATGTTTACCCAG
AAATGAGAAGATACAATAATACCGTTGCTTACGGGGAGCTTCGCAACAAC
AAGGCAAAAGACGTCTGCTTGGACCAGGGGCCGCTGGAGAACCACACAGC
AATATTGTATCCGTGCCATGGCTGGGGACCACAGCTTGCCCGCTACACCA
AGGAAGGCTTCCTGCACTTGGGTGCCCTGGGGACCACCACACTCCTCCCT
GACACCCGCTGCCTGGTGGACAACTCCAAGAGTCGGCTGCCCCAGCTCCT
GGACTGCGACAAGGTCAAGAGCAGCCTGTACAAGCGCTGGAACTTCATCC
AGAATGGAGCCATCATGAACAAGGGCACGGGACGCTGCCTGGAGGTGGAG
AACCGGGGCCTGGCTGGCATCGACCTCATCCTCCGCAGCTGCACAGGTCA
GAGGTGGACCATTAAGAACTCCATCAAGTAGAGGGAGGGAGCTGGGGCAC
TGGAGCCTGGCCCCCAGGACATGGCTGCTCCCCCCAACATCTGGACCAGC
TGCCCTGGCGGAGAGACAGCAAGGGGCCGGCAGGTGCTCGATGGGCCCCC
CAGGGCTTCTCCAGGGCAGCACAGGGACCCCGGATGAAGACTCTGTCCCC
CCTCAGGCATTCAGCTGCCCACAAGTTTCCTGCACCCTGGAAAAGCCCCC
CACCCTTCCTCTGGGAAACTGACAGCTGTCTTCCACAGCCTCTGATGTGG
ACCTGGTACTGAGGAGCAAGACTGTCCAGTTCTCCTCCACATCTCCCATC
CCAGAATCAGGATCTGGGACTGGCAGGGTCCCCTCCTGTGTCTCATCTCT
TGCAGCAGCAGCTGCTGAACTCCAGCCATCAACACGGTGGGAGGCAGCGG
GGGCTTCAGCCATGTCCTAGCTCCCCGCCCTAAAAGGAGGCAGTGAGGAC
CAGGCACTATTTCCTCCGAGGTTACTTCTACCCAGATGACACCTGCCTGT
TCACGCCCCAAGGCAGCTACTGCCCCTAACCCTTCCCACCAGGGTAGCTT
TGGGCACTGCAGCTCTGGACTTTTCTGGCCCCTCCTGAGATGACCTGATG
GAGCTGATGCTTTCTCTCCTAATCCCTGGGCACTAGGCTCTTATCAGTGT
GCTTGGGCCAGCTCTCCTGCCTGTGTCTAGAGGAAGCCAGAGACAGAAAT
AGGCTAAGCCTGCAGTAGGATCTCAGCCACAAGGGCCCCGCAGGATGGAG
CTGGGTCAAGGACCAGGGAGCCCTGACTCCCAGAGGCTGCCACCGGGGAG
AAGCAGCGGTCCTCCATCCAGAACCTAAGGGCTGAAGCAAAGGCTGCCAG
GACCCTTGAAGATGCTTTTGGCTCACCTCATTTCACCCCACGCTCTGCTG
GCTGGCAGAGGAGAAGGCAGTCGTTTCCTCTCTGAAGAGTATTTTTTTCG
ATTGCCCTCTGGTTAGGGTGCACATATAAATCAGAGTTAATATATGAACG
CGTGTGCATGCACAAGTGTGTGTGTGCCTGCGTGCTGTGCGTGGCAGGGT
GTGTGTGTGTGTGTCTGGCTGTGCGTTCCGGAGTGTGTGACGATGCTGAC
CTAGCTGTGCGGCCTTGGGCTTGCTGCTTCATTACTCACCTGGATGGGGA
CGAGGGATGAGAAGGGTGTGGGTTTGGCCCCATGTCACTGGCCGGAAGGA
TGTGTCTCAGCCCTGCCCTGTGGGGTGCCCCCGATGGGAGGCTGTCCCAT
CTCCCAGTCCCCATCTCTTTTTCCCCACACTGTCCCTGGCCAAGCCCTGC
CCAGAGCTGAACCCTGTAGCTGCCCCCTTGCCCTGTGTGGGATTCGCAGT
GTCTCATTTGGTGACGTCTTACTGGTGATCATCTCCTCACCCCATCTCCC
ACCTTGTGGAATAAATACATGTTAGCACTTCCCAGAGCAGCCTCCTTTGT
GTCTTGATTTCTCCAGAACTGGAGGTGGGGAGGGGAGTGATGGAGACATA
GGAGGAGAGCTTCTTTGGCTTTGAGGGTTTAGTGTTACTTATTTATCTAT
TTATTCGAGATGGGGTCTTGCTCTGTGGCCCAGGCTGGAGTGCAGTGGTG
CAATCATGA

| | | | |
|---|---|---|---|
| NOV6e, | | | MW at |
| SNP13379833 of | | | 53596.1 kD |
| CG124553-01, | | | SNP Change: |
| Protein Sequence | SEQ ID NO: 70 | 468 aa | no change |

MKRRSGTPTTLCKELKYSKDPPQISIIFIFVNEALSVILRSVHSAVNHTP
THLLKEIILVDDNSDEEELKVPLEEYVHKRYPGLVKVVRNQKREGLIRAR
IEGWKVATGQVTGFFDAHVEFTAGWAEPVLSRIQENRKRVILPSIDNIKQ
DNFEVQRYENSAHGYSWELWCMYISPPKDWWDAGDPSLPIRTPAMIGCSF
VVNRKFFGEIGLLDPGMDVYGGEMIELGIKVWLCGGSMEVLPCSRVAHIE
RKKKPYNSNIGFYTKRNALRVAEVWMDDYKSHVYIAWNLPLENPGIDIGD
VSERRALRKSLKCKNFQWYLDHVYPEMRRYNNTVAYGELRNNKAKDVCLD
QGPLENHTAILYPCHGWGPQLARYTKEGFLHLGALGTTTLLPDTRCLVDN
SKSRLPQLLDCDKVKSSLYKRWNFIQNGAIMNKGTGRCLEVENRGLAGID
LILRSCTGQRWTIKNSIK

A ClustalW comparison of the above protein sequences yields the following sequence alignment shown in Table 6B.

TABLE 6B
Comparison of the NOV6 protein sequences.

| | |
|---|---|
| NOV6a | MKRRSGTPTTLCKELKYSKDPPQISIIFIFVNEALSVILRSVHSAVNHTPTHLLKEIILV |
| NOV6b | ------------------------------------TRSRSVHSAVNHTPTHLLKEIILV |
| NOV6a | DDNSDEEELKVPLEEYVHKRYPGLVKVVRNQKREGLIRARIEGWKVATGQVTGFFDAHVE |
| NOV6b | DDNSDEEELKVPLEEYVHKRYPGLVKVVRNQKREGLIRARIEGWKVATGQVTGFFDAHVE |
| NOV6a | FTAGWAEPVLSRIQENRKRVILPSIDNIKQDNFEVQRYENSAHGYSWELWCMYISPPKDW |
| NOV6b | FTAGWAEPVLSRIQENRKRVILPSIDNIKQDNFEVQRYENSAHGYSWELWCMYISPPKDW |
| NOV6a | WDAGDPSLPIRTPAMIGCSFVVNRKFFGEIGLLDPGMDVYGGENIELGIKVWLCGGSMEV |
| NOV6b | WDAGDPSLPIRTPAMIGCSFVVNRKFFGEIGLLDPGMDVYGGENIELGIKVWLCGGSMEV |
| NOV6a | LPCSRVAHIERKKKPYNSNIGFYTKRNALRVAEVWMDDYKSHVYIAWNLPLENPGIDIGD |
| NOV6b | LPCSRVAHIERKKKPYNSNIGFYTKRNALRVAEVWMDDYKSHVYIAWNLPLENPGIDIGD |
| NOV6a | VSERRALRKSLKCKNFQWYLDHVYPEMRRYNNTVAYGELRNNKAKDVCLDQGPLENHTAI |
| NOV6b | VSERRALRKSLKCKNFQWYLDHVYPEMRRYNNTVAYGELRNNKAKDVCLDQOPLENHTAI |
| NOV6a | LYPCHGWGPQLARYTKEGFLHLGALGTTTLLPDTRCLVDNSKSRLPQLLDCDKVKSSLYK |
| NOV6b | LYPCHGWGPQLARYTKEGFLHLGALGTTTLLPDTRCLVDNSKSRLPQLLDCDKVKSSLYK |
| NOV6a | RWNFIQNGAIMNKGTGRCLEVENRGLAGIDLILRSCTGQRWTIKNSIK--- |
| NOV6b | RWNFIQNGAIMNKGTGRCLEVENRGLAGIDLILRSCTGQRWTIKNSIKLEG |
| NOV6a | (SEQ ID NO: 62) |
| NOV6b | (SEQ ID NO: 64) |

Further analysis of the NOV6a protein yielded the following properties shown in Table 6C.

TABLE 6C
Protein Sequence Properties NOV6a

| SignalP | No Known Signal Sequence Predicted | | | |
|---|---|---|---|---|
| analysis: | | | | |
| PSORT II | PSG: a new signal peptide prediction method | | | |
| analysis: | N-region: length 4; pos. chg 3; neg. chg 0 | | | |
| | H-region: length 8; peak value 5.12 | | | |
| | PSG score: 0.72 | | | |
| | GvH: von Heijne's method for signal seq. recognition | | | |
| | GvH score (threshold: −2.1): −5.89 | | | |
| | possible cleavage site: between 36 and 37 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 1 | | | |
| | Number of TMS(s) for threshold 0.5: 1 | | | |
| | INTEGRAL | Likelihood = | −3.66 | Transmembrane 23-39 |
| | PERIPHERAL | Likelihood = | 3.98 (at 225) | |
| | ALOM score: −3.66 (number of TMSs: 1) | | | |
| | MTOP: Prediction of membrane topology (Hartmann et al.) | | | |
| | Center position for calculation: 30 | | | |
| | Charge difference: −0.5 C(0.5)-N(1.0) | | | |
| | N >= C: N-terminal side will be inside | | | |
| | >>> membrane topology: type 2 (cytoplasmic tail 1 to 23) | | | |
| | MITDISC: discrimination of mitochondrial targeting seq | | | |
| | R content: | 2 | Hyd Moment (75): | 11.77 |
| | Hyd Moment (95): | 7.04 | G content: | 1 |
| | D/E content: | 2 | S/T content: | 5 |
| | Score: | −3.29 | | |

```
Gavel: prediction of cleavage sites for mitochondrial preseq
R-2 motif at 14 RRS|GT
NUCDISC: discrimination of nuclear localization signals
pat4: RKKK (5) at 251
pat4: KKKP (4) at 252
pat7: none
bipartite: none
content of basic residues: 13.9%
NLS Score: 0.09
KDEL: ER retention motif in the C-terminus: none
ER Membrane Retention Signals:
XXRR-like motif in the N-terminus: KRRS
KKXX-like motif in the C-terminus: KNSI
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: 16
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 89
COIL: Lupas's algorithm to detect coiled-coil regions
total: 0 residues
Final Results (k = 9/23):
39.1%: mitochondrial
30.4%: cytoplasmic
  8.7%: Golgi
  8.7%: nuclear
  4.3%: vacuolar
  4.3%: vesicles of secretory system
  4.3%: endoplasmic reticulum
>> prediction for CG124553-01 is mit (k = 23)
```

A search of the NOV6a protein against the Geneseq database, a proprietary database that contains sequences published in patents and patent publication, yielded several homologous proteins shown in Table 6D.

TABLE 6D
Geneseq Results for NOV6a

| | | NOV6a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG32380 | Novel human enzyme, NZMS-10, | 10 . . . 468 | 457/459 (99%) | 0.0 |
| | Incyte 70861047CD1 - Homo | 140 . . . 598 | 457/459 (99%) | |
| | sapiens, 598 aa. [WO200264795-A2, | | | |
| | 22 AUG. 2002] | | | |
| ABP53502 | Human pp-GaNTase 10 protein SEQ | 10 . . . 468 | 457/459 (99%) | 0.0 |
| | ID NO: 3 - Homo sapiens, 598 aa. | 140 . . . 598 | 457/459 (99%) | |
| | [EP1243660-A2, 25 SEP. 2002] | | | |
| AAM41675 | Human polypeptide SEQ ID NO 6606- | 10 . . . 468 | 457/459 (99%) | 0.0 |
| | Homo sapiens, 560 aa. | 102 . . . 560 | 457/459 (99%) | |
| | [WO200153312-A1, 26 JUL. 2001] | | | |
| ABG73283 | Human 47174 protein, a | 12 . . . 468 | 303/459 (66%) | 0.0 |
| | glycosyltransferase - Homo sapiens, | 141 . . . 599 | 378/459 (82%) | |
| | 603 aa. [US2002164746-A1, | | | |
| | 07 NOV. 2002] | | | |
| AAM40865 | Human polypeptide SEQ ID NO 5796- | 167 . . . 468 | 302/302 (100%) | 0.0 |
| | Homo sapiens, 358 aa. | 5 . . . 306 | 302/302 (100%) | |
| | [WO200153312-A1, 26 JUL. 2001] | | | |

In a BLAST search of public sequence databases, the NOV6a protein was found to have homology to the proteins shown in the BLASTP data in Table 6E.

TABLE 6E
Public BLASTP Results for NOV6a

| | | NOV6a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8NFV9 | Putative polypeptide | 10 . . . 468 | 457/459 (99%) | 0.0 |
| | N-acetylgalactosaminyltransferase - | 140 . . . 598 | 457/459 (99%) | |
| | Homo sapiens (Human), 598 aa. | | | |
| AAH52469 | Hypothetical protein - Mus musculus | 10 . . . 468 | 450/459 (98%) | 0.0 |
| | (Mouse), 598 aa. | 140 . . . 598 | 453/459 (98%) | |
| Q8K483 | Williams-Beuren syndrome critical | 10 . . . 468 | 447/459 (97%) | 0.0 |
| | region gene 17 - Mus musculus (Mouse), | 140 . . . 596 | 450/459 (97%) | |
| | 596 aa. | | | |
| Q9GM01 | UDP-GalNAc: polypeptide | 12 . . . 468 | 303/459 (66%) | 0.0 |
| | N-acetylgalactosaminyltransferase - | 144 . . . 602 | 377/459 (82%) | |
| | Macaca fascicularis (Crab eating | | | |
| | macaque) (Cynomolgus monkey), 606 aa. | | | |
| Q9HCQ5 | UDP-GalNAc: polypeptide | 12 . . . 468 | 302/459 (65%) | 0.0 |
| | N-acetylgalactosaminyltransferase - | 141 . . . 599 | 377/459 (81%) | |
| | Homo sapiens (Human), 603 aa. | | | |

PFam analysis predicts that the NOV6a protein contains the domains shown in the Table 6F.

TABLE 6F
Domain Analysis of NOV6a

| | | Identities/ | |
|---|---|---|---|
| | | Similarities | |
| Pfam | NOV6a Match | for the | Expect |
| Domain | Region | Matched Region | Value |
| Glycos_transf_2 | 25 . . . 211 | 44/189 (23%) | 4e−31 |
| | | 142/189 (75%) | |
| Ricin_B_lectin | 428 . . . 466 | 14/47 (30%) | 0.085 |
| | | 29/47 (62%) | |

### Example 7

The NOV7 clone was analyzed, and the nucleotide and encoded polypeptide sequences are shown in Table 7A.

TABLE 7A
NOV7 Sequence Analysis

| | | |
|---|---|---|
| NOV7a, | SEQ ID NO: 71 | 1066 bp |
| CG187738-02 | ORF Start: | ORF Stop: |
| DNA Sequence | at 2 | at 1058 |

CACCGGATCCATGAAGGATTGCAGTAACGGATGCTCCGCAGAGTGTACCG
GAGAAGGAGGATCAAAAGAGGTGGTGGGGACTTTTAAGGCTAAAGACCTA
ATAGTCACACCAGCTACCATTTTAAAGGAAAAACCAGACCCCAATAATCT
GGTTTTTGGAACTGTGTTCACGGATCATATGCTGACGGTGGAGTGGTCCT
CAGAGTTTGGATGGGAGAAACCTCATATCAAGCCTCTTCAGAACCTGTCA
TTGCACCCTGGCTCATCAGCTTTGCACTATGCAGTGGAAGTATTTGACAA
AGAAGAGCTCTTAGAGTGTATTCAACAGCTTGTGAAATTGGATCAAGAAT
GGGTCCCATATTCAACATCTGCTAGTCTGTATATTCGTCCTACATTCATT
GGAACTGAGCCTTCTCTTGGAGTCAAGAAGCCTACCAAAGCCCTGCTCTT
TGTACTCTTGAGCCCAGTGGGACCTTATTTTTCAAGTGGAACCTTTAATC
CAGTGTCCCTGTGGGCCAATCCCAAGTATGTAAGAGCCTGGAAAGGTGGA
ACTGGGGACTGCAAGATGGGAGGGAATTACGGCTCATCTCTTTTTGCCCA
ATGTGAAGCAGTAGATAATGGGTGTCAGCAGGTCCTGTGGCTCTATGGAG
AGGACCATCAGATCACTGAAGTGGGAACTATGAATCTTTTTCTTTACTGG
ATAAATGAAGATGGAGAAGAAGAACTGGCAACTCCTCCACTAGATGGCAT
CATTCTTCCAGGAGTGACAAGGCGGTGCATTCTGGACCTGGCACATCAGT
GGGGTGAATTTAAGGTGTCAGAGAGATACCTCACCATGGATGACTTGACA
ACAGCCCTGGAGGGGAACAGAGTGAGAGAGATGTTTGGCTCTGGTACAGC
CTGTGTTGTTTGCCCAGTTTCTGATATACTGTACAAAGGCGAGACAATAC
ACATTCCAACTATGGAGAATGGTCCTAAGCTGGCAAGCCGCATCTTGAGC
AAATTAACTGATATCCAGTATGGAAGAGAAGAGAGCGACTGGACAATTGT
GCTATCCGTCGACGGC

| | | | |
|---|---|---|---|
| NOV7a, CG187738-02 | | | MW at |
| Protein Sequence | SEQ ID NO: 72 | 352 aa | 38889.9 kD |

TGSMKDCSNGCSAECTGEGGSKEVVGTFKAKDLIVTPATILKEKPDPNNL
VFGTVFTDHMLTVEWSSEFGWEKPHIKPLQNLSLHPGSSALHYAVEVFDK
EELLECIQQLVKLDQEWVPYSTSASLYIRPTFIGTEPSLGVKKPTKALLF
VLLSPVGPYFSSGTFNPVSLWANPKYVRAWKGGTGDCKMGGNYGSSLFAQ
CEAVDNGCQQVLWLYGEDHQITEVGTMNLFLYWINEDGEEELATPPLDGI
ILPGVTRRCILDLAHQWGEFKVSERYLTMDDLTTALEGNRVREMFGSGTA
CVVCPVSDILYKGETIHIPTMENGPKLASRILSKLTDIQYGREESDWTIV
LS

| | | |
|---|---|---|
| NOV7b, | SEQ ID NO: 73 | 2043 bp |
| CG187738-01 | ORF Start: | ORF Stop: |
| DNA Sequence | ATG at 528 | TGA at 1686 |

AGTAGGGAGGTGGGCAGGAGCCAGTGATGACGGAATGGCAATCACATTTG
ACCTCTGATCTGTTTATTTCCTCCTCCTTGACGTCTCCATATAAATGTTA
CACGGGCATCCCCACACTCGGATACGCACCCACAGTGGCTGATTCGGGGG
TAACCGTGTCATTTGCTTGCAACACTGGCACCTCTGCCCTGCACCCCGGG
AGTGAGCAGTGAGTGAGGCTCGGGTCTGGGCGCTGGCTCCGAATCTTCGG
GCTGGGAGAGACTCCACCATCTGGGGGCGGCCTGGGGGAGCAGCCTTAGT
GTCTTCCTGCTGATGCAATCCGCTAGGTCGCGAGTCTCCGCCGCGAGAGG
GCCGGTCTGCAATCCAGCCCGCCACGTGTACTCGCCGCCGCCTCGGGCAC
TGCCCCAGGTCTTGCTGCAGCCGGGACCGCGCTCTGCAGCCGCAGACCCG
GTCCACACGGCCAGGGGCTACGACCCTTGGGATCTGCCCTCCGCTCAGCT
CGAGCTTCCCTCGTGGCCGACGGAACAATGAAGGATTGCAGTAACGGATG
CTCCGCAGAGTGTACCGGAGAAGGAGGATCAAAAGAGGTGGTGGGGACTT
TTAAGGCTAAAGACCTAATAGTCACACCAGCTACCATTTTAAAGGAAAAA
CCAGACCCCAATAATCTGGTTTTTGGAACTGTGTTCACGGATCATATGCT
GACGGTGGAGTGGTCCTCAGAGTTTGGATGGGAGAAACCTCATATCAAGC
CTCTTCAGAACCTGTCATTGCACCCTGGCTCATCAGCTTTGCACTATGCA
GTGGAATTATTTGAAGGATTGAAGGCATTTCGAGGAGTAGATAATAAAAT
TCGACTGTTTCAGCCAAACCTCAACATGGATAGAATGTATCGCTCTGCTG
TGAGGGCAACTCTGCCGGTATTTGACAAAGAAGAGCTCTTAGAGTGTATT
CAACAGCTTGTGAAATTGGATCAAGAATGGGTCCCATATTCAACATCTGC
TAGTCTGTATATTCGTCCTACATTCATTGGAACTGAGCCTTCTCTTGGAG
TCAAGAAGCCTACCAAAGCCCTGCTCTTTGTACTCTTGAGCCCAGTGGGA
CCTTATTTTTCAAGTGGAACCTTTAATCCAGTGTCCCTGTGGGCCAATCC
CAAGTATGTAAGAGCCTGGAAAGGTGGAACTGGGGACTGCAAGATGGGAG
GGAATTACGGCTCATCTCTTTTTGCCCAATGTGAAGCAGTAGATAATGGG
TGTCAGCAGGTCCTGTGGCTCTATGGAGAGGACCATCAGATCACTGAAGT
GGGAACTATGAATCTTTTTCTTTACTGGATAAATGAAGATGGAGAAGAAG
AACTGGCAACTCCTCCACTAGATGGCATCATTCTTCCAGGAGTGACAAGG
CGGTGCATTCTGGACCTGGCACATCAGTGGGGTGAATTTAAGGTGTCAGA
GAGATACCTCACCATGGATGACTTGTCAACAGCCCTGGAGGGGAACAGAG
TGAGAGAGATGTTTGGCTCTGGTACAGCCTGTGTTGTTTGCCCAGTTTCT
GATATACTGTACAAAGGCGAGACAATACACATTCCAACTATGGAGAATGG
TCCTAAGCTGGCAAGCCGCATCTTGAGCAAATTAACTGATATCCAGTATG
GAAGAGAAGAGAGCGACTGGACAATTGTGCTATCCTGAATGGAAAATAGA
GGATACAATGGAAAATAGAGGATACCAACTGTATGCTACTGGGACAGACT
GTTGCATTTGAATTGTGATAGATTTCTTTGGCTACCTGTGCATAATGTAG
TTTGTAGTATCAATGTGTTACAAGAGTGATTGTTTCTTCATGCCAGAGAA
AATGAATTGCAATCATCAAATGGTGTTTCATAACTTGGTAGTAGTAACTT
ACCTTACCTTACCTAGAAAAATATTAATGTAAGCCATATAACATGGGATT
TTCCTCAATGATTTTAGTGCCTCCTTTTGTACTTCACTCAGATACTAAAT
AGTAGTTTATTCTTTAATATAAGTTACATTCTGCTCCTCAACC

| | | | |
|---|---|---|---|
| NOV7b, CG187738-01 | | | MW at |
| Protein Sequence | SEQ ID NO: 74 | 386 aa | 42951.7 kD |

MKDCSNGCSAECTGEGGSKEVVGTFKAKDLIVTPATILKEKPDPNNLVFG
TVFTDHMLTVEWSSEFGWEKPHIKPLQNLSLHPGSSALHYAVELFEGLKA
FRGVDNKIRLFQPNLNMDRMYRSAVRATLPVFDKEELLECIQQLVKLDQE
WVPYSTSASLYIRPTFIGTEPSLGVKKPTKALLFVLLSPVGPYFSSGTFN
PVSLWANPKYVRAWKGGTGDCKMGGNYGSSLFAQCEAVDNGCQQVLWLYG
EDHQITEVGTMNLFLYWINEDGEEELATPPLDGIILPGVTRRCILDLAHQ
WGEFKVSERYLTMDDLSTALEGNRVREMFGSGTACVVCPVSDILYKGETI
HIPTMENGPKLASRILSKLTDIQYGREESDWTIVLS

| | | |
|---|---|---|
| NOV7c, | SEQ ID NO: 75 | 1177 bp |
| CG187738-03 | ORF Start: | ORF Stop: |
| DNA Sequence | ATG at 11 | at 1169 |

CACCGGATCCATGAAGGATTGCAGTAACGGATGCTCCGCAGAGTGTACCG
GAGAAGGAGGATCAAAAGAGGTGGTGGGGACTTTTAAGGCTAAAGACCTA
ATAGTCACACCAGCTACCATTTTAAAGGAAAAACCAGACCCCAATAATCT
GGTTTTTGGAACTGTGTTCACGGATCATATGCTGACGGTGGAGTGGTCCT
CAGAGTTTGGATGGGAGAAACCTCATATCAAGCCTCTTCAGAACCTGTCA
TTGCACCCTGGCTCATCAGCTTTGCACTATGCAGTGGAATTATTTGAAGG
ATTGAAGGCATTTCGAGGAGTAGATAATAAAATTCGACTGTTTCAGCCAA
ACCTCAACATGGATAGAATGTATCGCTCTGCTGTGAGGGCAACTCTGCCG
GTATTTGACAAAGAAGAGCTCTTAGAGTGTATTCAACAGCTTGTGAAATT
GGATCAAGAATGGGTCCCATATTCAACATCTGCTAGTCTGTATATTCGTC
CTACATTCATTGGAACTGAGCCTTCTCTTGGAGTCAAGAAGCCTACCAAA
GCCCTGCTCTTTGTACTCTTGAGCCCAGTGGGACCTTATTTTTCAAGTGG
AACCTTTAATCCAGTGTCCCTGTGGGCCAATCCCAAGTATGTAAGAGCCT
GGAAAGGTGGAACTGGGGACTGCAAGATGGGAGGGAATTACGGCTCATCT
CTTTTTGCCCAATGTGAAGCAGTAGATAATGGGTGTCAGCAGGTCCTGTG
GCTCTATGGAGAGGACCATCAGATCACTGAAGTGGGAACTATGAATCTTT
TTCTTTACTGGATAAATGAAGATGGAGAAGAAGAACTGGCAACTCCTCCA
CTAGATGGCATCATTCTTCCAGGAGTGACAAGGCGGTGCATTCTGGACCT
GGCACATCAGTGGGGTGAATTTAAGGTGTCAGAGAGATACCTCACCATGG
ATGACTTGACAACAGCCCTGGAGGGGAACAGAGTGAGAGAGATGTTTGGC
TCTGGTACAGCCTGTGTTGTTTGCCCAGTTTCTGATATACTGTACAAAGG
CGAGACAATACACATTCCAACTATGGAGAATGGTCCTAAGCTGGCAAGCC
GCATCTTGAGCAAATTAACTGATATCCAGTATGGAAGAGAAGAGAGCGAC
TGGACAATTGTGCTATCCGTCGACGGC

| | | | |
|---|---|---|---|
| NOV7c, CG187738-03 | | | MW at |
| Protein Sequence | SEQ ID NO: 76 | 386 aa | 42965.7 kD |

MKDCSNGCSAECTGEGGSKEVVGTFKAKDLIVTPATILKEKPDPNNLVFG
TVFTDHMLTVEWSSEFGWEKPHIKPLQNLSLHPGSSALHYAVELFEGLKA
FRGVDNKIRLFQPNLNNDRNYRSAVRATLPVFDKEELLECIQQLVKLDQE
WVPYSTSASLYIRPTFIGTEPSLGVKKPTKALLFVLLSPVGPYFSSGTFN
PVSLWANPKYVRAWKGGTGDCKMGGNYGSSLFAQCEAVDNGCQQVLWLYG
EDHQITEVGTMNLFLYWINEDGEEELATPPLDGIILPGVTRRCILDLAHQ
WGEFKVSERYLTMDDLTTALEGNRVREMFGSGTACVVCPVSDILYKGETI
HIPTMENGPKLASRILSKLTDIQYGREESDWTIVLS

A ClustalW comparison of the above protein sequences yields the following sequence alignment shown in Table 7B.

TABLE 7B
Comparison of the NOV7 protein sequences.

| | |
|---|---|
| NOV7a | TGSMKDCSNGCSAECTGEGGSKEVVGTFKAKDLIVTPATILKEKPDPNNLVFGTVFTDHM |
| NOV7b | ---MKDCSNGCSAECTGEGGSKEVVGTFKAKDLIVTPATILKEKPDPNNLVFGTVFTDHM |
| NOV7c | ---MKDCSNGCSAECTGEGGSKEVVGTFKAKDLIVTPATILKEKPDPNNLVFGTVFTDHM |
| NOV7a | LTVEWSSEFGWEKPHIKPLQNLSLHPGSSALHYAVEVFD--------------------- |
| NOV7b | LTVEWSSEFGWEKPHIKPLQNLSLHPGSSALHYAVELFEGLKAFRGVDNKIRLFQPNLNM |
| NOV7c | LTVEWSSEFGWEKPHIKPLQNLSLHPGSSALHYAVELFEGLKAFRGVDNKIRLFQPNLNM |
| NOV7a | ----------------KEELLECIQQLVKLDQEWVPYSTSASLYIRPTFIGTEPSLGVKK |
| NOV7b | DRMYRSAVRATLPVFDKEELLECIQQLVKLDQEWVPYSTSASLYIRPTFIGTEPSLGVKK |
| NOV7c | DRMYRSAVRATLPVFDKEELLECIQQLVKLDQEWVPYSTSASLYIRPTFIGTEPSLGVKK |
| NOV7a | PTKALLFVLLSPVGPYFSSGTFNPVSLWANPKYVRAWKGGTGDCKMGGNYGSSLFAQCEA |
| NOV7b | PTKALLFVLLSPVGPYFSSGTFNPVSLWANPKYVRAWKGGTGDCKMGGNYGSSLFAQCEA |
| NOV7c | PTKALLFVLLSPVGPYFSSGTFNPVSLWANPKYVRAWKGGTGDCKMGGNYGSSLFAQCEA |
| NOV7a | VDNGCQQVLWLYGEDHQITEVGTMNLFLYWINEDGEEELATPPLDGIILPGVTRRCILDL |
| NOV7b | VDNGCQQVLWLYGEDHQITEVGTMNLFLYWINEDGEEELATPPLDGIILPGVTRRCILDL |
| NOV7c | VDNGCQQVLWLYGEDHQITEVGTMNLFLYWINEDGEEELATPPLDGIILPGVTRRCILDL |
| NOV7a | AHQWGEFKVSERYLTMDDLTTALEGNRVREMFGSGTACVVCPVSDILYKGETIHIPTMEN |
| NOV7b | AHQWGEFKVSERYLTMDDLSTALEGNRVREMFGSGTACVVCPVSDILYKGETIHIPTMEN |
| NOV7c | AHQWGEFKVSERYLTMDDLTTALEGNRVREMFGSGTACVVCPVSDILYKGETIHIPTMEN |
| NOV7a | GPKLASRILSKLTDIQYGREESDWTIVLS |
| NOV7b | GPKLASRILSKLTDIQYGREESDWTIVLS |
| NOV7c | GPKLASRILSKLTDIQYGREESDWTIVLS |
| NOV7a | (SEQ ID NO: 72) |
| NOV7b | (SEQ ID NO: 74) |
| NOV7c | (SEQ ID NO: 76) |

Further analysis of the NOV7a protein yielded the following properties shown in Table 7C.

TABLE 7C
Protein Sequence Properties NOV7a

| SignalP | No Known Signal Sequence Predicted | | | |
|---|---|---|---|---|
| analysis: | | | | |
| PSORT II | PSG: a new signal peptide prediction method | | | |
| analysis: | N-region: length 6; pos. chg 1; neg. chg 1 | | | |
| | H-region: length 7; peak value −2.71 | | | |
| | PSG score: −7.11 | | | |
| | GvH: von Heijne's method for signal seq. recognition | | | |
| | GvH score (threshold: −2.1): −10.56 | | | |
| | possible cleavage site: between 13 and 14 | | | |
| | >>> Seems to have no N-terminal signal peptide | | | |
| | ALOM: Klein et al' s method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 1.43 (at 294) | |
| | ALOM score: 1.43 (number of TMSs: 0) | | | |
| | MITDISC: discrimination of mitochondrial targeting seq | | | |
| | R content: | 0 | Hyd Moment (75): | 8.66 |
| | Hyd Moment(95): | 8.28 | G content: | 2 |
| | D/E content: | 2 | S/T content: | 4 |
| | Score: | −6.18 | | |

Gavel: prediction of cleavage sites for mitochondrial preseq
cleavage site motif not found
NUCDISC: discrimination of nuclear localization signals
pat4: none
pat7: none
bipartite: none
content of basic residues: 8.2%
NLS Score: −0.47
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
52.2%: cytoplasmic
30.4%: nuclear
13.0%: mitochondrial
4.3%: Golgi
>> prediction for CG187738-02 is cyt (k = 23)

A search of the NOV7a protein against the Geneseq database, a proprietary database that contains sequences published in patents and patent publication, yielded several homologous proteins shown in Table 7D.

TABLE 7D
Geneseq Results for NOV7a

| | | NOV7a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABU03468 | Angiogenesis-associated human protein | 6 . . . 352 | 342/384 (89%) | 0.0 |
| | sequence #13 - Homo sapiens, 384 aa. | 2 . . . 384 | 342/384 (89%) | |
| | [WO200279492-A2, 10 OCT. 2002] | | | |
| ABB57125 | Mouse ischaemic condition related | 4 . . . 245 | 201/279 (72%) | e−117 |
| | protein sequence SEQ ID NO:287 - | 1 . . . 279 | 222/279 (79%) | |
| | Mus musculus, 280 aa. | | | |
| | [WO200188188-A2, 22 NOV. 2001] | | | |
| AAE36351 | Human branched chain | 19 . . . 347 | 181/367 (49%) | 1e−99 |
| | aminotransferase alternative splice | 23 . . . 376 | 235/367 (63%) | |
| | form protein - Homo sapiens, 380 aa. | | | |
| | [WO200297044-A2, 05 DEC. 2002] | | | |
| AAY40000 | S. pombe ECA39 gene protein - | 35 . . . 349 | 154/359 (42%) | 2e−71 |
| | Schizosaccharomyces pombe, 380 aa. | 20 . . . 376 | 200/359 (54%) | |
| | [WO9950422-A2, 07 OCT. 1999] | | | |
| ABB58364 | Drosophila melanogaster polypeptide | 70 . . . 291 | 123/259 (47%) | 1e−67 |
| | SEQ ID NO 1884 - Drosophila | 12 . . . 270 | 164/259 (62%) | |
| | melanogaster, 283 aa. | | | |
| | [WO200171042-A2, 27 SEP. 2001] | | | |

In a BLAST search of public sequence databases, the NOV7a protein was found to have homology to the proteins shown in the BLASTP data in Table 7E.

TABLE 7E
Public BLASTP Results for NOV7a

| | | NOV7a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q96MY9 | Hypothetical protein FLJ31693 - Homo | 4 . . . 352 | 348/386 (90%) | 0.0 |
| | sapiens (Human), 386 aa. | 1 . . . 386 | 349/386 (90%) | |
| P54687 | Branched-chain amino acid | 6 . . . 352 | 342/384 (89%) | 0.0 |
| | aminotransferase, cytosolic (EC | 2 . . . 384 | 342/384 (89%) | |
| | 2.6.1.42) (BCAT(c)) (ECA39 protein) - | | | |
| | Homo sapiens (Human), 384 aa. | | | |
| Q9GKM4 | Cytosolic branched-chain amino acid | 6 . . . 352 | 314/384 (81%) | 0.0 |
| | aminotransferase (EC 2.6.1.42) - Ovis | 2 . . . 385 | 331/384 (85%) | |
| | aries (Sheep), 385 aa. | | | |
| Q8CBC8 | Branched chain aminotransferase 1 - | 1 . . . 351 | 294/388 (75%) | e−174 |
| | Mus musculus (Mouse), 453 aa. | 65 . . . 452 | 323/388 (82%) | |
| Q9CXX6 | Branched chain aminotransferase 1, | 1 . . . 351 | 294/388 (75%) | e−174 |
| | cytosolic - Mus musculus (Mouse), 398 | 10 . . . 397 | 323/388 (82%) | |
| | aa. | | | |

PFam analysis predicts that the NOV7a protein contains the domains shown in the Table 7F.

TABLE 7F
Domain Analysis of NOV7a

| | | Identities/ | |
|---|---|---|---|
| | | Similarities | |
| | NOV7a Match | for the | |
| Pfam Domain | Region | Matched Region | Expect Value |
| aminotran_4 | 73 . . . 336 | 139/310 (45%) | 4.9e−131 |
| | | 254/310 (82%) | |

### Example 8

The NOV8 clone was analyzed, and the nucleotide and encoded polypeptide sequences are shown in Table 8A.

TABLE 8A
NOV8 Sequence Analysis

| | | |
|---|---|---|
| NOV8a, | SEQ ID NO: 77 | 1057 bp |
| CG55676-04 | ORF Start: | ORF Stop: |
| DNA Sequence | ATG at 11 | at 1049 |

CACCAGATCTATGTACAACGGGTCGTGCTGCCGCATCGAGGGGGACACCA
TCTCCCAGGTGATGCCGCCGCTGCTCATTGTGGCCTTTGTGCTGGGCGCA
CTAGGCAATGGGGTCGCCCTGTGTGGTTTCTGCTTCCACATGAAGACCTG
GAAGCCCAGCACTGTTTACCTTTTCAATTTGGCCGTGGCTGATTTCCTCC
TTATGATCTGCCTGCCTTTTCGGACAGACTATTACCTCAGACGTAGACAC
TGGGCTTTTGGGGACATTCCCTGCCGAGTGGGGCTCTTCACGTTGGCCAT
GAACAGGGCCGGGAGCATCGTGTTCCTTACGGTGGTGGCTGCGGACAGGT
ATTTCAAAGTGGTCCACCCCCACCACGCGGTGAACACTATCTCCACCCGG
GTGGCGGCTGGCATCGTCTGCACCCTGTGGGCCCTGGTCATCCTGGGAAC
AGTGTATCTTTTGCTGGAGAACCATCTCTGCGTGCAAGAGACGGCCGTCT
CCTGTGAGAGCTTCATCATGGAGTCGGCCAATGGCTGGCATGACATCATG
TTCCAGCTGGAGTTCTTTATGCCCCTCGGCATCATCTTATTTTGCTCCTT
CAAGATTGTTTGGAGCCTGAGGCGGAGGCAGCAGCTGGCCAGACAGGCTC
GGATGAAGAAGGCGACCCGGTTCATCATGGTGGTGGCAATTGTGTTCATC
ACATGCTACCTGCCCAGCGTGTCTGCTAGACTCTATTTCCTCTGGACGGT
GCCCTCGAGTGCCTGCGATCCCTCTGTCCATGGGGCCCTGCACATAACCC
TCAGCTTCACCTACATGAACAGCATGCTGGATCCCCTGGTGTATTATTTT
TCAAGCCCCTCCTTTCCCAAATTCTACAACAAGCTCAAAATCTGCAGTCT
GAAACCCAAGCAGCCAGGACACTCAAAAACACAAAGGCCGGAAGAGATGC
CAATTTCGAACCTCGGTCGCAGGAGTTGCATCAGTGTGGCAAATAGTTTC
CAAAGCCAGTCTGATGGGCAATGGGATCCCCACATTGTTGAGTGGCACAA
GCTTGGC

| | | | |
|---|---|---|---|
| NOV8a, CG55676-04 | | | MW at |
| Protein Sequence | SEQ ID NO: 78 | 346 aa | 39294.8 kD |

MYNGSCCRIEGDTISQVMPPLLIVAFVLGALGNGVALCGFCFHMKTWKPS
TVYLFNLAVADFLLMICLPFRTDYYLRRRHWAFGDIPCRVGLFTLAMNRA
GSIVFLTVVAADRYFKVVHPHHAVNTISTRVAAGIVCTLWALVILGTVYL
LLENHLCVQETAVSCESFIMESANGWHDIMFQLEFFMPLGIILFCSFKIV
WSLRRRQQLARQARMKKATRFIMVVAIVFITCYLPSVSARLYFLWTVPSS
ACDPSVHGALHITLSFTYMNSMLDPLVYYFSSPSFPKFYNKLKICSLKPK
QPGHSKTQRPEEMPISNLGRRSCISVANSFQSQSDGQWDPHIVEWH

| | | |
|---|---|---|
| NOV8b, | SEQ ID NO: 79 | 961 bp |
| 304967299 | ORF Start: | ORF Stop: |
| DNA Sequence | at 2 | end of sequence |

CACCTCGCGAAATGGGGTCGCCCTGTGTGGTTTCTGCTTCCACATGAAGA
CCTGGAAGCCCAGCACTGTTTACCTTTTCAATTTGGCCGTGGCTGATTTC
CTCCTTATGATCTGCCTGCCTTTTCGGACAGACTATTACCTCAGACGTAG
ACACTGGGCTTTTGGGGACATTCCCTGCCGAGTGGGGCTCTTCACGTTGG
CCATGAACAGGGCCGGGAGCATCGTGTTCCTTACGGTGGTGGCTGCGGAC
AGGTATTTCAAAGTGGTCCACCCCCACCACGCGGTGAACACTATCTCCAC
CCGGGTGGCGGCTGGCATCGTCTGCACCCTGTGGGCCCTGGTCATCCTGG
GAACAGTGTATCTTTTGCTGGAGAACCATCTCTGCGTGCAAGAGACGGCC
GTCTCCTGTGAGAGCTTCATCATGGAGTCGGCCAATGGCTGGCATGACAT
CATGTTCCAGCTGGAGTTCTTTATGCCCCTCGGCATCATCTTATTTTGCT
CCTTCAAGATTGTTTGGAGCCTGAGGCGGAGGCAGCAGCTGGCCAGACAG
GCTCGGATGAAGAAGGCGACCCGGTTCATCATGGTGGTGGCAATTGTGTT
CATCACATGCTACCTGCCCAGCGTGTCTGCTAGACTCTATTTCCTCTGGA
CGGTGCCCTCGAGTGCCTGCGATCCCTCTGTCCATGGGGCCCTGCACATA
ACCCTCAGCTTCACCTACATGAACAGCATGCTGGATCCCCTGGTGTATTA
TTTTTCAAGCCCCTCCTTTCCCAAATTCTACAACAAGCTCAAAATCTGCA
GTCTGAAACCCAAGCAGCCAGGACACTCAAAAACACAAAGGCCGGAAGAG
ATGCCAATTTCGAACCTCGGTCGCAGGAGTTGCATCAGTGTGGCAAATAG
TTTCCAAAGCCAGTCTGATGGGCAATGGGATCCCCACATTGTTGAGTGGC
ACGTCGACGGC

| | | | |
|---|---|---|---|
| NOV8b, 304967299 | | | MW at |
| Protein Sequence | SEQ ID NO: 80 | 320 aa | 36559.5 kD |

TSRNGVALCGFCFHMKTWKPSTVYLFNLAVADFLLMICLPFRTDYYLRRR
HWAFGDIPCRVGLFTLAMNRAGSIVFLTVVAADRYFKVVHPHHAVNTIST
RVAAGIVCTLWALVILGTVYLLLENHLCVQETAVSCESFIMESANGWNDI
MFQLEFFMPLGIILFCSFKIVWSLRRRQQLARQARMKKATRFIMVVAIVF
ITCYLPSVSARLYFLWTVPSSACDPSVHGALHITLSFTYMNSMLDPLVYY
FSSPSFPKFYNKLKICSLKPKQPGHSKTQRPEEMPISNLGRRSCISVANS
FQSQSDGQWDPHIVEWHVDG

| | | |
|---|---|---|
| NOV8c, | SEQ ID NO: 81 | 1050 bp |
| CG55676-01 | ORF Start: | ORF Stop: |
| DNA Sequence | ATG at 6 | TGA at 1044 |

TCGCCATGTACAACGGGTCGTGCTGCCGCATCGAGGGGGACACCATCTCC
CAGGTGATGCCGCCGCTGCTCATTGTGGCCTTTGTGCTGGGCGCACTAGG
CAATGGGGTCGCCCTGTGTGGTTTCTGCTTCCACATGAAGACCTGGAAGC
CCAGCACTGTTTACCTTTTCAATTTGGCCGTGGCTGATTTCCTCCTTATG
ATCTGCCTGCCTTTTCGGACAGACTATTACCTCAGACGTAGACACTGGGC
TTTTGGGGACATTCCCTGCCGAGTGGGGCTCTTCACGTTGGCCATGAACA
GGGCCGGGAGCATCGTGTTCCTTACGGTGGTGGCTGCGGACAGGTATTTC
AAAGTGGTCCACCCCCACCACGCGGTGAACACTATCTCCACCCGGGTGGC
GGCTGGCATCGTCTGCACCCTGTGGGCCCTGGTCATCCTGGGAACAGTGT
ATCTTTTGCTGGAGAACCATCTCTGCGTGCAAGAGACGGCCGTCTCCTGT
GAGAGCTTCATCATGGAGTCGGCCAATGGCTGGCATGACATCATGTTCCA
GCTGGAGTTCTTTATGCCCCTCGGCATCATCTTATTTTGCTCCTTCAAGA
TTGTTTGGAGCCTGAGGCGGAGGCAGCAGCTGGCCAGACAGGCTCGGATG
AAGAAGGCGACCCGGTTCATCATGGTGGTGGCAATTGTGTTCATCACATG
CTACCTGCCCAGCGTGTCTGCTAGACTCTATTTCCTCTGGACGGTGCCCT
CGAGTGCCTGCGATCCCTCTGTCCATGGGGCCCTGCACATAACCCTCAGC
TTCACCTACATGAACAGCATGCTGGATCCCCTGGTGTATTATTTTTCAAG
CCCCTCCTTTCCCAAATTCTACAACAAGCTCAAAATCTGCAGTCTGAAAC
CCAAGCAGCCAGGACACTCAAAAACACAAAGGCCGGAAGAGATGCCAATT
TCGAACCTCGGTCGCAGGAGTTGCATCAGTGTGGCAAATAGTTTCCAAAG
CCAGTCTGATGGGCAATGGGATCCCCACATTGTTGAGTGGCACTGAACAA

| | | | |
|---|---|---|---|
| NOV8c, CG55676-01 | | | MW at |
| Protein Sequence | SEQ ID NO: 82 | 346 aa | 39294.8 kD |

MYNGSCCRIEGDTISQVMPPLLIVAFVLGALGNGVALCGFCFHMKTWKPS
TVYLFNLAVADFLLMICLPFRTDYYLRRRHWAFGDIPCRVGLFTLANNRA
GSIVFLTVVAADRYFKVVHPHHAVNTISTRVAAGIVCTLWALVILGTVYL
LLENHLCVQETAVSCESFIMESANGWHDIMFQLEFFMPLGIILFCSFKIV
WSLRRRQQLARQARMKKATRFIMVVAIVFITCYLPSVSARLYFLWTVPSS
ACDPSVHGALHITLSFTYNNSMLDPLVYYFSSPSFPKFYNKLKICSLKPK
QPGHSKTQRPEEMPISNLGRRSCISVANSFQSQSDGQWDPHIVEWH

| | | |
|---|---|---|
| NOV8d, | SEQ ID NO: 83 | 1104 bp |
| CG55676-02 | ORF Start: | ORF Stop: |
| DNA Sequence | ATG at 60 | TG at 1098 |

GTGCCATTGTGGGGACTCCCTGGGCTGCTCTGCACCCGGACACTTGCTCT
GTCCCCGCCATGTACAACGGGTCGTGCTGCCGCATCGAGGGGGACACCAT
CTCCCAGGTGATGCCGCCGCTGCTCATTGTGGCCTTTGTGCTGGGCGCAC
TAGGCAATGGGGTCGCCCTGTGTGGTTTCTGCTTCCACATGAAGACCTGG
AAGCCCAGCACTGTTTACCTTTTCAATTTGGCCGTGGCTGATTTCCTCCT
TATGATCTGCCTGCCTTTTCGGACAGACTATTACCTCAGACGTAGACACT
GGGCTTTTGGGGACATTCCCTGCCGAGTGGGGCTCTTCACGTTGGCCATG
AACAGGGCCGGGAGCATCGTGTTCCTTACGGTGGTGGCTGCGGGCAGGTA
TTTCAAAGTGGTCCACCCCCACCACGCGGTGAACACTATCTCCACCCGGG
TGGCGGCTGGCATCGTCTGCACCCTGTGGGCCCTGGTCATCCTGGGAACA
GTGTATCTTTTGCTGGAGAACCATCTCTGCGTGCAAGAGACGGCCGTCTC
CTGTGAGAGCTTCATCATGGAGTCGGCCAATGGCTGGCATGACATCATGT
TCCAGCTGGAGTTCTTTATGCCCCTCGGCATCATCTTATTTTGCTCCTTC
AAGATTGTTTGGAGCCTGAGGCGGAGGCAGCAGCTGGCCAGACAGGCTCG
GATGAAGAAGGCGACCCGGTTCATCATGGTGGTGGCAATTGTGTTCATCA
CATGCTACCTGCCCAGCGTGTCTGCTAGACTCTATTTCCTCTGGACGGTG
CCCTCGAGTGCCTGCGATCCCTCTGTCCATGGGGCCCTGCACATAACCCT
CAGCTTCACCTACATGAACAGCATGCTGGATCCCCTGGTGTATTATTTTT
CAAGCCCCTCCTTTCCCAAATTCTACAACAAGCTCAAAATCTGCAGTCTG
AAACCCAAGCAGCCAGGACACTCAAAAACACAAAGGCCGGAAGAGATGCC
AATTTCGAACCTCGGTCGCAGGAGTTGCATCAGTGTGGCAAATAGTTTCC
AAAGCCAGTCTGATGGGCAATGGGATCCCCACATTGTTGAGTGGCACTGA
ACAA

| | | | |
|---|---|---|---|
| NOV8d, CG55676-02 | | | MW at |
| Protein Sequence | SEQ ID NO: 84 | 346 aa | 39236.8 kD |

MYNGSCCRIEGDTISQVMPPLLIVAFVLGALGNGVALCGFCFHMKTWKPS
TVYLFNLAVADFLLMICLPFRTDYYLRRRHWAFGDIPCRVGLFTLAMNRA
GSIVFLTVVAAGRYFKVVHPHHAVNTISTRVAAGIVCTLWALVILGTVYL
LLENHLCVQETAVSCESFIMESANGWHDIMFQLEFFMPLGIILFCSFKIV
WSLRRRQQLARQARMKKATRFIMVVAIVFITCYLPSVSARLYFLWTVPSS
ACDPSVHGALHITLSFTYMNSMLDPLVYYFSSPSFPKFYNKLKICSLKPK
QPGHSKTQRPEEMPISNLGRRSCISVANSFQSQSDGQWDPHIVEWH

| | | |
|---|---|---|
| NOV8e, | SEQ ID NO: 85 | 1104 bp |
| CG55676-03 | ORF Start: | ORF Stop: |
| DNA Sequence | ATG at 60 | TGA at 1098 |

GTGCCATTGTGGGGACTCCCTGGGCTGCTCTGCACCCGGACACTTGCTCT
GTCCCCGCCATGTACAACGGGTCGTGCTGCCGCATCGAGGGGGACACCAT
CTCCCAGGTGATGCCGCCGCTGCTCATTGTGGCCTTTGTGCTGGGCGCAC
TAGACAATGGGGTCGCCCTGTGTGGTTTCTGCTTCCACATGAAGACCTGG
AAGCCCAGCACTGTTTACCTTTTCAATTTGGCCGTGGCTGATTTCCTCCT
TATGATCTGCCTGCCTTTTCGGACAGACTATTACCTCAGACGTAGACACT
GGGCTTTTGGGGACATTCCCTGCCGAGTGGGGCTCTTCACGTTGGCCATG
AACAGGGCCGGGAGCATCGTGTTCCTTACGGTGGTGGCTGCGGGCAGGTA
TTTCAAAGTGGTCCACCCCCACCACGCGGTGAACACTATCTCCACCCGGG
TGGCGGCTGGCATCGTCTGCACCCTGTGGGCCCTGGTCATCCTGGGAACA
GTGTATCTTTTGCTGGAGAACCATCTCTGCGTGCAAGAGACGGCCGTCTC
CTGTGAGAGCTTCATCATGGAGTCGGCCAATGGCTGGCATGACATCATGT
TCCAGCTGGAGTTCTTTATGCCCCTCGGCATCATCTTATTTTGCTCCTTC
AAGATTGTTTGGAGCCTGAGGCGGAGGCAGCAGCTGGCCAGACAGGCTCG
GATGAAGAAGGCGACCCGGTTCATCATGGTGGTGGCAATTGTGTTCATCA
CATGCTACCTGCCCAGCGTGTCTGCTAGACTCTATTTCCTCTGGACGGTG
CCCTCGAGTGCCTGCGATCCCTCTGTCCATGGGGCCCTGCACATAACCCT
CAGCTTCACCTACATGAACAGCATGCTGGATCCCCTGGTGTATTATTTTT
CAAGCCCCTCCTTTCCCAAATTCTACAACAAGCTCAAAATCTGCAGTCTG
AAACCCAAGCAGCCAGGACACTCAAAAACACAAAGGCCGGAAGAGATGCC
AATTTCGAACCTCGGTCGCAGGAGTTGCATCAGTGTGGCAAATAGTTTCC
AAAGCCAGTCTGATGGGCAATGGGATCCCCACATTGTTGAGTGGCACTGA
ACAA

| | | | |
|---|---|---|---|
| NOV8e, CG55676-03 | | | MW at |
| Protein Sequence | SEQ ID NO: 86 | 346 aa | 39294.8 kD |

MYNGSCCRIEGDTISQVMPPLLIVAFVLGALDNGVALCGFCFHMKTWKPS
TVYLFNLAVADFLLMICLPFRTDYYLRRRHWAFGDIPCRVGLFTLANNRA
GSIVFLTVVAAGRYFKVVHPHHAVNTISTRVAAGIVCTLWALVILGTVYL
LLENHLCVQETAVSCESFIMESANGWHDIMFQLEFFMPLGIILFCSFKIV
WSLRRRQQLARQARMKKATRFIMVVAIVFITCYLPSVSARLYFLWTVPSS
ACDPSVHGALHITLSFTYMNSMLDPLVYYFSSPSFPKFYNKLKICSLKPK
QPGHSKTQRPEEMPISNLGRRSCISVANSFQSQSDGQWDPHIVEWH

| | | |
|---|---|---|
| NOV8f, | SEQ ID NO: 87 | 961 bp |
| CG55676-05 | ORF Start: | ORF Stop: |
| DNA Sequence | at 11 | at 953 |

CACCAGATCTAATGGGGTCGCCCTGTGTGGTTTCTGCTTCCACATGAAGA
CCTGGAAGCCCAGCACTGTTTACCTTTTCAATTTGGCCGTGGCTGATTTC
CTCCTTATGATCTGCCTGCCTTTTCGGACAGACTATTACCTCAGACGTAG
ACACTGGGCTTTTGGGGACATTCCCTGCCGAGTGGGGCTCTTCACGTTGG
CCATGAACAGGGCCGGGAGCATCGTGTTCCTTACGGTGGTGGCTGCGGAC
AGGTATTTCAAAGTGGTCCACCCCCACCACGCGGTGAACACTATCTCCAC
CCGGGTGGCGGCTGGCATCGTCTGCACCCTGTGGGCCCTGGTCATCCTGG
GAACAGTGTATCTTTTGCTGGAGAACCATCTCTGCGTGCAAGAGACGGCC
GTCTCCTGTGAGAGCTTCATCATGGAGTCGGCCAATGGCTGGCATGACAT
CATGTTCCAGCTGGAGTTCTTTATGCCCCTCGGCATCATCTTATTTTGCT
CCTTCAAGATTGTTTGGAGCCTGAGGCGGAGGCAGCAGCTGGCCAGACAG
GCTCGGATGAAGAAGGCGACCCGGTTCATCATGGTGGTGGCAATTGTGTT
CATCACATGCTACCTGCCCAGCGTGTCTGCTAGACTCTATTTCCTCTGGA
CGGTGCCCTCGAGTGCCTGCGATCCCTCTGTCCATGGGGCCCTGCACATA
ACCCTCAGCTTCACCTACATGAACAGCATGCTGGATCCCCTGGTGTATTA
TTTTTCAAGCCCCTCCTTTCCCAAATTCTACAACAAGCTCAAAATCTGCA
GTCTGAAACCCAAGCAGCCAGGACACTCAAAAACACAAAGGCCGGAAGAG
ATGCCAATTTCGAACCTCGGTCGCAGGAGTTGCATCAGTGTGGCAAATAG
TTTCCAAAGCCAGTCTGATGGGCAATGGGATCCCCACATTGTTGAGTGGC
ACAAGCTTGGC

| | | | |
|---|---|---|---|
| NOV8f, CG55676-05 | | | MW at |
| Protein Sequence | SEQ ID NO: 88 | 314 aa | 35943.9 kD |

NGVALCGFCFHMKTWKPSTVYLFNLAVADFLLMICLPFRTDYYLRRRHWA
FGDIPCRVGLFTLANNRAGSIVFLTVVAADRYFKVVHPHHAVNTISTRVA
AGIVCTLWALVILGTVYLLLENHLCVQETAVSCESFIMESANGWNDIMFQ
LEFFMPLGIILFCSFKIVWSLRRRQQLARQARMKKATRFIMVVAIVFITC
YLPSVSARLYFLWTVPSSACDPSVHGALHITLSFTYMNSMLDPLVYYFSS
PSFPKFYNKLKICSLKPKQPGHSKTQRPEEMPISNLGRRSCISVANSFQS
QSDGQWDPHIVEWH

| | | |
|---|---|---|
| NOV8g, | SEQ ID NO: 89 | 1060 bp |
| CG55676-06 | ORF Start: | ORF Stop: |
| DNA Sequence | ATG at 14 | at 1052 |

CACCTCGCGAACCATGTACAACGGGTCGTGCTGCCGCATCGAGGGGGACA
CCATCTCCCAGGTGATGCCGCCGCTGCTCATTGTGGCCTTTGTGCTGGGC
GCACTAGGCAATGGGGTCGCCCTGTGTGGTTTCTGCTTCCACATGAAGAC
CTGGAAGCCCAGCACTGTTTACCTTTTCAATTTGGCCGTGGCTGATTTCC
TCCTTATGATCTGCCTGCCTTTTCGGACAGACTATTACCTCAGACGTAGA
CACTGGGCTTTTGGGGACATTCCCTGCCGAGTGGGGCTCTTCACGTTGGC
CATGAACAGGGCCGGGAGCATCGTGTTCCTTACGGTGGTGGCTGCGGACA
GGTATTTCAAAGTGGTCCACCCCCACCACGCGGTGAACACTATCTCCACC
CGGGTGGCGGCTGGCATCGTCTGCACCCTGTGGGCCCTGGTCATCCTGGG
AACAGTGTATCTTTTGCTGGAGAACCATCTCTGCGTGCAAGAGACGGCCG
TCTCCTGTGAGAGCTTCATCATGGAGTCGGCCAATGGCTGGCATGACATC
ATGTTCCAGCTGGAGTTCTTTATGCCCCTCGGCATCATCTTATTTTGCTC
CTTCAAGATTGTTTGGAGCCTGAGGCGGAGGCAGCAGCTGGCCAGACAGG
CTCGGATGAAGAAGGCGACCCGGTTCATCATGGTGGTGGCAATTGTGTTC
ATCACATGCTACCTGCCCAGCGTGTCTGCTAGACTCTATTTCCTCTGGAC
GGTGCCCTCGAGTGCCTGCGATCCCTCTGTCCATGGGGCCCTGCACATAA
CCCTCAGCTTCACCTACATGAACAGCATGCTGGATCCCCTGGTGTATTAT
TTTTCAAGCCCCTCCTTTCCCAAATTCTACAACAAGCTCAPAATCTGCAG
TCTGAAACCCAAGCAGCCAGGACACTCAAAAACACAAAGGCCGGAAGAGA
TGCCAATTTCGAACCTCGGTCGCAGGAGTTGCATCAGTGTGGCAAATAGT
TTCCAAAGCCAGTCTGATGGGCAATGGGATCCCCACATTGTTGAGTGGCA
CGTCGACGGC

| | | | |
|---|---|---|---|
| NOV8g, CG55676-06 | | | MW at |
| Protein Sequence | SEQ ID NO: 90 | 346 aa | 39294.8 kD |

MYNGSCCRIEGDTISQVMPPLLIVAFVLGALGNGVALCGFCFHMKTWKPS
TVYLFNLAVADFLLMICLPFRTDYYLRRRHWAFGDIPCRVGLFTLAMNRA
GSIVFLTVVAADRYFKVVHPHHAVNTISTRVAAGIVCTLWALVILGTVYL
LLENHLCVQETAVSCESFIMESANGWHDIMFQLEFFMPLGIILFCSFKIV
WSLRRRQQLARQARMKKATRFIMVVAIVFITCYLPSVSARLYFLWTVPSS
ACDPSVHGALHITLSFTYMNSMLDPLVYYFSSPSFPKFYNKLKICSLKPK
QPGHSKTQRPEEMPISNLGRRSCISVANSFQSQSDGQWDPHIVEWH

| | | |
|---|---|---|
| NOV8h, | SEQ ID NO: 91 | 1961 bp |
| CG55676-07 | ORF Start: | ORF Stop: |
| DNA Sequence | at 2 | end of sequence |

CACCTCGCGAAATGGGGTCGCCCTGTGTGGTTTCTGCTTCCACATGAAGA
CCTGGAAGCCCAGCACTGTTTACCTTTTCAATTTGGCCGTGGCTGATTTC
CTCCTTATGATCTGCCTGCCTTTTCGGACAGACTATTACCTCAGACGTAG
ACACTGGGCTTTTGGGGACATTCCCTGCCGAGTGGGGCTCTTCACGTTGG
CCATGAACAGGGCCGGGAGCATCGTGTTCCTTACGGTGGTGGCTGCGGAC
AGGTATTTCAAAGTGGTCCACCCCCACCACGCGGTGAACACTATCTCCAC
CCGGGTGGCGGCTGGCATCGTCTGCACCCTGTGGGCCCTGGTCATCCTGG
GAACAGTGTATCTTTTGCTGGAGAACCATCTCTGCGTGCAAGAGACGGCC
GTCTCCTGTGAGAGCTTCATCATGGAGTCGGCCAATGGCTGGCATGACAT
CATGTTCCAGCTGGAGTTCTTTATGCCCCTCGGCATCATCTTATTTTGCT
CCTTCAAGATTGTTTGGAGCCTGAGGCGGAGGCAGCAGCTGGCCAGACAG
GCTCGGATGAAGAAGGCGACCCGGTTCATCATGGTGGTGGCAATTGTGTT
CATCACATGCTACCTGCCCAGCGTGTCTGCTAGACTCTATTTCCTCTGGA
CGGTGCCCTCGAGTGCCTGCGATCCCTCTGTCCATGGGGCCCTGCACATA
ACCCTCAGCTTCACCTACATGAACAGCATGCTGGATCCCCTGGTGTATTA
TTTTTCAAGCCCCTCCTTTCCCAAATTCTACAACAAGCTCAAAATCTGCA
GTCTGAAACCCAAGCAGCCAGGACACTCAAAAACACAAAGGCCGGAAGAG
ATGCCAATTTCGAACCTCGGTCGCAGGAGTTGCATCAGTGTGGCAAATAG
TTTCCAAAGCCAGTCTGATGGGCAATGGGATCCCCACATTGTTGAGTGGC
ACGTCGACGGC

| | | | |
|---|---|---|---|
| NOV8h, CG55676-07 | | | MW at |
| Protein Sequence | SEQ ID NO: 92 | 320 aa | 36559.5 kD |

TSRNGVALCGFCFHMKTWKPSTVYLFNLAVADFLLMICLPFRTDYYLRRR
HWAFGDIPCRVGLFTLAMNRAGSIVFLTVVAADRYFKVVHPHHAVNTIST
RVAAGIVCTLWALVILGTVYLLLENHLCVQETAVSCESFIMESANGWHDI
MFQLEFFMPLGIILFCSFKIVWSLRRRQQLARQARMKKATRFIMVVAIVF
ITCYLPSVSARLYFLWTVPSSACDPSVHGALHITLSFTYMNSMLDPLVYY
FSSPSFPKFYNKLKICSLKPKQPGHSKTQRPEEMPISNLGRRSCISVANS
FQSQSDGQWDPHIVEWHVDG

| | | |
|---|---|---|
| NOV8i, | SEQ ID NO: 93 | 1044 bp |
| CG55676-08 | ORF Start: | ORF Stop: |
| DNA Sequence | at 1 | at 1036 |

TACAACGGGTCGTGCTGCCGCATCGAGGGGGACACCATCTCCCAGGTGAT
GCCGCCGCTGCTCATTGTGGCCTTTGTGCTGGGCGCACTAGGCAATGGGG
TCGCCCTGTGTGGTTTCTGCTTCCACATGAAGACCTGGAAGCCCAGCACT
GTTTACCTTTTCAATTTGGCCGTGGCTGATTTCCTCCTTATGATCTGCCT
GCCTTTTCGGACAGACTATTACCTCAGACGTAGACACTGGGCTTTTGGGG
ACATTCCCTGCCGAGTGGGGCTCTTCACGTTGGCCATGAACAGGGCCGGG
AGCATCGTGTTCCTTACGGTGGTGGCTGCGGACAGGTATTTCAAAGTGGT
CCACCCCCACCACGCGGTGAACACTATCTCCACCCGGGTGGCGGCTGGCA
TCGTCTGCACCCTGTGGGCCCTGGTCATCCTGGGAACAGTGTATCTTTTG
CTGGAGAACCATCTCTGCGTGCAAGAGACGGCCGTCTCCTGTGAGAGCTT
CATCATGGAGTCGGCCAATGGCTGGCATGACATCATGTTCCAGCTGGAGT
TCTTTATGCCCCTCGGCATCATCTTATTTTGCTCCTTCAAGATTGTTTGG
AGCCTGAGGCGGAGGCAGCAGCTGGCCAGACAGGCTCGGATGAAGAAGGC
GACCCGGTTCATCATGGTGGTGGCAATTGTGTTCATCACATGCTACCTGC
CCAGCGTGTCTGCTAGACTCTATTTCCTCTGGACGGTGCCCTCGAGTGCC
TGCGATCCCTCTGTCCATGGGGCCCTGCACATAACCCTCAGCTTCACCTA
CATGAACAGCATGCTGGATCCCCTGGTGTATTATTTTTCAAGCCCCTCCT
TTCCCAAATTCTACAACAAGCTCAAAATCTGCAGTCTGAAACCCAAGCAG
CCAGGACACTCAAAAACACAAAGGCCGGAAGAGATGCCAATTTCGAACCT
CGGTCGCAGGAGTTGCATCAGTGTGGCAAATAGTTTCCAAAGCCAGTCTG
ATGGGCAATGGGATCCCCACATTGTTGAGTGGCACGAATTCGGC

| | | | |
|---|---|---|---|
| NOV8i, CG55676-08 | | | MW at |
| Protein Sequence | SEQ ID NO: 94 | 345 aa | 39163.6 kD |

YNGSCCRIEGDTISQVMPPLLIVAFVLGALGNGVALCGFCFHMKTWKPST
VYLFNLAVADFLLMICLPFRTDYYLRRRHWAFGDIPCRVGLFTLANNRAG
SIVFLTVVAADRYFKVVHPHHAVNTISTRVAAGIVCTLWALVILGTVYLL
LENHLCVQETAVSCESFIMESANGWHDIMFQLEFFMPLGIILFCSFKIVW
SLRRRQQLARQARMKKATRFIMVVAIVFITCYLPSVSARLYFLWTVPSSA
CDPSVHGALHITLSFTYMNSMLDPLVYYFSSPSFPKFYNKLKICSLKPKQ
PGHSKTQRPEEMPISNLGRRSCISVANSFQSQSDGQWDPHIVEWH

A ClustalW comparison of the above protein sequences yields the following sequence alignment shown in Table 8B.

TABLE 8B
Comparison of the NOV8 protein sequences.

| | |
|---|---|
| NOV8a | MYNGSCCRIEGDTISQVMPPLLIVAFVLGALGNGVALCGFCFHMKTWKPSTVYLFNLAVA |
| NOV8b | -----------------------------TSRNGVALCGFCFHMKTWKPSTVYLFNLAVA |
| NOV8c | MYNGSCCRIEGDTISQVMPPLLIVAFVLGALGNGVALCGFCFHMKTWKPSTVYLFNLAVA |
| NOV8d | MYNGSCCRIEGDTISQVMPPLLIVAFVLGALGNGVALCGFCFHMKTWKPSTVYLFNLAVA |
| NOV8e | MYNGSCCRIEGDTISQVMPPLLIVAFVLGALDNGVALCGFCFHMKTWKPSTVYLFNLAVA |
| NOV8f | --------------------------------NGVALCGFCFHMKTWKPSTVYLFNLAVA |
| NOV8g | MYNGSCCRIEGDTISQVMPPLLIVAFVLGALGNGVALCGFCFHMKTWKPSTVYLFNLAVA |
| NOV8h | -----------------------------TSRNGVALCGFCFHMKTWKPSTVYLFNLAVA |
| NOV8i | -YNGSCCRIEGDTISQVMPPLLIVAFVLGALGNGVALCGFCFHMKTWKPSTVYLFNLAVA |
| NOV8a | DFLLMICLPFRTDYYLRRRHWAFGDIPCRVGLFTLAMNRAGSIVFLTVVAADRYFKVVHP |
| NOV8b | DFLLMICLPFRTDYYLRRRHWAFGDIPCRVGLFTLAMNRAGSIVFLTVVAADRYFKVVHP |
| NOV8c | DFLLMICLPFRTDYYLRRRHWAFGDIPCRVGLFTLAMNRAGSIVFLTVVAADRYFKVVHP |
| NOV8d | DFLLMICLPFRTDYYLRRRHWAFGDIPCRVGLFTLANNRAGSIVFLTVVAAGRYFKVVHP |
| NOV8e | DFLLMICLPFRTDYYLRRRHWAFGDIPCRVGLFTLAMNRAGSIVFLTVVAAGRYFKVVHP |
| NOV8f | DFLLMICLPFRTDYYLRRRHWAFGDIPCRVGLFTLANNRAGSIVFLTVVAADRYFKVVHP |
| NOV8g | DFLLMICLPFRTDYYLRRRHWAFGDIPCRVGLFTLAMNRAGSIVFLTVVAADRYFKVVHP |
| NOV8h | DFLLMICLPFRTDYYLRRRHWAFGDIPCRVGLFTLAMNRAGSIVFLTVVAADRYFKVVHP |
| NOV8i | DFLLMICLPFRTDYYLRRRHWAFGDIPCRVGLFTLANNRAGSIVFLTVVAADRYFKVVHP |
| NOV8a | HHAVNTISTRVAAGIVCTLWALVILGTVYLLLENHLCVQETAVSCESFIMESANGWHDIM |
| NOV8b | HHAVNTISTRVAAGIVCTLWALVILGTVYLLLENHLCVQETAVSCESFIMESANGWHDIM |
| NOV8c | HHAVNTISTRVAAGIVCTLWALVILGTVYLLLENHLCVQETAVSCESFIMESANGWHDIM |
| NOV8d | HHAVNTISTRVAAGIVCTLWALVILGTVYLLLENHLCVQETAVSCESFIMESANGWHDIM |
| NOV8e | HHAVNTISTRVAAGIVCTLWALVILGTVYLLLENHLCVQETAVSCESFIMESANGWHDIM |
| NOV8f | HHAVNTISTRVAAGIVCTLWALVILGTVYLLLENHLCVQETAVSCESFIMESANGWHDIM |
| NOV8g | HHAVNTISTRVAAGIVCTLWALVILGTVYLLLENHLCVQETAVSCESFIMESANGWHDIM |
| NOV8h | HHAVNTISTRVAAGIVCTLWALVILGTVYLLLENHLCVQETAVSCESFIMESANGWHDIM |
| NOV8i | HHAVNTISTRVAAGIVCTLWALVILGTVYLLLENHLCVQETAVSCESFIMESANGWHDIM |
| NOV8a | FQLEFFMPLGIILFCSFKIVWSLRRRQQLARQARMKKATRFIMVVAIVFITCYLPSVSAR |
| NOV8b | FQLEFFMPLGIILFCSFKIVWSLRRRQQLARQARMKKATRFIMVVAIVFITCYLPSVSAR |
| NOV8c | FQLEFFMPLGIILFCSFKIVWSLRRRQQLARQARMKKATRFIMVVAIVFITCYLPSVSAR |
| NOV8d | FQLEFFMPLGIILFCSFKIVWSLRRRQQLARQARMKKATRFIMVVAIVFITCYLPSVSAR |
| NOV8e | FQLEFFMPLGIILFCSFKIVWSLRRRQQLARQARMKKATRFIMVVAIVFITCYLPSVSAR |
| NOV8f | FQLEFFMPLGIILFCSFKIVWSLRRRQQLARQARMKKATRFIMVVAIVFITCYLPSVSAR |
| NOV8g | FQLEFFMPLGIILFCSFKIVWSLRRRQQLARQARMKKATRFIMVVAIVFITCYLPSVSAR |
| NOV8h | FQLEFFMPLGIILFCSFKIVWSLRRRQQLARQARMKKATRFIMVVAIVFITCYLPSVSAR |
| NOV8i | FQLEFFMPLGIILFCSFKIVWSLRRRQQLARQARMKKATRFIMVVAIVFITCYLPSVSAR |
| NOV8a | LYFLWTVPSSACDPSVHGALHITLSFTYMNSMLDPLVYYFSSPSFPKFYNKLKICSLKPK |
| NOV8b | LYFLWTVPSSACDPSVHGALHITLSFTYMNSMLDPLVYYFSSPSFPKFYNKLKICSLKPK |
| NOV8c | LYFLWTVPSSACDPSVHGALHITLSFTYMNSMLDPLVYYFSSPSFPKFYNKLKICSLKPK |
| NOV8d | LYFLWTVPSSACDPSVHGALHITLSFTYMNSMLDPLVYYFSSPSFPKFYNKLKICSLKPK |
| NOV8e | LYFLWTVPSSACDPSVHGALHITLSFTYMNSMLDPLVYYFSSPSFPKFYNKLKICSLKPK |
| NOV8f | LYFLWTVPSSACDPSVHGALHITLSFTYMNSMLDPLVYYFSSPSFPKFYNKLKICSLKPK |
| NOV8g | LYFLWTVPSSACDPSVHGALHITLSFTYMNSMLDPLVYYFSSPSFPKFYNKLKICSLKPK |
| NOV8h | LYFLWTVPSSACDPSVHGALHITLSFTYMNSMLDPLVYYFSSPSFPKFYNKLKICSLKPK |
| NOV8i | LYFLWTVPSSACDPSVHGALHITLSFTYMNSMLDPLVYYFSSPSFPKFYNKLKICSLKPK |
| NOV8a | QPGHSKTQRPEEMPISNLGRRSCISVANSFQSQSDGQWDPHIVEWH--- |
| NOV8b | QPGHSKTQRPEEMPISNLGRRSCISVANSFQSQSDGQWDPHIVEWHVDG |
| NOV8c | QPGHSKTQRPEEMPISNLGRRSCISVANSFQSQSDGQWDPHIVEWH--- |
| NOV8d | QPGHSKTQRPEEMPISNLGRRSCISVANSFQSQSDGQWDPHIVEWH--- |
| NOV8e | QPGHSKTQRPEEMPISNLGRRSCISVANSFQSQSDGQWDPHIVEWH--- |
| NOV8f | QPGHSKTQRPEEMPISNLGRRSCISVANSFQSQSDGQWDPHIVEWH--- |
| NOV8g | QPGHSKTQRPEEMPISNLGRRSCISVANSFQSQSDGQWDPHIVEWH--- |
| NOV8h | QPGHSKTQRPEEMPISNLGRRSCISVANSFQSQSDGQWDPHIVEWHVDG |
| NOV8i | QPGHSKTQRPEEMPISNLGRRSCISVANSFQSQSDGQWDPHIVEWH--- |
| NOV8a | (SEQ ID NO: 78) |
| NOV8b | (SEQ ID NO: 80) |
| NOV8c | (SEQ ID NO: 82) |
| NOV8d | (SEQ ID NO: 84) |
| NOV8e | (SEQ ID NO: 86) |
| NOV8f | (SEQ ID NO: 88) |
| NOV8g | (SEQ ID NO: 90) |
| NOV8h | (SEQ ID NO: 92) |
| NOV8i | (SEQ ID NO: 94) |

Further analysis of the NOV8a protein yielded the following properties shown in Table 8C.

TABLE 8C
Protein Sequence Properties NOV8a

| SignalP | Cleavage site between residues 33 and 34 | | | | |
|---|---|---|---|---|---|
| analysis: | | | | | |
| PSORT II | PSG: a new signal peptide prediction method | | | | |
| analysis: | N-region: length 10; pos. chg 1; neg. chg 1 | | | | |
| | H-region: length 1; peak value −0.74 | | | | |
| | PSG score: −5.14 | | | | |
| | GvH: von Heijne's method for signal seq. recognition | | | | |
| | GvH score (threshold: −2.1): −0.50 | | | | |
| | possible cleavage site: between 32 and 33 | | | | |
| | >>> Seems to have no N-terminal signal peptide | | | | |
| | ALOM: Klein et al's method for TM region allocation | | | | |
| | Init position for calculation: 1 | | | | |
| | Tentative number of TMS(s) for the threshold 0.5: 6 | | | | |
| | INTEGRAL | Likelihood = | −6.74 | Transmembrane | 21-37 |
| | INTEGRAL | Likelihood = | −5.47 | Transmembrane | 52-68 |
| | INTEGRAL | Likelihood = | 0.10 | Transmembrane | 95-111 |
| | INTEGRAL | Likelihood = | −7.43 | Transmembrane | 135-151 |
| | INTEGRAL | Likelihood = | −3.45 | Transmembrane | 179-195 |
| | INTEGRAL | Likelihood = | −7.75 | Transmembrane | 221-237 |

| PERIPHERAL | Likelihood = | 7.64 (at 262) | |
|---|---|---|---|
| ALOM score: −7.75 (number of TMSs: 6) | | | |
| MTOP: Prediction of membrane topology (Hartmann et al.) | | | |
| Center position for calculation: 28 | | | |
| Charge difference: 2.5 C(2.5)-N(0.0) | | | |
| C > N: C-terminal side will be inside | | | |
| >>> membrane topology: type 3b | | | |
| MITDISC: discrimination of mitochondrial targeting seq | | | |
| R content: | 1 | Hyd Moment (75): | 11.18 |
| Hyd Moment(95): | 5.39 | G content: | 2 |
| D/E content: | 2 | S/T content: | 1 |
| Score: | −6.17 | | |

Gavel: prediction of cleavage sites for mitochondrial preseq
R-2 motif at 18 CRI|EG
NUCDISC: discrimination of nuclear localization signals
pat4: RRRH (3) at 77
pat7: none
bipartite: RRRQQLARQARMKKATR at 204
content of basic residues: 9.0%
NLS Score: 0.21
KDEL: ER retention motif in the C-terminus: none
ER Membrane Retention Signals: none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
55.6%: endoplasmic reticulum
22.2%: vacuolar
11.1%: Golgi
11.1%: mitochondrial
>> prediction for CG55676-04 is end (k = 9)

A search of the NOV8a protein against the Geneseq database, a proprietary database that contains sequences published in patents and patent publication, yielded several homologous proteins shown in Table 8D.

TABLE 8D
Geneseq Results for NOV8a

| | | NOV8a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABP58453 | Human respiratory chemokine | 1 . . . 346 | 346/346 (100%) | 0.0 |
| | receptor - Homo sapiens, 346 aa. | 1 . . . 346 | 346/346 (100%) | |
| | [WO2003002604-A2, 09 JAN. 2003] | | | |
| ABP56751 | Human GAVE3 protein SEQ ID NO: 2 - | 1 . . . 346 | 346/346 (100%) | 0.0 |
| | Homo sapiens, 346 aa. | 1 . . . 346 | 346/346 (100%) | |
| | [WO2003000846-A2, 03 JAN. 2003] | | | |
| AAO26511 | Human G-Protein Coupled Receptor | 1 . . . 346 | 346/346 (100%) | 0.0 |
| | protein - Homo sapiens, 346 aa. | 1 . . . 346 | 346/346 (100%) | |
| | [WO200283736-A2, 24 OCT. 2002] | | | |
| ABP81747 | Human chemokine receptor | 1 . . . 346 | 346/346 (100%) | 0.0 |
| | FKSG80/GPR81 protein SEQ ID NO: | 1 . . . 346 | 346/346 (100%) | |
| | 668 - Homo sapiens, 346 aa. | | | |
| | [WO200261087-A2, 08 AUG. 2002] | | | |
| AAE17077 | Human G-protein coupled receptor | 1 . . . 346 | 346/346 (100%) | 0.0 |
| | (GPCRxl4) protein - Homo sapiens, | 1 . . . 346 | 346/346 (100%) | |
| | 346 aa. [WO200198330-A2, | | | |
| | 27 DEC. 2001] | | | |

In a BLAST search of public sequence databases, the NOV8a protein was found to have homology to the proteins shown in the BLASTP data in Table 8E.

TABLE 8E
Public BLASTP Results for NOV8a

| | | NOV8a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q9BXC0 | Putative chemokine receptor (G | 1 . . . 346 | 346/346 (100%) | 0.0 |
| | protein-coupled receptor) (Putative | 1 . . . 346 | 346/346 (100%) | |
| | G-protein coupled receptor) (Seven | | | |
| | transmembrane helix receptor) - Homo | | | |
| | sapiens (Human), 346 aa. | | | |
| Q8C131 | Similar to putative chemokine receptor - | 1 . . . 339 | 276/339 (81%) | e−165 |
| | Mus musculus (Mouse), 343 aa. | 1 . . . 339 | 295/339 (86%) | |
| CAD62066 | Sequence 3 from Patent WO02083736 - | 1 . . . 339 | 276/339 (81%) | e−164 |
| | Mus musculus (Mouse), 351 aa. | 9 . . . 347 | 295/339 (86%) | |
| CAD62069 | Sequence 5 from Patent WO02083736 - | 1 . . . 338 | 276/338 (81%) | e−164 |
| | Rattus norvegicus (Rat), 351 aa. | 9 . . . 346 | 298/338 (87%) | |
| Q8TDS4 | Putative G-protein coupled receptor | 5 . . . 340 | 180/341 (52%) | 9e−94 |
| | (Seven transmembrane helix receptor) - | 17 . . . 355 | 227/341 (65%) | |
| | Homo sapiens (Human), 363 aa. | | | |

PFam analysis predicts that the NOV8a protein contains the domains shown in the Table 8F.

TABLE 8F
Domain Analysis of NOV8a

| | | Identities/ | |
|---|---|---|---|
| | | Similarities | |
| Pfam | NOV8a | for the | Expect |
| Domain | Match Region | Matched Region | Value |
| 7tm_1 | 32 . . . 278 | 74/276 (27%) | 2.9e−38 |
| | | 174/276 (63%) | |
| V1R | 42 . . . 291 | 56/283 (20%) | 0.4 |
| | | 123/283 (43%) | |

### Example 9

The NOV9 clone was analyzed, and the nucleotide and encoded polypeptide sequences are shown in Table 9A.

TABLE 9A
NOV9 Sequence Analysis

| | | |
|---|---|---|
| NOV9a, | SEQ ID NO: 95 | 1254 bp |
| CG57042-01 | ORF Start: | ORF Stop: |
| DNA Sequence | ATG at 7 | TAG at 1000 |

CTAAAGATGAAACGGCTGGTTTGTGTGCTCTTGGTGTGCTCCTCTGCAGT
GGCACAGTTGCATAAAGATCCTACCCTGGATCACCACTGGCATCTCTGGA
AGAAAACCTATGGCAAACAATACAAGGAAAAGAATGAAGAAGCAGTACGA
CGTCTCATCTGGGAAAAGAATCTAAAGTTTGTGATGCTTCACAACCTGGA
GCATTCAATGGGAATGCACTCATACGATCTGGGCATGAACCACCTGGGAG
ACATGACCAGTGAAGAAGTGATGTCTTTGATGAGTTCCCTGAGAGTTCCC
AGCCAGTGGCAGAGAAATATCACATATAAGTCAAACCCTAATCGGATATT
GCCTGATTCTGTGGACTGGAGAGAGAAAGGGTGTGTTACTGAAGTGAAAT
ATCAAGGTTCTTGTGGTGCTTGCTGGGCTTTCAGTGCTGTGGGGGCCCTG
GAAGCACAGCTGAAGCTGAAAACAGGAAAGCTGGTGTCTCTCAGTGCCCA
GAACCTGGTGGATTGCTCAACTGAAAAATATGGAAACAAAGGCTGCAATG
GTGGCTTCATGACAACGGCTTTCCAGTACATCATTGATAACAAGGGCATC
GACTCAGACGCTTCCTATCCCTACAAAGCCATGGATCAGAAATGTCAATA
TGACTCATAATATCGTGCTGCCACATGTTCAAAGTACACTGAACTTCCTT
ATGGCAGAGAAGATGTCCTGAAAGAAGCTGTGGCCAATAGAGGCCCAGTG
TCTGTTGGTGTAGATGCGCGTCATCCTTCCTTCTTCCTCTACAGAAGTGG
TGTCTACTATGAACCATCCTGTACTCAGAATGTGAATCATGGTGTACTTG
TGGTTGGCTATGGTGATCTTAATGGGAAAGAATACTGGCTTGTGAAAAAC
AGCTGGGGTCACAACTTTGGTGAAGAAGGATATATTCGGATGGCAAGAAA
TAAAGGAAATCATTGTGGGATTGCTAGCTTTCCCTCTTACCCAGAAATCT
AGAGGATCTCTCCTTTTTATAACAAATCAAGAAATATGAAGCACTTTCTC
TTAACTTAATTTTTCCTGCTGTATCCAGAAGAAATAATTGTGTCATGATT
AATGTGTATTTACTGTACTAATTAGAAAATATAGTTTGAGGCCGGGCACG
GTGGCTCACGCCTGTAATCCCAGTACTTGGGAGGCCAAGGCAGGCATATC
AACTTGAGGCCAGGAGTTAAAGAGCAGCCTGGCTAACTGTGAAACCCCTC
TACT

| | | | |
|---|---|---|---|
| NOV9a, CG57042-01 | | | MW at |
| Protein Sequence | SEQ ID NO: 96 | 331 aa | 37523.4 kD |

MKRLVCVLLVCSSAVAQLHKDPTLDHHWHLWKKTYGKQYKEKNEEAVRRL
IWEKNLKFVMLHNLEHSMGMHSYDLGMNHLGDMTSEEVMSLMSSLRVPSQ
WQRNITYKSNPNRILPDSVDWREKGCVTEVKYQGSCGACWAFSAVGALEA
QLKLKTGKLVSLSAQNLVDCSTEKYGNKGCNGGFMTTAFQYIIDNKGIDS
DASYPYKAMDQKCQYDSKYRAATCSKYTELPYGREDVLKEAVANRGPVSV
GVDARHPSFFLYRSGVYYEPSCTQNVNHGVLVVGYGDLNGKEYWLVKNSW
GHNFGEEGYIRMARNKGNHCGIASFPSYPEI

| | | |
|---|---|---|
| NOV9b, | SEQ ID NO: 97 | 667 bp |
| 275620530 | ORF Start: | ORF Stop: |
| DNA Sequence | at 2 | end of sequence |

CACCAAGCTTTTGCCTGATTCTGTGGACTGGAGAGAGAAAGGGTGTGTTA
CTGAAGTGAAATATCAAGGTTCTTGTGGTGCTTGCTGGGCTTTCAGTGCT
GTGGGGGCCCTGGAAGCACAGCTGAAGCTGAAAACAGGAAAGCTGGTGTC
TCTCAGTGCCCAGAACCTGGTGGATTGCTCAACTGAAAAATATGGAAACA
AAGGCTGCAATGGTGGCTTCATGACAACGGCTTTCCAGTACATCATTGAT
AACAAGGGCATCGACTCAGACGCTTCCTATCCCTACAAAGCCATGGATCA
GAAATGTCAATATGACTCAAAATATCGTGCTGCCACATGTTCAAAGTACA
CTGAACTTCCTTATGGCAGAGAAGATGTCCTGAAAGAAGCTGTGGCCAAT
AGAGGCCCAGTGTCTGTTGGTGTAGATGCGCGTCATCCTTCCTTCTTCCT
CTACAGAAGTGGTGTCTACTATGAACCATCCTGTACTCAGAATGTGAATC
ATGGTGTACTTGTGGTTGGCTATGGTGATCTTAATGGGAAAGAATACTGG
CTTGTGAAAAACAGCTGGGGTCACAACTTTGGTGAAGAAGGATATATTCG
GATGGCAAGAAATAAAGGAAATCATTGTGGGATTGCTAGCTTTCCCTCTT
ACCCAGAAGTCGACGGC

| | | | |
|---|---|---|---|
| Nov9b, 275620530 | | | MW at |
| Protein Sequence | SEQ ID NO: 98 | 222 aa | 24507.3 kD |

TKLLPDSVDWREKGCVTEVKYQGSCGACWAFSAVGALEAQLKLKTGKLVS
LSAQNLVDCSTEKYGNKGCNGGFMTTAFQYIIDNKGIDSDASYPYKAMDQ
KCQYDSKYRAATCSKYTELPYGREDVLKEAVANRGPVSVGVDARHPSFFL
YRSGVYYEPSCTQNVNHGVLVVGYGDLNGKEYWLVKNSWGHNFGEEGYIR
MARNKGNHCGIASFPSYPEVDG

A ClustalW comparison of the above protein sequences yields the following sequence alignment shown in Table 9B.

TABLE 9B
C mparis n of the NOV9 protein sequences.

| | |
|---|---|
| NOV9a | MKRLVCVLLVCSSAVAQLHKDPTLDHHWHLWKKTYGKQYKEKNEEAVRRLIWEKNLKFVM |
| NOV9b | ------------------------------------------------------------ |
| NOV9a | LHNLEHSMGMHSYDLGNNNLGDMTSEEVMSLMSSLRVPSQWQRNITYKSNPNRILPDSVD |
| NOV9b | ---------------------------------------------------TKLLPDSVD |
| NOV9a | WREKGCVTEVKYQGSCGACWAFSAVGALEAQLKLKTGKLVSLSAQNLVDCSTEKYGNKGC |
| NOV9b | WREKGCVTEVKYQGSCGACWAFSAVGALEAQLKLKTGKLVSLSAQNLVDCSTEKYGNKGC |
| NOV9a | NGGFMTTAFQYIIDNKGIDSDASYPYKAMDQKCQYDSKYRAATCSKYTELPYGREDVLKE |
| NOV9b | NGGFMTTAFQYIIDNKGIDSDASYPYKAMDQKCQYDSKYRAATCSKYTELPYGREDVLKE |
| NOV9a | AVANRGPVSVGVDARHPSFFLYRSGVYYEPSCTQNVNHGVLVVGYGDLNGKEYWLVKNSW |
| NOV9b | AVANRGPVSVGVDARHPSFFLYRSGVYYEPSCTQNVNHGVLVVGYGDLNGKEYWLVKNSW |
| NOV9a | GHNFGEEGYIRMARNKGNNCGIASFPSYPEI-- |
| NOV9b | GHNFGEEGYIRMARNKGNHCGIASFPSYPEVDG |
| NOV9a | (SEQ ID NO: 96) |
| NOV9b | (SEQ ID NO: 98) |

Further analysis of the NOV9a protein yielded the following properties shown in Table 9C.

TABLE 9C
Protein Sequence Properties NOV9a

| SignalP | Cleavage site between residues 17 and 18 | | | |
|---|---|---|---|---|
| analysis: | | | | |
| PSORT II | PSG: a new signal peptide prediction method | | | |
| analysis: | N-region: length 3; pos. chg 2; neg. chg 0 | | | |
| | H-region: length 16; peak value 10.58 | | | |
| | PSG score: 6.18 | | | |
| | GvH: von Heijne's method for signal seq. recognition | | | |
| | GvH score (threshold: −2.1): 2.34 | | | |
| | possible cleavage site: between 16 and 17 | | | |
| | >>> Seems to have a cleavable signal peptide (1 to 16) | | | |
| | ALOM: Klein et al's method for TM region allocation | | | |
| | Init position for calculation: 17 | | | |
| | Tentative number of TMS(s) for the threshold 0.5: 0 | | | |
| | number of TMS(s) . . . fixed | | | |
| | PERIPHERAL | Likelihood = | 4.14 (at 136) | |
| | ALOM score: 4.14 (number of TMSs: 0) | | | |
| | MTOP: Prediction of membrane topology (Hartmann et al.) | | | |
| | Center position for calculation: 8 | | | |
| | Charge difference: 0.0 C(3.0)-N(3.0) | | | |
| | N >= C: N-terminal side will be inside | | | |
| | MITDISC: discrimination of mitochondrial targeting seq | | | |
| | R content: | 1 | Hyd Moment(75): | 9.54 |
| | Hyd Moment(95): | 10.80 | G content: | 0 |
| | D/E content: | 1 | S/T content: | 2 |

Score: −2.46
Gavel: prediction of cleavage sites for mitochondrial preseq
R-2 motif at 13 KRL|VC
NUCDISC: discrimination of nuclear localization signals
pat4: none
pat7: none
bipartite: none
content of basic residues: 12.1%
NLS Score: −0.47
KDEL: ER retention motif in the C-terminus: none
ER Membrane Retention Signals:
XXRR-like motif in the N-terminus: KRLV none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 70.6
COIL: Lupas's algorithm to detect coiled-coil regions
total: 0 residues
Final Results (k = 9/23):
33.3%: endoplasmic reticulum
22.2%: vacuolar
22.2%: extracellular, including cell wall
11.1%: mitochondrial
11.1%: Golgi
>> prediction for CG57042-01 is end (k = 9)

A search of the NOV9a protein against the Geneseq database, a proprietary database that contains sequences published in patents and patent publication, yielded several homologous proteins shown in Table 9D.

TABLE 9D
Geneseq Results for NOV9a

| | | NOV9a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAY59634 | Human Cathepsin S amino acid | 1 . . . 331 | 329/331 (99%) | 0.0 |
| | sequence - Homo sapiens, 331 aa. | 1 . . . 331 | 330/331 (99%) | |
| | [WO9963115-A2, 09 DEC. 1999] | | | |
| ABG73437 | Human Cathepsin S polypeptide - | 1 . . . 331 | 285/331 (86%) | e−177 |
| | Homo sapiens, 331 aa. | 1 . . . 331 | 304/331 (91%) | |
| | [US2002164765-A1, 07 NOV. 2002] | | | |
| AAW77071 | Rat Cathepsin K polypeptide - Rattus | 18 . . . 331 | 177/317 (55%) | e−103 |
| | sp, 329 aa. [EP861898-A1, | 16 . . . 329 | 223/317 (69%) | |
| | 02 SEP. 1998] | | | |
| AAY81000 | Human procathepsin K - Homo | 18 . . . 331 | 178/317 (56%) | e−102 |
| | sapiens, 329 aa. [WO200009653-A2, | 16 . . . 329 | 224/317 (70%) | |
| | 24 FEB. 2000] | | | |
| AAB57115 | Human prostate cancer antigen protein | 18 . . . 331 | 178/317 (56%) | e−102 |
| | sequence SEQ ID NO: 1693 - Homo | 48 . . . 361 | 224/317 (70%) | |
| | sapiens, 361 aa. [WO200055174-A1, | | | |
| | 21 SEP. 2000] | | | |

In a BLAST search of public sequence databases, the NOV9a protein was found to have homology to the proteins shown in the BLASTP data in Table 9E.

TABLE 9E
Public BLASTP Results for NOV9a

| | | NOV9a | Identities/ | |
|---|---|---|---|---|
| Protein | | Residues/ | Similarities | |
| Accession | | Match | for the | Expect |
| Number | Protein/Organism/Length | Residues | Matched Portion | Value |
| P25774 | Cathepsin S precursor (EC 3.4.22.27) - | 1 . . . 331 | 329/331 (99%) | 0.0 |
| | Homo sapiens (Human), 331 aa. | 1 . . . 331 | 331/331 (99%) | |
| A42482 | cathepsin S (EC 3.4.22.27) precursor - | 1 . . . 331 | 328/331 (99%) | 0.0 |
| | human, 331 aa. | 1 . . . 331 | 330/331 (99%) | |
| Q8HY82 | Cathepsin S preproprotein precursor - | 1 . . . 331 | 310/331 (93%) | 0.0 |
| | Saimiri boliviensis (Bolivian squirrel | 1 . . . 330 | 322/331 (96%) | |
| | monkey), 330 aa. | | | |
| Q8HY81 | Cathepsin S preproprotein precursor - | 1 . . . 331 | 290/331 (87%) | e−178 |
| | Canis familiaris (Dog), 331 aa. | 1 . . . 331 | 310/331 (93%) | |
| Q8BSZ5 | Cathepsin S - Mus musculus (Mouse), | 9 . . . 331 | 243/324 (75%) | e−151 |
| | 342 aa. | 19 . . . 342 | 283/324 (87%) | |

PFam analysis predicts that the NOV9a protein contains the domains shown in the Table 9F.

TABLE 9F
Domain Analysis of NOV9a

| | | Identities/ | |
|---|---|---|---|
| | | Similarities | |
| | NOV9a | for the | Expect |
| Pfam Domain | Match Region | Matched Region | Value |
| Peptidase_C1 | 115 . . . 330 | 124/332 (37%) | 8.4e−122 |
| | | 197/332 (59%) | |

### Example 10

The NOV10 clone was analyzed, and the nucleotide and encoded polypeptide sequences are shown in Table 10A.

TABLE 10A
NOV10 Sequence Analysis

| | | |
|---|---|---|
| NOV10a, | SEQ ID NO: 99 | 1222 bp |
| CG57589-01 | ORF Start: | ORF Stop: |
| DNA Sequence | ATG at 67 | TAA at 1156 |

ATTGCGGGCGGCGGCGTTCGGAGTCGCCGGGAGCTGCCAGGCTGTCCGCG
CCGCCGCTGCGGGGCCATGATCCGGAACGGGCGCGGGGCTGCAGGCGGC
GCAGAGCAGCCGGGCCCGGGGGGCAGGCGCGCCGTGAGGGTGTGGTGCGA
TGGCTGCTATGACATGGTGCATTACGGCCACTCCAACCAGCTGCGCCAGG
CACGGGCCATGGGTGACTACCTCATCGTAGGCGTGCACACCGATGAGGAG
ATCGCCAAGCACAAGGGGCCCCCGGTGTTCACTCAGGAGGAGAGATACAA
GATGGTGCAGGCCATCAAATGGGTGGACGAGGTGGTGCCAGCGGCTCCCT
ACGTCACTACACTAGAGACCCTGGACAAATACAACTGTGACTTCTGTGTT
CACGGCAATGACATCACCCTGACTGTAGATGGCCGGGACACCTATGAGGA
AGTAAAGCAGGCTGGGAGGTACAGAGAATGCAAGCGCACGCAAGGGGTGT
CCACCACAGACCTCGTGGGCCGCATGCTGCTGGTAACCAAAGCCCATCAC
AGCAGCCAGGAGATGTCCTCTGAGTACCGGGAGTATGCAGACAGTTTTGG
CAAGTGCCCTGGTGGGCGGAACCCCTGGACCGGGGTATCCCAGTTCCTGC
AGACATCTCAGAAGATCATCCAGTTTGCTTCTGGGAAGGAGCCCCAGCCA
GGGGAGACAGTCATCTATGTGGCTGGTGCCTTCGACCTGTTCCACATCGG
GCATGTGGACTTCCTGGAGAAGGTGCACAGGCTGGCAGAGAGGCCCTACA
TCATCGCGGGCTTACACTTTGACCAGTACGTGTCAGAAGTGGTGATTGGA
GCCCCGTACGCGGTCACAGCAGAGCTCCTAAGTCACTTCAAGGTGGACCT
GGTGTGTCACGGCAAGACAGGAATTATCCCTGACAGGGATGGCTCCGACC
CATACCAGGAGCCCAAGAGAAGGGGCATCTTCCGTCAGATTGACAGTGGC
AGCAACCTCACCACAGACCTCATCGTCCAGCGGATCATCACCAACAGGTT
GGAGTATGAGGCGCGAAACCAGAAGAAGGAAGCCAAGGAGCTGGCCTTCC
TGGAGGCTGCCAGGCAGCAGGCGGCACAGCCCCTGGGGGAGCGCGATGGT
GACTTCTAACCTGGCAGAGGCCCTGGCCGGCCCTCCCCCTGCTCTGCTTC
TGCGCCTTCTGCGTTTGGACATA

| | | | |
|---|---|---|---|
| NOV10a, CG57589-01 | | | MW at |
| Protein Sequence | SEQ ID NO: 100 | 363 aa | 40694.5 kD |

MIRNGRGAAGGAEQPGPGGRRAVRVWCDGCYDMVHYGHSNQLRQARAMGD
YLIVGVHTDEEIAKHKGPPVFTQEERYKMVQAIKWVDEVVPAAPYVTTLE
TLDKYNCDFCVHGNDITLTVDGRDTYEEVKQAGRYRECKRTQGVSTTDLV
GRMLLVTKAHHSSQEMSSEYREYADSFGKCPGGRNPWTGVSQFLQTSQKI
IQFASGKEPQPGETVIYVAGAFDLFHIGHVDFLEKVHRLAERPYIIAGLH
FDQYVSEVVIGAPYAVTAELLSHFKVDLVCHGKTGIIPDRDGSDPYQEPK
RRGIFRQIDSGSNLTTDLIVQRIITNRLEYEARNQKKEAKELAFLEAARQ
QAAQPLGERDGDF

| | | |
|---|---|---|
| NOV10b, | SEQ ID NO: 101 | 1243 bp |
| 283866181 | ORF Start: | ORF Stop: |
| DNA Sequence | at 2 | end of sequence |

CACCGGATCCACCATGATCCGGAACGGGCGCGGGGCTGCAGGCGGCGCAG
AGCAGCCGGGCCCGGGGGGCAGGCGCGCCGTGAGGGTGTGGTGCGATGGC
TGCTATGACATGGTGCATTACGGCCACTCCAACCAGCTGCGCCAGGCACG
GGCCATGGGTGACTACCTCATCGTAGGCGTGCACACCGATGAGGAGATCG
CCAAGCACAAGGGGCCCCCGGTGTTCACTCAGGAGGAGAGATACAAGATG
GTGCAGGCCATCAAATGGGTGGACGAGGTGGTGCCAGCGGCTCCCTACGT
CACTACACTAGAGACCCTGGACAAATACAACTGTGACTTCTGTGTTCACG
GCAATGACATCACCCTGACTGTAGATGGCCGGGACACCTATGAGGAAGTA
AAGCAGGCTGGGAGGTACAGAGAATGCAAGCGCACGCAAGGGGTGTCCAC
CACAGACCTCGTGGGCCGCATGCTGCTGGTAACCAAAGCCCATCACAGCA
GCCAGGAGATGTCCTCTGAGTACCGGGAGTATGCAGACAGTTTTGGCAAG
CCCCCTCACCCGATACCCGCCGGGGACATACTTTCCTCAGAAGGCTGCTC
CCAGTGCCCTGGTGGGCGGAACCCCTGGACCGGGGTATCCCAGTTCCTGC
AGACATCTCAGAAGATCATCCAGTTTGCTTCTGGGAAGGAGCCCCAGCCA
GGGGAGACAGTCATCTATGTGGCTGGTGCCTTCGACCTGTTCCACATCGG
GCATGTGGACTTCCTGGAGAAGGTGCACAGGCTGGCAGAGAGGCCCTACA
TCATCGCGGGCTTACACTTTGACCAGGAGGTCAATCACTACAAGGGGAAG
AACTACCCCATCATGAATCTGCATGAACGGACTCTGAGCGTGCTGGCCTG
CCGGTACGTGTCAGAAGTGGTGATTGGAGCCCCGTACGCGGTCACAGCAG
AGCTCCTAAGTCACTTCAAGGTGGACCTGGTGTGTCACGGCAAGACAGAA
ATTATCCCTGACAGGGATGGCTCCGACCCATACCAGGAGCCCAAGAGAAG
GGGCATCTTCCGTCAGATTGACAGTGGCAGCAACCTCACCACAGACCTCA
TCGTCCAGCGGATCATCACCAACAGGTTGGAGTATGAGGCGCGAAACCAG
AAGAAGGAAGCCAAGGAGCTGGCCTTCCTGGAGGCTGCCAGGCAGCAGGC
GGCACAGCCCCTGGGGGAGCGCGATGGTGACTTCCTCGAGGGC

| | | | |
|---|---|---|---|
| NOV10b, 283866181 | | | MW at |
| Protein Sequence | SEQ ID NO: 102 | 414 aa | 46267.7 kD |

TGSTMIRNGRGAAGGAEQPGPGGRRAVRVWCDGCYDMVHYGHSNQLRQAR
AMGDYLIVGVHTDEEIAKHKGPPVFTQEERYKMVQAIKWVDEVVPAAPYV
TTLETLDKYNCDFCVHGNDITLTVDGRDTYEEVKQAGRYRECKRTQGVST
TDLVGRMLLVTKAHHSSQEMSSEYREYADSFGKPPHPIPAGDILSSEGCS
QCPGGRNPWTGVSQFLQTSQKIIQFASGKEPQPGETVIYVAGAFDLFHIG
HVDFLEKVHRLAERPYIIAGLHFDQEVNHYKGKNYPIMNLHERTLSVLAC
RYVSEVVIGAPYAVTAELLSHFKVDLVCHGKTEIIPDRDGSDPYQEPKRR
GIFRQIDSGSNLTTDLIVQRIITNRLEYEARNQKKEAKELAFLEAARQQA
AQPLGERDGDFLEG

| | | |
|---|---|---|
| NOV10c, | SEQ ID NO: 103 | 1237 bp |
| CG57589-03 | ORF Start: | ORF Stop: |
| DNA Sequence | ATG at 14 | TAA at 1235 |

CACCGGATCCACCATGATCCGGAACGGGCGCGGGGCTGCAGGCGGCGCAG
AGCAGCCGGGCCCGGGGGGCAGGCGCGCCGTGAGGGTGTGGTGCGATGGC
TGCTATGACATGGTGCATTACGGCCACTCCAACCAGCTGCGCCAGGCACG
GGCCATGGGTGACTACCTCATCGTAGGCGTGCACACCGATGAGGAGATCG
CCAAGCACAAGGGGCCCCCGGTGTTCACTCAGGAGGAGAGATACAAGATG
GTGCAGGCCATCAAATGGGTGGACGAGGTGGTGCCAGCGGCTCCCTACGT
CACTACACTAGAGACCCTGGACAAATACAACTGTGACTTCTGTGTTCACG
GCAATGACATCACCCTGACTGTAGATGGCCGGGACACCTATGAGGAAGTA
AAGCAGGCTGGGAGGTACAGAGAATGCAAGCGCACGCAAGGGGTGTCCAC
CACAGACCTCGTGGGCCGCATGCTGCTGGTAACCAAAGCCCATCACAGCA
GCCAGGAGATGTCCTCTGAGTACCGGGAGTATGCAGACAGTTTTGGCAAG
CCCCCTCACCCGATACCCGCCGGGGACATACTTTCCTCAGAAGGCTGCTC
CCAGTGCCCTGGTGGGCGGAACCCCTGGACCGGGGTATCCCAGTTCCTGC
AGACATCTCAGAAGATCATCCAGTTTGCTTCTGGGAAGGAGCCCCAGCCA
GGGGAGACAGTCATCTATGTGGCTGGTGCCTTCGACCTGTTCCACATCGG
GCATGTGGACTTCCTGGAGAAGGTGCACAGGCTGGCAGAGAGGCCCTACA
TCATCGCGGGCTTACACTTTGACCAGGAGGTCAATCACTACAAGGGGAAG
AACTACCCCATCATGAATCTGCATGAACGGACTCTGAGCGTGCTGGCCTG
CCGGTACGTGTCAGAAGTGGTGATTGGAGCCCCGTACGCGGTCACAGCAG
AGCTCCTAAGTCACTTCAAGGTGGACCTGGTGTGTCACGGCAAGACAGAA
ATTATCCCTGACAGGGATGGCTCCGACCCATACCAGGAGCCCAAGAGAAG
GGGCATCTTCCGTCAGATTGACAGTGGCAGCAACCTCACCACAGACCTCA
TCGTCCAGCGGATCATCACCAACAGGTTGGAGTATGAGGCGCGAAACCAG
AAGAAGGAAGCCAAGGAGCTGGCCTTCCTGGAGGCTGCCAGGCAGCAGGC
GGCACAGCCCCTGGGGGAGCGCGATGGTGACTTCTAA

| | | | |
|---|---|---|---|
| NOV10c, CG57589-03 | | | MW at |
| Protein Sequence | SEQ ID NO: 104 | 407 aa | 45622.1 kD |

MIRNGRGAAGGAEQPGPGGRRAVRVWCDGCYDMVHYGHSNQLRQARAMGD
YLIVGVHTDEEIAKHKGPPVFTQEERYKMVQAIKWVDEVVPAAPYVTTLE
TLDKYNCDFCVHGNDITLTVDGRDTYEEVKQAGRYRECKRTQGVSTTDLV
GRMLLVTKAHHSSQEMSSEYREYADSFGKPPHPIPAGDILSSEGCSQCPG
GRNPWTGVSQFLQTSQKIIQFASGKEPQPGETVIYVAGAFDLFHIGHVDF
LEKVHRLAERPYIIAGLHFDQEVNHYKGKNYPIMNLHERTLSVLACRYVS
EVVIGAPYAVTAELLSHFKVDLVCHGKTEIIPDRDGSDPYQEPKRRGIFR
QIDSGSNLTTDLIVQRIITNRLEYEARNQKKEAKELAFLEAARQQAAQPL
GERDGDF

| | | |
|---|---|---|
| NOV10d, | SEQ ID NO: 105 | 1856 bp |
| CG57589-02 | ORF Start: | ORF Stop: |
| DNA Sequence | ATG at 67 | TAA at 1234 |

ATTGCGGGCGGCGGCGTTCGGAGTCGCCGGGAGCTGCCAGGCTGTCCGCG
CCGCCGCTGCGGGGCCATGATCCGGAACGGGCGCGGGGCTGCAGGCGGCG
CAGAGCAGCCGGGCCCGGGGGGCAGGCGCGCCGTGAGGGTGTGGTGCGAT
GGCTGCTATGACATGGTGCATTACGGCCACTCCAACCAGCTGCGCCAGGC
ACGGGCCATGGGTGACTACCTCATCGTAGGCGTGCACACCGATGAGGAGA
TCGCCAAGCACAAGGGGCCCCCGGTGTTCACTCAGGAGGAGAGATACAAG
ATGGTGCAGGCCATCAAATGGGTGGACGAGGTGGTGCCAGCGGCTCCCTA
CGTCACTACACTAGAGACCCTGGACAAATACAACTGTGACTTCTGTGTTC
ACGGCAATGACATCACCCTGACTGTAGATGGCCGGGACACCTATGAGGAA
GTAAAGCAGGCTGGGAGGTACAGAGAATGCAAGCGCACGCAAGGGGTGTC
CACCACAGACCTCGTGGGCCGCATGCTGCTGGTAACCAAAGCCCATCACA
GCAGCCAGGAGATGTCCTCTGAGTACCGGGAGTATGCAGACAGTTTTGGC
AAGTGCCCTGGTGGGCGGAACCCCTGGACCGGGGTATCCCAGTTCCTGCA
GACATCTCAGAAGATCATCCAGTTTGCTTCTGGGAAGGAGCCCCAGCCAG
GGGAGACAGTCATCTATGTGGCTGGTGCCTTCGACCTGTTCCACATCGGG
CATGTGGACTTCCTGGAGAAGGTGCACAGGCTGGCAGAGAGGCCCTACAT
CATCGCGGGCTTACACTTTGACCAGGAGGTCAATCACTACAAGGGGAAGA
ACTACCCCATCATGAATCTGCATGAACGGACTCTGAGCGTGCTGGCCTGC
CGGTACGTGTCAGAAGTGGTGATTGGAGCCCCGTACGCGGTCACAGCAGA
GCTCCTAAGTCACTTCAAGGTGGACCTGGTGTGTCACGGCAAGACAGAAA
TTATCCCTGACAGGGATGGCTCCGACCCATACCAGGAGCCCAAGAGAAGG
GGCATCTTCCGTCAGATTGACAGTGGCAGCAACCTCACCACAGACCTCAT
CGTCCAGCGGATCATCACCAACAGGTTGGAGTATGAGGCGCGAAACCAGA
AGAAGGAAGCCAAGGAGCTGGCCTTCCTGGAGGCTGCCAGGCAGCAGGCG
GCACAGCCCCTGGGGGAGCGCGATGGTGACTTCTAACCTGGCAGAGGCCC
TGGCCGGCCCTCCCCCTGCTCTGCTTCTGCGCCTTCTGCGTTTGGACATA
GGACTCTGCAGGGCCGCCCTCTCTAACTGGCCTGGCTCTGGAAGGGCTGG
TGAGGACTCTGCCTCCTTGCCTGCCTACAAGGTGCCTGGTTTGCAGCAGG
CTCTCCGCTCTTTCCAGCAAAGCTGCTCAGAGAGGGTGTCCAGCACAGTG
GAGAGGCCGGAAGTGAGACGGGCAGACGGCACCTGCAGCCTGAAACGCAC
CGCTCCTGCGTGCGCCCCCACCTGGTCCCCGGATGCCCCCACCACCTGGA
CAGAGGCCACACTGACTGCCCACCCAGCTGTGGCGGGAGGTGCAGAGCAG
GGGGCTTTAGGGAGCAGTGACTGCGGTCACCCCTTTAGTTCTCTGGGTGT
AGACCACACCACCTCCCACTGGGCACCCCCCAACACGGTGTCCTGCCACC
CAGCGCCTGGCTCCAGGAAAACACGCTTGCCTTCCTTCCCGGCAGCTTCG
CCACTCTCCTTATGGACTCTGTTCTGTTTGTACATGGCTGACGGAAATCT
CTTTGGTACAACCGAATAAAGCCTGGTGGCAGTGCTGCGCGGGGCTCCCA
GCCAAT

| | | | |
|---|---|---|---|
| NOV10d, CG57589-02 | | | MW at |
| Protein Sequence | SEQ ID NO: 106 | 389 aa | 43835.1 kD |

MIRNGRGAAGGAEQPGPGGRRAVRVWCDGCYDMVHYGHSNQLRQARAMGD
YLIVGVHTDEEIAKHKGPPVFTQEERYKMVQAIKWVDEVVPAAPYVTTLE
TLDKYNCDFCVHGNDITLTVDGRDTYEEVKQAGRYRECKRTQGVSTTDLV
GRMLLVTKAHHSSQENSSEYREYADSFGKCPGGRNPWTGVSQFLQTSQKI
IQFASGKEPQPGETVIYVAGAFDLFHIGHVDFLEKVHRLAERPYIIAGLH
FDQEVNHYKGKNYPIMNLHERTLSVLACRYVSEVVIGAPYAVTAELLSHF
KVDLVCHGKTEIIPDRDGSDPYQEPKRRGIFRQIDSGSNLTTDLIVQRII
TNRLEYEARNQKKEAKELAFLEAARQQAAQPLGERDGDF

A ClustalW comparison of the above protein sequences yields the following sequence alignment shown in Table 10B.

TABLE 10B!Comparison of the NOV10 protein sequences.

| | |
|---|---|
| NOV10a | ----MIRNGRGAAGGAEQPGPGGRRAVRVWCDGCYDMVHYGHSNQLRQARANGDYLIVGV |
| NOV10b | TGSTMIRNGRGAAGGAEQPGPGGRRAVRVWCDGCYDMVHYGHSNQLRQARANGDYLIVGV |
| NOV10c | ----MIRNGRGAAGGAEQPGPGGRRAVRVWCDGCYDMVHYGHSNQLRQARANGDYLIVGV |
| NOV10d | ----MIRNGRGAAGGAEQPGPGGRRAVRVWCDGCYDMVHYGHSNQLRQARANGDYLIVGV |
| NOV10a | HTDEEIAKHKGPPVFTQEERYKMVQAIKWVDEVVPAAPYVTTLETLDKYNCDFCVHGNDI |
| NOV10b | HTDEEIAKHKGPPVFTQEERYKMVQAIKWVDEVVPAAPYVTTLETLDKYNCDFCVHGNDI |
| NOV10c | HTDEEIAKHKGPPVFTQEERYKMVQAIKWVDEVVPAAPYVTTLETLDKYNCDFCVHGNDI |
| NOV10d | HTDEEIAKHKGPPVFTQEERYKMVQAIKWVDEVVPAAPYVTTLETLDKYNCDFCVHGNDI |
| NOV10a | TLTVDGRDTYEEVKQAGRYRECKRTQGVSTTDLVGRMLLVTKAHHSSQEMSSEYREYADS |
| NOV10b | TLTVDGRDTYEEVKQAGRYRECKRTQGVSTTDLVGRMLLVTKAHHSSQEMSSEYREYADS |
| NOV10c | TLTVDGRDTYEEVKQAGRYRECKRTQGVSTTDLVGRMLLVTKAHHSSQEMSSEYREYADS |
| NOV10d | TLTVDGRDTYEEVKQAGRYRECKRTQGVSTTDLVGRMLLVTKAHHSSQEMSSEYREYADS |
| NOV10a | FG------------------KCPGGRNPWTGVSQFLQTSQKIIQFASGKEPQPGETVIYV |
| NOV10b | FGKPPHPIPAGDILSSEGCSQCPGGRNPWTGVSQFLQTSQKIIQFASGKEPQPGETVIYV |
| NOV10c | FGKPPHPIPAGDILSSEGCSQCPGGRNPWTGVSQFLQTSQKIIQFASGKEPQPGETVIYV |
| NOV10d | FG------------------KCPGGRNPWTGVSQFLQTSQKIIQFASGKEPQPGETVIYV |
| NOV10a | AGAFDLFHIGHVDFLEKVHRLAERPYIIAGLHFD--------Q----------------- |
| NOV10b | AGAFDLFHIGHVDFLEKVHRLAERPYIIAGLHFDQEVNHYKGKNYPIMNLHERTLSVLAC |
| NOV10c | AGAFDLFHIGHVDFLEKVHRLAERPYIIAGLHFDQEVNHYKGKNYPIMNLHERTLSVLAC |
| NOV10d | AGAFDLFHIGHVDFLEKVHRLAERPYIIAGLHFDQEVNHYKGKNYPIMNLHERTLSVLAC |
| NOV10a | -YVSEVVIGAPYAVTAELLSHFKVDLVCHGKTGIIPDRDGSDPYQEPKRRGIFRQIDSGS |
| NOV10b | RYVSEVVIGAPYAVTAELLSHFKVDLVCHGKTEIIPDRDGSDPYQEPKRRGIFRQIDSGS |
| NOV10c | RYVSEVVIGAPYAVTAELLSHFKVDLVCHGKTEIIPDRDGSDPYQEPKRRGIFRQIDSGS |
| NOV10d | RYVSEVVIGAPYAVTAELLSHFKVDLVCHGKTEIIPDRDGSDPYQEPKRRGIFRQIDSGS |
| NOV10a | NLTTDLIVQRIITNRLEYEARNQKKEAKELAFLEAARQQAAQPLGERDGDF--- |
| NOV10b | NLTTDLIVQRIITNRLEYEARNQKKEAKELAFLEAARQQAAQPLGERDGDFLEG |
| NOV10c | NLTTDLIVQRIITNRLEYEARNQKKEAKELAFLEAARQQAAQPLGERDGDF--- |
| NOV10d | NLTTDLIVQRIITNRLEYEARNQKKEAKELAFLEAARQQAAQPLGERDGDF--- |
| NOV10a | (SEQ ID NO: 100) |
| NOV10b | (SEQ ID NO: 102) |
| NOV10c | (SEQ ID NO: 104) |
| NOV10d | (SEQ ID NO: 106) |

Further analysis of the NOV 10a protein yielded the following properties shown in Table 10C.

TABLE 10C
Protein Sequence Properties NOV10a

| SignalP | No Known Signal Sequence Predicted | | | |
|---|---|---|---|---|
| analysis: | | | | |
| PSORT II | PSG: a new signal peptide prediction method | | | |
| analysis: | N-region: length 6; pos. chg 2; neg. chg 0 | | | |
| | H-region: length 6; peak value −2.35 | | | |
| | PSG score: −6.75 | | | |
| | GvH: von Heijne's method for signal seg. recognition | | | |
| | GvH score (threshold: −2.1): −10.63 | | | |
| | possible cleavage site: between 58 and 59 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 2.33 (at 255) | |
| | ALOM score: 2.33 (number of TMSs: 0) | | | |
| | MITDISC: discrimination of mitochondrial targeting seq | | | |
| | R content: | 5 | Hyd Moment(75): | 3.05 |
| | Hyd Moment(95): | 10.66 | G content: | 7 |
| | D/E content: | 2 | S/T content: | 0 |

```
Score: −5.71
Gavel: prediction of cleavage sites for mitochondrial preseq
R-2 motif at 34 VRV|WC
NUCDISC: discrimination of nuclear localization signals
pat4: PKRR (4) at 299
pat7: PKRRGIF (5) at 299
bipartite: none
content of basic residues: 12.1%
NLS Score: 0.21
KDEL: ER retention motif in the C-terminus: none
ER Membrane Retention Signals:
XXRR-like motif in the N-terminus: IRNG none
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: cytoplasmic
Reliability: 94.1
COIL: Lupas's algorithm to detect coiled-coil regions
total: 0 residues
Final Results (k = 9/23):
73.9%: cytoplasmic
  8.7%: nuclear
  4.3%: vacuolar
  4.3%: mitochondrial
  4.3%: plasma membrane
  4.3%: peroxisomal
>> prediction for CG57589-01 is cyt (k = 23)
```

A search of the NOV10a protein against the Geneseq database, a proprietary database that contains sequences published in patents and patent publication, yielded several homologous proteins shown in Table 10D.

TABLE 10D
Geneseq Results for NOV10a

| | | NOV10a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABU52623 | Human NOVX protein, NOV29 - | 1 . . . 363 | 363/363 (100%) | 0.0 |
| | Homo sapiens, 363 aa. | 1 . . . 363 | 363/363 (100%) | |
| | [WO200281518-A2, 17 OCT. 2002] | | | |
| ABB61630 | Drosophila melanogaster polypeptide | 33 . . . 351 | 200/360 (55%) | e−110 |
| | SEQ ID NO 11682 - Drosophila | 1 . . .356 | 250/360 (68%) | |
| | melanogaster, 362 aa. | | | |
| | [WO200171042-A2, 27 SEP. 2001] | | | |
| AAG09521 | Arabidopsis thaliana protein fragment | 20 . . . 346 | 153/364 (42%) | 3e−77 |
| | SEQ ID NO: 7488 - Arabidopsis | 52 . . . 413 | 215/364 (59%) | |
| | thaliana, 421 aa. [EP1033405-A2, | | | |
| | 06 SEP. 2000] | | | |
| AAG09522 | Arabidopsis thaliana protein fragment | 28 . . . 346 | 149/356 (41%) | 7e−75 |
| | SEQ ID NO: 7489 - Arabidopsis | 2 . . . 355 | 209/356 (57%) | |
| | thaliana, 363 aa. [EP1033405-A2, | | | |
| | 06 SEP. 2000] | | | |
| AAG38354 | Arabidopsis thaliana protein fragment | 20 . . . 333 | 146/351 (41%) | 1e−73 |
| | SEQ ID NO: 47306 - Arabidopsis | 52 . . . 400 | 208/351 (58%) | |
| | thaliana, 401 aa. [EP1033405-A2, | | | |
| | 06 SEP. 2000] | | | |

In a BLAST search of public sequence databases, the NOV10a protein was found to have homology to the proteins shown in the BLASTP data in Table 10E.

TABLE 10E
Public BLASTP Results for NOV10a

| | | NOV10a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q99447 | Ethanolamine-phosphate | 1 . . . 363 | 362/389 (93%) | 0.0 |
| | cytidylyltransferase (EC 2.7.7.14) | 1 . . . 389 | 362/389 (93%) | |
| | (Phosphorylethanolamine transferase) | | | |
| | (CTP: phosphoethanolamine | | | |
| | cytidylyltransferase) - Homo sapiens | | | |
| | (Human), 389 aa. | | | |
| O88637 | Ethanolamine-phosphate | 1 . . . 360 | 332/404 (82%) | 0.0 |
| | cytidylyltransferase (EC 2.7.7.14) | 1 . . . 404 | 346/404 (85%) | |
| | (Phosphorylethanolamine transferase) | | | |
| | (CTP: phosphoethanolamine | | | |
| | cytidylyltransferase) - Rattus norvegicus | | | |
| | (Rat), 404 aa. | | | |
| Q922E4 | Ethanolamine-phosphate | 1 . . . 360 | 330/404 (81%) | 0.0 |
| | cytidylyltransferase (EC 2.7.7.14) | 1 . . . 404 | 344/404 (84%) | |
| | (Phosphorylethanolamine transferase) | | | |
| | (CTP: phosphoethanolamine | | | |
| | cytidylyltransferase) - Mus musculus | | | |
| | (Mouse), 404 aa. | | | |
| Q86PD6 | RE62261p - Drosophila melanogaster | 20 . . . 351 | 212/361 (58%) | e−120 |
| | (Fruit fly), 381 aa. | 18 . . . 374 | 264/361 (72%) | |
| EAA14927 | ENSANGP00000012337 - Anopheles | 3 . . . 350 | 212/375 (56%) | e−116 |
| | gambiae str. PEST, 480 aa (fragment). | 107 . . . 472 | 262/375 (69%) | |

PFam analysis predicts that the NOV10a protein contains the domains shown in the Table 10F.

TABLE 10F
Domain Analysis of NOV10a

| | | Identities/ | |
|---|---|---|---|
| | | Similarities | |
| | NOV10a | for the | Expect |
| Pfam Domain | Match Region | Matched Region | Value |
| CTP_transf_2 | 26 . . . 218 | 51/228 (22%) | 2.2e−34 |
| | | 150/228 (66%) | |

### Example 11

The NOV11 clone was analyzed, and the nucleotide and encoded polypeptide sequences are shown in Table 11A.

TABLE 11A
NOV11 Sequence Analysis

| | | |
|---|---|---|
| NOV11a, | SEQ ID NO: 107 | 1908 bp |
| CG91149-03 | ORF Start: | ORF Stop: |
| DNA Sequence | ATG at 24 | TAA at 1719 |

GCAGACTCAGTTCCTGGAGAAAGATGGCGACAGCCGAGAAGCAGAAACAC
GACGGGCGGGTGAAGATCGGCCACTACATTCTGGGTGACACGCTGGGGGT
CGGCACCTTCGGCAAAGTGAAGGTTGGCAAACATGAATTGACTGGGCATA
AAGTAGCTGTGAAGATACTCAATCGACAGAAGATTCGGAGCCTTGATGTG
GTAGGAAAAATCCGCAGAGAAATTCAGAACCTCAAGCTTTTCAGGCATCC
TCATATAATTAAACTGTACCAGGTCATCAGTACACCATCTGATATTTTCA
TGGTGATGGAATATGTCTCAGGAGGAGAGCTATTTGATTATATCTGTAAG
AATGGAAGGAAATCTGATGTACCTGGAGTAGTAAAAACAGGCTCCACGAA
GGAGCTGGATGAAAAAGAAAGTCGGCGTCTGTTCCAACAGATCCTTTCTG
GTGTGGATTATTGTCACAGGCATATGGTGGTCCATAGAGATTTGAAACCT
GAAAATGTCCTGCTTGATGCACACATGAATGCAAAGATAGCTGATTTTGG
TCTTTCAAACATGATGTCAGATGGTGAATTTTTAAGAACAAGTTGTGGCT
CACCCAACTATGCTGCACCAGAAGTAATTTCAGGAAGATTGTATGCAGGC
CCAGAGGTAGATATATGGAGCAGTGGGGTTATTCTCTATGCTTTATTATG
TGGAACCCTTCCATTTGATGATGACCATGTGCCAACTCTTTTTAAGAAGA
TATGTGATGGGATCTTCTATACCCCTCAATATTTAAATCCTTCTGTGATT
AGCCTTTTGAAACATATGCTGCAGGTGGATCCCATGAAGAGGGCCTCAAT
CAAAGATATCAGGGAACATGAATGGTTTAAACAGGACCTTCCAAAATATC
TCTTTCCTGAGGATCCATCATATAGTTCAACCATGATTGATGATGAAGCC
TTAAAAGAAGTATGTGAAAAGTTTGAGTGCTCAGAAGAGGAAGTTCTCAG
CTGTCTTTACAACAGAAATCACCAGGATCCTTTGGCAGTTGCCTACCATC
TCATAATAGATAACAGGAGAATAATGAATGAAGCCAAAGATTTCTATTTG
GCGACAAGCCCACCTGATTCTTTTCTTGATGATCATCACCTGACTCGGCC
CCATCCTGAAAGAGTACCATTCTTGGTTGCTGAAACACCAAGGGCACGCC
ATACCCTTGATGAATTAAATCCACAGAAATCCAAACACCAAGGTGTAAGG
AAAGCAAAATGGCATTTAGGAATTAGAAGTCAAAGTCGACCAAATGATAT
TATGGCAGAAGTATGTAGAGCAATCAAACAATTGGATTATGAATGGAAGG
TTGTAAACCCATATTATTTGCGTGTACGAAGGAAGAATCCTGTGACAAGC
ACTTACTCCAAAATGAGTCTACAGTTATACCAAGTGGATAGTAGAACTTA
TCTACTGGATTTCCGTAGTATTGATGATGAAATTACAGAAGCCAAATCAG
GGACTGCTACTCCACAGAGATCGGGATCAGTTAGCAACTATCGATCTTGC
CAAAGGAGTGATTCAGATGCTGAGGCTCAAGGAAAATCCTCAGAAGTTTC
TCTTACCTCATCTGTGACCTCACTTGACTCTTCTCCTGTTGACCTAACTC
CAAGACCTGGAAGTCACACAATAGAATTTTTTGAGATGTGTGCAAATCTA
ATTAAAATTCTTGCACAATAAACAGAAAACTTTGCTTATTTCTTTTGCAG
CAATAAGCATGCATAATAAGTCACAGCCAAATGCTTCCATTTGTAATCAA
GTTATACATAATTATAACCGAGGGCTGGCGTTTTGGAATCGAATTTCGAC
AGGGATTGGAACATGATTTATAGTTAAAAGCCTAATATCGAGAAATGAAT
TAAGATCA

| | | | |
|---|---|---|---|
| NOV11a, CG91149-03 | | | MW at |
| Protein Sequence | SEQ ID NO: 108 | 565 aa | 64306.7 kD |

MATAEKQKHDGRVKIGHYILGDTLGVGTFGKVKVGKHELTGHKVAVKILN
RQKIRSLDVVGKIRREIQNLKLFRHPHIIKLYQVISTPSDIFMVMEYVSG
GELFDYICKNGRKSDVPGVVKTGSTKELDEKESRRLFQQILSGVDYCHRH
MVVHRDLKPENVLLDAHMNAKIADFGLSNMMSDGEFLRTSCGSPNYAAPE
VISGRLYAGPEVDIWSSGVILYALLCGTLPFDDDHVPTLFKKICDGIFYT
PQYLNPSVISLLKHMLQVDPMKRASIKDIREHEWFKQDLPKYLFPEDPSY
SSTMIDDEALKEVCEKFECSEEEVLSCLYNRNHQDPLAVAYHLIIDNRRI
MNEAKDFYLATSPPDSFLDDHHLTRPHPERVPFLVAETPRARHTLDELNP
QKSKHQGVRKAKWHLGIRSQSRPNDIMAEVCRAIKQLDYEWKVVNPYYLR
VRRKNPVTSTYSKMSLQLYQVDSRTYLLDFRSIDDEITEAKSGTATPQRS
GSVSNYRSCQRSDSDAEAQGKSSEVSLTSSVTSLDSSPVDLTPRPGSHTI
EFFEMCANLIKILAQ

| | | |
|---|---|---|
| NOV11b, | SEQ ID NO: 109 | 1863 bp |
| CG91149-01 | ORF Start: | ORF Stop: |
| DNA Sequence | ATG at 24 | TAA at 1674 |

GCAGACTCAGTTCCTGGAGAAAGATGGCGACAGCCGAGAAGCAGAAACAC
GACGGGCGGGTGAAGATCGGCCACTACATTCTGGGTGACACGCTGGGGGT
CGGCACCTTCGGCAAAGTGAAGGTTGGCAAACATGAATTGACTGGGCATA
AAGTAGCTGTGAAGATACTCAATCGACAGAAGATTCGGAGCCTTGATGTG
GTAGGAAAAATCCGCAGAGAAATTCAGAACCTCAAGCTTTTCAGGCATCC
TCATATAATTAAACTGTACCAGGTCATCAGTACACCATCTGATATTTTCA
TGGTGATGGAATATGTCTCAGGAGGAGAGCTATTTGATTATATCTGTAAG
AATGGAAGGCTGGATGAAAAAGAAAGTCGGCGTCTGTTCCAACAGATCCT
TTCTGGTGTGGATTATTGTCACAGGCATATGGTGGTCCATAGAGATTTGA
AACCTGAAAATGTCCTGCTTGATGCACACATGAATGCAAAGATAGCTGAT
TTTGGTCTTTCAAACATGATGTCAGATGGTGAATTTTTAAGAACAAGTTG
TGGCTCACCCAACTATGCTGCACCAGAAGTAATTTCAGGAAGATTGTATG
CAGGCCCAGAGGTAGATATATGGAGCAGTGGGGTTATTCTCTATGCTTTA
TTATGTGGAACCCTTCCATTTGATGATGACCATGTGCCAACTCTTTTTAA
GAAGATATGTGATGGGATCTTCTATACCCCTCAATATTTAAATCCTTCTG
TGATTAGCCTTTTGAAACATATGCTGCAGGTGGATCCCATGAAGAGGGCC
TCAATCAAAGATATCAGGGAACATGAATGGTTTAAACAGGACCTTCCAAA
ATATCTCTTTCCTGAGGATCCATCATATAGTTCAACCATGATTGATGATG
AAGCCTTAAAAGAAGTATGTGAAAAGTTTGAGTGCTCAGAAGAGGAAGTT
CTCAGCTGTCTTTACAACAGAAATCACCAGGATCCTTTGGCAGTTGCCTA
CCATCTCATAATAGATAACAGGAGAATAATGAATGAAGCCAAAGATTTCT
ATTTGGCGACAAGCCCACCTGATTCTTTTCTTGATGATCATCACCTGACT
CGGCCCCATCCTGAAAGAGTACCATTCTTGGTTGCTGAAACACCAAGGGC
ACGCCATACCCTTGATGAATTAAATCCACAGAAATCCAAACACCAAGGTG
TAAGGAAAGCAAAATGGCATTTAGGAATTAGAAGTCAAAGTCGACCAAAT
GATATTATGGCAGAAGTATGTAGAGCAATCAAACAATTGGATTATGAATG
GAAGGTTGTAAACCCATATTATTTGCGTGTACGAAGGAAGAATCCTGTGA
CAAGCACTTACTCCAAAATGAGTCTACAGTTATACCAAGTGGATAGTAGA
ACTTATCTACTGGATTTCCGTAGTATTGATGATGAAATTACAGAAGCCAA
ATCAGGGACTGCTACTCCACAGAGATCGGGATCAGTTAGCAACTATCGAT
CTTGCCAAAGGAGTGATTCAGATGCTGAGGCTCAAGGAAAATCCTCAGAA
GTTTCTCTTACCTCATCTGTGACCTCACTTGACTCTTCTCCTGTTGACCT
AACTCCAAGACCTGGAAGTCACACAATAGAATTTTTTGAGATGTGTGCAA
ATCTAATTAAAATTCTTGCACAATAAACAGAAAACTTTGCTTATTTCTTT
TGCAGCAATAAGCATGCATAATAAGTCACAGCCAAATGCTTCCATTTGTA
ATCAAGTTATACATAATTATAACCGAGGGCTGGCGTTTTGGAATCGAATT
TCGACAGGGATTGGAACATGATTTATAGTTAAAAGCCTAATATCGAGAAA
TGAATTAAGATCA

| | | | |
|---|---|---|---|
| NOV11b, CG91149-01 | | | MW at |
| Protein Sequence | SEQ ID NO: 110 | 550 aa | 62793.0 kD |

MATAEKQKHDGRVKIGHYILGDTLGVGTFGKVKVGKHELTGHKVAVKILN
RQKIRSLDVVGKIRREIQNLKLFRHPHIIKLYQVISTPSDIFMVMEYVSG
GELFDYICKNGRLDEKESRRLFQQILSGVDYCHRHMVVHRDLKPENVLLD
AHMNAKIADFGLSNMMSDGEFLRTSCGSPNYAAPEVISGRLYAGPEVDIW
SSGVILYALLCGTLPFDDDHVPTLFKKICDGIFYTPQYLNPSVISLLKHM
LQVDPMKRASIKDIREHEWFKQDLPKYLFPEDPSYSSTMIDDEALKEVCE
KFECSEEEVLSCLYNRNHQDPLAVAYHLIIDNRRIMNEAKDFYLATSPPD
SFLDDHHLTRPHPERVPFLVAETPRARHTLDELNPQKSKHQGVRKAKWHL
GIRSQSRPNDIMAEVCRAIKQLDYEWKVVNPYYLRVRRKNPVTSTYSKMS
LQLYQVDSRTYLLDFRSIDDEITEAKSGTATPQRSGSVSNYRSCQRSDSD
AEAQGKSSEVSLTSSVTSLDSSPVDLTPRPGSHTIEFFEMCANLIKILAQ

| | | |
|---|---|---|
| NOV11c, | SEQ ID NO: 111 | 1863 bp |
| CG91149-02 | ORF Start: | ORF Stop: |
| DNA Sequence | ATG at 24 | TAA at 1674 |

GCAGACTCAGTTCCTGGAGAAAGATGGCGACAGCCGAGAAGCAGAAACAC
GACGGGCGGGTGAAGATCGGCCACTACATTCTGGGTGACACGCTGGGGGT
CGGCACCTTCGGCAAAGTGAAGGTTGGCAAACATGAATTGACTGGGCATA
AAGTAGCTGTGAAGATACTCAATCGACAGAAGATTCGGAGCCTTGATGTG
GTAGGAAAAATCCGCAGAGAAATTCAGAACCTCAAGCTTTTCAGGCATCC
TCATATAATTAAACTGTACCAGGTCATCAGTACACCATCTGATATTTTCA
TGGTGATGGAATATGTCTCAGGAGGAGAGCTATTTGATTATATCTGTAAG
AATGGAAGGCTGGATGAAAAAGAAAGTCGGCGTCTGTTCCAACAGATCCT
TTCTGGTGTGGATTATTGTCACAGGCATATGGTGGTCCATAGAGATTTGA
AACCTGAAAATGTCCTGCTTGATGCACACATGAATGCAAAGATAGCTGAT
TTTGGTCTTTCAAACATGATGTCAGATGGTGAATTTTTAAGAACAAGTTG
TGGCTCACCCAACTATGCTGCACCAGAAGTAATTTCAGGAAGATTGTATG
CAGGCCCAGAGGTAGATATATGGAGCAGTGGGGTTATTCTCTATGCTTTA
TTATGTGGAACCCTTCCATTTGATGATGACCATGTGCCAACTCTTTTTAA
GAAGATATGTGATGGGATCTTCTATACCCCTCAATATTTAAATCCTTCTG
TGATTAGCCTTTTGAAACATATGCTGCAGGTGGATCCCATGAAGAGGGCC
TCAATCAAAGATATCAGGGAACATGAATGGTTTAAACAGGACCTTCCAAA
ATATCTCTTTCCTGAGGATCCATCATATAGTTCAACCATGATTGATGATG
AAGCCTTAAAAGAAGTATGTGAAAAGTTTGAGTGCTCAGAAGAGGAAGTT
CTCAGCTGTCTTTACAACAGAAATCACCAGGATCCTTTGGCAGTTGCCTA
CCATCTCATAATAGATAACAGGAGAATAATGAATGAAGCCAAAGATTTCT
ATTTGGCGACAAGCCCACCTGATTCTTTTCTTGATGATCATCACCTGACT
CGGCCCCATCCTGAAAGAGTACCATTCTTGGTTGCTGAAACACCAAGGGC
ACGCCATACCCTTGATGAATTAAATCCACAGAAATCCAAACACCAAGGTG
TAAGGAAAGCAAAATGGCATTTAGGAATTAGAAGTCAAAGTCGACCAAAT
GATATTATGGCAGAAGTATGTAGAGCAATCAAACAATTGGATTATGAATG
GAAGGTTGTAAACCCATATTATTTGCGTGTACGAAGGAAGAATCCTGTGA
CAAGCACTTACTCCAAAATGAGTCTACAGTTATACCAAGTGGATAGTAGA
ACTTATCTACTGGATTTCCGTAGTATTGATGATGAAATTACAGAAGCCAA
ATCAGGGACTGCTACTCCACAGAGATCGGGATCAGTTAGCAACTATCGAT
CTTGCCAAAGGAGTGATTCAGATGCTGAGGCTCAAGGAAAATCCTCAGAA
GTTTCTCTTACCTCATCTGTGACCTCACTTGACTCTTCTCCTGTTGACCT
AACTCCAAGACCTGGAAGTCACACAATAGAATTTTTTGAGATGTGTGCAA
ATCTAATTAAAATTCTTGCACAATAAACAGAAAACTTTGCTTATTTCTTT
TGCAGCAATAAGCATGCATAATAAGTCACAGCCAAATGCTTCCATTTGTA
ATCAAGTTATACATAATTATAACCGAGGGCTGGCGTTTTGGAATCGAATT
TCGACAGGGATTGGAACATGATTTATAGTTAAAAGCCTAATATCGAGAAA
TGAATTAAGATCA

| | | | |
|---|---|---|---|
| NOV11c, CG91149-02 | | | MW at |
| Protein Sequence | SEQ ID NO: 112 | 550 aa | 62793.0 kD |

MATAEKQKHDGRVKIGHYILGDTLGVGTFGKVKVGKHELTGHKVAVKILN
RQKIRSLDVVGKIRREIQNLKLFRHPHIIKLYQVISTPSDIFMVMEYVSG
GELFDYICKNGRLDEKESRRLFQQILSGVDYCHRHMVVHRDLKPENVLLD
AHMNAKIADFGLSNNMSDGEFLRTSCGSPNYAAPEVISGRLYAGPEVDIW
SSGVILYALLCGTLPFDDDHVPTLFKKICDGIFYTPQYLNPSVISLLKHM
LQVDPMKRASIKDIREHEWFKQDLPKYLFPEDPSYSSTMIDDEALKEVCE
KFECSEEEVLSCLYNRNHQDPLAVAYHLIIDNRRIMNEAKDFYLATSPPD
SFLDDHHLTRPHPERVPFLVAETPRARHTLDELNPQKSKHQGVRKAKWHL
GIRSQSRPNDIMAEVCRAIKQLDYEWKVVNPYYLRVRRKNPVTSTYSKMS
LQLYQVDSRTYLLDFRSIDDEITEAKSGTATPQRSGSVSNYRSCQRSDSD
AEAQGKSSEVSLTSSVTSLDSSPVDLTPRPGSHTIEFFEMCANLIKILAQ

A ClustalW comparison of the above protein sequences yields the following sequence alignment shown in Table 11B.

TABLE 11B
Comparison of the NOV11 protein sequences.

| | |
|---|---|
| NOV11a | MATAEKQKHDGRVKIGHYILGDTLGVGTFGKVKVGKHELTGHKVAVKILNRQKIRSLDVV |
| NOV11b | MATAEKQKHDGRVKIGHYILGDTLGVGTFGKVKVGKHELTGHKVAVKILNRQKIRSLDVV |
| NOV11c | MATAEKQKHDGRVKIGHYILGDTLGVGTFGKVKVGKHELTGHKVAVKILNRQKIRSLDVV |
| NOV11a | GKIRREIQNLKLFRHPHIIKLYQVISTPSDIFMVMEYVSGGELFDYICKNGRKSDVPGVV |
| NOV11b | GKIRREIQNLKLFRHPHIIKLYQVISTPSDIFMVMEYVSGGELFDYICKNGR-------- |
| NOV11c | GKIRREIQNLKLFRHPHIIKLYQVISTPSDIFMVMEYVSGGELFDYICKNGR-------- |
| NOV11a | KTGSTKELDEKESRRLFQQILSGVDYCHRHMVVHRDLKPENVLLDAHMNAKIADFGLSNM |
| NOV11b | -------LDEKESRRLFQQILSGVDYCHRHMVVHRDLKPENVLLDAHMNAKIADFGLSNM |
| NOV11c | -------LDEKESRRLFQQILSGVDYCHRHMVVHRDLKPENVLLDAHMNAKIADFGLSNM |
| NOV11a | MSDGEFLRTSCGSPNYAAPEVISGRLYAGPEVDIWSSGVILYALLCGTLPFDDDHVPTLF |
| NOV11b | MSDGEFLRTSCGSPNYAAPEVISGRLYAGPEVDIWSSGVILYALLCGTLPFDDDHVPTLF |
| NOV11c | MSDGEFLRTSCGSPNYAAPEVISGRLYAGPEVDIWSSGVILYALLCGTLPFDDDHVPTLF |
| NOV11a | KKICDGIFYTPQYLNPSVISLLKHMLQVDPMKRASIKDIREHEWFKQDLPKYLFPEDPSY |
| NOV11b | KKICDGIFYTPQYLNPSVISLLKHMLQVDPMKRASIKDIREHEWFKQDLPKYLFPEDPSY |
| NOV11c | KKICDGIFYTPQYLNPSVISLLKHMLQVDPMKRASIKDIREHEWFKQDLPKYLFPEDPSY |
| NOV11a | SSTMIDDEALKEVCEKFECSEEEVLSCLYNRNHQDPLAVAYHLIIDNRRIMNEAKDFYLA |
| NOV11b | SSTMIDDEALKEVCEKFECSEEEVLSCLYNRNHQDPLAVAYHLIIDNRRIMNEAKDFYLA |
| NOV11c | SSTMIDDEALKEVCEKFECSEEEVLSCLYNRNHQDPLAVAYHLIIDNRRIMNEAKDFYLA |
| NOV11a | TSPPDSFLDDHHLTRPHPERVPFLVAETPRARHTLDELNPQKSKHQGVRKAKWHLGIRSQ |
| NOV11b | TSPPDSFLDDHHLTRPHPERVPFLVAETPRARHTLDELNPQKSKHQGVRKAKWHLGIRSQ |
| NOV11c | TSPPDSFLDDHHLTRPHPERVPFLVAETPRARHTLDELNPQKSKHQGVRKAKWHLGIRSQ |
| NOV11a | SRPNDIMAEVCRAIKQLDYEWKVVNPYYLRVRRKNPVTSTYSKMSLQLYQVDSRTYLLDF |
| NOV11b | SRPNDIMAEVCRAIKQLDYEWKVVNPYYLRVRRKNPVTSTYSKMSLQLYQVDSRTYLLDF |
| NOV11c | SRPNDIMAEVCRAIKQLDYEWKVVNPYYLRVRRKNPVTSTYSKMSLQLYQVDSRTYLLDF |
| NOV11a | RSIDDEITEAKSGTATPQRSGSVSNYRSCQRSDSDAEAQGKSSEVSLTSSVTSLDSSPVD |
| NOV11b | RSIDDEITEAKSGTATPQRSGSVSNYRSCQRSDSDAEAQGKSSEVSLTSSVTSLDSSPVD |
| NOV11c | RSIDDEITEAKSGTATPQRSGSVSNYRSCQRSDSDAEAQGKSSEVSLTSSVTSLDSSPVD |
| NOV11a | LTPRPGSHTIEFFEMCANLIKILAQ |
| NOV11b | LTPRPGSHTIEFFEMCANLIKILAQ |
| NOV11c | LTPRPGSHTIEFFEMCANLIKILAQ |
| NOV11a | (SEQ ID NO: 108) |
| NOV11b | (SEQ ID NO: 110) |
| NOV11c | (SEQ ID NO: 112) |

Further analysis of the NOV11a protein yielded the following properties shown in Table 11C.

TABLE 11C
Protein Sequence Properties NOV11a

| SignalP | No Known Signal Sequence Predicted | | | |
|---|---|---|---|---|
| analysis: | | | | |
| PSORT II | PSG: a new signal peptide prediction method | | | |
| analysis: | N-region: length 10; pos. chg 2; neg. chg 2 | | | |
| | H-region: length 1; peak value −8.94 | | | |
| | PSG score: −13.34 | | | |
| | GvH: von Heijne's method for signal seq. recognition | | | |
| | GvH score (threshold: −2.1): −7.87 | | | |
| | possible cleavage site: between 30 and 31 | | | |
| | >>> Seems to have no N-terminal signal peptide | | | |
| | ALOM: Klein et al's method for TM region allocation | | | |
| | Init position for calculation: 1 | | | |
| | Tentative number of TMS(s) for the threshold 0.5: 0 | | | |
| | number of TMS(s) . . . fixed | | | |
| | PERIPHERAL | Likelihood = | 0.58 (at 214) | |
| | ALOM score: 0.58 (number of TMSs: 0) | | | |
| | MITDISC: discrimination of mitochondrial targeting seq | | | |
| | R content: | 0 | Hyd Moment(75): | 1.32 |
| | Hyd Moment(95): | 1.79 | G content: | 0 |
| | D/E content: | 2 | S/T content: | 1 |

Score: −7.91
Gavel: prediction of cleavage sites for mitochondrial preseq
cleavage site motif not found
NUCDISC: discrimination of nuclear localization signals
pat4: none
pat7: none
bipartite: none
content of basic residues: 13.3%
NLS Score: −0.47
KDEL: ER retention motif in the C-terminus: none
ER Membrane Retention Signals:
KKXX-like motif in the C-terminus: KILA
SKL: peroxisomal targeting signal in the C-terminus: none
PTS2: 2nd peroxisomal targeting signal: none
VAC: possible vacuolar targeting motif: none
RNA-binding motif: none
Actinin-type actin-binding motif:
type 1: none
type 2: none
NMYR: N-myristoylation pattern: none
Prenylation motif: none
memYQRL: transport motif from cell surface to Golgi: none
Tyrosines in the tail: none
Dileucine motif in the tail: none
checking 63 PROSITE DNA binding motifs: none
checking 71 PROSITE ribosomal protein motifs: none
checking 33 PROSITE prokaryotic DNA binding motifs: none
NNCN: Reinhardt's method for Cytoplasmic/Nuclear discrimination
Prediction: nuclear
Reliability: 55.5
COIL: Lupas's algorithm to detect coiled-coil regions
total: 0 residues
Final Results (k = 9/23):
65.2%: nuclear
17.4%: mitochondrial
13.0%: cytoplasmic
4.3%: peroxisomal
>> prediction for CG91149-03 is nuc (k = 23)

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| AAR64312 | Rat liver adenosine monophosphate | 4 . . . 565 | 416/568 (73%) | 0.0 |
| | protein kinase - Rattus rattus, 552 aa. | 2 . . . 552 | 475/568 (83%) | |
| | [WO9428116-A1, 08 DEC. 1994] | | | |
| AAW29894 | Mammalian AMPK-alphal subunit | 4 . . . 364 | 343/361 (95%) | 0.0 |
| | active peptide 1 - Mammalia, 345 aa. | 2 . . . 345 | 344/361 (95%) | |
| | [WO9725341-A1, 17 JUL. 1997] | | | |
| ABB59603 | Drosophila melanogaster polypeptide | 13 . . . 565 | 338/605 (55%) | e−175 |
| | SEQ ID NO 5601 - Drosophila | 23 . . . 582 | 407/605 (66%) | |
| | melanogaster, 582 aa. | | | |
| | [WO200171042-A2, 27 SEP. 2001] | | | |
| AAW29899 | Mammalian AMPK-alphal subunit | 16 . . . 287 | 256/272 (94%) | e−147 |
| | active peptide 6 - Mammalia, 257 aa. | 1 . . . 257 | 257/272 (94%) | |
| | [WO9725341-A1, 17 JUL. 1997] | | | |
| ABR40709 | Zea mays oil trait related protein | 18 . . . 452 | 227/442 (51%) | e−121 |
| | sequence SEQ ID NO: 238 - Zea mays, | 90 . . . 501 | 305/442 (68%) | |
| | 579 aa. [WO2003002751-A2, | | | |
| | 09 JAN. 2003] | | | |

In a BLAST search of public sequence databases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q86VS1 | PRKAA1 protein - Homo sapiens | 1 . . . 565 | 563/565 (99%) | 0.0 |
| | (Human), 574 aa. | 10 . . . 574 | 565/565 (99%) | |
| Q13131 | 5′-AMP-activated protein kinase, | 1 . . . 565 | 550/565 (97%) | 0.0 |
| | catalytic alpha-1 chain (EC 2.7.1.-) | 1 . . . 550 | 550/565 (97%) | |
| | (AMPK alpha-1 chain) - Homo | | | |
| | sapiens (Human), 550 aa. | | | |
| AAH37303 | AMP-activated protein kinase - Homo | 1 . . . 565 | 549/565 (97%) | 0.0 |
| | sapiens (Human), 550 aa. | 1 . . . 550 | 550/565 (97%) | |
| P54645 | 5′-AMP-activated protein kinase, | 4 . . . 565 | 541/562 (96%) | 0.0 |
| | catalytic alpha-1 chain (EC 2.7.1.-) | 2 . . . 548 | 545/562 (96%) | |
| | (AMPK alpha-1 chain) - Rattus | | | |
| | norvegicus (Rat), 548 aa. | | | |
| Q8UVW8 | SNF1-like protein AMPK - Xenopus | 4 . . . 565 | 491/565 (86%) | 0.0 |
| | laevis (African clawed frog), 560 aa. | 12 . . . 560 | 523/565 (91%) | |

PFam analysis predicts that the NOV11a protein contains the domains shown in the Table 11F.

TABLE 11F
Domain Analysis of NOV11a

| | | Identities/ | |
|---|---|---|---|
| | | Similarities | |
| | NOV11a | for the | Expect |
| Pfam Domain | Match Region | Matched Region | Value |
| pkinase | 18 . . . 285 | 117/313 (37%) | 4.8e−94 |
| | | 213/313 (68%) | |

### Example B

### Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below: cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E. coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains Ni 06′ and YULH (U.S. Pat. Nos. 6,057,101 and 6,083,693).

4. RACE: Techniques based on the polymerase chain reaction such as rapid amplification of cDNA ends (RACE), were used to isolate or complete the sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C

### Quantitative Expression Analysis of Clones in Various Cells and Tissues

The quantitative expression of various NOV genes was assessed using microtiter plates containing RNA samples from a variety of normal and pathology-derived cells, cell lines and tissues using real time quantitative PCR (RTQ-PCR) performed on an Applied Biosystems (Foster City, Calif.) ABI PRISM® 7700 or an ABI PRISM® 7900 HT Sequence Detection System.

RNA integrity of all samples was determined by visual assessment of agarose gel electropherograms using 28S and 18S ribosomal RNA staining intensity ratio as a guide (2:1 to 2.5:1 28s: 18s) and the absence of low molecular weight RNAs (degradation products). Control samples to detect genomic DNA contamination included RTQ-PCR reactions run in the absence of reverse transcriptase using probe and primer sets designed to amplify across the span of a single exon.

RNA samples were normalized in reference to nucleic acids encoding constitutively expressed genes (i.e., β-actin and GAPDH). Alternatively, non-normalized RNA samples were converted to single strand cDNA (sscDNA) using Superscript II (Invitrogen Corporation, Carlsbad, Calif., Catalog No. 18064-147) and random hexamers according to the manufacturer's instructions. Reactions containing up to 10 μg of total RNA in a volume of 20 μl or were scaled up to contain 50 μg of total RNA in a volume of 100 μl and were incubated for 60 minutes at 42° C. sscDNA samples were then normalized in reference to nucleic acids as described above.

Probes and primers were designed according to Applied Biosystems Primer Express Software package (version I for Apple Computer's Macintosh Power PC) or a similar algorithm using the target sequence as input. Default reaction condition settings and the following parameters were set before selecting primers: 250 nM primer concentration; 58°-60° C. primer melting temperature (Tm) range; 59° C. primer optimal Tm; 2° C. maximum primer difference (if probe does not have 5′ G, probe Tm must be 10° C greater than primer Tm; and 75 bp to 100 bp amplicon size. The selected probes and primers were synthesized by Synthegen (Houston, Tex.). Probes were double purified by HPLC to remove uncoupled dye and evaluated by mass spectroscopy to verify coupling of reporter and quencher dyes to the 5′ and 3′ ends of the probe, respectively. Their final concentrations were: 900 nM forward and reverse primers, and 200 nM probe.

Normalized RNA was spotted in individual wells of a 96 or 384-well PCR plate (Applied Biosystems, Foster City, Calif.). PCR cocktails included a single gene-specific probe and primers set or two multiplexed probe and primers sets. PCR reactions were done using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles: 95° C. 10 min, then 40 cycles at 95° C. for 15 seconds, followed by 60° C. for 1 minute. Results were recorded as CT values (cycle at which a given sample crosses a threshold level of fluorescence) and plotted using a log scale, with the difference in RNA concentration between a given sample and the sample with the lowest CT value being represented as 2 to the power of delta CT. The percent relative expression was the reciprocal of the RNA difference multiplied by 100. CT values below 28 indicate high expression, between 28 and 32 indicate moderate expression, between 32 and 35 indicate low expression and above 35 reflect levels of expression that were too low to be measured reliably.

Normalized sscDNA was analyzed by RTQ-PCR using 1× TaqMan® Universal Master mix (Applied Biosystems; catalog No. 4324020), following the manufacturer's instructions. PCR amplification and analysis were done as described above.

Panels 1, 1.1, 1.2, and 1.3D

Panels 1, 1.1, 1.2 and 1.3D included 2 control wells (genomic DNA control and chemistry control) and 94 wells of cDNA samples from cultured cell lines and primary normal tissues. Cell lines were derived from carcinomas (ca) including: lung, small cell (s cell var), non small cell (non-s or non-sm); breast; melanoma; colon; prostate; glioma (glio), astrocytoma (astro) and neuroblastoma (neuro); squamous cell (squam); ovarian; liver; renal; gastric and pancreatic from the American Type Culture Collection (ATCC, Bethesda, Md.). Normal tissues were obtained from individual adults or fetuses and included: adult and fetal skeletal muscle, adult and fetal heart, adult and fetal kidney, adult and fetal liver, adult and fetal lung, brain, spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. The following abbreviations are used in reporting the results: metastasis (met); pleural effusion (pl. eff or pl effusion) and * indicates established from metastasis.

General_screening_panel_v1.4, v1.5, v1.6 and v1.7

Panels 1.4, 1.5, 1.6 and 1.7 were as described for Panels 1, 1.1, 1.2 and 1.3D, above except that normal tissue samples were pooled from 2 to 5 different adults or fetuses.

Panels 2D, 2.2, 2.3 and 2.4

Panels 2D, 2.2, 2.3 and 2.4 included 2 control wells and 94 wells containing RNA or cDNA from human surgical specimens procured through the National Cancer Institute's Cooperative Human Tissue Network (CHTN) or the National Disease Research Initiative (NDRI), Ardais (Lexington, Mass.) or Clinomics BioSciences (Frederick, Md.). Tissues included human malignancies and in some cases matched adjacent normal tissue (NAT). Information regarding histopathological assessment of tumor differentiation grade as well as the clinical stage of the patient from which samples were obtained was generally available. Normal tissue RNA and cDNA samples were purchased from various commercial sources such as Clontech (Palo Alto, Calif.), Research Genetics and Invitrogen (Carlsbad, Calif.).

HASS Panel v 1.0

The HASS Panel v1.0 included 93 cDNA samples and two controls including: 81 samples of cultured human cancer cell lines subjected to serum starvation, acidosis and anoxia according to established procedures for various lengths of time; 3 human primary cells; 9 malignant brain cancers (4 medulloblastomas and 5 glioblastomas); and 2 controls. Cancer cell lines (ATCC) were cultured using recommended conditions and included: breast, prostate, bladder, pancreatic and CNS. Primary human cells were obtained from Clonetics (Walkersville, Md.). Malignant brain samples were gifts from the Henry Ford Cancer Center.

ARDAIS Panel v1.0 and v1.1

The ARDAIS Panel v1.0 and v1.1 included 2 controls and 22 test samples including: human lung adenocarcinomas, lung squamous cell carcinomas, and in some cases matched adjacent normal tissues (NAT) obtained from Ardais (Lexington, Mass.). Unmatched malignant and non-malignant RNA samples from lungs with gross histopathological assessment of tumor differentiation grade and stage and clinical state of the patient were obtained from Ardais.

ARDAIS Prostate v1.0

ARDAIS Prostate v1.0 panel included 2 controls and 68 test samples of human prostate malignancies and in some cases matched adjacent normal tissues (NAT) obtained from Ardais (Lexington, Mass.). RNA from unmatched malignant and non-malignant prostate samples with gross histopathological assessment of tumor differentiation grade and stage and clinical state of the patient were also obtained from Ardais.

ARDAIS Kidney v1.0

ARDAIS Kidney v1.0 panel included 2 control wells and 44 test samples of human renal cell carcinoma and in some cases matched adjacent normal tissue (NAT) obtained from Ardais (Lexington, Mass.). RNA from unmatched renal cell carcinoma and normal tissue with gross histopathological assessment of tumor differentiation grade and stage and clinical state of the patient were also obtained from Ardais.

ARDAIS Breast v1.0

ARDAIS Breast v1.0 panel included 2 control wells and 71 test samples of human breast malignancies and in some cases matched adjacent normal tissue (NAT) obtained from Ardais (Lexington, Mass.). RNA from unmatched malignant and non-malignant breast samples with gross histopathological assessment of tumor differentiation grade and stage and clinical state of the patient were also obtained from Ardais.

Panel 3D, 3.1 and 3.2

Panels 3D, 3.1, and 3.2 included two controls, 92 cDNA samples of cultured human cancer cell lines and 2 samples of human primary cerebellum. Cell lines (ATCC, National Cancer Institute (NCI), German tumor cell bank) were cultured as recommended and were derived from: squamous cell carcinoma of the tongue, melanoma, sarcoma, leukemia, lymphoma, and epidermoid, bladder, pancreas, kidney, breast, prostate, ovary, uterus, cervix, stomach, colon, lung and CNS carcinomas.

Panels 4D, 4R, and 4.1D

Panels 4D, 4R, and 4.1 D included 2 control wells and 94 test samples of RNA (Panel 4R) or cDNA (Panels 4D and 4.1 D) from human cell lines or tissues related to inflammatory conditions. Controls included total RNA from normal tissues such as colon, lung (Stratagene, La Jolla, Calif.), thymus and kidney (Clontech, Palo Alto, Calif.). Total RNA from cirrhotic and lupus kidney was obtained from BioChain Institute, Inc., (Hayward, Calif.). Crohn's intestinal and ulcerative colitis samples were obtained from the National Disease Research Interchange (NDRI, Philadelphia, Pa.). Cells purchased from Clonetics (Walkersville, Md.) included: astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, and human umbilical vein endothelial. These primary is cell types were activated by incubating with various cytokines (IL-1 beta 1-5 ng/ml, TNF alpha ˜5-10 ng/ml, IFN gamma ˜20-50 ng/ml, IL-4 ˜5-10 ng/ml, IL-9 ˜5-10 ng/ml, IL-13 5-10 ng/ml) or combinations of cytokines as indicated. Starved endothelial cells were cultured in the basal media (Clonetics, Walkersville, Md.) with 0.1% serum.

Mononuclear cells were prepared from blood donations using Ficoll. LAK cells were cultured in culture media [DMEM, 5% FCS (Hyclone, Logan, Utah), 100 mM non essential amino acids (Gibco/Life Technologies, Rockville, Md.), 1 mM sodium pyruvate (Gibco), mercaptoethanol 5.5×10-5 M (Gibco), and 10 mM Hepes (Gibco)] and interleukin 2 for 4-6 days. Cells were activated with 10-20 ng/ml PMA and 1-2 μg/ml ionomycin, 5-10 ng/ml IL-12, 20-50 ng/ml IFN gamma or 5-10 ng/ml IL-18 for 6 hours. In some cases, mononuclear cells were cultured for 4-5 days in culture media with 5 mg/ml PHA (phytohemagglutinin) or PWM (pokeweed mitogen; Sigma-Aldrich Corp., St. Louis, Mo.). Samples were taken at 24, 48 and 72 hours for RNA preparation. MLR (mixed lymphocyte reaction) samples were obtained by taking blood from two donors, isolating the mononuclear cells using Ficoll and mixing them 1:1 at a final concentration of 2×10⁶ cells/ml in culture media. The MLR samples were taken at various time points from 1-7 days for RNA preparation.

Monocytes were isolated from mononuclear cells using CD14 Miltenyi Beads, +ve VS selection columns and a Vario Magnet (Miltenyi Biotec, Auburn, Calif.) according to the manufacturer's instructions. Monocytes were differentiated into dendritic cells by culturing in culture media with 50 ng/ml GMCSF and 5 ng/ml IL-4 for 5-7 days. Macrophages were prepared by culturing monocytes for 5-7 days in culture media with ˜50 ng/ml 10% type AB Human Serum (Life technologies, Rockville, Md.) or MCSF (Macrophage colony stimulating factor; R&D, Minneapolis, Minn.). Monocytes, macrophages and dendritic cells were stimulated for 6 or 12-14 hours with 100 ng/ml lipopolysaccharide (LPS). Dendritic cells were also stimulated with 10 μg/ml anti-CD40 monoclonal antibody (Pharmingen, San Diego, Calif.) for 6 or 12-14 hours.

CD4+ lymphocytes, CD8+lymphocytes and NK cells were also isolated from mononuclear cells using CD4, CD8 and CD56 Miltenyi beads, positive VS selection columns and a Vario Magnet (Miltenyi Biotec, Auburn, Calif.) according to the manufacturer's instructions. CD45+RA and CD45+RO CD4+lymphocytes were isolated by depleting mononuclear cells of CD8+, CD56+, CD14+ and CD19+cells using CD8, CD56, CD14 and CD19 Miltenyi beads and positive selection. CD45RO Miltenyi beads were then used to separate the CD45+RO CD4+lymphocytes from CD45+RA CD4+lymphocytes. CD45+RA CD4+, CD45+RO CD4+ and CD8+ lymphocytes were cultured in culture media at 10⁶ cells/ml in culture plates precoated overnight with 0.5 mg/ml anti-CD28 (Pharmingen, San Diego, Calif.) and 3 μg/ml anti-CD3 (OKT3, ATCC) in PBS. After 6 and 24 hours, the cells were harvested for RNA preparation. To prepare chronically activated CD8+ lymphocytes, isolated CD8+ lymphocytes were activated for 4 days on anti-CD28, anti-CD3 coated plates and then harvested and expanded in culture media with IL-2 (1 ng/ml). These CD8+ cells were activated again with plate bound anti-CD3 and anti-CD28 for 4 days and expanded as described above. RNA was isolated 6 and 24 hours after the second activation and after 4 days of the second expansion culture. Isolated NK cells were cultured in culture media with 1 ng/ml IL-2 for 4-6 days before RNA was prepared.

B cells were prepared from minced and sieved tonsil tissue (NDRI). Tonsil cells were pelleted and resupended at 10⁶ cells/ml in culture media. Cells were activated using 5 μg/ml PWM (Sigma-Aldrich Corp., St. Louis, Mo.) or 0 μg/ml anti-CD40 (Pharmingen, San Diego, Calif.) and 5-10 ng/ml IL-4. Cells were harvested for RNA preparation after 24, 48 and 72 hours.

To prepare primary and secondary Th1/Th2 and Tr1 cells, umbilical cord blood CD4+ lymphocytes (Poietic Systems, German Town, Md.) were cultured at 10⁵-10⁶ cells/ml in culture media with IL-2 (4 ng/ml) in 6-well Falcon plates (precoated overnight with 10 μg/ml anti-CD28 (Pharmingen) and 2 μg/ml anti-CD3 (OKT3; ATCC) then washed twice with PBS).

To stimulate Th1 phenotype differentiation, IL-12 (5 ng/ml) and anti-IL4 (1 μg/ml) were used; for Th2 phenotype differentiation, IL-4 (5 ng/ml) and anti-IFN gamma (1 μg/ml) were used; and for Tr1 phenotype differentiation, IL-10 (5 ng/ml) was used. After 4-5 days, the activated Th1, Th2 and Tr1 lymphocytes were washed once with DMEM and expanded for 4-7 days in culture media with IL-2 (1 ng/ml). Activated Th1, Th2 and Tr1 lymphocytes were re-stimulated for 5 days with anti-CD28/CD3 and cytokines as described above with the addition of anti-CD95L (1 μg/ml) to prevent apoptosis. After 4-5 days, the Th1, Th2 and Tr1 lymphocytes were washed and expanded in culture media with IL-2 for 4-7 days. Activated Th1 and Th2 lymphocytes were maintained for a maximum of three cycles. RNA was prepared from primary and secondary Th1, Th2 and Tr1 after 6 and 24 hours following the second and third activations with plate-bound anti-CD3 and anti-CD28 mAbs and 4 days into the second and third expansion cultures.

Leukocyte cells lines Ramos, EOL-1, KU-812 were obtained from the ATCC. EOL-1 cells were further differentiated by culturing in culture media at 5×10⁵ cells/ml with 0.1 mM dbcAMP for 8 days, changing the media every 3 days and adjusting the cell concentration to 5×10⁵ cells/ml. RNA was prepared from resting cells or cells activated with PMA (10 ng/ml) and ionomycin (1 μg/ml) for 6 and 14 hours. RNA was prepared from resting CCD 1106 keratinocyte cell line (ATCC) or from cells activated with ˜5 ng/ml TNF alpha and 1 ng/ml IL-1 beta. RNA was prepared from resting NCI-H292, airway epithelial tumor cell line (ATCC) or from cells activated for 6 and 14 hours in culture media with 5 ng/ml IL-4, 5 ng/ml IL-9, 5 ng/ml IL-13, and 25 ng/ml IFN gamma.

RNA was prepared by lysing approximately 10⁷ cells/ml using Trizol (Gibco BRL) then adding 1/10 volume of bromochloropropane (Molecular Research Corporation, Cincinnati, Ohio), vortexing, incubating for 10 minutes at room temperature and then spinning at 14,000 rpm in a Sorvall SS34 rotor. The aqueous phase was placed in a 15 ml Falcon Tube and an equal volume of isopropanol was added and left at −20° C. overnight. The precipitated RNA was spun down at 9,000 rpm for 15 min and washed in 70% ethanol. The pellet was redissolved in 300 μl of RNAse-free water with 35 ml buffer (Promega, Madison, Wis.) 5 μl DTT, 7 μl RNAsin and 8 μl DNAse and incubated at 37° C. for 30 minutes to remove contaminating genomic DNA, extracted once with phenol chloroform and re-precipitated with 1/10 volume of 3 M sodium acetate and 2 volumes of 100% ethanol. The RNA was spun down, placed in RNAse free water and stored at −80° C.

AI_Comprehensive Panel_v1.0

Autoimmunity (AI) comprehensive panel v1.0 included two controls and 89 cDNA test samples isolated from male (M) and female (F) surgical and postmortem human tissues that were obtained from the Backus Hospital and Clinomics (Frederick, Md.). Tissue samples included: normal, adjacent (Adj); matched normal adjacent (match control); joint tissues (synovial (Syn) fluid, synovium, bone and cartilage, osteoarthritis (OA), rheumatoid arthritis (RA)); psoriatic; ulcerative colitis colon; Crohns disease colon; and emphysmatic, asthmatic, allergic and chronic obstructive pulmonary disease (COPD) lung.

Pulmonary and General Inflammation (PGI) Panel v1.0

Pulmonary and General inflammation (PGI) panel v1.0 included two controls and 39 test samples isolated as surgical or postmortem samples. Tissue samples include: five normal lung samples obtained from Maryland Brain and Tissue Bank, University of Maryland (Baltimore, Md.), International Bioresource systems, IBS (Tuscon, Ariz.), and Asterand (Detroit, Mich.), five normal adjacent intestine tissues (NAT) from Ardais (Lexington, Mass.), ulcerative colitis samples (UC) from Ardais (Lexington, Mass.); Crohns disease colon from NDRI, National Disease Research Interchange (Philadelphia, Pa.); emphysematous tissue samples from Ardais (Lexington, Mass.) and Genomic Collaborative Inc. (Cambridge, Mass.), asthmatic tissue from Maryland Brain and Tissue Bank, University of Maryland (Baltimore, Md.) and Genomic Collaborative Inc (Cambridge, Mass.) and fibrotic tissue from Ardais (Lexinton, Mass.) and Genomic Collaborative (Cambridge, Mass.).

Cellular OA/RA Panel

Cellular OA.RA panel includes 2 control wells and 35 test samples comprised of cDNA generated from total RNA isolated from human cell lines or primary cells representative of the human joint and its inflammatory condition. Cell types included normal human osteoblasts (Nhost) from Clonetics (Cambrex, East Rutherford, N.J.), human chondrosarcoma SW1353 cells from ATCC (Manossas, Va.)), human fibroblast-like synoviocytes from Cell Applications, Inc. (San Diego, Calif.) and MH7A cell line (a rheumatoid fibroblast-like synoviocytes transformed with SV40 T antigen) from Riken Cell bank (Tsukuba Science City, Japan). These cell types were activated by incubating with various cytokines (IL-1 beta 1-10 ng/ml, TNF alpha 5-50 ng/ml, or prostaglandin E2 for Nhost cells) for 1, 6, 18 or 24 h. All these cells were starved for at least 5 h and cultured in their corresponding basal medium with 0.1 to 1% FBS.

Minitissue OA/RA Panel

The OA/RA mini panel includes two control wells and 31 test samples comprised of cDNA generated from total RNA isolated from surgical and postmortem human tissues obtained from the University of Calgary (Alberta, Canada), NDRI (Philadelphia, Pa.), and Ardais Corporation (Lexington, Mass.). Joint tissue samples include synovium, bone and cartilage from osteoarthritic and rheumatoid arthritis patients undergoing reconstructive knee surgery, as well as, normal synovium samples (RNA and tissue). Visceral normal tissues were pooled from 2-5 different adults and included adrenal gland, heart, kidney, brain, colon, lung, stomach, small intestine, skeletal muscle, and ovary.

AI.05 Chondrosarcoma

AI.05 chondrosarcoma plates included SW1353 cells (ATCC) subjected to serum starvation and treated for 6 and 18 h with cytokines that are known to induce MMP (1, 3 and 13) synthesis (e.g. IL1beta). These treatments included: IL-1beta (10 ng/ml), IL-1beta+TNF-alpha (50 ng/ml), IL-1 beta+Oncostatin (50 ng/ml) and PMA (100 ng/ml). Supernatants were collected and analyzed for MMP 1, 3 and 13 production. RNA was prepared from these samples using standard procedures.

Panels 5D and 5I

Panel 5D and 5I included two controls and cDNAs isolated from human tissues, human pancreatic islets cells, cell lines, metabolic tissues obtained from patients enrolled in the Gestational Diabetes study (described below), and cells from different stages of adipocyte differentiation, including differentiated (AD), midway differentiated (AM), and undifferentiated (U; human mesenchymal stem cells).

Gestational Diabetes study subjects were young (18-40 years), otherwise healthy women with and without gestational diabetes undergoing routine (elective) Caesarean section. Uterine wall smooth muscle (UT), visceral (Vis) adipose, skeletal muscle (SK), placenta (PI) greater omentum adipose (GO Adipose) and subcutaneous (SubQ) adipose samples (less than 1 cc) were collected, rinsed in sterile saline, blotted and flash frozen in liquid nitrogen. Patients included: Patient 2, an overweight diabetic Hispanic not on insulin; Patient 7-9, obese non-diabetic Caucasians with body mass index (BMI) greater than 30; Patient 10, an overweight diabetic Hispanic, on insulin; Patient 11, an overweight nondiabetic African American; and Patient 12, a diabetic Hispanic on insulin.

Differentiated adipocytes were obtained from induced donor progenitor cells (Clonetics, Walkersville, Md.). Differentiated human mesenchymal stem cells (HuMSCs) were prepared as described in Mark F. Pittenger, et al., Multilineage Potential of Adult Human Mesenchymal Stem Cells Science Apr. 2 1999: 143-147. mRNA was isolated and sscDNA was produced from Trizol lysates or frozen pellets. Human cell lines (ATCC, NCI or German tumor cell bank) included: kidney proximal convoluted tubule, uterine smooth muscle cells, small intestine, liver HepG2 cancer cells, heart primary stromal cells and adrenal cortical adenoma cells. Cells were cultured, RNA extracted and sscDNA was produced using standard procedures.

Panel 5I also contains pancreatic islets (Diabetes Research Institute at the University of Miami School of Medicine).

Human Metabolic RTQ-PCR Panel

Human Metabolic RTQ-PCR Panel included two controls (genomic DNA control and chemistry control) and 211 cDNAs isolated from human tissues and cell lines relevant to metabolic diseases. This panel identifies genes that play a role in the etiology and pathogenesis of obesity and/or diabetes. Metabolic tissues including placenta (PI), uterine wall smooth muscle (Ut), visceral adipose, skeletal muscle (Sk) and subcutaneous (SubQ) adipose were obtained from the Gestational Diabetes study (described above). Included in the panel are: Patients 7 and 8, obese non-diabetic Caucasians; Patient 12 a diabetic Caucasian with unknown BMI, on insulin (treated); Patient 13, an overweight diabetic Caucasian, not on insulin (untreated); Patient 15, an obese, untreated, diabetic Caucasian; Patient 17 and 25, untreated diabetic Caucasians of normal weight; Patient 18, an obese, untreated, diabetic Hispanic; Patient 19, a non-diabetic Caucasian of normal weight; Patient 20, an overweight, treated diabetic Caucasian; Patient 21 and 23, overweight non-diabetic Caucasians; Patient 22, a treated diabetic Caucasian of normal weight; Patient 23, an overweight non-diabetic Caucasian; and Patients 26 and 27, obese, treated, diabetic Caucasians.

Total RNA was isolated from metabolic tissues including: hypothalamus, liver, pancreas, pancreatic islets, small intestine, psoas muscle, diaphragm muscle, visceral (Vis) adipose, subcutaneous (SubQ) adipose and greater omentum (Go) from 12 Type II diabetic (Diab) patients and 12 non diabetic (Norm) at autopsy. Control diabetic and non-diabetic subjects were matched where possible for: age; sex, male (M); female (F); ethnicity, Caucasian (CC); Hispanic (HI); African American (AA); Asian (AS); and BMI, 20-25 (Low BM), 26-30 (Med BM) or overweight (Overwt), BMI greater than 30 (Hi BMI) (obese).

RNA was extracted and ss cDNA was produced from cell lines (ATCC) by standard methods.

CNS Panels

CNS Panels CNSD.01, CNS Neurodegeneration V1.0 and CNS Neurodegeneration V2.0 included two controls and 46 to 94 test cDNA samples isolated from postmortem human brain tissue obtained from the Harvard Brain Tissue Resource Center (McLean Hospital). Brains were removed from calvaria of donors between 4 and 24 hours after death, and frozen at −80° C. in liquid nitrogen vapor.

Panel CNSD.01

Panel CNSD.01 included two specimens each from: Alzheimer's disease, Parkinson's disease, Huntington's disease, Progressive Supernuclear Palsy (PSP), Depression, and normal controls. Collected tissues included: cingulate gyrus (Cing Gyr), temporal pole (Temp Pole), globus palladus (Glob palladus), substantia nigra (Sub Nigra), primary motor strip (Brodman Area 4), parietal cortex (Brodman Area 7), prefrontal cortex (Brodman Area 9), and occipital cortex (Brodman area 17). Not all brain regions are represented in all cases.

Panel CNS Neurodegeneration V1.0

The CNS Neurodegeneration V1.0 panel included: six Alzheimer's disease (AD) brains and eight normals which included no dementia and no Alzheimer's like pathology (control) or no dementia but evidence of severe Alzheimer's like pathology (Control Path), specifically senile plaque load rated as level 3 on a scale of 0-3; 0 no evidence of plaques, 3 severe AD senile plaque load. Tissues collected included: hippocampus, temporal cortex (Brodman Area 21), parietal cortex (Brodman area 7), occipital cortex (Brodman area 17) superior temporal cortex (Sup Temporal Ctx) and inferior temporal cortex (Inf Temproal Ctx).

Gene expression was analyzed after normalization using a scaling factor calculated by subtracting the Well mean (CT average for the specific tissue) from the Grand mean (average CT value for all wells across all runs). The scaled CT value is the result of the raw CT value plus the scaling factor.

Panel CNS Neurodegeneration V2.0

The CNS Neurodegeneration V2.0 panel included sixteen cases of Alzheimer's disease (AD) and twenty-nine normal controls (no evidence of dementia prior to death) including fourteen controls (Control) with no dementia and no Alzheimer's like pathology and fifteen controls with no dementia but evidence of severe Alzheimer's like pathology (AH3), specifically senile plaque load rated as level 3 on a scale of 0-3; 0 no evidence of plaques, 3 severe AD senile plaque load. Tissues from the temporal cortex (Brodman Area 21) included the inferior and superior temporal cortex that was pooled from a given individual (Inf & Sup Temp Ctx Pool).

A. CG101025-01 and CG101025-07: Calcium/Calmodulin-Dependent Protein Kinase Type II Beta Chain

Expression of genes CG101025-01 and CG101025-07 was assessed using the primer-probe set Ag4967, described in Table AA. Results of the RTQ-PCR runs are shown in Tables AB, AC and AD.

TABLE AA

Probe Name Ag4967

| | | | |
|---|---|---|---|
| | | Start | SEQ |
| Primers | Length | Position | ID No |

| | | | | |
|---|---|---|---|---|
| Forward | 5′-aaagttcaatgccaggag | 22 | 900 | 113 |
| | aaag-3′ | | | |
| Probe | TET-5′-aagggagccatcct | 23 | 925 | 114 |
| | caccaccat-3′-TAMRA | | | |
| Reverse | 5′-aaactcttggctgagaaa | 22 | 959 | 115 |
| | ttcc-3′ | | | |

TABLE AB
General screening panel v1.5

| Tissue Name | A |
|---|---|
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.4 |
| Prostate ca.* (bone met) PC-3 | 1.1 |
| Prostate Pool | 0.5 |
| Placenta | 0.0 |
| Uterus Pool | 0.0 |
| Ovarian ca. OVCAR-3 | 0.1 |
| Ovarian ca. SK-OV-3 | 0.3 |
| Ovarian ca. OVCAR-4 | 0.1 |
| Ovarian ca. OVCAR-5 | 0.1 |
| Ovarian ca. IGROV-1 | 0.2 |
| Ovarian ca. OVCAR-8 | 0.1 |
| Ovary | 0.0 |
| Breast ca. MCF-7 | 0.6 |
| Breast ca. MDA-MB-231 | 0.2 |
| Breast ca. BT 549 | 0.0 |
| Breast ca. T47D | 0.4 |
| Breast ca. MDA-N | 0.0 |
| Breast Pool | 0.1 |
| Trachea | 0.0 |
| Lung | 0.0 |
| Fetal Lung | 0.6 |
| Lung ca. NCI-N417 | 0.0 |
| Lung ca. LX-1 | 0.0 |
| Lung ca. NCI-H146 | 3.0 |
| Lung ca. SHP-77 | 0.6 |
| Lung ca. A549 | 0.0 |
| Lung ca. NCI-H526 | 0.5 |
| Lung ca. NCI-H23 | 0.2 |
| Lung ca. NCI-H460 | 0.1 |
| Lung ca. HOP-62 | 0.0 |
| Lung ca. NCI-H522 | 0.4 |
| Liver | 0.3 |
| Fetal Liver | 0.0 |
| Liver ca. HepG2 | 0.0 |
| Kidney Pool | 0.0 |
| Fetal Kidney | 0.6 |
| Renal ca. 786-0 | 0.0 |
| Renal ca. A498 | 0.0 |
| Renal ca. ACHN | 0.8 |
| Renal ca. UO-31 | 0.4 |
| Renal ca. TK-10 | 0.0 |
| Bladder | 0.2 |
| Gastric ca. (liver met.) NCI-N87 | 0.0 |
| Gastric ca. KATO III | 0.0 |
| Colon ca. SW-948 | 0.0 |
| Colon ca. SW480 | 0.2 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.1 |
| Small Intestine Pool | 0.1 |
| Stomach Pool | 0.1 |
| Bone Marrow Pool | 0.0 |
| Fetal Heart | 52.1 |
| Heart Pool | 15.7 |
| Lymph Node Pool | 0.0 |
| Fetal Skeletal Muscle | 15.5 |
| Skeletal Muscle Pool | 21.6 |
| Spleen Pool | 0.2 |
| Thymus Pool | 0.0 |
| CNS cancer (glio/astro) U87-MG | 0.0 |
| CNS cancer (glio/astro) U-118-MG | 0.3 |
| CNS cancer (neuro; met) SK-N-AS | 0.2 |
| CNS cancer (astro) SF-539 | 0.0 |
| CNS cancer (astro) SNB-75 | 0.1 |
| CNS cancer (glio) SNB-19 | 0.1 |
| CNS cancer (glio) SF-295 | 0.0 |
| Brain (Amygdala) Pool | 6.1 |
| Brain (cerebellum) | 48.6 |
| Brain (fetal) | 100.0 |
| Brain (Hippocampus) Pool | 7.2 |
| Cerebral Cortex Pool | 10.1 |
| Brain (Substantia nigra) Pool | 6.4 |
| Brain (Thalamus) Pool | 12.8 |
| Brain (whole) | 21.8 |
| Spinal Cord Pool | 2.9 |
| Adrenal Gland | 2.0 |
| Pituitary gland Pool | 4.6 |
| Salivary Gland | 0.0 |
| Thyroid (female) | 0.4 |
| Pancreatic ca. CAPAN2 | 0.0 |
| Pancreas Pool | 0.1 |

Column A - Rel. Exp.(%) Ag496, Run 228926259

TABLE AC
Oncology cell line screening panel v3.1

| Tissue Name | A |
|---|---|
| 94905 Daoy Medulloblastoma/Cerebellum | 0.0 |
| 94906 TE671 Medulloblastom/Cerebellum | 3.8 |
| 94907 D283 Med Medulloblastoma/Cerebellum | 4.0 |
| 94908 PFSK-1 Primitive Neuroectodermal/Cerebellum | 1.7 |
| 94909 XF-498 CNS | 0.0 |
| 94910 SNB-78 CNS/glioma | 0.1 |
| 94911 SF-268 CNS/glioblastoma | 0.0 |
| 94912 T98G Glioblastoma | 0.0 |
| 96776 SK-N-SH Neuroblastoma (metastasis) | 0.4 |
| 94913 SF-295 CNS/glioblastoma | 0.0 |
| 94914 Cerebellum | 100.0 |
| 96777 Cerebellum | 92.0 |
| 94916 NCI-H292 Mucoepidermoid lung carcinoma | 0.0 |
| 94917 DMS-114 Small cell lung cancer | 1.4 |
| 94918 DMS-79 Small cell lung cancer/neuroendocrine | 18.7 |
| 94919 NCI-H146 Small cell lung cancer/neuroendocrine | 31.6 |
| 94920 NCI-H526 Small cell lung cancer/neuroendocrine | 5.7 |
| 94921 NCI-N417 Small cell lung cancer/neuroendocrine | 0.5 |
| 94923 NCI-H82 Small cell lung cancer/neuroendocrine | 2.5 |
| 94924 NCI-H157 Squamous cell lung cancer | 0.0 |
| (metastasis) | |
| 94925 NCI-H1155 Large cell lung cancer/ | 45.4 |
| neuroendocrine | |
| 94926 NCI-H1299 Large cell lung cancer/ | 0.0 |
| neuroendocrine | |
| 94927 NCI-H727 Lung carcinoid | 0.0 |
| 94928 NCI-UMC-11 Lung carcinoid | 2.1 |
| 94929 LX-1 Small cell lung cancer | 0.1 |
| 94930 Colo-205 Colon cancer | 0.0 |
| 94931 KM12 Colon cancer | 0.0 |
| 94932 KM20L2 Colon cancer | 0.0 |
| 94933 NCI-H716 Colon cancer | 10.0 |
| 94935 SW-48 Colon adenocarcinoma | 0.2 |
| 94936 SW1116 Colon adenocarcinoma | 0.1 |
| 94937 LS 174T Colon adenocarcinoma | 0.1 |
| 94938 SW-948 Colon adenocarcinoma | 0.0 |
| 94939 SW-480 Colon adenocarcinoma | 0.0 |
| 94940 NCI-SNU-5 Gastric carcinoma | 0.5 |
| 112197 KATO III Stomach | 0.0 |
| 94943 NCI-SNU-16 Gastric carcinoma | 0.0 |
| 94944 NCI-SNU-1 Gastric carcinoma | 0.1 |
| 94946 RF-1 Gastric adenocarcinoma | 0.1 |
| 94947 RF-48 Gastric adenocarcinoma | 0.0 |
| 96778 MKN-45 Gastric carcinoma | 1.3 |
| 94949 NCI-N87 Gastric carcinoma | 0.0 |
| 94951 OVCAR-5 Ovarian carcinoma | 0.0 |
| 94952 RL95-2 Uterine carcinoma | 0.0 |
| 94953 HelaS3 Cervical adenocarcinoma | 0.0 |
| 94954 Ca Ski Cervical epidermoid carcinoma | 0.0 |
| (metastasis | |
| 94955 ES-2 Ovarian clear cell carcinoma | 0.5 |
| 94957 Ramos Stimulated with PMA/ionomycin 6 h | 0.0 |
| 94958 Ramos Stimulated with PMA/ionomycin 14 h | 0.0 |
| 94962 MEG-01 Chronic myelogenous leukemia | 0.1 |
| (megokaryoblast) | |
| 94963 Raji Burkitt's lymphoma | 0.1 |
| 94964 Daudi Burkitt's lymphoma | 0.0 |
| 94965 U266 B-cell plasmacytoma/myeloma | 0.2 |
| 94968 CA46 Burkitt's lymphoma | 0.0 |
| 94970 RL non-Hodgkin's B-cell lymphoma | 0.0 |
| 94972 JM1 pre-B-cell lymphoma/leukemia | 0.0 |
| 94973 Jurkat T cell leukemia | 0.0 |
| 94974 TF-1 Erythroleukemia | 0.0 |
| 94975 HUT 78 T-cell lymphoma | 0.7 |
| 94977 U937 Histiocytic lymphoma | 0.0 |
| 94980 KU-812 Myelogenous leukemia | 0.0 |
| 769-P-Clear cell renal carcinoma | 0.1 |
| 94983 Caki-2 Clear cell renal carcinoma | 0.4 |
| 94984 SW 839 Clear cell renal carcinoma | 0.9 |
| 94986 G401 Wilms' tumor | 0.0 |
| 94987 Hs766T Pancreatic carcinoma (LN metastasis) | 0.0 |
| 94988 CAPAN-1 Pancreatic adenocarcinoma (liver | 0.9 |
| metastasis) | |
| 94989 SU86.86 Pancreatic carcinoma (liver | 0.0 |
| metastasis) | |
| 94990 BxPC-3 Pancreatic adenocarcinoma | 0.0 |
| 94991 HPAC Pancreatic adenocarcinoma | 0.0 |
| 94992 MIA PaCa-2 Pancreatic carcinoma | 0.0 |
| 94993 CFPAC-1 Pancreatic ductal adenocarcinoma | 0.0 |
| 94994 PANC-1 Pancreatic epithelioid ductal carcinoma | 0.0 |
| 94996 T24 Bladder carcinma (transitional cell | 0.0 |
| 5637- Bladder carcinoma | 0.0 |
| 94998 HT-1197 Bladder carcinoma | 0.0 |
| 94999 UM-UC-3 Bladder carcinma (transitional cell) | 0.2 |
| 95000 A204 Rhabdomyosarcoma | 0.0 |
| 95001 HT-1080 Fibrosarcoma | 0.0 |
| 95002 MG-63 Osteosarcoma (bone) | 1.7 |
| 95003 SK-LMS-1 Leiomyosarcoma (vulva) | 0.0 |
| 95004 SJRH30 Rhabdomyosarcoma (met to bone marrow) | 0.0 |
| 95005 A431 Epidermoid carcinoma | 0.0 |
| 95007 WM266-4 Melanoma | 0.1 |
| 112195 DU 145 Prostate | 0.2 |
| 95012 MDA-MB-468 Breast adenocarcinoma | 0.1 |
| 112196 SSC-4 Tongue | 0.0 |
| 112194 SSC-9 Tongue | 0.0 |
| 112191 SSC-15 Tongue | 0.0 |
| 95017 CAL 27 Squamous cell carcinoma of tongue | 0.0 |

Column A - Rel. Exp.(%) Ag4967, Run 22500931

TABLE AD
Panel 5 Islet

| Tissue Name | A |
|---|---|
| 97457 Patient-02go adipose | 0.0 |
| 97476 Patient-07sk skeletal muscle | 5.6 |
| 97477 Patient-07ut uterus | 0.0 |
| 97478 Patient-07pl placenta | 0.0 |
| 99167 Bayer Patient 1 | 32.1 |
| 97482 Patient-08ut uterus | 0.0 |
| 97483 Patient-08pl placenta | 0.0 |
| 97486 Patient-09sk skeletal muscle | 6.7 |
| 97487 Patient-09ut uterus | 0.0 |
| 97488 Patient-09pl placenta | 0.0 |
| 97492 Patient-10ut uterus | 0.0 |
| 97493 Patient-10pl placenta | 0.0 |
| 97495 Patient-11go adipose | 0.2 |
| 97496 Patient-11sk skeletal muscle | 52.5 |
| 97497 Patient- 11ut uterus | 0.0 |
| 97498 Patient- 11pl placenta | 0.0 |
| 97500 Patient- 12go adipose | 0.0 |
| 97501 Patient- 12sk skeletal muscle | 100.0 |
| 97502 Patient- 12ut uterus | 0.0 |
| 97503 Patient- 12pl placenta | 0.0 |
| 94721 Donor 2 U - A Mesenchymal Stem Cells | 0.0 |
| 94722 Donor 2 U - B Mesenchymal Stem Cells | 0.0 |
| 94723 Donor 2 U - C Mesenchymal Stem Cells | 0.0 |
| 94709 Donor 2 AM - A adipose | 0.0 |
| 94710 Donor 2 AM - B adipose | 0.0 |
| 94711 Donor 2 AM - C adipose | 0.0 |
| 94712 Donor 2 AD - A adipose | 0.0 |
| 94713 Donor 2 AD - B adipose | 0.0 |
| 94714 Donor 2 AD - C adipose | 0.0 |
| 94742 Donor 3 U - A Mesenchymal Stem Cells | 0.0 |
| 94743 Donor 3 U - B Mesenchymal Stem Cells | 0.0 |
| 94730 Donor 3 AM - A adipose | 0.0 |
| 94731 Donor 3 AM - B adipose | 0.0 |
| 94732 Donor 3 AM - C adipose | 0.0 |
| 94733 Donor 3 AD - A adipose | 0.0 |
| 94734 Donor 3 AD - B adipose | 0.0 |
| 94735 Donor 3 AD - C adipose | 0.0 |
| 77138 Liver HepG2untreated | 0.0 |
| 73556 Heart Cardiac stromal cells (primary) | 0.2 |
| 81735 Small Intestine | 0.6 |
| 72409 Kidney Proximal Convoluted Tubule | 0.2 |
| 82685 Small intestine Duodenum | 0.2 |
| 90650 Adrenal Adrenocortical adenoma | 0.0 |
| 72410 Kidney HRCE | 1.4 |
| 72411 Kidney HRE | 0.6 |
| 73139 Uterus Uterine smooth muscle cells | 0.0 |

Column A - Rl. Exp.(%) Ag4967, Run 240188656

General_screening_panel_v1.5 Summary: Ag4967 Highest expression of this gene was detected in fetal brain (Ct=27). High to moderate expression of this gene was seen in all regions of the central nervous system examined, including amygdala, hippocampus, substantia nigra, thalamus, cerebellum, cerebral cortex, and spinal cord. This gene codes for a splice variant of calcium/calmodulin-dependent-dependent kinase II. Calmodulin (CaM) is a major Ca2+-binding protein in the brain, where it plays an important role in the neuronal response to changes in the intracellular Ca2+ concentration. Calmodulin modulates numerous Ca2+-dependent enzymes and participates in relevant cellular functions. Among the different CaM-binding proteins, the Ca2+/CaM dependent protein kinase II and the phosphatase calcineurin are especially important in the brain because of their abundance and their participation in numerous neuronal functions (Sola et al., 2001, Int J Biochem Cell Biol 33(5):439-55, PMID: 11331200). Therapeutic modulation of this gene, expressed protein and/or use of antibodies or small molecule is drugs targeting the gene or gene product are useful in the treatment of neurological disorders such as Alzheimer's disease, Parkinson's disease, epilepsy, multiple sclerosis, schizophrenia and depression.

In addition, moderate expression of this gene was also seen in heart and skeletal muscle. Therapeutic modulation of this gene, expressed protein and/or use of antibodies or small molecule drugs targeting the gene or gene product are useful in the treatment of heart and muscle related diseases.

Oncology_cell_line_screening_panel_v3.1 Summary: Ag4967 Expression of this gene was highest in cerebellum (CT=28.6), consistent with what is observed in Panel 1.5. Please see Panel 1.5 for a description of the potential use of this gene in the treatment of central nervous system disorders.

This gene was also expressed at moderate levels in 5 out of 13 lung cancer cell lines. Therapeutic modulation of this gene, expressed protein and/or use of antibodies or small molecule drugs targeting the gene or gene product are useful in the treatment of lung cancer.

Panel 5 Islet Summary: Ag4967 Highest expression of the CG101025-01 gene was detected in skeletal muscle (CT=29.7). Moderate expression of this gene was also seen in pancreatic islet cells (Bayer patient 1). This gene codes for a splice variant of calcium/calmodulin-dependent-dependent kinase II (CaM kinase II). CaM kinase II plays an important role in insulin exocytosis in the beta-cell (Bhatt et al., 2000, Biochem Pharmacol 60(11):1655-63, PMID: 11077048). Inhibition of this enzyme suppresses calcium-dependent insulin secretion (Easom, 1999, Diabetes 48(4):675-84, PMID: 10102681). Therapeutic modulation of this gene or expressed protein can be used to increase insulin secretion in Type 2 diabetes. Therapeutic modulation of this gene, expressed protein and/or use of antibodies or small molecule drugs targeting the gene or gene product are useful in the treatment of muscle related diseases and other endocrine/metabolically related diseases, such as obesity.

B. CG101826-02: Adenylate Kinase

Expression of gene CG 101826-02 was assessed using the primer-probe set Ag5337, described in Table BA. Results of the RTQ-PCR runs are shown in Tables BB and BC.

TABLE BA

Probe Name Ag5337

| | | | |
|---|---|---|---|
| | | Start | SEQ |
| Primers | Length | Position | ID No |

| | | | | |
|---|---|---|---|---|
| Forward | 5′-tccatacagatcaccaac | 22 | 1556 | 116 |
| | tgtg-3′ | | | |
| Probe | TET-5′-cgtccgaactcttc | 25 | 1592 | 117 |
| | cccttgcttca-3′-TAMRA | | | |
| Reverse | 5′-agacattatggaacgtgg | 21 | 1718 | 118 |
| | aga-3′ | | | |

TABLE BB
General screening panel v1.5

| Tissue Name | A |
|---|---|
| Adipose | 0.1 |
| Melanoma* Hs688(A).T | 14.3 |
| Melanoma* Hs688(B).T | 8.9 |
| Melanoma* M14 | 40.3 |
| Melanoma* LOXIMVI | 1.2 |
| Melanoma* SK-MEL-5 | 0.0 |
| Squamous cell carcinoma SCC-4 | 0.1 |
| Testis Pool | 0.5 |
| Prostate ca.* (bone met) PC-3 | 2.3 |
| Prostate Pool | 0.6 |
| Placenta | 0.0 |
| Uterus Pool | 0.2 |
| Ovarian ca. OVCAR-3 | 0.1 |
| Ovarian ca. SK-OV-3 | 0.2 |
| Ovarian ca. OVCAR-4 | 1.4 |
| Ovarian ca. OVCAR-5 | 0.0 |
| Ovarian ca. IGROV-1 | 0.5 |
| Ovarian ca. OVCAR-8 | 1.1 |
| Ovary | 0.1 |
| Breast ca. MCF-7 | 0.0 |
| Breast ca. MDA-MB-231 | 0.4 |
| Breast ca. BT 549 | 4.5 |
| Breast ca. T47D | 0.0 |
| Breast ca. MDA-N | 0.3 |
| Breast Pool | 2.1 |
| Trachea | 0.6 |
| Lung | 0.3 |
| Fetal Lung | 0.2 |
| Lung ca. NCI-N417 | 0.5 |
| Lung ca. LX-1 | 0.0 |
| Lung ca. NCI-H146 | 0.7 |
| Lung ca. SHP-77 | 0.2 |
| Lung ca. A549 | 0.8 |
| Lung ca. NCI-H526 | 0.3 |
| Lung ca. NCI-H23 | 0.0 |
| Lung ca. NCI-H460 | 0.0 |
| Lung ca. HOP-62 | 0.9 |
| Lung ca. NCI-H522 | 1.6 |
| Liver | 0.0 |
| Fetal Liver | 0.0 |
| Liver ca. HepG2 | 0.0 |
| Kidney Pool | 0.5 |
| Fetal Kidney | 0.6 |
| Renal ca. 786-0 | 0.9 |
| Renal ca. A498 | 2.1 |
| Renal ca. ACHN | 16.7 |
| Renal ca. UO-31 | 2.4 |
| Renal ca. TK-10 | 0.7 |
| Bladder | 0.2 |
| Gastric ca. (liver met.) NCI-N87 | 0.0 |
| Gastric ca. KATO III | 0.0 |
| Colon ca. SW-948 | 0.0 |
| Colon ca. SW480 | 1.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.2 |
| Colon ca. SW1116 | 0.0 |
| Colon ca. Colo-205 | 0.0 |
| Colon ca. SW-48 | 0.0 |
| Colon Pool | 0.2 |
| Small Intestine Pool | 0.5 |
| Stomach Pool | 0.8 |
| Bone Marrow Pool | 0.1 |
| Fetal Heart | 0.2 |
| Heart Pool | 0.3 |
| Lymph Node Pool | 0.4 |
| Fetal Skeletal Muscle | 1.1 |
| Skeletal Muscle Pool | 0.3 |
| Spleen Pool | 0.3 |
| Thymus Pool | 0.2 |
| CNS cancer (glio/astro) U87-MG | 1.8 |
| CNS cancer (glio/astro) U-118-MG | 6.7 |
| CNS cancer (neuro; met) SK-N-AS | 2.3 |
| CNS cancer (astro) SF-539 | 4.7 |
| CNS cancer (astro) SNB-75 | 49.3 |
| CNS cancer (glio) SNB-19 | 0.5 |
| CNS cancer (glio) SF-295 | 1.1 |
| Brain (Amygdala) Pool | 59.9 |
| Brain (cerebellum) | 11.0 |
| Brain (fetal) | 7.7 |
| Brain (Hippocampus) Pool | 54.7 |
| Cerebral Cortex Pool | 100.0 |
| Brain (Substantia nigra) Pool | 52.5 |
| Brain (Thalamus) Pool | 88.3 |
| Brain (whole) | 66.0 |
| Spinal Cord Pool | 11.7 |
| Adrenal Gland | 0.2 |
| Pituitary gland Pool | 0.8 |
| Salivary Gland | 0.2 |
| Thyroid (female) | 0.1 |
| Pancreatic ca. CAPAN2 | 0.0 |
| Pancreas Pool | 0.1 |

Column A - Rel. Exp.(%) Ag533, Run 237370032

TABLE BC
Human Metabolic

| Tissue Name | A |
|---|---|
| 137857 psoas-AA.M.Diab.-hi BMI-6 | 0.0 |
| 135760 psoas-HI.M.Diab.-hi BMI-21 | 0.0 |
| 134827 psoas-CC.M.Diab.-hi BMI-4 | 0.0 |
| 137860 psoas-AA.M.Diab.-med BMI-8 | 0.0 |
| 137834 psoas-CC.M.Diab.-med BMI-2 | 0.2 |
| 137828 psoas-CC.M.Diab.-med BMI-1 | 0.0 |
| 135763 psoas-HI.M.Diab.-med BMI-23 | 0.0 |
| 142740 psoas-AS.M.Diab.-low BMI-20 | 0.1 |
| 134834 psoas-AA.M.Diab.-low BMI-17 | 0.0 |
| 137850 psoas-AS.M.Norm-hi BMI-34 | 0.0 |
| 135769 psoas-HI.M.Norm-hi BMI-31 | 0.0 |
| 135766 psoas-AA.M.Norm-hi BMI-25 | 0.0 |
| 142746 psoas-AA.M.Norm-med BMI-37 | 0.1 |
| 142745 psoas-HI.M.Norm-med BMI-35 | 0.1 |
| 137855 psoas-AA.M.Norm-med BMI-47 | 0.0 |
| 137844 psoas-CC.M.Norm-med BMI-26 | 0.0 |
| 142742 psoas-CC.M.Norm-low BMI-40 | 0.1 |
| 137873 psoas-AS.M.Norm-low BMI-28 | 0.1 |
| 137853 psoas-HI.M.Norm-low BMI-41 | 0.0 |
| 135775 psoas-CC.M.Norm-low BMI-39 | 0.0 |
| 137858 diaphragm-AA.M.Diab.-hi BMI-6 | 0.0 |
| 135772 diaphragm-AS.M.Diab-hi BMI-9 | 0.0 |
| 135761 diaphragm-HI.M.Diab.-hi BMI-21 | 0.1 |
| 134828 diaphragm-CC.M.Diab.-hi BMI-4 | 0.0 |
| 137835 diaphragm-CC.M.Diab.-med BMI-2 | 0.0 |
| 135764 diaphragm-HI.M.Diab.-med BMI-23 | 0.0 |
| 134835 diaphragm-AA.M.Diab.-low BMI-17 | 0.0 |
| 142738 diaphragm-CC.M.Norm-hi BMI-29 | 0.1 |
| 139517 diaphragm-AS.M.Norm-hi BMI-34 | 0.0 |
| 137848 diaphragm-HI.M.Norm-hi BMI-31 | 1.8 |
| 137843 diaphragm- AA.M.Norm-hi BMI-25 | 73.7 |
| 137879 diaphragm-AA.M.Norm-med BMI-47 | 0.0 |
| 137872 diaphragm-CC.M.Norm-med BMI-26 | 0.0 |
| 135773 diaphragm-HI.M.Norm-med BMI-35 | 0.0 |
| 139542 diaphragm-HI.M.Norm-low BMI-41 | 0.0 |
| 137877 diaphragm-CC.M.Norm-low BMI-39 | 0.0 |
| 137874 diaphragm-AS.M.Norm-low BMI-28 | 0.0 |
| 141340 subQadipose-AA.M.Diab.-hi BMI-6 | 1.9 |
| 137836 subQadipose-HI.M.Diab.-hi BMI-21 | 0.0 |
| 135771 subQadipose-AS.M.Diab-hi BMI-9 | 0.1 |
| 141329 pancreas-CC.M.Diab.-hi BMI-4 | 0.0 |
| 137862 subQadipose-CC.M.Diab.-med BMI-1 | 0.0 |
| 135762 subQadipose-HI.M.Diab.-med BMI-23 | 0.0 |
| 141338 subQadipose-AS.M.Diab.-low BMI-20 | 0.2 |
| 139547 subQadipose-HI.M.Diab.-low BMI-22 | 0.0 |
| 135757 subQadipose-CC.M.Diab.-low BMI-13 | 0.0 |
| 134832 subQadipose-AA.M.Diab.-low BMI-17 | 0.0 |
| 141332 subQadipose-HI.M.Norm-hi BMI-31 | 0.2 |
| 135767 subQadipose-CC.M.Norm-hi BMI-29 | 0.0 |
| 135765 subQadipose-AS.M.Norm-hi BMI-34 | 0.0 |
| 141339 subQadipose-HI.M.Norm-med BMI-35 | 0.5 |
| 141334 subQadipose-CC.M.Norm-med BMI-26 | 0.0 |
| 139544 subQadipose-AA.M.Norm-med BMI-47 | 0.0 |
| 137875 subQadipose-AA.M.Norm-med BMI-37 | 0.0 |
| 141331 subQadipose-AS.M.Norm-low BMI-28 | 0.0 |
| 137878 subQadipose-HI.M.Norm-low BMI-41 | 0.0 |
| 137876 subQadipose-CC.M.Norm-low BMI-39 | 0.0 |
| 137859 vis.adipose-AA.M.Diab.-hi BMI-6 | 0.0 |
| 135770 vis.adipose-AS.M.Diab-hi BMI-9 | 0.0 |
| 135759 vis.adipose-HI.M.Diab.-hi BMI-21 | 0.0 |
| 143502 vis.adipose-CC.M.Diab.-med BMI-2 | 0.0 |
| 139510 vis.adipose-AA.M.Diab.-med BMI-8 | 0.0 |
| 137861 vis.adipose-CC.M.Diab.-med-1 | 0.0 |
| 137839 vis.adipose-HI.M.Diab.-med BMI-23 | 0.0 |
| 139546 vis.adipose-HI.M.Diab.-low BMI-22 | 0.1 |
| 137831 vis.adipose-CC.M.Diab.-low BMI-13 | 0.0 |
| 139522 vis.adipose-HI.M.Norm-hi BMI-31 | 0.0 |
| 139516 vis.adipose-AS.M.Norm-hi BMI-34 | 0.0 |
| 137846 vis.adipose-CC.M.Norm-hi BMI-29 | 0.0 |
| 137841 vis.adipose-AA.M.Norm-hi BMI-25 | 0.0 |
| 139543 vis.adipose-AA.M.Norm-med BMI-47 | 0.1 |
| 139532 vis.adipose-AA.M.Norm-med BMI-37 | 0.1 |
| 139530 vis.adipose-HI.M.Norm-med BMI-35 | 0.0 |
| 139539 vis.adipose-HI.M.Norm-low BMI-41 | 0.0 |
| 139535 vis.adipose-CC.M.Norm-low BMI-40 | 0.2 |
| 137852 vis.adipose-CC.M.Norm-low BMI-39 | 0.0 |
| 135768 vis.adipose-AS.M.Norm-low BMI-28 | 0.0 |
| 141327 liver-CC.M.Diab.-hi BMI-4 | 0.0 |
| 139514 liver-HI.M.Diab.-hi BMI-21 | 0.0 |
| 139526 liver-CC.M.Diab.-med BMI-2 | 0.0 |
| 139511 liver-AA.MDiab.-med BMI-8 | 0.0 |
| 137840 liver-HI.M.Diab.-med BMI-23 | 0.0 |
| 137827 liver-CC.M.Diab.-med BMI-1 | 0.0 |
| 137838 liver-HI.M.Diab.-low BMI-22 | 0.0 |
| 135758 liver-CC.M.Diab.-low BMI-13 | 0.0 |
| 139519 liver-CC.M.Norm-hi BMI-29 | 0.0 |
| 139518 liver-AA.M.Norm-hi BMI-25 | 0.0 |
| 137849 liver-AS.M.Norm-hi BMI-34 | 0.0 |
| 137847 liver-HI.M.Norm-hi BMI-31 | 0.0 |
| 142741 liver-AA.M.Norm-med BMI-37 | 0.0 |
| 141341 liver-HI.M.Norm-med BMI-35 | 0.0 |
| 141335 liver-CC.M.Norm-med BMI-26 | 0.0 |
| 139540 liver-HI.M.Norm-low BMI-41 | 0.1 |
| 139534 liver-CC.M.Norm-low BMI-39 | 0.2 |
| 139521 liver-AS.M.Norm-low BMI-28 | 0.0 |
| 141328 pancreas-CC.M.Diab.-hi BMI-4 | 0.2 |
| 139525 pancreas-AS.M.Diab.-hi BMI-9 | 0.0 |
| 137856 pancreas-AA.M.Diab.-hi BMI-6 | 0.0 |
| 137837 pancreas-HI.M.Diab.-hi BMI-21 | 0.0 |
| 141337 pancreas-CC.M.Diab.-med BMI-2 | 0.0 |
| 139527 pancreas-CC.M.Diab.-med BMI-1 | 0.1 |
| 139515 pancreas-HI.M.Diab.-med BMI-23 | 0.3 |
| 139512 pancreas-AA.M.Diab.-med BMI-8 | 0.2 |
| 142739 pancreas-AS.M.Diab.-low BMI-20 | 0.0 |
| 139513 pancreas-CC.M.Diab.-low BMI-13 | 0.2 |
| 142743 pancreas-AA.M.Norm-hi BMI-25 | 0.1 |
| 139523 pancreas-HI.M.Norm-hi BMI-31 | 0.1 |
| 139520 pancreas-CC.M.Norm-hi BMI-29 | 0.0 |
| 142744 pancreas-HI.M.Norm-med BMI-35 | 0.0 |
| 139545 pancreas-AA.M.Norm-med BMI-47 | 0.3 |
| 139531 pancreas-AA.M.Norm-med BMI-37 | 0.0 |
| 137871 pancreas-CC.M.Norm-med BMI-26 | 0.0 |
| 139541 pancreas-Hi.M.Norm-low BMI-41 | 0.0 |
| 139537 pancreas-CC.M.Norm-low BMI-40 | 0.1 |
| 139533 pancreas-CC.M.Norm-low BMI-39 | 0.0 |
| 137845 pancreas-AS.M.Norm-low BMI-28 | 0.0 |
| 143530 small intestine-AA.M.Diab.-hi BMI-6 | 0.2 |
| 143529 small intestine-CC.M.Diab.-hi BMI-4 | 0.2 |
| 143538 small intestine-HI.M.Diab.-med BMI-23 | 1.6 |
| 143531 small intestine-AA.M.Diab.-med BMI-8 | 0.5 |
| 143528 small intestine-CC.M.Diab.-med BMI-2 | 0.2 |
| 143537 small intestine-HI.M.Diab.-low BMI-22 | 0.1 |
| 143535 small intestine-AS.M.Diab.-low BMI-20 | 0.0 |
| 143534 small intestine-AA.M.Diab.-low BMI-17 | 0.0 |
| 143544 small intestine-AS.M.Norm-hi BMI-34 | 0.1 |
| 143543 small intestine-HI.M.Norm-hi BMI-31 | 0.1 |
| 143542 small intestine-CC.M.Norm-hi BMI-29 | 0.1 |
| 143539 small intestine-AA.M.Norm-hi BMI-25 | 0.2 |
| 143548 small intestine-AA.M.Norm-med BMI-47 | 0.1 |
| 143547 small intestine-AA.M.Norm-med BMI-37 | 0.0 |
| 143540 small intestine-CC.M.Norm-med BMI-26 | 0.0 |
| 143550 small intestine-CC.M.Norm-low BMI-40 | 0.1 |
| 143549 small intestine-CC.M.Norm-low BMI-39 | 0.2 |
| 143546 small intestine-HI.M.Norm-low BMI-41 | 0.1 |
| 143525 hypothalamus-HI.M.Diab.-hi BMI-21 | 0.1 |
| 143515 hypothalamus-CC.M.Diab.-hi BMI-4 | 0.0 |
| 143513 hypothalamus-AA.M.Diab.-hi BMI-6 | 13.1 |
| 143507 hypothalamus-AS.M.Diab.-hi BMI-9 | 36.6 |
| 143506 hypothalamus-CC.M.Diab.-med BMI-1 | 100.0 |
| 143505 hypothalamus-HI.M.Diab.-med BMI-23 | 0.4 |
| 143509 hypothalamus-AA.M.Diab.-low BMI-17 | 80.7 |
| 143508 hypothalamus-CC.M.Diab.-low BMI-13 | 33.2 |
| 143503 hypothalamus-AS.M.Diab.-low BMI-20 | 42.6 |
| 143522 hypothalamus-HI.M.Norm-hi BMI-31 | 0.0 |
| 143516 hypothalamus-AS.M.Norm-hi BMI-34 | 0.0 |
| 143511 hypothalamus-CC.M.Norm-hi BMI-29 | 33.2 |
| 143504 hypothalamus-AA.M.Norm-hi BMI-25 | 13.5 |
| 143517 hypothalamus-AA.M.Norm-med BMI-47 | 0.1 |
| 143514 hypothalamus-HI.M.Norm-med BMI-35 | 3.0 |
| 143521 hypothalamus-AS.M.Norm-low BMI-28 | 0.0 |
| 143512 hypothalamus-CC.M.Norm-low BMI-40 | 0.1 |
| 145454 Patient-25pl (CC.Diab.low BMI.no insulin) | 0.0 |
| 110916 Patient-18pl (HI.Diab.obese.no insulin) | 0.0 |
| 110913 Patient-18go (HI.Diab.obese.no insulin) | 0.1 |
| 110911 Patient-17pl (CC.Diab.low BMI.no insulin) | 0.0 |
| 110908 Patient-17go (CC.Diab.low BMI.no insulin) | 0.1 |
| 100752 Patient-15sk (CC.Diab.obese.no insulin) | 0.1 |
| 97828 Patient-13pl (CC.Diab.overwt.no insulin) | 0.0 |
| 160114 Patient 27-ut (CC.Diab.obese.insulin) | 0.2 |
| 160113 Patient 27-pl (CC.Diab.obese.insulin) | 0.0 |
| 160112 Patient 27-sk (CC.Diab.obese.insulin) | 0.1 |
| 160111 Patient 27-go (CC.Diab.obese.insulin) | 0.0 |
| 145461 Patient-26sk (CC.Diab.obese.insulin) | 0.1 |
| 145441 Patient-22sk (CC.Diab.low BMI.insulin) | 0.1 |
| 145438 Patient-22pl (CC.Diab.low BMI.insulin) | 0.7 |
| 145427 Patient-20pl (CC.Diab.overwt.insulin) | 0.0 |
| 97503 Patient-12pl (CC.Diab.unknown BMI.insulin) | 0.0 |
| 145443 Patient-23pl (CC.Non-diab.overwt) | 0.0 |
| 145435 Patient-21pl (CC.Non-diab.overwt) | 0.0 |
| 110921 Patient-19pl (CC.Non-diab.low BMI) | 0.0 |
| 110918 Patient-19go (CC.Non-diab.low BMI) | 0.0 |
| 97481 Patient-08sk (CC.Non-diab.obese) | 0.1 |
| 97478 Patient-07pl (CC.Non-diab.obese) | 0.0 |
| 160117 Human Islets-male, obese | 0.0 |
| 145474 PANC1 (pancreas carcinoma) 1 | 1.6 |
| 154911 Capan2 (pancreas adenocarcinoma) | 0.0 |
| 141190 SW579 (thyroid carcinoma) | 6.0 |
| 145489 SK-N-MC (neuroblastoma) 1 | 0.2 |
| 145495 SK-N-SH (neuroblastoma) 1 | 2.6 |
| 145498 U87 MG (glioblastoma) 2 | 0.2 |
| 145484 HEp-2 (larynx carcinoma) 1 | 0.0 |
| 145479 A549 (lung carcinoma) | 0.9 |
| 145488 A427 (lung carcinoma) 2 | 0.0 |
| 145472 FHs 738Lu (normal lung) 1 | 8.4 |
| 141187 SKW6.4 (B lymphocytes) | 0.0 |
| 154644 IM-9 (immunoglobulin secreting lymphoblast) | 0.0 |
| 154645 MOLT-4 (acute lymphoblastic leukemia | 0.0 |
| derived from peripheral blood) | |
| 154648 U-937 (histiocystic lymphoma) | 1.6 |
| 154647 Daudi (Burkitt's lymphoma) | 0.0 |
| 145494 SK-MEL-2 (melanoma) 2 | 3.2 |
| 141176 A375 (melanoma) | 0.0 |
| 154642 SW 1353 (humerus chondrosarcoma) | 26.6 |
| 141179 HT-1080 (fibrosarcoma) | 1.8 |
| 145491 MG-63 (osteosarcoma) 1 | 5.2 |
| 141186 MCF7 (breast carcinoma) | 0.0 |
| 141193 T47D (breast carcinoma) | 0.0 |
| 154641 BT-20 (breast carcinoma) | 0.3 |
| 141175 293 (kidney transformed with adenovirus 5 | 12.7 |
| DNA) | |
| 141182 HUH hepatoma 1 | 0.0 |
| 141184 HUH7 hepatoma 1 | 0.0 |
| 145478 HT1376 (bladder carcinoma) | 0.0 |
| 145481 SCaBER (bladder carcinoma) | 0.0 |
| 141192 SW620 (lymph node metastatsis, colon | 0.0 |
| carcinoma) 2 | |
| 141180 HT29 (colon carcinoma) 1 | 0.0 |
| 141188 SW480 (colon carcinoma) 1 | 0.1 |
| 154646 CAOV-3 (ovary adenocarcinoma) | 0.0 |
| 141194 HeLa (cervix carcinoma)- 2 | 0.0 |
| 145482 HeLa S3 (cervix carcinoma) 1 | 0.0 |
| 145486 DU145 (prostate carcinoma) | 0.0 |
| 154643 PC-3 (prostate adenocarcinoma) | 1.7 |
| 154649 HCT-8 (ileocecal adenocarcinoma) | 0.0 |

Column A - Rel. Ex.(%) Ag5337, Run 323695066

General_screening_panel_v1.5 Summary: Ag5337 The highest expression of this gene was detected in the cerebral cortex (CT=25.7). In addition, high levels of expression were seen in thalamus, substantia nigra, thalamus and amygdala. Moderate levels of expression were seen in hippocampus, spinal cord, fetal brain, and a brain cancer cell line. This gene encodes a putative adenylate kinase 5, a brain specific enzyme involved in the synthesis of adenine nucleotides necessary for the homeostasis of engergy metabolism in cells (Van Rompay A R, Eur J Biochem 1999 April;261(2):509-17; Inouye S et. al J Neurochem 1998 July;71(1):125-33). This enzyme plays an important role in neuronal function and may be involved in neuronal maturation and regeneration (Inouye S. Biochem Biophys Res Commun 1999 Jan. 27;254(3):618-22). Therapeutic modulation of this gene, expressed protein and/or use of antibodies or small molecule drugs targeting the gene or gene product are useful in the treatment of neurodegenerative disorders such as Alzheimer's disease, Parkinson's disease, schizophrenia, multiple sclerosis, stroke and epilepsy.

Among tissues with metabolic function, this gene was expressed at low but significant levels in pituitary, adipose, adrenal gland, and adult and fetal skeletal muscle and heart. This widespread expression among these tissues suggests that this gene product plays a role in normal neuroendocrine and metabolic function and that disregulated expression of this gene contributes to neuroendocrine disorders or metabolic diseases, such as obesity and diabetes.

High to moderate levels of expression were seen in a cluster of samples derived from brain, renal and melanoma cancer cell lines. Thus, expression of this gene can be used as a marker to detect the presence of these cancers. Therapeutic modulation of this gene, expressed protein and/or use of antibodies or small molecule drugs targeting the gene or gene product are useful in the treatment of brain, renal and melanoma cancers.

Human Metabolic Summary: Ag5337 The highest expression of this gene was detected in hypothalamus of a diabetic patient (CT=23). The exression of this gene was higher in hypthalamus of diabetic patients than in controls. The hypothalamus is known to be involved in regulation of satiety. Therapeutic modulation of this gene, expressed protein and/or use of antibodies or small molecule drugs targeting the gene or gene product are useful in the treatment of obesity and diabetes.

C. CG105201-01: Hexokinase III Splice Variant

Expression of gene CG 105201-01 was assessed using the primer-probe set Ag4282, described in Table CA. Results of the RTQ-PCR runs are shown in Tables CB, CC, CD and CE.

TABLE CA

Probe Name Ag4282

| | | | |
|---|---|---|---|
| | | Start | SEQ |
| Primers | Length | Position | ID No |

| | | | | |
|---|---|---|---|---|
| Forward | 5′-cttcagcttctctttccc | 22 | 556 | 119 |
| | ttgt-3′ | | | |
| Probe | TET-5′-cttggacaggagca | 26 | 599 | 120 |
| | ccctcatttcct-3′-TAMRA | | | |
| Reverse | 5′-acaccactgcacctaaaa | 22 | 631 | 121 |
| | cctt-3′ | | | |

TABLE CB
AI comprehensive panel v1.0

| Tissue Name | A |
|---|---|
| 110967 COPD-F | 0.0 |
| 110980 COPD-F | 1.1 |
| 110968 COPD-M | 1.2 |
| 110977 COPD-M | 1.7 |
| 110989 Emphysema-F | 1.2 |
| 110992 Emphysema-F | 1.2 |
| 110993 Emphysema-F | 1.5 |
| 110994 Emphysema-F | 0.0 |
| 110995 Emphysema-F | 1.0 |
| 110996 Emphysema-F | 0.7 |
| 110997 Asthma-M | 0.0 |
| 111001 Asthma-F | 1.2 |
| 111002 Asthma-F | 1.4 |
| 111003 Atopic Asthma-F | 0.2 |
| 111004 Atopic Asthma-F | 0.7 |
| 111005 Atopic Asthma-F | 0.1 |
| 111006 Atopic Asthma-F | 0.0 |
| 111417 Allergy-M | 0.4 |
| 112347 Allergy-M | 0.1 |
| 112349 Normal Lung-F | 0.1 |
| 112357 Normal Lung-F | 1.2 |
| 112354 Normal Lung-M | 0.0 |
| 112374 Crohns-F | 0.8 |
| 112389 Match Control Crohns-F | 0.3 |
| 112375 Crohns-F | 3.4 |
| 112732 Match Control Crohns-F | 1.1 |
| 112725 Crohns-M | 0.2 |
| 112387 Match Control Crohns-M | 0.4 |
| 112378 Crohns-M | 0.1 |
| 112390 Match Control Crohns-M | 0.8 |
| 112726 Crohns-M | 2.6 |
| 112731 Match Control Crohns-M | 1.6 |
| 112380 Ulcer Col-F | 0.3 |
| 112734 Match Control Ulcer Col-F | 4.3 |
| 112384 Ulcer Col-F | 2.3 |
| 112737 Match Control Ulcer Col-F | 0.0 |
| 112386 Ulcer Col-F | 0.8 |
| 112738 Match Control Ulcer Col-F | 11.3 |
| 112381 Ulcer Col-M | 0.0 |
| 112735 Match Control Ulcer Col-M | 1.1 |
| 112382 Ulcer Col-M | 0.7 |
| 112394 Match Control Ulcer Col-M | 0.7 |
| 112383 Ulcer Col-M | 1.5 |
| 112736 Match Control Ulcer Col-M | 0.3 |
| 112423 Psoriasis-F | 1.6 |
| 112427 Match Control Psoriasis-F | 0.8 |
| 112418 Psoriasis-M | 1.5 |
| 112723 Match Control Psoriasis-M | 0.4 |
| 112419 Psoriasis-M | 0.8 |
| 112424 Match Control Psoriasis-M | 0.4 |
| 112420 Psoriasis-M | 1.3 |
| 112425 Match Control Psoriasis-M | 0.6 |
| 104689 (MF) OA Bone-Backus | 6.6 |
| 104690 (MF) Adj “Normal” Bone-Backus | 3.9 |
| 104691 (MF) OA Synovium-Backus | 17.1 |
| 104692 (BA) OA Cartilage-Backus | 2.9 |
| 104694 (BA) OA Bone-Backus | 8.5 |
| 104695 (BA) Adj “Normal” Bone-Backus | 2.5 |
| 104696 (BA) OA Synovium-Backus | 35.4 |
| 104700 (SS) OA Bone-Backus | 41.5 |
| 104701 (SS) Adj “Normal” Bone-Backus | 7.5 |
| 104702 (SS) OA Synovium-Backus | 12.8 |
| 117093 OA Cartilage Rep7 | 0.5 |
| 112672 OA Bone5 | 2.6 |
| 112673 OA Synovium5 | 0.8 |
| 112674 OA Synovial Fluid cells5 | 0.0 |
| 117100 OA Cartilage Rep14 | 0.4 |
| 112756 OA Bone9 | 1.6 |
| 112757 OA Synovium9 | 1.1 |
| 112758 OA Synovial Fluid Cells9 | 1.6 |
| 117125 RA Cartilage Rep2 | 2.7 |
| 113492 Bone2 RA | 100.0 |
| 113493 Synovium2 RA | 1.5 |
| 113494 Syn Fluid Cells RA | 8.1 |
| 113499 Cartilage4 RA | 6.3 |
| 113500 Bone4 RA | 13.8 |
| 113501 Synovium4 RA | 4.6 |
| 113502 Syn Fluid Cells4 RA | 2.4 |
| 113495 Cartilage3 RA | 6.3 |
| 113496 Bone3 RA | 4.5 |
| 113497 Synovium3 RA | 4.2 |
| 113498 Syn Fluid Cells3 RA | 8.7 |
| 117106 Normal Cartilage Rep20 | 0.1 |
| 113663 Bone3 Normal | 1.9 |
| 113664 Synovium3 Normal | 0.0 |
| 113665 Syn Fluid Cells3 Normal | 0.0 |
| 117107 Normal Cartilage Rep22 | 0.2 |
| 113667 Bone4 Normal | 0.5 |
| 113668 Synovium4 Normal | 0.4 |
| 113669 Syn Fluid Cells4 Normal | 0.0 |

Column A - Rel. Exp.(%) Ag428, Run 219421258

TABLE CC
General screening panel v1.4

| Tissue Name | A | B | C | D |
|---|---|---|---|---|
| Adipose | 4.6 | 22.4 | 38.4 | 13.8 |
| Melanoma* Hs688(A).T | 0.0 | 0.0 | 0.0 | 0.0 |
| Melanoma* Hs688(B).T | 0.0 | 0.0 | 0.0 | 0.5 |
| Melanoma* M14 | 0.0 | 0.0 | 0.0 | 0.0 |
| Melanoma* LOXIMVI | 0.0 | 0.0 | 0.0 | 0.0 |
| Melanoma* SK-MEL-5 | 0.0 | 0.0 | 0.0 | 0.0 |
| Squamous cell carcinoma SCC-4 | 0.0 | 0.0 | 0.0 | 0.0 |
| Testis Pool | 0.9 | 2.6 | 9.5 | 4.2 |
| Prostate ca.* (bone met) PC-3 | 0.0 | 0.0 | 0.0 | 0.0 |
| Prostate Pool | 0.6 | 0.5 | 3.1 | 2.3 |
| Placenta | 4.2 | 7.7 | 6.9 | 7.1 |
| Uterus Pool | 0.3 | 0.8 | 0.0 | 2.3 |
| Ovarian ca. OVCAR-3 | 0.0 | 0.0 | 0.0 | 0.0 |
| Ovarian ca. SK-OV-3 | 0.0 | 0.5 | 1.6 | 0.0 |
| Ovarian ca. OVCAR-4 | 0.0 | 0.0 | 0.0 | 0.0 |
| Ovarian ca. OVCAR-5 | 0.0 | 0.0 | 0.0 | 0.0 |
| Ovarian ca. IGROV-1 | 0.0 | 0.6 | 4.0 | 0.0 |
| Ovarian ca. OVCAR-8 | 0.0 | 0.0 | 0.0 | 0.4 |
| Ovary | 1.3 | 3.4 | 9.7 | 2.9 |
| Breast ca. MCF-7 | 0.0 | 0.0 | 1.7 | 0.0 |
| Breast ca. MDA-MB-231 | 0.2 | 0.0 | 0.0 | 0.0 |
| Breast ca. BT 549 | 0.0 | 0.0 | 1.2 | 1.6 |
| Breast ca. T47D | 0.7 | 0.0 | 1.2 | 1.0 |
| Breast ca. MDA-N | 0.0 | 0.0 | 0.0 | 0.0 |
| Breast Pool | 0.8 | 5.5 | 12.8 | 5.1 |
| Trachea | 1.4 | 3.6 | 6.3 | 4.9 |
| Lung | 0.1 | 0.0 | 1.8 | 1.8 |
| Fetal Lung | 5.7 | 14.9 | 18.0 | 17.2 |
| Lung ca. NCI-N417 | 100.0 | 0.0 | 0.0 | 0.0 |
| Lung ca. LX-1 | 0.0 | 0.0 | 0.0 | 0.0 |
| Lung ca. NCI-H146 | 0.0 | 0.2 | 0.0 | 0.6 |
| Lung ca. SHP-77 | 0.0 | 0.0 | 0.0 | 0.0 |
| Lung ca. A549 | 0.0 | 0.9 | 0.8 | 0.0 |
| Lung ca. NCI-H526 | 0.0 | 0.0 | 0.0 | 0.0 |
| Lung ca. NCI-H23 | 0.2 | 0.0 | 0.0 | 0.5 |
| Lung ca. NCI-H460 | 0.0 | 0.3 | 0.0 | 0.0 |
| Lung ca. HOP-62 | 0.0 | 0.0 | 0.0 | 0.0 |
| Lung ca. NCI-H522 | 0.0 | 0.0 | 0.0 | 0.0 |
| Liver | 1.9 | 8.4 | 13.6 | 3.8 |
| Fetal Liver | 7.7 | 24.1 | 20.2 | 15.9 |
| Liver ca. HepG2 | 0.0 | 0.3 | 0.0 | 0.0 |
| Kidney Pool | 1.5 | 7.2 | 10.1 | 6.4 |
| Fetal Kidney | 0.5 | 1.3 | 3.2 | 0.5 |
| Renal ca. 786-0 | 0.0 | 0.0 | 0.0 | 0.0 |
| Renal ca. A498 | 0.0 | 0.0 | 0.0 | 0.0 |
| Renal ca. ACHN | 0.0 | 0.0 | 0.0 | 0.0 |
| Renal ca. UO-31 | 0.2 | 0.5 | 0.0 | 0.0 |
| Renal ca. TK-10 | 0.2 | 1.0 | 0.0 | 0.5 |
| Bladder | 13.2 | 32.5 | 100.0 | 25.7 |
| Gastric ca. (liver met.) NCI-N87 | 0.0 | 0.0 | 0.0 | 0.0 |
| Gastric ca. KATO III | 0.0 | 0.0 | 0.0 | 0.0 |
| Colon ca. SW-948 | 0.0 | 0.0 | 0.0 | 0.0 |
| Colon ca. SW480 | 0.3 | 0.0 | 0.0 | 0.0 |
| Colon ca.* (SW480 met) SW620 | 0.0 | 0.0 | 1.2 | 0.0 |
| Colon ca. HT29 | 0.0 | 0.0 | 0.0 | 0.0 |
| Colon ca. HCT-116 | 0.2 | 0.0 | 2.4 | 0.0 |
| Colon ca. CaCo-2 | 0.2 | 0.7 | 0.0 | 0.0 |
| Colon cancer tissue | 45.4 | 89.5 | 98.6 | 100.0 |
| Colon ca. SW1116 | 0.0 | 0.0 | 0.0 | 0.6 |
| Colon ca. Colo-205 | 0.0 | 0.0 | 0.0 | 0.0 |
| Colon ca. SW-48 | 0.0 | 0.0 | 0.0 | 0.0 |
| Colon Pool | 0.1 | 6.8 | 25.3 | 5.3 |
| Small Intestine Pool | 0.6 | 2.1 | 5.3 | 1.3 |
| Stomach Pool | 3.0 | 6.8 | 10.2 | 9.0 |
| Bone Marrow Pool | 0.6 | 2.8 | 3.4 | 1.0 |
| Fetal Heart | 0.5 | 2.5 | 4.1 | 1.4 |
| Heart Pool | 1.6 | 1.2 | 1.7 | 2.5 |
| Lymph Node Pool | 1.0 | 4.5 | 5.8 | 3.9 |
| Fetal Skeletal Muscle | 0.7 | 1.3 | 2.4 | 0.7 |
| Skeletal Muscle Pool | 2.5 | 9.3 | 10.6 | 8.0 |
| Spleen Pool | 33.9 | 100.0 | 87.7 | 69.7 |
| Thymus Pool | 8.6 | 24.3 | 53.2 | 17.2 |
| CNS cancer (glio/astro) U87-MG | 0.0 | 0.0 | 0.0 | 0.5 |
| CNS cancer (glio/astro) U-118-MG | 0.0 | 0.0 | 0.0 | 0.0 |
| CNS cancer (neuro; met) SK-N-AS | 0.3 | 0.0 | 0.0 | 0.0 |
| CNS cancer (astro) SF-539 | 0.0 | 0.0 | 0.0 | 0.0 |
| CNS cancer (astro) SNB-75 | 0.2 | 0.0 | 0.0 | 0.5 |
| CNS cancer (glio) SNB-19 | 0.6 | 2.1 | 0.0 | 1.6 |
| CNS cancer (glio) SF-295 | 0.5 | 0.8 | 2.5 | 0.0 |
| Brain (Amygdala) Pool | 0.4 | 1.6 | 0.0 | 1.4 |
| Brain (cerebellum) | 0.0 | 0.9 | 0.0 | 0.5 |
| Brain (fetal) | 0.1 | 0.6 | 1.8 | 0.7 |
| Brain (Hippocampus) Pool | 0.1 | 1.4 | 1.4 | 1.3 |
| Cerebral Cortex Pool | 0.3 | 2.7 | 4.3 | 0.0 |
| Brain (Substantia nigra) Pool | 0.1 | 0.8 | 1.0 | 3.5 |
| Brain (Thalamus) Pool | 0.6 | 0.3 | 0.2 | 1.6 |
| Brain (whole) | 1.0 | 1.8 | 1.3 | 1.6 |
| Spinal Cord Pool | 0.5 | 1.4 | 1.2 | 2.7 |
| Adrenal Gland | 4.8 | 10.0 | 26.8 | 15.0 |
| Pituitary gland Pool | 0.1 | 0.5 | 2.9 | 0.0 |
| Salivary Gland | 0.3 | 0.4 | 0.4 | 0.4 |
| Thyroid (female) | 1.4 | 1.6 | 9.3 | 5.1 |
| Pancreatic ca. CAPAN2 | 0.0 | 0.0 | 0.0 | 0.0 |
| Pancreas Pool | 2.6 | 8.0 | 22.1 | 5.0 |

Column A - Rel. Exp.(%) Ag4282 Run 219274018
Column B - Rel. Exp.(%) Ag4282, Run 219288065
Column C - Rel. Exp.(%) Ag4282, Run 220238791
Column D - Rel. Exp.(%) Ag4282, Run 221997754

TABLE CD
Panel 4.1D

| Tissue Name | A |
|---|---|
| Secondary Th1 act | 0.0 |
| Secondary Th2 act | 0.0 |
| Secondary Tr1 act | 0.1 |
| Secondary Th1 rest | 0.1 |
| Secondary Th2 rest | 0.2 |
| Secondary Tr1 rest | 0.0 |
| Primary Th1 act | 0.0 |
| Primary Th2 act | 0.1 |
| Primary Tr1 act | 0.0 |
| Primary Th1 rest | 0.0 |
| Primary Th2 rest | 0.1 |
| Primary Tr1 rest | 0.0 |
| CD45RA CD4 lymphocyte act | 0.1 |
| CD45RO CD4 lymphocyte act | 0.0 |
| CD8 lymphocyte act | 0.0 |
| Secondary CD8 lymphocyte rest | 0.3 |
| Secondary CD8 lymphocyte act | 0.0 |
| CD4 lymphocyte none | 0.3 |
| 2ry Th1/Th2/Tr1 anti-CD95 CH11 | 0.0 |
| LAK cells rest | 29.3 |
| LAK cells IL-2 | 0.2 |
| LAK cells IL-2 + IL-12 | 1.1 |
| LAK cells IL-2 + IFN gamma | 1.6 |
| LAK cells IL-2 + IL-18 | 1.1 |
| LAK cells PMA/ionomycin | 16.7 |
| NK Cells IL-2 rest | 0.1 |
| Two Way MLR 3 day | 5.8 |
| Two Way MLR 5 day | 8.4 |
| Two Way MLR 7 day | 4.7 |
| PBMC rest | 7.1 |
| PBMC PWM | 0.2 |
| PBMC PHA-L | 1.7 |
| Ramos (B cell) none | 0.0 |
| Ramos (B cell) ionomycin | 0.0 |
| B lymphocytes PWM | 0.0 |
| B lymphocytes CD40L and IL-4 | 0.4 |
| EOL-1 dbcAMP | 0.3 |
| EOL-1 dbcAMP PMA/ionomycin | 0.0 |
| Dendritic cells none | 36.9 |
| Dendritic cells LPS | 27.0 |
| Dendritic cells anti-CD40 | 40.9 |
| Monocytes rest | 47.6 |
| Monocytes LPS | 9.9 |
| Macrophages rest | 100.0 |
| Macrophages LPS | 39.8 |
| HUVEC none | 0.0 |
| HUVEC starved | 0.0 |
| HUVEC IL-1beta | 0.0 |
| HUVEC IFN gamma | 0.1 |
| HUVEC TNF alpha + IFN gamma | 0.0 |
| HUVEC TNF alpha + IL4 | 0.0 |
| HUVEC IL-11 | 0.6 |
| Lung Microvascular EC none | 0.2 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.1 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.4 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.1 |
| Dermal fibroblast IFN gamma | 0.1 |
| Dermal fibroblast IL-4 | 0.6 |
| Dermal Fibroblasts rest | 0.3 |
| Neutrophils TNFa + LPS | 2.3 |
| Neutrophils rest | 7.3 |
| Colon | 0.1 |
| Lung | 1.2 |
| Thymus | 0.2 |
| Kidney | 0.0 |

Column A - Rel. Exp.(%) Ag4282, Run 181080868

TABLE CE
general oncology screening panel v 2.4

| Tissue Name | A |
|---|---|
| Colon cancer 1 | 20.4 |
| Colon NAT 1 | 18.9 |
| Colon cancer 2 | 37.9 |
| Colon NAT 2 | 4.7 |
| Colon cancer 3 | 24.7 |
| Colon NAT 3 | 2.0 |
| Colon malignant cancer 4 | 43.8 |
| Colon NAT 4 | 1.7 |
| Lung cancer 1 | 53.2 |
| Lung NAT 1 | 9.9 |
| Lung cancer 2 | 43.8 |
| Lung NAT 2 | 17.3 |
| Squamous cell carcinoma 3 | 45.7 |
| Lung NAT 3 | 3.6 |
| Metastatic melanoma 1 | 4.9 |
| Melanoma 2 | 0.0 |
| Melanoma 3 | 0.0 |
| Metastatic melanoma 4 | 9.0 |
| Metastatic melanoma 5 | 28.7 |
| Bladder cancer 1 | 3.1 |
| Bladder NAT 1 | 0.0 |
| Bladder cancer 2 | 5.0 |
| Bladder NAT 2 | 0.4 |
| Bladder NAT 3 | 0.0 |
| Bladder NAT 4 | 0.5 |
| Prostate adenocarcinoma 1 | 0.0 |
| Prostate adenocarcinoma 2 | 0.6 |
| Prostate adenocarcinoma 3 | 0.5 |
| Prostate adenocarcinoma 4 | 11.8 |
| Prostate NAT 5 | 2.7 |
| Prostate adenocarcinoma 6 | 0.0 |
| Prostate adenocarcinoma 7 | 0.7 |
| Prostate adenocarcinoma 8 | 0.5 |
| Prostate adenocarcinoma 9 | 4.9 |
| Prostate NAT 10 | 0.0 |
| Kidney cancer 1 | 20.9 |
| Kidney NAT 1 | 5.1 |
| Kidney cancer 2 | 100.0 |
| Kidney NAT 2 | 2.0 |
| Kidney cancer 3 | 7.2 |
| Kidney NAT 3 | 1.6 |
| Kidney cancer 4 | 8.9 |
| Kidney NAT 4 | 3.7 |

Column A - Rel. Exp.(%) Ag4282, Run 26280402

AI_comprehensive panel_v1.0 Summary: Ag4282 Highest expression of this gene was detected in sample derived from rheumatoid arthritis (RA) bone (CT=27). High to moderate levels of expression of this gene was also seen in samples derived from osteoarthritic (OA) bone and adjacent bone, OA cartilage, OA synovium and OA synovial fluid samples, as well as from cartilage, bone, synovium and synovial fluid samples from RA patients. Low level expression was also detected in samples derived from normal lung samples, COPD lung, emphysema, atopic asthma, asthma, allergy, Crohn's disease (normal matched control and diseased), ulcerative colitis (normal matched control and diseased), and psoriasis (normal matched control and diseased). Therapeutic modulation of this gene, expressed protein and/or use of antibodies or small molecule drugs targeting the gene or gene product are useful in the treatment of autoimmune and inflammatory disorders including psoriasis, allergy, asthma, inflammatory bowel disease, rheumatoid arthritis and osteoarthritis.

General_screening_panel_v1.4 Summary: Ag4282 The highest expression of this gene was detected in spleen (CTs=29). High levels of expression were also seen in Thymus. Therapeutic modulation of this gene, expressed protein and/or use of antibodies or small molecule drugs targeting the gene or gene product are useful in the treatment of allergies, autoimmune diseases, and inflammatory diseases.

Among tissues with metabolic or endocrine function, this gene was expressed at low to moderate levels in pancreas, adipose, adrenal gland, skeletal muscle, heart, and liver. Glucose phosphorylation, catalyzed by hexokinase, is the first committed step in glucose uptake in skeletal muscle and adipose. Glucose uptake into these tissues is compromised in obesity-related insulin resistance and Type 2 diabetes. Pharmacologic enhancement of hexokinase activity may be a treatment for the prevention and/or treatment of Type 2 diabetes. (Jimenez-Chillaron J C, Metabolism. 2002 January;51(1):121-6). Therapeutic modulation of this gene, expressed protein and/or use of antibodies or small molecule drugs targeting the gene or gene product are useful in the treatment of endocrine/metabolically related diseases, such as obesity and diabetes.

Panel 4.1D Summary: Ag4282 Highest expression of the CG105201-01 gene was detected in resting macrophage (CT=26). High to moderate levels of expression of this gene was also seen in neutrophils, macrophage, monocytes, dendritic cells, two way MLRs, PBMC and LAK cells. Therapeutic modulation of this gene, expressed protein and/or use of antibodies or small molecule drugs targeting the gene or gene product are useful in the treatment of autoimmune and inflammatory diseases including Crohn's disease, ulcerative colitis, multiple sclerosis, chronic obstructive pulmonary disease, asthma, emphysema, rheumatoid arthritis, lupus erythematosus, or psoriasis.

general oncology screening panel_v_{—}2.4 Summary: Ag4282 Highest expression of the CG105201-01 gene was detected in kidney cancer (CT=31.6). Higher expression of this gene was seen in colon, lung, kidney, prostate and metastatic melanoma cancer samples as compared to the adjacent normal tissue. Therefore, expression of this gene is useful as diagnostic marker for these cancers. Therapeutic modulation of this gene, expressed protein and/or use of antibodies or small molecule drugs targeting the gene or gene product are useful in the treatment of colon, lung, kidney, prostate and metastatic melanoma cancers.

D. CG106773-01: Calmodulin-Dependent Protein Kinase II-Delta

Expression of gene CG106773-01 was assessed using the primer-probe set Ag4336, described in Table DA. Results of the RTQ-PCR runs are shown in Tables DB and DC.

TABLE DA

Probe Name Ag4336

| | | | |
|---|---|---|---|
| | | Start | SEQ |
| Primers | Length | Position | ID No |

| | | | | |
|---|---|---|---|---|
| Forward | 5′-tagaatctgccgtctttt | 22 | 186 | 122 |
| | gaag-3′ | | | |
| Probe | TET-5′-caccctaatattgt | 26 | 208 | 123 |
| | gcgacttcatga-3′-TAMRA | | | |
| Reverse | 5′-ccaagtagtgaaagccct | 22 | 244 | 124 |
| | cttc-3′ | | | |

TABLE DB
General screening panel v1.4

| Tissue Name | A |
|---|---|
| Adipose | 11.9 |
| Melanoma* Hs688(A).T | 23.7 |
| Melanoma* Hs688(B).T | 24.1 |
| Melanoma* M14 | 11.9 |
| Melanoma* LOXIMVI | 6.9 |
| Melanoma* SK-MEL-5 | 9.3 |
| Squamous cell carcinoma SCC-4 | 7.8 |
| Testis Pool | 6.5 |
| Prostate ca.* (bone met) PC-3 | 26.4 |
| Prostate Pool | 8.4 |
| Placenta | 1.0 |
| Uterus Pool | 6.7 |
| Ovarian ca. OVCAR-3 | 2.1 |
| Ovarian ca. SK-OV-3 | 12.2 |
| Ovarian ca. OVCAR-4 | 5.9 |
| Ovarian ca. OVCAR-5 | 12.6 |
| Ovarian ca. IGROV-1 | 21.5 |
| Ovarian ca. OVCAR-8 | 12.9 |
| Ovary | 5.4 |
| Breast ca. MCF-7 | 0.2 |
| Breast ca. MDA-MB-231 | 15.6 |
| Breast ca. BT 549 | 100.0 |
| Breast ca. T47D | 20.9 |
| Breast ca. MDA-N | 10.2 |
| Breast Pool | 10.0 |
| Trachea | 11.0 |
| Lung | 3.0 |
| Fetal Lung | 21.5 |
| Lung ca. NCI-N417 | 4.0 |
| Lung ca. LX-1 | 20.2 |
| Lung ca. NCI-H146 | 5.3 |
| Lung ca. SHP-77 | 7.9 |
| Lung ca. A549 | 33.2 |
| Lung ca. NCI-H526 | 9.6 |
| Lung ca. NCI-H23 | 9.9 |
| Lung ca. NCI-H460 | 15.7 |
| Lung ca. HOP-62 | 5.4 |
| Lung ca. NCI-H522 | 0.5 |
| Liver | 0.9 |
| Fetal Liver | 12.5 |
| Liver ca. HepG2 | 10.0 |
| Kidney Pool | 16.0 |
| Fetal Kidney | 12.2 |
| Renal ca. 786-0 | 9.4 |
| Renal ca. A498 | 4.7 |
| Renal ca. ACHN | 20.2 |
| Renal ca. UO-31 | 6.4 |
| Renal ca. TK-10 | 9.7 |
| Bladder | 13.2 |
| Gastric ca. (liver met.) NCI-N87 | 46.0 |
| Gastric ca. KATO III | 19.1 |
| Colon ca. SW-948 | 4.8 |
| Colon ca. SW480 | 4.6 |
| Colon ca.* (SW480 met) SW620 | 12.7 |
| Colon ca. HT29 | 21.9 |
| Colon ca. HCT-116 | 37.9 |
| Colon ca. CaCo-2 | 28.9 |
| Colon cancer tissue | 11.3 |
| Colon ca. SW1116 | 1.7 |
| Colon ca. Colo-205 | 4.5 |
| Colon ca. SW-48 | 3.7 |
| Colon Pool | 9.5 |
| Small Intestine Pool | 13.8 |
| Stomach Pool | 9.0 |
| Bone Marrow Pool | 4.4 |
| Fetal Heart | 32.1 |
| Heart Pool | 15.1 |
| Lymph Node Pool | 10.8 |
| Fetal Skeletal Muscle | 12.4 |
| Skeletal Muscle Pool | 22.5 |
| Spleen Pool | 7.8 |
| Thymus Pool | 10.7 |
| CNS cancer (glio/astro) U87-MG | 39.0 |
| CNS cancer (glio/astro) U-118-MG | 51.4 |
| CNS cancer (neuro; met) SK-N-AS | 31.2 |
| CNS cancer (astro) SF-539 | 10.7 |
| CNS cancer (astro) SNB-75 | 48.6 |
| CNS cancer (glio) SNB-19 | 24.7 |
| CNS cancer (glio) SF-295 | 27.5 |
| Brain (Amygdala) Pool | 14.9 |
| Brain (cerebellum) | 12.2 |
| Brain (fetal) | 18.7 |
| Brain (Hippocampus) Pool | 23.2 |
| Cerebral Cortex Pool | 14.6 |
| Brain (Substantia nigra) Pool | 13.4 |
| Brain (Thalamus) Pool | 29.3 |
| Brain (whole) | 23.3 |
| Spinal Cord Pool | 7.9 |
| Adrenal Gland | 8.0 |
| Pituitary gland Pool | 1.4 |
| Salivary Gland | 2.0 |
| Thyroid (female) | 3.0 |
| Pancreatic ca. CAPAN2 | 6.2 |
| Pancreas Pool | 10.2 |

Column A - Rel. Exp.(%) Ag433, Run 222550704

TABLE DC
Panel 4.1D

| Tissue Name | A |
|---|---|
| Secondary Th1 act | 28.3 |
| Secondary Th2 act | 15.7 |
| Secondary Tr1 act | 12.8 |
| Secondary Th1 rest | 11.2 |
| Secondary Th2 rest | 19.2 |
| Secondary Tr1 rest | 9.8 |
| Primary Th1 act | 15.0 |
| Primary Th2 act | 38.4 |
| Primary Tr1 act | 24.5 |
| Primary Th1 rest | 6.7 |
| Primary Th2 rest | 8.4 |
| Primary Tr1 rest | 15.0 |
| CD45RA CD4 lymphocyte act | 20.0 |
| CD45RO CD4 lymphocyte act | 32.1 |
| CD8 lymphocyte act | 21.6 |
| Secondary CD8 lymphocyte rest | 37.4 |
| Secondary CD8 lymphocyte act | 7.3 |
| CD4 lymphocyte none | 7.5 |
| 2ry Th1/Th2/Tr1 anti-CD95 CH11 | 17.2 |
| LAK cells rest | 40.3 |
| LAK cells IL-2 | 12.6 |
| LAK cells IL-2 + IL-12 | 13.7 |
| LAK cells IL-2 + IFN gamma | 11.4 |
| LAK cells IL-2 + IL-18 | 16.8 |
| LAK cells PMA/ionomycin | 14.4 |
| NK Cells IL-2 rest | 23.8 |
| Two Way MLR 3 day | 14.2 |
| Two Way MLR 5 day | 14.0 |
| Two Way MLR 7 day | 10.4 |
| PBMC rest | 6.6 |
| PBMC PWM | 22.2 |
| PBMC PHA-L | 19.1 |
| Ramos (B cell) none | 25.2 |
| Ramos (B cell) ionomycin | 53.2 |
| B lymphocytes PWM | 16.5 |
| B lymphocytes CD40L and IL-4 | 40.9 |
| EOL-1 dbcAMP | 1.6 |
| EOL-1 dbcAMP PMA/ionomycin | 3.9 |
| Dendritic cells none | 23.0 |
| Dendritic cells LPS | 39.0 |
| Dendritic cells anti-CD40 | 21.2 |
| Monocytes rest | 15.2 |
| Monocytes LPS | 3.0 |
| Macrophages rest | 9.4 |
| Macrophages LPS | 4.6 |
| HUVEC none | 6.2 |
| HUVEC starved | 9.5 |
| HUVEC IL-1beta | 8.5 |
| HUVEC IFN gamma | 22.1 |
| HUVEC TNF alpha + IFN gamma | 10.6 |
| HUVEC TNF alpha + IL4 | 2.4 |
| HUVEC IL-11 | 7.1 |
| Lung Microvascular EC none | 15.5 |
| Lung Microvascular EC TNFalpha + IL-1beta | 9.0 |
| Microvascular Dermal EC none | 13.9 |
| Microsvasular Dermal EC TNFalpha + IL-1beta | 7.1 |
| Bronchial epithelium TNFalpha + IL1beta | 10.0 |
| Small airway epithelium none | 5.8 |
| Small airway epithelium TNFalpha + IL-1beta | 6.3 |
| Coronery artery SMC rest | 7.6 |
| Coronery artery SMC TNFalpha + IL-1beta | 9.0 |
| Astrocytes rest | 19.9 |
| Astrocytes TNFalpha + IL-1beta | 9.7 |
| KU-812 (Basophil) rest | 0.1 |
| KU-812 (Basophil) PMA/ionomycin | 0.0 |
| CCD1106 (Keratinocytes) none | 7.3 |
| CCD1106 (Keratinocytes) TNFalpha + IL-1beta | 11.8 |
| Liver cirrhosis | 8.7 |
| NCI-H292 none | 3.9 |
| NCI-H292 IL-4 | 5.6 |
| NCI-H292 IL-9 | 7.1 |
| NCI-H292 IL-13 | 8.0 |
| NCI-H292 IFN gamma | 6.9 |
| HPAEC none | 7.4 |
| HPAEC TNF alpha + IL-1 beta | 10.2 |
| Lung fibroblast none | 45.4 |
| Lung fibroblast TNF alpha + IL-1 beta | 14.0 |
| Lung fibroblast IL-4 | 43.5 |
| Lung fibroblast IL-9 | 100.0 |
| Lung fibroblast IL-13 | 55.9 |
| Lung fibroblast IFN gamma | 75.3 |
| Dermal fibroblast CCD1070 rest | 29.9 |
| Dermal fibroblast CCD1070 TNF alpha | 40.3 |
| Dermal fibroblast CCD1070 IL-1 beta | 13.6 |
| Dermal fibroblast IFN gamma | 30.1 |
| Dermal fibroblast IL-4 | 45.7 |
| Dermal Fibroblasts rest | 37.9 |
| Neutrophils TNFa + LPS | 0.2 |
| Neutrophils rest | 1.0 |
| Colon | 8.5 |
| Lung | 9.9 |
| Thymus | 14.4 |
| Kidney | 8.7 |

Column A - Rel. Exp.(%) Ag4336, Run 184798102

General_screening_panel_v1.4 Summary: Ag4336 Highest expression of the CG106773-01 gene was detected in breast cancer BT 549 cell line (CT=25). Moderate to high levels of expression of this gene was also seen in cluster of cancer cell lines derived from pancreatic, gastric, colon, lung, renal, breast, ovarian, prostate, squamous cell carcinoma, melanoma and brain cancers. Thus, expression of this gene is useful as a marker to detect the presence of these cancers. Therapeutic modulation of this gene, expressed protein and/or use of antibodies or small molecule drugs targeting the gene or gene product are useful in the treatment of gastric, colon, lung, renal, breast, ovarian, prostate, squamous cell carcinoma, melanoma and brain cancers. The CG106773-01 gene codes for a splice variant of calcium/calmodulin-dependent protein kinase type II delta chain (CaMK II). CaMK-II has been implicated in diverse neuronal and non-neuronal functions, including cell growth control. Splice variants of CaMK-II have been shown to be differentially expressed in tumor cells, especially neuroblastoma and mammary tumor is 5 cells (Tombes R M, Krystal G W., 1997, Biochim Biophys Acta 1355(3):281-92, PMID: 9060999). Therefore, therapeutic modulation of this gene through the use of a small molecule drug are useful in the treatment of neuroblastoma and mammary tumors.

Among tissues with metabolic or endocrine function, this gene was expressed at moderate levels in pancreas, adipose, adrenal gland, thyroid, pituitary gland, skeletal muscle, heart, liver and the gastrointestinal tract. CaMK-II is known to be highly expressed in pancreatic islets and associated with insulin secretion vesicles. Also, the suppression of CaMK II disturbs insulin secretion and insulin gene expression (Rochlitz et al., 2000, Diabetologia 43(4):465-73, PMID: 10819240). Activation of CaMK II and the concomitant phosphorylation of synapsin I contribute to insulin secretion from pancreatic beta-cells (Tabuchi et al., 2000, Endocrinology 141(7):2350-60, PMID: 10875234). Therapeutic modulation of this gene, expressed protein and/or use of antibodies or small molecule drugs targeting the gene or gene product are useful in the treatment of endocrine/metabolically related diseases, such as obesity and diabetes.

This gene was expressed at high levels in all regions of the central nervous system examined, including amygdala, hippocampus, substantia nigra, thalamus, cerebellum, cerebral cortex, and spinal cord. Calmodulin (CaM) is a major Ca2+-binding protein in the brain, where it plays an important role in the neuronal response to changes in the intracellular Ca2+ concentration. Calmodulin modulates numerous Ca2+-dependent enzymes and participates in relevant cellular functions. Among the different CaM-binding proteins, the Ca2+/CaM dependent protein kinase II and the phosphatase calcineurin are especially important in the brain because of their abundance and their participation in numerous neuronal functions (Sola et al., 2001, Int J Biochem Cell Biol 33(5):439-55, PMID: 11331200). In addition, the alpha-CaMK II knock-out mouse and transgenic mice expressing a mutant form of CaMK II clearly demonstrate that CaMK II plays a prominent role in hippocampal LTP and hippocampus-dependent memory (Fukunaga K, Miyamoto E., 2000, Neurosci Res 38(1):3-17, PMID: 10997573). Therapeutic modulation of this gene, expressed protein and/or use of antibodies or small molecule drugs targeting the gene or gene product are useful in the treatment of neurological disorders such as memmory loss, Alzheimer's disease, Parkinson's disease, epilepsy, multiple sclerosis, schizophrenia and depression.

Panel 4.1D Summary: Ag4336 Highest expression of the CG 106773-01 gene was detected in IL-9 treated lung fibroblast cells (CT=27.3). This gene was expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therapeutic modulation of this gene, expressed protein and/or use of antibodies or small molecule drugs targeting the gene or gene product will alter functions associated with these cell types and will improve of the symptoms of patients suffering from autoimmune and inflammatory diseases such as asthma, allergies, inflammatory bowel disease, lupus erythematosus, psoriasis, rheumatoid arthritis, and osteoarthritis.

E. CG119621-02: p59 HCK (Hemopoietic Cell Protein-Tyrosine Kinase)

Expression of gene CG119621-02 was assessed using the primer-probe set Ag7705, described in Table EA. Results of the RTQ-PCR runs are shown in Table EB.

TABLE EA

Probe Name Ag7705

| | | | |
|---|---|---|---|
| | | Start | SEQ |
| Primers | Length | Position | ID No |

| | | | | |
|---|---|---|---|---|
| Forward | 5′-aaggcctccaccgacat- | 17 | 688 | 125 |
| | 3′ | | | |
| Probe | TET-5′-atcgtcttcactgc | 25 | 718 | 126 |
| | caccttggtgt-3′-TAMRA | | | |
| Reverse | 5′-gaccactacaagaccacc | 24 | 747 | 127 |
| | tacaac-3′ | | | |

TABLE EB
General screening panel v1.7

| Tissue Name | A |
|---|---|
| Adipose | 28.9 |
| HUVEC | 0.0 |
| Melanoma* Hs688(A).T | 0.0 |
| Melanoma* Hs688(B).T | 0.0 |
| Melanoma (met) SK-MEL-5 | 0.0 |
| Testis | 2.0 |
| Prostate ca. (bone met) PC-3 | 0.0 |
| Prostate ca. DU145 | 0.0 |
| Prostate pool | 1.7 |
| Uterus pool | 0.0 |
| Ovarian ca. OVCAR-3 | 11.2 |
| Ovarian ca. (ascites) SK-OV-3 | 0.0 |
| Ovarian ca. OVCAR-4 | 1.2 |
| Ovarian ca. OVCAR-5 | 0.5 |
| Ovarian ca. IGROV-1 | 1.1 |
| Ovarian ca. OVCAR-8 | 0.0 |
| Ovary | 7.4 |
| Breast ca. MCF-7 | 4.9 |
| Breast ca. MDA-MB-231 | 0.0 |
| Breast ca. BT 549 | 0.0 |
| Breast ca. T47D | 0.0 |
| 113452 mammary gland | 0.3 |
| Trachea | 12.2 |
| Lung | 100.0 |
| Fetal Lung | 11.5 |
| Lung ca. NCI-N417 | 0.0 |
| Lung ca. LX-1 | 0.0 |
| Lung ca. NCI-H146 | 1.7 |
| Lung ca. SHP-77 | 0.4 |
| Lung ca. NCI-H23 | 3.6 |
| Lung ca. NCI-H460 | 0.0 |
| Lung ca. HOP-62 | 0.0 |
| Lung ca. NCI-H522 | 0.5 |
| Lung ca. DMS-114 | 2.5 |
| Liver | 4.8 |
| Fetal Liver | 14.0 |
| Kidney pool | 5.6 |
| Fetal Kidney | 1.9 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 10.0 |
| Gastric ca. (liver met.) NCI-N87 | 0.0 |
| Stomach | 0.0 |
| Colon ca. SW-948 | 0.0 |
| Colon ca. SW480 | 0.0 |
| Colon ca. (SW480 met) SW620 | 0.2 |
| Colon ca. HT29 | 0.0 |
| Colon ca. HCT-116 | 0.0 |
| Colon cancer tissue | 1.6 |
| Colon ca. SW1116 | 0.0 |
| Colon ca. Colo-205 | 0.0 |
| Colon ca. SW-48 | 0.0 |
| Colon | 6.4 |
| Small Intestine | 0.1 |
| Fetal Heart | 11.9 |
| Heart | 0.6 |
| Lymph Node Pool | 1.0 |
| Lymph Node pool 2 | 53.6 |
| Fetal Skeletal Muscle | 7.3 |
| Skeletal Muscle pool | 0.2 |
| Skeletal Muscle | 2.1 |
| Spleen | 15.0 |
| Thymus | 1.9 |
| CNS cancer (glio/astro) SF-268 | 1.0 |
| CNS cancer (glio/astro) T98G | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) | 3.9 |
| Brain (Cerebellum) | 3.8 |
| Brain (Fetal) | 8.9 |
| Brain (Hippocampus) | 7.0 |
| Cerebral Cortex pool | 2.0 |
| Brain (Substantia nigra) | 1.7 |
| Brain (Thalamus) | 3.7 |
| Brain (Whole) | 7.0 |
| Spinal Cord | 2.4 |
| Adrenal Gland | 31.2 |
| Pituitary Gland | 2.8 |
| Salivary Gland | 2.0 |
| Thyroid | 5.2 |
| Pancreatic ca. PANC-1 | 0.5 |
| Pancreas pool | 0.6 |

Column A - Rel. Exp.(%) Ag770, Run 318009747

General_screening_panel_v1.7 Summary: Ag7705 The highest expression of this gene was detected in lung (CT=29.5). It is also expressed at low levels in fetal lung, adipose, fetal liver, kidney, colon, spleen, thymus, and brain.

This gene was expressed at moderate levels in adipose. Therapeutic modulation of this gene, expressed protein and/or use of antibodies or small molecule drugs targeting the gene or gene product are useful in the treatment of obesity.

F. CG124553-01: Polypeptide N-Acetylgalactosaminyltransferase

Expression of gene CG124553-01 was assessed using the primer-probe set Ag4669, described in Table FA. Results of the RTQ-PCR runs are shown in Table FB.

TABLE FA

Probe Name Ag4669

| | | | |
|---|---|---|---|
| | | Start | SEQ |
| Primers | Length | Position | ID No |

| | | | | |
|---|---|---|---|---|
| Forward | 5′-gcggaagaagaagccata | 22 | 824 | 128 |
| | taat-3′ | | | |
| Probe | TET-5′-tacaccaagaggaa | 26 | 861 | 129 |
| | tgctcttcgcgt-3′-TAMRA | | | |
| Reverse | 5′-gagacttgtaatcgtcca | 22 | 897 | 130 |
| | tcca-3′ | | | |

TABLE FB
General screening panel v1.4

| Tissue Name | A |
|---|---|
| Adipose | 0.2 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 1.5 |
| Prostate ca.* (bone met) PC-3 | 0.0 |
| Prostate Pool | 2.3 |
| Placenta | 0.1 |
| Uterus Pool | 1.5 |
| Ovarian ca. OVCAR-3 | 10.4 |
| Ovarian ca. SK-OV-3 | 3.5 |
| Ovarian ca. OVCAR-4 | 0.0 |
| Ovarian ca. OVCAR-5 | 0.0 |
| Ovarian ca. IGROV-1 | 12.3 |
| Ovarian ca. OVCAR-8 | 6.0 |
| Ovary | 5.1 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 10.7 |
| Trachea | 1.4 |
| Lung | 2.2 |
| Fetal Lung | 15.9 |
| Lung ca. NCI-N417 | 0.7 |
| Lung ca. LX-1 | 0.0 |
| Lung ca. NCI-H146 | 0.2 |
| Lung ca. SHP-77 | 0.0 |
| Lung ca. A549 | 0.0 |
| Lung ca. NCI-H526 | 0.0 |
| Lung ca. NCI-H23 | 0.3 |
| Lung ca. NCI-H460 | 4.9 |
| Lung ca. HOP-62 | 0.0 |
| Lung ca. NCI-H522 | 0.4 |
| Liver | 0.0 |
| Fetal Liver | 0.2 |
| Liver ca. HepG2 | 0.0 |
| Kidney Pool | 5.3 |
| Fetal Kidney | 1.3 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.3 |
| Colon ca. SW1116 | 0.0 |
| Colon ca. Colo-205 | 0.0 |
| Colon ca. SW-48 | 0.0 |
| Colon Pool | 14.3 |
| Small Intestine Pool | 1.2 |
| Stomach Pool | 2.7 |
| Bone Marrow Pool | 1.9 |
| Fetal Heart | 5.4 |
| Heart Pool | 4.2 |
| Lymph Node Pool | 9.5 |
| Fetal Skeletal Muscle | 11.2 |
| Skeletal Muscle Pool | 0.0 |
| Spleen Pool | 2.2 |
| Thymus Pool | 4.9 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 12.0 |
| CNS cancer (glio) SF-295 | 0.0 |
| Brain (Amygdala) Pool | 22.5 |
| Brain (cerebellum) | 66.9 |
| Brain (fetal) | 19.8 |
| Brain (Hippocampus) Pool | 22.5 |
| Cerebral Cortex Pool | 36.3 |
| Brain (Substantia nigra) Pool | 54.0 |
| Brain (Thalamus) Pool | 35.8 |
| Brain (whole) | 100.0 |
| Spinal Cord Pool | 1.9 |
| Adrenal Gland | 4.7 |
| Pituitary gland Pool | 2.0 |
| Salivary Gland | 0.8 |
| Thyroid (female) | 2.6 |
| Pancreatic ca. CAPAN2 | 0.0 |
| Pancreas Pool | 5.7 |

Column A - Rel. Exp.(%) Ag466, Run 222811492

General_screening_panel_v1.4 Summary: Ag4669 The highest expression on this panel was observed in the whole brain (CT=25), with high levels of expression seen throughout the CNS. This expression suggests that that this putative enzyme may catalyze O-glycosylation in the brain. Therapeutic modulation of this gene, expressed protein and/or use of antibodies or small molecule drugs targeting the gene or gene product are useful in the treatment of neurological disorders, such as Alzheimer's disease, Parkinson's disease, schizophrenia, multiple sclerosis, stroke and epilepsy.

Among tissues with metabolic function, this gene was expressed at moderate to low levels in pituitary, adipose, adrenal gland, pancreas, thyroid, fetal liver and skeletal muscle, and adult and fetal heart. Therapeutic modulation of this gene, expressed protein and/or use of antibodies or small molecule drugs targeting the gene or gene product are useful in the treatment of endocrine/metabolically related diseases, such as obesity and diabetes.

G. CG187738-02: Cytosolic Branched Chain Aminotransferase

Expression of gene CG187738-02 was assessed using the primer-probe sets Ag7879 and Ag7805, described in Tables GA and GB. Results of the RTQ-PCR runs are shown in Table GC.

TABLE GA

Probe Name Ag7879

| | | | |
|---|---|---|---|
| | | Start | SEQ |
| Primers | Length | Position | ID No |

| | | | | |
|---|---|---|---|---|
| Forward | 5′-cctgtgttgtttgcccag | 20 | 148 | 131 |
| | tt-3′ | | | |
| Probe | TET-5′-aaggcgagacaata | 26 | 106 | 132 |
| | cacattccaact-3′-TAMRA | | | |
| Reverse | 5′-cttgccagcttaggacca | 20 | 80 | 133 |
| | tt-3′ | | | |

TABLE GB

Probe Name Ag7805

| | | | |
|---|---|---|---|
| | | Start | SEQ |
| Primers | Length | Position | ID No |

| | | | | |
|---|---|---|---|---|
| Forward | 5′-agctcttctttgtcaaat | 25 | 758 | 134 |
| | acttcca-3′ | | | |
| Probe | TET-5′-accctggctcatca | 26 | 787 | 135 |
| | gctttgcactat-3′-TAMRA | | | |
| Reverse | 5′-agaaacctcatatcaagc | 24 | 828 | 136 |
| | ctcttc-3′ | | | |

TABLE GC
General screening panel v1.7

| Tissue Name | A | B |
|---|---|---|
| Adipose | 2.9 | 6.6 |
| HUVEC | 80.1 | 94.0 |
| Melanoma* Hs688(A).T | 0.0 | 0.0 |
| Melanoma* Hs688(B).T | 32.8 | 63.7 |
| Melanoma (met) SK-MEL-5 | 0.0 | 0.0 |
| Testis | 1.5 | 5.0 |
| Prostate ca. (bone met) PC-3 | 0.2 | 0.0 |
| Prostate ca. DU145 | 17.9 | 17.0 |
| Prostate pool | 0.1 | 0.2 |
| Uterus pool | 0.2 | 0.3 |
| Ovarian ca. OVCAR-3 | 3.4 | 5.5 |
| Ovarian ca. (ascites) SK-OV-3 | 0.6 | 1.7 |
| Ovarian ca. OVCAR-4 | 26.8 | 41.8 |
| Ovarian ca. OVCAR-5 | 0.0 | 0.0 |
| Ovarian ca. IGROV-1 | 0.2 | 0.4 |
| Ovarian ca. OVCAR-8 | 41.5 | 62.0 |
| Ovary | 1.4 | 3.5 |
| Breast ca. MCF-7 | 0.0 | 0.0 |
| Breast ca. MDA-MB-231 | 44.8 | 47.0 |
| Breast ca. BT 549 | 2.3 | 5.8 |
| Breast ca. T47D | 0.0 | 0.0 |
| 113452 mammary gland | 0.6 | 0.0 |
| Trachea | 1.9 | 5.3 |
| Lung | 4.2 | 9.6 |
| Fetal Lung | 0.9 | 2.3 |
| Lung ca. NCI-N417 | 6.0 | 10.1 |
| Lung ca. LX-1 | 0.0 | 0.2 |
| Lung ca. NCI-H146 | 0.0 | 0.0 |
| Lung ca. SHP-77 | 0.0 | 0.0 |
| Lung ca. NCI-H23 | 2.3 | 1.8 |
| Lung ca. NCI-H460 | 27.2 | 21.0 |
| Lung ca. HOP-62 | 55.1 | 100.0 |
| Lung ca. NCI-H522 | 0.4 | 0.3 |
| Lung ca. DMS-114 | 0.1 | 0.3 |
| Liver | 0.0 | 0.1 |
| Fetal Liver | 1.1 | 1.1 |
| Kidney pool | 0.5 | 0.0 |
| Fetal Kidney | 1.5 | 2.9 |
| Renal ca. 786-0 | 17.3 | 16.2 |
| Renal ca. A498 | 2.5 | 2.8 |
| Renal ca. ACHN | 13.1 | 41.5 |
| Renal ca. UO-31 | 7.9 | 14.3 |
| Renal ca. TK-10 | 12.3 | 9.2 |
| Bladder | 1.0 | 2.0 |
| Gastric ca. (liver met.) NCI-N87 | 0.0 | 0.0 |
| Stomach | 0.2 | 0.4 |
| Colon ca. SW-948 | 0.0 | 0.0 |
| Colon ca. SW480 | 0.0 | 0.0 |
| Colon ca. (SW480 met) SW620 | 0.0 | 0.2 |
| Colon ca. HT29 | 0.0 | 0.0 |
| Colon ca. HCT-116 | 0.0 | 0.0 |
| Colon cancer tissue | 0.2 | 0.5 |
| Colon ca. SW1116 | 0.0 | 0.0 |
| Colon ca. Colo-205 | 0.0 | 0.0 |
| Colon ca. SW-48 | 0.0 | 0.0 |
| Colon | 0.4 | 0.7 |
| Small Intestine | 0.2 | 1.0 |
| Fetal Heart | 0.5 | 2.2 |
| Heart | 0.1 | 0.6 |
| Lymph Node Pool | 0.5 | 0.7 |
| Lymph Node pool 2 | 2.6 | |
| Fetal Skeletal Muscle | 0.6 | 2.7 |
| Skeletal Muscle pool | 0.1 | 0.1 |
| Skeletal Muscle | 0.1 | 0.3 |
| Spleen | 0.5 | 0.8 |
| Thymus | 0.4 | 1.2 |
| CNS cancer (glio/astro) SF-268 | 24.1 | 25.9 |
| CNS cancer (glio/astro) T98G | 9.0 | 12.3 |
| CNS cancer (neuro; met) SK-N-AS | 1.3 | 0.1 |
| CNS cancer (astro) SF-539 | 100.0 | 74.7 |
| CNS cancer (astro) SNB-75 | 19.5 | 19.6 |
| CNS cancer (glio) SNB-19 | 18.3 | 17.1 |
| CNS cancer (glio) SF-295 | 5.1 | 4.1 |
| Brain (Amygdala) | 1.2 | 4.5 |
| Brain (Cerebellum) | 0.6 | 2.1 |
| Brain (Fetal) | 1.4 | 6.7 |
| Brain (Hippocampus) | 0.5 | 2.8 |
| Cerebral Cortex pool | 0.9 | 2.9 |
| Brain (Substantia nigra) | 0.1 | 1.2 |
| Brain (Thalamus) | 0.7 | 4.5 |
| Brain (Whole) | 2.9 | 15.9 |
| Spinal Cord | 0.3 | 1.3 |
| Adrenal Gland | 1.2 | 3.0 |
| Pituitary Gland | 3.1 | 7.5 |
| Salivary Gland | 0.1 | 0.4 |
| Thyroid | 0.3 | 1.1 |
| Pancreatic ca. PANC-1 | 0.2 | 0.1 |
| Pancreas pool | 1.5 | 4.5 |

Column A - Rel. Exp.(%) Ag7805 Run 319066029
Column B - Rel. Exp.(%) Ag7879, Run 318008710

General_screening panel_v1.7 Summary: Ag7805/Ag7879 The highest expression of this gene was detected in CNS cancer (astrocytoma) and lung cancer cell lines (CT=25-26). This gene was overexpresssed in several cancer cell lines including melanoma, prostate, ovarian, breast, lung renal and CNS cancers. Therapeutic modulation of this gene, expressed protein and/or use of antibodies or small molecule drugs targeting the gene or gene product are useful in the treatment of these cancers.

Among tissues with metabolic or endocrine function, this gene was expressed at high to moderate levels in pancreas, adipose, adrenal gland, thyroid, pituitary gland, skeletal muscle, heart, liver and the gastrointestinal tract. Therapeutic modulation of this gene, expressed protein and/or use of antibodies or small molecule drugs targeting the gene or gene product are useful in the treatment of endocrine/metabolically related diseases, such as obesity and diabetes.

H. CG57589-01 and CG57589-03: Cholinephosphate Cytidylyltransferase

Expression of genes CG57589-01 and CG57589-03 was assessed using the primer-probe set Ag4146, described in Table HA. Results of the RTQ-PCR runs are shown in Table HB.

TABLE HA

Probe Name Ag4146

| | | | |
|---|---|---|---|
| | | Start | SEQ |
| Primers | Length | Position | ID No |

| | | | | |
|---|---|---|---|---|
| Forward | 5′-catcttccgtcagattga | 22 | 975 | 137 |
| | cagt-3′ | | | |
| Probe | TET-5′-agcaacctcaccac | 26 | 1000 | 138 |
| | agacctcatcgt-3′-TAMRA | | | |
| Reverse | 5′-atactccaacctgttggt | 22 | 1035 | 139 |
| | gatg-3′ | | | |

TABLE HB
General screening panel v1.4

| Tissue Name | A |
|---|---|
| Adipose | 0.8 |
| Melanoma* Hs688(A).T | 4.7 |
| Melanoma* Hs688(B).T | 4.8 |
| Melanoma* M14 | 11.1 |
| Melanoma* LOXIMVI | 5.0 |
| Melanoma* SK-MEL-5 | 21.5 |
| Squamous cell carcinoma SCC-4 | 4.7 |
| Testis Pool | 27.9 |
| Prostate ca.* (bone met) PC-3 | 3.3 |
| Prostate Pool | 2.3 |
| Placenta | 2.7 |
| Uterus Pool | 0.4 |
| Ovarian ca. OVCAR-3 | 15.4 |
| Ovarian ca. SK-OV-3 | 11.4 |
| Ovarian ca. OVCAR-4 | 12.4 |
| Ovarian ca. OVCAR-5 | 32.3 |
| Ovarian ca. IGROV-1 | 11.0 |
| Ovarian ca. OVCAR-8 | 5.1 |
| Ovary | 2.3 |
| Breast ca. MCF-7 | 14.0 |
| Breast ca. MDA-MB-231 | 21.9 |
| Breast ca. BT 549 | 11.5 |
| Breast ca. T47D | 100.0 |
| Breast ca. MDA-N | 10.9 |
| Breast Pool | 1.6 |
| Trachea | 4.1 |
| Lung | 0.4 |
| Fetal Lung | 3.9 |
| Lung ca. NCI-N417 | 2.6 |
| Lung ca. LX-1 | 13.7 |
| Lung ca. NCI-H146 | 1.8 |
| Lung ca. SHP-77 | 7.4 |
| Lung ca. A549 | 5.4 |
| Lung ca. NCI-H526 | 5.5 |
| Lung ca. NCI-H23 | 2.2 |
| Lung ca. NCI-H460 | 1.2 |
| Lung ca. HOP-62 | 5.8 |
| Lung ca. NCI-H522 | 6.3 |
| Liver | 7.3 |
| Fetal Liver | 14.2 |
| Liver ca. HepG2 | 29.5 |
| Kidney Pool | 2.8 |
| Fetal Kidney | 3.3 |
| Renal ca. 786-0 | 11.3 |
| Renal ca. A498 | 4.9 |
| Renal ca. ACHN | 9.9 |
| Renal ca. UO-31 | 4.2 |
| Renal ca. TK-10 | 19.3 |
| Bladder | 4.2 |
| Gastric ca. (liver met.) NCI-N87 | 22.7 |
| Gastric ca. KATO III | 49.3 |
| Colon ca. SW-948 | 16.2 |
| Colon ca. SW480 | 24.1 |
| Colon ca.* (SW480 met) SW620 | 12.2 |
| Colon ca. HT29 | 13.8 |
| Colon ca. HCT-116 | 25.0 |
| Colon ca. CaCo-2 | 26.2 |
| Colon cancer tissue | 2.2 |
| Colon ca. SW1116 | 4.9 |
| Colon ca. Colo-205 | 12.0 |
| Colon ca. SW-48 | 6.5 |
| Colon Pool | 1.8 |
| Small Intestine Pool | 0.9 |
| Stomach Pool | 1.0 |
| Bone Marrow Pool | 0.6 |
| Fetal Heart | 2.9 |
| Heart Pool | 1.7 |
| Lymph Node Pool | 2.1 |
| Fetal Skeletal Muscle | 0.8 |
| Skeletal Muscle Pool | 3.7 |
| Spleen Pool | 0.8 |
| Thymus Pool | 1.6 |
| CNS cancer (glio/astro) U87-MG | 26.8 |
| CNS cancer (glio/astro) U-118-MG | 15.0 |
| CNS cancer (neuro; met) SK-N-AS | 12.5 |
| CNS cancer (astro) SF-539 | 22.4 |
| CNS cancer (astro) SNB-75 | 17.6 |
| CNS cancer (glio) SNB-19 | 12.2 |
| CNS cancer (glio) SF-295 | 10.9 |
| Brain (Amygdala) Pool | 6.1 |
| Brain (cerebellum) | 12.6 |
| Brain (fetal) | 7.1 |
| Brain (Hippocampus) Pool | 6.2 |
| Cerebral Cortex Pool | 6.8 |
| Brain (Substantia nigra) Pool | 10.1 |
| Brain (Thalamus) Pool | 10.4 |
| Brain (whole) | 8.9 |
| Spinal Cord Pool | 5.3 |
| Adrenal Gland | 3.1 |
| Pituitary gland Pool | 0.7 |
| Salivary Gland | 1.8 |
| Thyroid (female) | 2.0 |
| Pancreatic ca. CAPAN2 | 12.5 |
| Pancreas Pool | 2.8 |

Column A - Rel. Exp.(%) Ag4146 Run 221290908

General_screening_panel_v1.4 Summary: Ag4146 The highest expression of this gene was detected in a breast cancer cell line (T47D)(CT=27). There was significant expression in other samples derived from breast cancer cell lines, ovarian cancer cell lines, brain cancer cell lines and colon cancer cell lines. Therapeutic modulation of this gene, expressed protein and/or use of antibodies or small molecule drugs targeting the gene or gene product are useful in the treatment of breast, ovarian, brain or colon cancer.

Among metabolic tissues, this gene had low levels of expression in adipose, heart, skeletal muscle, adrenal, pituitary, thyroid, and pancreas. Dysregulated levels of phosphatidylcholine may be involved in the pathogenesis of disease in these tissues. Therapeutic modulation of this gene, expressed protein and/or use of antibodies or small molecule drugs targeting the gene or gene product are useful in the treatment of metabolic and endocrine disease, including Types 1 and 2 diabetes and obesity.

This gene, a cholinephosphate cytidylyltransferase homolog, was also expressed at is moderate levels in all CNS regions examined. This protein has been shown to be upregulated in the brain during triethyltin-induced cerebral edema (Mages F. et al., Pharmacol Toxicol 1989 October;65(4):302-5). Therapeutic modulation of this gene, expressed protein and/or use of antibodies or small molecule drugs targeting the gene or gene product are useful in the treatment of cerebral edema.

I. CG91149-03: AMP-Activated Protein Kinase

Expression of gene CG91149-03 was assessed using the primer-probe sets ag3673 and Ag7438, described in Tables IA and IB. Results of the RTQ-PCR runs are shown in Tables IC and ID.

TABLE IA

Probe Name ag3673

| | | | |
|---|---|---|---|
| | | Start | SEQ |
| Primers | Length | Position | ID No |

| | | | | |
|---|---|---|---|---|
| Forward | 5′-aacagaaatcaccaggat | 22 | 1011 | 140 |
| | cctt-3′ | | | |
| Probe | TET-5′-tggcagttgcctac | 26 | 1033 | 141 |
| | catctcataata-3′-TAMRA | | | |
| Reverse | 5′-tgtcgccaaatagaaatc | 22 | 1085 | 142 |
| | tttg-3′ | | | |

TABLE IB

Probe Name Ag7438

| | | | |
|---|---|---|---|
| | | Start | SEQ |
| Primers | Length | Position | ID No |

| | | | | |
|---|---|---|---|---|
| Forward | 5′-accatctgatattttcat | 24 | 284 | 143 |
| | ggtgat-3′ | | | |
| Probe | TET-5′-tcaaatagctctcc | 30 | 308 | 144 |
| | tcctgagacatattcc- | | | |
| | 3′-TAMRA | | | |
| Reverse | 5′-gtacatcagatttccttc | 27 | 346 | 145 |
| | cattcttac-3′ | | | |

TABLE IC
General screening panel v1.4

| Tissue Name | A |
|---|---|
| Adipose | 10.7 |
| Melanoma* Hs688(A).T | 41.5 |
| Melanoma* Hs688(B).T | 39.0 |
| Melanoma* M14 | 23.3 |
| Melanoma* LOXIMVI | 47.6 |
| Melanoma* SK-MEL-5 | 48.3 |
| Squamous cell carcinoma SCC-4 | 39.5 |
| Testis Pool | 24.5 |
| Prostate ca.* (bone met) PC-3 | 49.7 |
| Prostate Pool | 12.7 |
| Placenta | 3.2 |
| Uterus Pool | 7.6 |
| Ovarian ca. OVCAR-3 | 28.5 |
| Ovarian ca. SK-OV-3 | 41.5 |
| Ovarian ca. OVCAR-4 | 11.9 |
| Ovarian ca. OVCAR-5 | 55.5 |
| Ovarian ca. IGROV-1 | 24.5 |
| Ovarian ca. OVCAR-8 | 12.7 |
| Ovary | 12.4 |
| Breast ca. MCF-7 | 19.8 |
| Breast ca. MDA-MB-231 | 56.3 |
| Breast ca. BT 549 | 69.7 |
| Breast ca. T47D | 100.0 |
| Breast ca. MDA-N | 17.0 |
| Breast Pool | 26.6 |
| Trachea | 21.2 |
| Lung | 6.7 |
| Fetal Lung | 66.4 |
| Lung ca. NCI-N417 | 6.6 |
| Lung ca. LX-1 | 42.3 |
| Lung ca. NCI-H146 | 6.2 |
| Lung ca. SHP-77 | 41.5 |
| Lung ca. A549 | 46.7 |
| Lung ca. NCI-H526 | 7.8 |
| Lung ca. NCI-H23 | 65.5 |
| Lung ca. NCI-H460 | 27.4 |
| Lung ca. HOP-62 | 21.8 |
| Lung ca. NCI-H522 | 31.6 |
| Liver | 1.8 |
| Fetal Liver | 37.1 |
| Liver ca. HepG2 | 19.5 |
| Kidney Pool | 41.2 |
| Fetal Kidney | 41.5 |
| Renal ca. 786-0 | 68.3 |
| Renal ca. A498 | 16.5 |
| Renal ca. ACHN | 16.5 |
| Renal ca. UO-31 | 30.8 |
| Renal ca. TK-10 | 29.3 |
| Bladder | 30.1 |
| Gastric ca. (liver met.) NCI-N87 | 72.2 |
| Gastric ca. KATO III | 61.6 |
| Colon ca. SW-948 | 9.5 |
| Colon ca. SW480 | 37.6 |
| Colon ca.* (SW480 met) SW620 | 40.6 |
| Colon ca. HT29 | 48.3 |
| Colon ca. HCT-116 | 41.2 |
| Colon ca. CaCo-2 | 50.0 |
| Colon cancer tissue | 25.5 |
| Colon ca. SW1116 | 5.6 |
| Colon ca. Colo-205 | 8.7 |
| Colon ca. SW-48 | 13.8 |
| Colon Pool | 29.3 |
| Small Intestine Pool | 25.0 |
| Stomach Pool | 15.7 |
| Bone Marrow Pool | 13.5 |
| Fetal Heart | 19.5 |
| Heart Pool | 13.4 |
| Lymph Node Pool | 34.4 |
| Fetal Skeletal Muscle | 9.6 |
| Skeletal Muscle Pool | 8.8 |
| Spleen Pool | 17.1 |
| Thymus Pool | 20.0 |
| CNS cancer (glio/astro) U87-MG | 35.6 |
| CNS cancer (glio/astro) U-118-MG | 73.7 |
| CNS cancer (neuro; met) SK-N-AS | 34.9 |
| CNS cancer (astro) SF-539 | 15.4 |
| CNS cancer (astro) SNB-75 | 69.3 |
| CNS cancer (glio) SNB-19 | 27.0 |
| CNS cancer (glio) SF-295 | 79.0 |
| Brain (Amygdala) Pool | 7.9 |
| Brain (cerebellum) | 14.1 |
| Brain (fetal) | 10.3 |
| Brain (Hippocampus) Pool | 7.8 |
| Cerebral Cortex Pool | 12.2 |
| Brain (Substantia nigra) Pool | 8.1 |
| Brain (Thalamus) Pool | 15.5 |
| Brain (whole) | 8.5 |
| Spinal Cord Pool | 10.2 |
| Adrenal Gland | 11.7 |
| Pituitary gland Pool | 6.0 |
| Salivary Gland | 6.2 |
| Thyroid (female) | 8.3 |
| Pancreatic ca. CAPAN2 | 34.2 |
| Pancreas Pool | 30.8 |

Column A - Rel. Exp.(%) Ag367, Run 218901791

TABLE ID
Panel 4.1D

| Tissue Name | A |
|---|---|
| Secondary Th1 act | 39.0 |
| Secondary Th2 act | 84.1 |
| Secondary Tr1 act | 30.1 |
| Secondary Th1 rest | 3.3 |
| Secondary Th2 rest | 6.3 |
| Secondary Tr1 rest | 8.8 |
| Primary Th1 act | 3.0 |
| Primary Th2 act | 77.4 |
| Primary Tr1 act | 43.5 |
| Primary Th1 rest | 3.3 |
| Primary Th2 rest | 4.5 |
| Primary Tr1 rest | 25.5 |
| CD45RA CD4 lymphocyte act | 51.8 |
| CD45RO CD4 lymphocyte act | 57.8 |
| CD8 lymphocyte act | 8.0 |
| Secondary CD8 lymphocyte rest | 7.7 |
| Secondary CD8 lymphocyte act | 10.6 |
| CD4 lymphocyte none | 11.7 |
| 2ry Th1/Th2/Tr1 anti-CD95 CH11 | 7.8 |
| LAK cells rest | 30.1 |
| LAK cells IL-2 | 17.4 |
| LAK cells IL-2 + IL-12 | 2.1 |
| LAK cells IL-2 + IFN gamma | 16.8 |
| LAK cells IL-2 + IL-18 | 11.6 |
| LAK cells PMA/ionomycin | 35.4 |
| NK Cells IL-2 rest | 88.9 |
| Two Way MLR 3 day | 16.7 |
| Two Way MLR 5 day | 9.2 |
| Two Way MLR 7 day | 8.9 |
| PBMC rest | 9.4 |
| PBMC PWM | 13.8 |
| PBMC PHA-L | 6.3 |
| Ramos (B cell) none | 13.3 |
| Ramos (B cell) ionomycin | 33.2 |
| B lymphocytes PWM | 26.2 |
| B lymphocytes CD40L and IL-4 | 54.3 |
| EOL-1 dbcAMP | 58.6 |
| EOL-1 dbcAMP PMA/ionomycin | 2.4 |
| Dendritic cells none | 8.2 |
| Dendritic cells LPS | 6.7 |
| Dendritic cells anti-CD40 | 9.3 |
| Monocytes rest | 14.4 |
| Monocytes LPS | 18.7 |
| Macrophages rest | 7.3 |
| Macrophages LPS | 0.0 |
| HUVEC none | 11.7 |
| HUVEC starved | 24.3 |
| HUVEC IL-1beta | 36.3 |
| HUVEC IFN gamma | 66.4 |
| HUVEC TNF alpha + IFN gamma | 10.3 |
| HUVEC TNF alpha + IL4 | 5.2 |
| HUVEC IL-11 | 17.7 |
| Lung Microvascular EC none | 81.2 |
| Lung Microvascular EC TNFalpha + IL-1beta | 14.6 |
| Microvascular Dermal EC none | 11.2 |
| Microsvasular Dermal EC TNFalpha + IL-1beta | 10.6 |
| Bronchial epithelium TNFalpha + IL1beta | 16.3 |
| Small airway epithelium none | 8.3 |
| Small airway epithelium TNFalpha + IL-1beta | 30.8 |
| Coronery artery SMC rest | 27.0 |
| Coronery artery SMC TNFalpha + IL-1beta | 27.2 |
| Astrocytes rest | 1.2 |
| Astrocytes TNFalpha + IL-1beta | 3.6 |
| KU-812 (Basophil) rest | 34.4 |
| KU-812 (Basophil) PMA/ionomycin | 47.6 |
| CCD1106 (Keratinocytes) none | 63.3 |
| CCD1106 (Keratinocytes) TNFalpha + IL-1beta | 9.0 |
| Liver cirrhosis | 15.6 |
| NCI-H292 none | 100.0 |
| NCI-H292 IL-4 | 52.5 |
| NCI-H292 IL-9 | 55.9 |
| NCI-H292 IL-13 | 61.1 |
| NCI-H292 IFN gamma | 19.6 |
| HPAEC none | 6.8 |
| HPAEC TNF alpha + IL-1 beta | 59.9 |
| Lung fibroblast none | 15.4 |
| Lung fibroblast TNF alpha + IL-1 beta | 36.9 |
| Lung fibroblast IL-4 | 26.4 |
| Lung fibroblast IL-9 | 13.7 |
| Lung fibroblast IL-13 | 15.3 |
| Lung fibroblast IFN gamma | 32.8 |
| Dermal fibroblast CCD1070 rest | 26.1 |
| Dermal fibroblast CCD1070 TNF alpha | 62.4 |
| Dermal fibroblast CCD1070 IL-1 beta | 25.7 |
| Dermal fibroblast IFN gamma | 23.3 |
| Dermal fibroblast IL-4 | 36.1 |
| Dermal Fibroblasts rest | 17.8 |
| Neutrophils TNFa + LPS | 12.1 |
| Neutrophils rest | 85.9 |
| Colon | 6.9 |
| Lung | 13.5 |
| Thymus | 4.1 |
| Kidney | 31.9 |

Column A -Rel. Exp.(%) Ag7438, Run 305901959

General_screening_panel_v1.4 Summary: Ag3673 The highest expression of this gene was detected in the breast cancer cell line T47D (CT=27). This gene was upregulated in cell lines derived from melanoma, breast cancer, lung cancer, colon cancer, ovarian cancer, and various brain cancers. Therapeutic modulation of this gene, expressed protein and/or use of antibodies or small molecule drugs targeting the gene or gene product are useful in the treatment of these cancers.

Among tissues with metabolic or endocrine function, this gene was expressed at moderate to low levels in pancreas, adipose, adrenal gland, thyroid, pituitary gland, skeletal muscle, heart, liver and the gastrointestinal tract. Therefore, therapeutic modulation of the activity of this gene is useful in the treatment of endocrine/metabolically related diseases, such as obesity and diabetes.

Panel 4.1D Summary: Ag7438 The highest expression of this gene was detected in NCI-H292 cells. This transcript was upregulated in chronically activated Th1, Th2 and Tr1 cells. Macrophages and dendritic cells also express the transcript. Thus, this transcript or the protein it encodes can be used to detect hematopoietically-derived cells. Therapeutic modulation of this gene, expressed protein and/or use of antibodies or small molecule drugs targeting the gene or gene product are useful in the regulation of the function of antigen presenting cells (macrophages and dendritic cells) or T cells, and are useful in the treatment of asthma, emphysema, psoriasis, arthritis, and IBD.

### Example D

### Gene Expression Analysis Using CuraChip in Human Tissues from Tumors and from Equivalent Normal Tissues

Background: CuraGen has developed a gene microarray (CuraChip 1.2) for target identification. It provides a high-throughput means of global mRNA expression analyses of CuraGen's collection of cDNA sequences representing the Pharmaceutically Tractable Genome (PTG). This sequence set includes genes which can be developed into protein therapeutics, or used to develop antibody or small molecule therapeutics. CuraChip 1.2 contains ˜11,000 oligos representing approximately 8,500 gene loci, including (but not restricted to) kinases, ion channels, G-protein coupled receptors (GPCRs), nuclear hormone receptors, proteases, transporters, metabolic enzymes, hormones, growth factors, chemokines, cytokines, complement and coagulation factors, and cell surface receptors.

The CuraChip cDNAs were represented as 30-mer oligodeoxyribonucleotides (oligos) on a glass microchip. Hybridization methods using the longer CuraChip oligos are more specific compared to methods using 25-mer oligos. CuraChip oligos were synthesized with a linker, purified to remove truncated oligos (which can influence hybridization strength and specificity), and spotted on a glass slide. Oligo-dT primers were used to generate cRNA probes for hybridization from samples of interest. A biotin-avidin conjugation system was used to detect hybridized probes with a fluorophore-labeled secondary antibody. Gene expression was analyzed using clustering and correlation bioinformatics tools such as Spotfire® (Spotfire, Inc., 212 μm Street, Somerville, Mass. 02144) and statistical tools such as multivariate analysis (MVA).

Normalization Method Used in CuraChip Software

The median fluorescence intensity of each spot and a background for each spot is read on a scale from 0 to 65,000. CuraGen's CuraChip software, developed in-house, has the capability to present the user with either the raw data (median intensities) or normalized data. If normalized data is chosen, the CuraChip software uses the following method to do mean normalization. The normalization is based on each slide to allow comparison across slides. Given the following:
fg_median is signal/foreground median for each hybridization spot;
bg_median is background median for each hybridization spot;
signal_value is the difference between fg_median and bg_median;
flag indicates whether the experiment was a success or failure, 0 means success and 1 means failure;
foreground is the raw foreground value for each slide/experiment;
background is the raw background value for each slide/experiment;
nSpots is the number of spots in each slide/experiment
nSlides is the number of slides in each analysis (samples grouped by scientist)
fg_mean is trimmed foreground mean for each slide/experiment;
bg_mean is trimmed background mean for each slide/experiment;
norm_constant is the constant 1120.0, which is the value of the mean of the 90^{th} percentile value for all samples (excluding controls).
normalized_value is the final normalized value;
coeff is the normalization coefficient.
MAX_VALUE is the constant, currently 65,000.

For each slide/experiment, the trimmed foreground mean and the trimmed background mean is first calculated for all nSpots:

For each successful spot where flag equals 0 (whether a spot passed or failed is determined by the lab), the foreground and background values are set,

signal_value = fg_median − bg_median;
if (flag = = 0)
{

foreground = signal_value;
background = bg_median;

| } | |
|---|---|
| else | // we can not use the data |
| { | |

foreground = 0.0;
background = 0.0;

}

After that, there are nSpot number of foreground values and background values for each is slide/experiment. Each list is then sorted:

foreground[1], foreground[2], ..., foreground[N]; N = 1, nSpots;
background[1], background[2], ..., background[N]; N = 1, nSpots;
Next, the 90^{th} percentile value data is calculated:
if nspots * 0.90 is an integer

90^{th} percentile value =

foreground[nspots * 0.90];

else the integer closest to nspots * 0.90 but less than nspots *
0.9 is defined as a,

90^{th} percentile value =

foreground[a] + 0.90 * (foreground[a+1] − foreground[a])

This calculation is done at the time of the data upload. This allows us to make the analyses comparable to one another without rerunning the calculations for all of the previously loaded data each time new data is loaded.

For each slide/experiment, normalization is done as follows
signal_value = fg_median − bg_median;
coeff =

norm_constant/90^{th} percentile value of this slide

normalized_value =

coeff * min(signal_value, coeff);

normalized_value will be the final (normalized) value in the data
list for each
slide/experiment.
for log₂ transformation
log₂(normalized_value) = log₂(max(1.0, normalized_value))

Threshhold for CuraChip Data Analysis

A number of control spots are present on CuraChip 1.2 for efficiency calculations and to provide alternative normalization methods. For example, CuraChip 1.2 contains a number of empty or negative control spots, as well as positive control spots containing a dilution series of oligos that detect the highly-expressed genes Ubiquitin and glyceraldehyde-3-phosphate dehydrogenase (GAPD). An analysis of spot signal level was performed using raw data from 67 hybridizations using all oligos. The maximum signal intensity for each oligo across all 67 hybridizations was determined, and the fold-over-background for this maximum signal was calculated (i.e. if the background reading is 20 and the raw spot intensity is 100, then the fold-over-background for that spot is 5×). The negative control or empty spots do occasionally “fire” or give a signal over the background level; however, they do not fire very strongly, with 77.1% of empty spots firing <3× over background and 91.7%<5×. The positive control spots (Ubiquitin and GAPD) always fired at >100× background. The experimental oligos (CuraOligos) fired over the entire range of intensities, with some at low fold-over-background intensities. Since the negative control spots do fire occasionally at low levels, we have set a suggested threshhold for data analysis at >5× background.

Expression analysis of CG57042-01 using PTG Chip 1.2: Approximately 561 samples of RNA from tissues obtained from surgically dissected disease- and non-disease tissues, and treated and untreated cell lines, were used to generate labelled nucleic acid which was hybridized to PTG Chip 1.2. An oligo (optg2_{—}0400024, TCTTACCCAGAAATCTAGAGGATCTCTCCT) that corresponds to CG57042-01 on the PTG Chip 1.2 was analyzed for its expression profile.

| | Signal |
|---|---|
| Definition | value |
| G1C4E09B12-1_Patient-06pl | 593.38 |
| G1C4E09B12-10_vis.adipose-CMD-low-1 | 1360 |
| G1C4E09B12-11_vis.adipose-HMD-hi-1 | 783.66 |
| G1C4E09B12-12_psoas-HMD-hi-1 | 1291.33 |
| G1C4E09B12-13_psoas-HMD-med-1 | 0 |
| G1C4E09B12-14_vis.adipose-AMND-low-1 | 360 |
| G1C4E09B12-15_psoas-HMND-hi-1 | 208.65 |
| G1C4E09B12-16_vis.adipose-AMD-hi-1 | 1573.04 |
| G1C4E09B12-17_vis.adipose-CMND-low-1 | 607.18 |
| G1C4E09B12-18_psoas-CMND-low-1 | 233.3 |
| G1C4E09B12-19_psoas-CC.M.Diab.-med-1 | 0 |
| G1C4E09B12-2_Patient-07pl | 749.41 |
| G1C4E09B12-20_psoas-CC.M.Diab.-low-1 | 0 |
| G1C4E09B12-21_psoas-CC.M.Diab.-med-2 | 327.34 |
| G1C4E09B12-22_vis.adipose-HI.M.Diab.-med-1 | 0 |
| G1C4E09B12-23_psoas-CC.M.Norm-med-1 | 536.67 |
| G1C4E09B12-24_psoas-AS.M.Norm.-hi-1 | 10.18 |
| G1C4E09B12-25_psoas-CC.M.Norm-low-1 | 15.03 |
| G1C4E09B12-26_vis.adipose-CC.M.Norm-low-1 | 458.3 |
| G1C4E09B12-27_psoas-HI.M.Norm.-low-1 | 25.45 |
| G1C4E09B12-28_psoas-AA.M.Norm-med-2 | 0 |
| G1C4E09B12-29_psoas-AA.M.Diab.-hi-1 | 25.56 |
| G1C4E09B12-3_Patient-08pl | 369.36 |
| G1C4E09B12-31_psoas-AA.M.Diab.-med-1 | 84.87 |
| G1C4E09B12-32_vis.adipose-AS.M.Diab.-low-1 | 701.69 |
| G1C4E09B12-33_psoas-HI.M.Diab.-low-1 | 0 |
| G1C4E09B12-34_psoas-AS.M.Norm-low-1 | 111.07 |
| G1C4E09B12-35_psoas-AS.M.Diab.-hi-1 | 13.58 |
| G1C4E09B12-36_vis.adipose-CC.M.Diabe-hi-1 | 446.41 |
| G1C4E09B12-37_vis.adipose-AA.M.Diab-med-1 | 289.07 |
| G1C4E09B12-38_vis.adipose-AS.M.Norm-hi-1 | 175.74 |
| G1C4E09B12-39_vis.adipose-HI.M.Norm-hi-1 | 784 |
| G1C4E09B12-4_Patient-12go | 607.1 |
| G1C4E09B12-41_vis.adipose-HI.M.Norm-med-1 | 395.79 |
| G1C4E09B12-42_vis.adipose-AA.M.Norm-med-1 | 672.41 |
| G1C4E09B12-43_vis.adipose-CC.M.Norm-low-2 | 269.05 |
| G1C4E09B12-44_vis.adipose-HI.M.Norm-low-1 | 676.23 |
| G1C4E09B12-46_vis.adipose-AA.M.Norm-med-2 | 211.55 |
| G1C4E09B12-47_vis.adipose-HI.M.Diab-low-1 | 315.16 |
| G1C4E09B12-48_vis.adipose-CC.M.Norm-med-1 | 524.31 |
| G1C4E09B12-49_psoas-AS.M.Diab-low-1 | 213.77 |
| G1C4E09B12-5_Patient-12sk | 510.61 |
| G1C4E09B12-50_psoas-CC.M.Norm-low-2 | 337.63 |
| G1C4E09B12-51_vis.adipose-CC.M.Diab-med-2 | 46.67 |
| G1C4E09B12-6_Patient-12pl | 443.01 |
| G1C4E09B12-7_psoas-CMD-hi-1 | 0 |
| G1C4E09B12-8_vis.adipose-AAMD-hi-1 | 1018.18 |
| G1C4E09B12-9_psoas-AAMD-low-1 | 0 |
| G1C4I05B19-10_small intestine-HI.M.Diab-low-1 | 831.18 |
| G1C4I05B19-11_small intestine-HI.M.Diab-med-1 | 99.73 |
| G1C4I05B19-12_small intestine-AA.M.Norm-hi-1 | 177.67 |
| G1C4I05B19-13_small intestine-CC.M.Norm-med-1 | 1189.8 |
| G1C4I05B19-14_small intestine-AS.M.Norm-low-1 | 249.02 |
| G1C4I05B19-15_small intestine-CC.M.Norm-hi-1 | 224.93 |
| G1C4I05B19-16_small intestine-HI.M.Norm-hi-1 | 1001.56 |
| G1C4I05B19-17_small intestine-AS.M.Norm-hi-1 | 469.11 |
| G1C4I05B19-18_small intestine-HI.M.Norm-low-1 | 339.88 |
| G1C4I05B19-19_small intestine-AA.M.Norm-med-1 | 141.85 |
| G1C4I05B19-20_small intestine-AA.M.Norm-med-2 | 292.92 |
| G1C4I05B19-21_small intestine-CC.M.Norm-low-1 | 336.31 |
| G1C4I05B19-22_small intestine-CC.M.Norm-low-2 | 164.27 |
| G1C4I05B19-23_Mergen brain control | 161.49 |
| G1C4I05B19-24_liver-CMD-low-1 | 759.56 |
| G1C4I05B19-25_liver-CC.M.Diab.-med-1 | 258.62 |
| G1C4I05B19-26_liver-HI.M.Diab.-low-1 | 91.3 |
| G1C4I05B19-27_liver-HI.M.Diab.-med-1 | 513.5 |
| G1C4I05B19-28_liver-HI.M.Norm-hi-1 | 582.24 |
| G1C4I05B19-29_liver-AS.M.Norm.-hi-1 | 222.65 |
| G1C4I05B19-30_liver-AA.M.Diab-med-1 | 597.1 |
| G1C4I05B19-31_liver-HI.M.Diab-hi-1 | 348.65 |
| G1C4I05B19-32_liver-AA.M.Norm-hi-1 | 620.13 |
| G1C4I05B19-33_liver-CC.M.Norm-hi-1 | 265.43 |
| G1C4I05B19-34_liver-AS.M.Norm-low-1 | 368.05 |
| G1C4I05B19-35_liver-CC.M.Diab-med-2 | 341.5 |
| G1C4I05B19-36_liver-CC.M.Norm-low-1 | 672.22 |
| G1C4I05B19-37_liver-HI.M.Norm-low-1 | 129.64 |
| G1C4I05B19-38_liver-CC.M.Norm-med-1 | 2104.58 |
| G1C4I05B19-39_liver-AA.M.Norm-med-1 | 348.21 |
| G1C4I05B19-4_small intestine-CC.M.Diab-med-2 | 255.73 |
| G1C4I05B19-41_psoas-AAMND-hi-1 | 371.91 |
| G1C4I05B19-42_pancreas-HI.M.Diab.-hi-1 | 822.02 |
| G1C4I05B19-43_pancreas-AS.M.Norm-low-1 | 351.86 |
| G1C4I05B19-44_pancreas-AA.M.Diab.-hi-1 | 295.51 |
| G1C4I05B19-45_pancreas-AA.M.Diab-low-1 | 495.69 |
| G1C4I05B19-46_pancreas-AA.M.Diab-med-1 | 460.3 |
| G1C4I05B19-47_pancreas-CC.M.Diab-low-1 | 247.41 |
| G1C4I05B19-48_pancreas-HI.M.Diab-med-1 | 171.39 |
| G1C4I05B19-49_pancreas-CC.M.Norm-hi-1 | 248.02 |
| G1C4I05B19-5_small intestine-CC.M.Diab-hi-1 | 270.77 |
| G1C4I05B19-50_pancreas-HI.M.Norm-hi-1 | 212.15 |
| G1C4I05B19-51_pancreas-AS.M.Diab-hi-1 | 84.78 |
| G1C4I05B19-52_pancreas-CC.M.Diab-med-1 | 546.98 |
| G1C4I05B19-53_pancreas-AA.M.Norm-med-1 | 461.2 |
| G1C4I05B19-54_pancreas-CC.M.Norm-low-1 | 524.74 |
| G1C4I05B19-55_pancreas-CC.M.Norm-low-2 | 145.35 |
| G1C4I05B19-56_pancreas-Hi.M.Norm-low-1 | 118.52 |
| G1C4I05B19-57_pancreas-AA.M.Norm-med-2 | 39.52 |
| G1C4I05B19-58_pancreas-CC.M.Diab-hi-1 | 77.07 |
| G1C4I05B19-59_pancreas-CC.M.Diab-med-2 | 130.75 |
| G1C4I05B19-6_small intestine-AA.M.Diab-hi-1 | 690.57 |
| G1C4I05B19-61_subQadipose-AAMD-low-1 | 0 |
| G1C4I05B19-62_subQadipose-CMD-low-1 | 1443.2 |
| G1C4I05B19-63_subQadipose-HMD-med-1 | 1294.16 |
| G1C4I05B19-64_subQadipose-AMND-hi-1 | 1483.03 |
| G1C4I05B19-65_subQadipose-CMND-hi-1 | 1368.04 |
| G1C4I05B19-66_subQadipose-AMD-hi-1 | 2293.53 |
| G1C4I05B19-67_subQadipose-CC.M.Diab.-med-1 | 891.06 |
| G1C4I05B19-68_subQadipose-AA.M.Norm-med-1 | 566.02 |
| G1C4I05B19-69_subQadipose-CC.M.Norm-low-1 | 363.67 |
| G1C4I05B19-7_small intestine-AA.M.Diab-med-1 | 155.25 |
| G1C4I05B19-70_subQadipose-HI.M.Norm-low-1 | 1528.14 |
| G1C4I05B19-71_subQadipose-CC.M.Diab-hi-1 | 176.03 |
| G1C4I05B19-72_subQadipose-AA.M.Norm-med-2 | 515.43 |
| G1C4I05B19-73_subQadipose-HI.M.Diab-low-1 | 2025.35 |
| G1C4I05B19-74_subQadipose-AA.M.Diab-med-1 | 554.2 |
| G1C4I05B19-75_subQadipose-HI.M.Norm-hi-1 | 822.44 |
| G1C4I05B19-76_subQadipose-AS.M.Diab-low-1 | 2185.94 |
| G1C4I05B19-77_subQadipose-HI.M.Norm-med-1 | 674.08 |
| G1C4I05B19-78_subQadipose-AA.M.Diab-hi-1 | 776.46 |
| G1C4I05B19-8_small intestine-AA.M.Diab-low-1 | 448.12 |
| G1C4I05B19-9_small intestine-AS.M.Diab-low-1 | 411.38 |
| G1C4I11B20-40_hypothalamus-AS.M.Diab-low-1 | 49.93 |
| G1C4I11B20-41_hypothalamus-AA.M.Norm-hi-1 | 146.85 |
| G1C4I11B20-42_hypothalamus-HI.M.Diab-med-1 | 263.86 |
| G1C4I11B20-43_hypothalamus-CC.M.Diab-med-1 | 49.73 |
| G1C4I11B20-44_hypothalamus-AS.M.Diab-hi-1 | 53.87 |
| G1C4I11B20-45_hypothalamus-CC.M.Diab-low-1 | 81.68 |
| G1C4I11B20-46_hypothalamus-AA.M.Diab-low-1 | 75.56 |
| G1C4I11B20-47_hypothalamus-CC.M.Norm-hi-1 | 74.61 |
| G1C4I11B20-48_hypothalamus-CC.M.Norm-low-2 | 367.81 |
| G1C4I11B20-49_hypothalamus-CC.M.Diab-hi-1 | 447.37 |
| G1C4I11B20-50_hypothalamus-AS.M.Norm-hi-1 | 2825.45 |
| G1C4I11B20-51_hypothalamus-AA.M.Norm-med-2 | 474.32 |
| G1C4I11B20-52_hypothalamus-AS.M.Norm-low-1 | 435.51 |
| G1C4I11B20-53_hypothalamus-HI.M.Norm-hi-1 | 249.17 |
| G1C4I11B20-54_hypothalamus-HI.M.Diab-hi-1 | 368.01 |
| G1C4D21B11-13_Normal Lung 4 | 6384.08 |
| G1C4D21B11-14_Normal Lung 5 | 4386.57 |
| G1C4D21B11-19_Normal Lung 1 | 2077.94 |
| G1C4D21B11-24_Normal Lung 2 | 3301.64 |
| G1C4D21B11-30_Normal Lung 3 | 4530.43 |
| G1C4E23B15-52_SW1353 resting 1 h | 82.4 |
| G1C4E23B15-53_SW1353 resting 6 h | 80.76 |
| G1C4E23B15-54_SW1353 resting 16 h | 50.63 |
| G1C4E23B15-55_SW1353 IL-1b (1 ng/) 1 h | 81.19 |
| G1C4E23B15-56_SW1353 IL-1b (1 ng/) 6 h | 235.74 |
| G1C4E23B15-57_SW1353 IL-1b (1 ng/) 16 h | 371.48 |
| G1C4E23B15-58_SW1353 FGF20 (1 ug/) 1 h | 63.52 |
| G1C4E23B15-59_SW1353 FGF20 (1 ug/) 16 h | 9.73 |
| G1C4E23B15-61_SW1353 FGF20 (5 ug/) 1 h | 104.12 |
| G1C4E23B15-62_SW1353 FGF20 (5 ug/) 6 h | 105.42 |
| G1C4E23B15-63_SW1353 FGF20 (5 ug/) 16 h | 87.24 |
| G1C4E23B15-64_SW1353 FGF20 (1 ug/) IL-1b | 345.57 |
| (1 ng/) 6 h | |
| G1C4E23B15-65_SW1353 FGF20 (1 ug/) IL-1b | 561.99 |
| (1 ng/) 16 h | |
| G1C4E23B15-66_SW1353 FGF20 (5 ug/) IL-1b | 103.77 |
| (1 ng/) 1 h | |
| G1C4E23B15-67_SW1353 FGF20 (5 ug/) IL-1b | 285.66 |
| (1 ng/) 6 h | |
| G1C4E23B15-69_THP-1 aCD40 (1 ug/) 1 h | 2027.99 |
| G1C4E23B15-70_THP-1 aCD40 (1 ug/) 6 h | 1772.63 |
| G1C4E23B15-71_THP-1 LPS (100 ng/) 1 h | 1157.04 |
| G1C4E23B15-72_THP-1 LPS (100 ng/) 6 h | 1094.47 |
| G1C4E23B15-73_CCD1070SK TNFa (5 ng/) 6 h | 11.41 |
| G1C4E23B15-74_CCD1070SK TNFa (5 ng/) 24 h | 217.93 |
| G1C4E23B15-75_CCD1070SK IL-1b (1 ng/) 24 h | 251.59 |
| G1C4E23B15-76_THP-1 resting | 1371.23 |
| G1C4E23B15-77_THP-1 aCD40 (1 ug/) 24 h | 1416.62 |
| G1C4E23B15-78_THP-1 LPS (100 ng/) 24 h | 2804.63 |
| G1C4E23B15-79_CCD1070SK IL-1b (1 ng/) 6 h | 78.12 |
| G1C4F06B17-28_LC 18 hr | 803.02 |
| G1C4F06B17-29_LC-IL-! 18 hr | 142.43 |
| G1C4F06B17-34_Astrocyte_IL1B_1 hr_a | 0 |
| G1C4F06B17-35_Astrocyte_IL1B_6 hr_a | 28.19 |
| G1C4F06B17-36_Astrocyte_IL1B_24 hr_a | 532.41 |
| G1C4F06B17-40_SHSY 5Y Undifferentiated | 0 |
| G1C4F06B17-41_SHSY 5Y Differentiated | 3.98 |
| G1C4F06B17-5_LC 0 hr | 19.84 |
| G1C4F06B17-50_Normal Fetal Kidney | 0 |
| G1C4F06B17-52_Normal Liver | 119.23 |
| G1C4F06B17-53_Normal Fetal Liver | 657.3 |
| G1C4F06B17-54_Normal Fetal Lung | 298.38 |
| G1C4F06B17-55_Normal Salivary Gland | 850.28 |
| G1C4F06B17-56_Normal Fetal Skeletal Muscle | 52.16 |
| G1C4F06B17-58_Normal Thyroid | 512.86 |
| G1C4F06B17-59_Normal Trachea | 2229.92 |
| G1C4F06B17-6_LC-IL-1 0 hr | 52.14 |
| G1C4F06B17-60_Heart pool | 38.74 |
| G1C4F06B17-61_Pituitary Pool | 48.55 |
| G1C4F06B17-62_Spleen Pool | 1977.03 |
| G1C4F06B17-63_Stomach Pool | 293.06 |
| G1C4F06B17-64_Testis Pool | 23.71 |
| G1C4F06B17-65_Thymus Pool | 337.98 |
| G1C4F06B17-66_Small Intestine- 5 donor pool | 1333.59 |
| G1C4F06B17-67_Lymph node- 5 donor pool | 2346.57 |
| G1C4F06B17-68_Kidney- 5 donor pool | 275.22 |
| G1C4I11B20-34_Jurkat Resting | 0 |
| G1C4I11B20-35_Jurkat CD3 (500 ng/ml) 6 hr A | 1.67 |
| G1C4I11B20-36_Jurkat CD3 (500 ng/ml) 24 hr A | 0 |
| G1C4I11B20-37_Jurkat CD3 (500 ng/ml) + | 0 |
| CD28(1 ug/ml) 6 hr A | |
| G1C4I11B20-38_Jurkat CD3 (500 ng/ml) + | 0 |
| CD28(1 ug/ml) 24 hr A | |
| G1C4I11B20-55_control (no treatment)_1 hr | 2.6 |
| G1C4I11B20-56_10 ng/ml IL-1b_1 hr | 3.1 |
| G1C4I11B20-57_10 ng/ml TNF-a_1 hr | 0 |
| G1C4I11B20-58_200 uM BzATP_1 hr | 2.46 |
| G1C4I11B20-59_control (no treatment)_5 hr | 0 |
| G1C4I11B20-60_10 ng/ml IL-1b_5 hr | 3.83 |
| G1C4I11B20-61_10 ng/ml TNF-a_5 hr | 12.57 |
| G1C4I11B20-62_200 uM BzATP_5 hr | 7.34 |
| G1C4I11B20-63_control (no treatment)_24 hr | 0 |
| G1C4I11B20-64_10 ng/ml IL-1b_24 hr | 7.33 |
| G1C4I11B20-65_10 ng/ml TNF-a_24 hr | 41 |
| G1C4I11B20-66_200 uM BzATP_24 hr | 0 |
| G1C4I12B21-72_#689 Control Lung | 7899.8 |
| G1C4I12B21-73_#812 Asthma Lung | 4224.14 |
| G1C4I12B21-74_#1078 Control Lung | 2353.33 |
| G1C4D21B11-39_Alzheimer's disease B4951 | 184.08 |
| G1C4D21B11-40_Alzheimer's disease B4953 | 113.79 |
| G1C4D21B11-41_Alzheimer's disease B5018 | 292.11 |
| G1C4D21B11-43_Alzheimer's disease B5019 | 146.82 |
| G1C4D21B11-44_Alzheimer's disease B5086 | 446.36 |
| G1C4D21B11-51_Alzheimer's disease B5096 | 197.58 |
| G1C4D21B11-52_Alzheimer's disease B5098 | 0 |
| G1C4D21B11-54_Alzheimer's disease B5129 | 97.95 |
| G1C4D21B11-55_Alzheimer's disease B5210 | 309.22 |
| G1C4D21B11-56_Control B4810 | 116.01 |
| G1C4D21B11-57_Control B4825 | 128.87 |
| G1C4D21B11-58_Control B4930 | 201.12 |
| G1C4D21B11-59_Control B4932 | 127.01 |
| G1C4D21B11-60_Control B5024 | 144.66 |
| G1C4D21B11-61_Control B5113 | 103.25 |
| G1C4D21B11-62_Control B5140 | 159.13 |
| G1C4D21B11-63_Control B5190 | 179.7 |
| G1C4D21B11-64_Control B5220 | 54.05 |
| G1C4D21B11-65_Control B5245 | 74.58 |
| G1C4D21B11-66_AH3 B3791 | 70.24 |
| G1C4D21B11-67_AH3 B3855 | 68.42 |
| G1C4D21B11-68_AH3 B3877 | 75.97 |
| G1C4D21B11-69_AH3 B3893 | 91.79 |
| G1C4D21B11-70_AH3 B3894 | 73.98 |
| G1C4D21B11-71_AH3 B3949 | 109.57 |
| G1C4D21B11-72_AH3 B4477 | 186.29 |
| G1C4D21B11-73_AH3 B4540 | 65.93 |
| G1C4D21B11-74_AH3 B4577 | 75.32 |
| G1C4D21B11-75_AH3 B4639 | 138.27 |
| G1C4E19B13-45_Schizophrenia hippocampus 683 | 33.1 |
| G1C4E19B13-46_Depression hippocampus 487 | 42.78 |
| G1C4E19B13-47_Depression hippocampus 600 | 85.31 |
| G1C4E19B13-48_Normal hippocampus 2407a | 12.28 |
| G1C4E19B13-49_Normal hippocampus 1042 | 772.96 |
| G1C4E19B13-50_Depression hippocampus 2767 | 71.44 |
| G1C4E19B13-51_Depression hippocampus 567 | 278.49 |
| G1C4E19B13-52_Control hippocampus 3175 | 39.9 |
| G1C4E19B13-53_Depression hippocampus 3096 | 175.51 |
| G1C4E19B13-54_Depression hippocampus 1491 | 378.62 |
| G1C4E19B13-55_Depression hippocampus 2540 | 153.81 |
| G1C4E19B13-56_Schizophrenia hippocampus 2798 | 38.7 |
| G1C4E19B13-57_Control hippocampus 1973 | 164.3 |
| G1C4E19B13-58_Normal hippocampus and amygdala | 338.21 |
| 2601 | |
| G1C4E19B13-59_Schizophrenia hippocampus 2785 | 12.43 |
| G1C4E19B13-60_Schizophrenia hippocampus 484 | 8.74 |
| G1C4E19B13-61_Normal hippocampus 2556 | 199.66 |
| G1C4E19B13-62_Depression hippocampus 1158 | 69.15 |
| G1C4E19B13-63_Control hippocampus 552 | 27 |
| G1C4E19B13-64_Schizophrenia hippocampus 1737 | 103.7 |
| G1C4E19B13-65_Normal hippocampus 1239 | 333.36 |
| G1C4E19B13-66_Normal hippocampus 1465 | 161.2 |
| G1C4E19B13-67_Normal hippocampus 3080 | 2.62 |
| G1C4E19B13-68_Normal hippocampus 738 | 17.8 |
| G1C4E19B13-69_Schizophrenia hippocampus 2586 | 135.53 |
| G1C4E19B13-70_Normal hippocampus 2551 | 360.82 |
| G1C4E19B13-71_Depression hippocampus 588 | 12.73 |
| G1C4E19B13-72_Depression hippocampus 529 | 97.96 |
| G1C4E19B13-73_Depression hippocampus and | 106.29 |
| dentate gyrus | |
| G1C4E21B14-41_Schizophrenia amygdala 2586 | 141.57 |
| G1C4E21B14-42_Normal substantia nigra 234 | 0 |
| G1C4E21B14-43_Normal substantia nigra 1065 | 0 |
| G1C4E21B14-44_Normal substantia nigra 3236 | 171.88 |
| G1C4E21B14-45_Normal substantia nigra 2551 | 474.99 |
| G1C4E21B14-46_Normal substantia nigra 1597 | 0 |
| G1C4E21B14-47_Control thalamus 552 | 0 |
| G1C4E21B14-48_Control thalamus 566 | 0 |
| G1C4E21B14-49_Control thalamus 606 | 102.72 |
| G1C4E21B14-50_Control thalamus 738 | 19.7 |
| G1C4E21B14-51_Control thalamus 1065 | 78.16 |
| G1C4E21B14-52_Control thalamus 1092 | 154.25 |
| G1C4E21B14-53_Control thalamus 1597 | 84.36 |
| G1C4E21B14-54_Control thalamus 2253 | 236.73 |
| G1C4E21B14-55_Control thalamus 2551 | 199.22 |
| G1C4E21B14-56_Depression thalamus 588 | 37.01 |
| G1C4E21B14-57_Depression thalamus 600 | 0 |
| G1C4E21B14-58_Depression thalamus 721 | 6.09 |
| G1C4E21B14-59_Depression thalamus 728 | 22.22 |
| G1C4E21B14-60_Depression thalamus 759 | 12.28 |
| G1C4E21B14-61_Depression thalamus 881 | 5.36 |
| G1C4E21B14-62_Schizophrenia thalamus 477 | 150.72 |
| G1C4E21B14-63_Schizophrenia thalamus 532 | 75.78 |
| G1C4E21B14-64_Schizophrenia thalamus 683 | 52.04 |
| G1C4E21B14-65_Schizophrenia thalamus 544 | 43.06 |
| G1C4E21B14-66_Schizophrenia thalamus 1671 | 95.08 |
| G1C4E21B14-67_Schizophrenia thalamus 1737 | 133.27 |
| G1C4E21B14-68_Schizophrenia thalamus 2464 | 105.85 |
| G1C4E21B14-69_Schizophrenia thalamus 2586 | 429.05 |
| G1C4E23B15-1_Depression amygdala 600 | 0 |
| G1C4E23B15-10_Depression amygdala 759 | 88.05 |
| G1C4E23B15-11_Depression anterior cingulate | 0 |
| 759 | |
| G1C4E23B15-12_Control amygdala 552 | 28.33 |
| G1C4E23B15-14_Control anterior cingulate 482 | 74.06 |
| G1C4E23B15-15_Depression anterior cingulate | 0 |
| 721 | |
| G1C4E23B15-16_Control amygdala 3175 | 74.74 |
| G1C4E23B15-17_Depression anterior cingulate | 3.49 |
| 600 | |
| G1C4E23B15-18_Depression anterior cingulate | 39.15 |
| 588 | |
| G1C4E23B15-19_Control anterior cingulate 3175 | 50.78 |
| G1C4E23B15-2_Control anterior cingulate 606 | 55.8 |
| G1C4E23B15-20_Depression anterior cingulate | 67.28 |
| 567 | |
| G1C4E23B15-21_Depression amygdala 588 | 2.83 |
| G1C4E23B15-22_Control anterior cingulate 3080 | 82.7 |
| G1C4E23B15-23_Control anterior cingulate 2601 | 120.27 |
| G1C4E23B15-24_Control anterior cingulate 1042 | 113.92 |
| G1C4E23B15-25_Control anterior cingulate 3236 | 103.53 |
| G1C4E23B15-26_Control amygdala 1502 | 74.4 |
| G1C4E23B15-27_Control anterior cingulate 807 | 28.01 |
| G1C4E23B15-28_Control amygdala 1597 | 50.62 |
| G1C4E23B15-29_Parkinson's substantia nigra | 208.81 |
| 2842 | |
| G1C4E23B15-3_Parkinson's substantia nigra | 5.91 |
| 2917 | |
| G1C4E23B15-4_Schizophrenia amygdala 544 | 0 |
| G1C4E23B15-5_Schizophrenia amygdala 532 | 0 |
| G1C4E23B15-7_Depression amygdala 2540 | NA |
| G1C4E23B15-8_Parkinson's substantia nigra | 36.88 |
| 2899 | |
| G1C4E23B15-9_Depression anterior cingulate | 20.9 |
| 881 | |
| G1C4D21B11-01_Lung cancer(35C) | 391.05 |
| G1C4D21B11-02_Lung NAT(36A) | 4519.37 |
| G1C4D21B11-03_Lung cancer(35E) | 581.82 |
| G1C4D21B11-04_Lung cancer(365) | 3333.9 |
| G1C4D21B11-05_Lung cancer(368) | 3828.14 |
| G1C4D21B11-06_Lung cancer(369) | 581.72 |
| G1C4D21B11-07_Lung cancer(36E) | 2727.64 |
| G1C4D21B11-08_Lung NAT(36F) | 4947.97 |
| G1C4D21B11-09_Lung cancer(370) | 2536.33 |
| G1C4D21B11-10_Lung cancer(376) | 515.64 |
| G1C4D21B11-11_Lung cancer(378) | 254.21 |
| G1C4D21B11-12_Lung cancer(37A) | 540.89 |
| G1C4D21B11-13_Normal Lung 4 | 6384.08 |
| G1C4D21B11-14_Normal Lung 5 | 4386.57 |
| G1C4D21B11-16_5.Melanoma | 990.79 |
| G1C4D21B11-17_6.Melanoma | 468.09 |
| G1C4D21B11-18_Melanoma (19585) | 65.5 |
| G1C4D21B11-19_Normal Lung 1 | 2077.94 |
| G1C4D21B11-20_Lung cancer(372) | 1004.29 |
| G1C4D21B11-21_Lung NAT(35D) | 6845.66 |
| G1C4D21B11-22_Lung NAT(361) | 1190.48 |
| G1C4D21B11-23_1.Melanoma | 358.68 |
| G1C4D21B11-24_Normal Lung 2 | 3301.64 |
| G1C4D21B11-25_Lung cancer(374) | 2251.39 |
| G1C4D21B11-26_Lung cancer(36B) | 2254.81 |
| G1C4D21B11-27_Lung cancer(362) | 942.79 |
| G1C4D21B11-28_Lung cancer(358) | 5685.54 |
| G1C4D21B11-29_2.Melanoma | 701.25 |
| G1C4D21B11-30_Normal Lung 3 | 4530.43 |
| G1C4D21B11-31_Lung NAT(375) | 2290.69 |
| G1C4D21B11-32_Lung cancer(36D) | 2184.42 |
| G1C4D21B11-33_Lung NAT(363) | 18253.24 |
| G1C4D21B11-34_Lung cancer(35A) | 5546.91 |
| G1C4D21B11-35_4.Melanoma | 300.08 |
| G1C4E09B12-54_Prostate cancer(B8B) | 593.11 |
| G1C4E09B12-55_Prostate cancer(B88) | 1165.94 |
| G1C4E09B12-56_Prostate NAT(B93) | 1736.69 |
| G1C4E09B12-57_Prostate cancer(B8C) | 999.74 |
| G1C4E09B12-58_Prostate cancer(AD5) | 1034.4 |
| G1C4E09B12-59_Prostate NAT(AD6) | 1163.11 |
| G1C4E09B12-60_Prostate cancer(AD7) | 1061.36 |
| G1C4E09B12-61_Prostate NAT(AD8) | 321.1 |
| G1C4E09B12-62_Prostate cancer(ADA) | 522.63 |
| G1C4E09B12-63_Prostate NAT(AD9) | 1057.24 |
| G1C4E09B12-64_Prostate cancer(9E7) | 162.64 |
| G1C4E09B12-65_Prostate NAT(A0B) | 1175.57 |
| G1C4E09B12-66_Prostate cancer(A0A) | 2481.92 |
| G1C4E09B12-67_Prostate cancer(9E2) | 272.87 |
| G1C4E09B12-68_Pancreatic cancer(9E4) | 5193.45 |
| G1C4E09B12-69_Pancreatic cancer(9D8) | 363.31 |
| G1C4E09B12-70_Pancreatic cancer(9D4) | 789.49 |
| G1C4E09B12-71_Pancreatic cancer(9BE) | 1116.99 |
| G1C4E09B12-73_Pancreatic NAT(ADB) | 710.29 |
| G1C4E09B12-74_Pancreatic NAT(ADC) | 509.99 |
| G1C4E09B12-76_Pancreatic NAT(ADD) | 961.83 |
| G1C4E09B12-77_Pancreatic NAT(AED) | 3020.87 |
| G1C4E19B13-10_Colon NAT(8B6) | 6908.12 |
| G1C4E19B13-12_Colon NAT(9F1) | 8374.61 |
| G1C4E19B13-13_Colon cancer(9F2) | 4474.56 |
| G1C4E19B13-14_Colon NAT(A1D) | 5114.53 |
| G1C4E19B13-15_Colon cancer(9DB) | 875.76 |
| G1C4E19B13-16_Colon NAT(A15) | 6410.06 |
| G1C4E19B13-17_Colon cancer(A14) | 7498.25 |
| G1C4E19B13-18_Colon NAT(ACB) | 5660.67 |
| G1C4E19B13-19_Colon cancer(AC0) | 4990.29 |
| G1C4E19B13-2_Colon cancer(8A4) | 4005.13 |
| G1C4E19B13-20_Colon NAT(ACD) | 7885.45 |
| G1C4E19B13-21_Colon cancer(AC4) | 4176.13 |
| G1C4E19B13-22_Colon NAT(AC2) | 4156.94 |
| G1C4E19B13-23_Colon cancer(AC1) | 1320.63 |
| G1C4E19B13-24_Colon NAT(ACC) | 7532.7 |
| G1C4E19B13-25_Colon cancer(AC3) | 6203.78 |
| G1C4E19B13-26_Breast cancer(9B7) | 308.73 |
| G1C4E19B13-27_Breast NAT(9CF) | 933.33 |
| G1C4E19B13-28_Breast cancer(9B6) | 1453.29 |
| G1C4E19B13-29_Breast cancer(9C7) | 4631.17 |
| G1C4E19B13-3_Colon cancer(8A6) | 2856.23 |
| G1C4E19B13-30_Breast NAT(A11) | 957.19 |
| G1C4E19B13-31_Breast cancer(A1A) | 2885.01 |
| G1C4E19B13-32_Breast cancer(9F3) | 1434.31 |
| G1C4E19B13-33_Breast cancer(9B8) | 1855.65 |
| G1C4E19B13-34_Breast NAT(9C4) | 832.45 |
| G1C4E19B13-35_Breast cancer(9EF) | 2066.72 |
| G1C4E19B13-36_Breast cancer(9F0) | 1079.32 |
| G1C4E19B13-37_Breast cancer(9B4) | 2621.99 |
| G1C4E19B13-38_Breast cancer(9EC) | 4288.65 |
| G1C4E19B13-4_Colon cancer(8A7) | 2161.81 |
| G1C4E19B13-44_Colon cancer(8B7) | 5418.15 |
| G1C4E19B13-5_Colon cancer(8A9) | 5758.74 |
| G1C4E19B13-6_Colon cancer(8AB) | 4229.59 |
| G1C4E19B13-7_Colon cancer(8AC) | 9674.16 |
| G1C4E19B13-8_Colon NAT(8AD) | 6070.31 |
| G1C4E19B13-9_Colon cancer(8B5) | 3120.19 |
| G1C4E21B14-1_Cervical cancer(B08) | 1248.61 |
| G1C4E21B14-10_Brain cancer(9F8) | 341.74 |
| G1C4E21B14-11_Brain cancer(9C0) | 1042.76 |
| G1C4E21B14-12_Brain cancer(9F7) | 387.16 |
| G1C4E21B14-13_Brain cancer(A00) | 608.17 |
| G1C4E21B14-14_Brain NAT(A01) | 83.55 |
| G1C4E21B14-15_Brain cancer(9DA) | 624.84 |
| G1C4E21B14-16_Brain cancer(9FE) | 544.18 |
| G1C4E21B14-17_Brain cancer(9C6) | 274.85 |
| G1C4E21B14-18_Brain cancer(9F6) | 41.1 |
| G1C4E21B14-2_Cervical NAT(AEB) | 0 |
| G1C4E21B14-21_Bladder NAT(23954) | 0 |
| G1C4E21B14-22_Urinary cancer(AF6) | 44.8 |
| G1C4E21B14-23_Urinary cancer(B0C) | 3572.66 |
| G1C4E21B14-24_Urinary cancer(AE4) | 92.52 |
| G1C4E21B14-25_Urinary NAT(B20) | 140 |
| G1C4E21B14-26_Urinary cancer(AE6) | 475.47 |
| G1C4E21B14-27_Urinary NAT(B04) | 9 |
| G1C4E21B14-28_Urinary cancer(B07) | 0 |
| G1C4E21B14-29_Urinary NAT(AF8) | 1250.67 |
| G1C4E21B14-3_Cervical cancer(AFF) | 1216.46 |
| G1C4E21B14-30_Ovarian cancer(9D7) | 140.49 |
| G1C4E21B14-31_Urinary cancer(AF7) | 649.6 |
| G1C4E21B14-32_Ovarian cancer(9F5) | 0 |
| G1C4E21B14-33_Ovarian cancer(A05) | 338.01 |
| G1C4E21B14-34_Ovarian cancer(9BC) | 0 |
| G1C4E21B14-35_Ovarian cancer(9C2) | 116.78 |
| G1C4E21B14-36_Ovarian cancer(9D9) | 457.23 |
| G1C4E21B14-37_Ovarian NAT(AC7) | 0 |
| G1C4E21B14-38_Ovarian NAT(AC9) | 0 |
| G1C4E21B14-39_Ovarian NAT(ACA) | 0 |
| G1C4E21B14-4_Cervical NAT(B1E) | 285.22 |
| G1C4E21B14-40_Ovarian NAT(AC5) | 0 |
| G1C4E21B14-6_Cervical NAT(AFA) | 187.88 |
| G1C4E21B14-7_Cervical cancer(B1F) | 2350.34 |
| G1C4E21B14-8_Cervical NAT(B1C) | 245 |
| G1C4E23B15-32_Breast cancer(D34) | 2692.24 |
| G1C4E23B15-33_Breast cancer(D35) | 2698.45 |
| G1C4E23B15-34_Breast cancer(D36) | 2101.27 |
| G1C4E23B15-35_Breast cancer(D37) | 1884.11 |
| G1C4E23B15-36_Breast cancer(D38) | 5899.82 |
| G1C4E23B15-37_Breast cancer(D39) | 2398.03 |
| G1C4E23B15-38_Breast cancer(D3A) | 2061.78 |
| G1C4E23B15-39_Breast cancer(D3B) | 929.27 |
| G1C4E23B15-40_Breast cancer(D3C) | 2929.77 |
| G1C4E23B15-41_Breast cancer(D3D) | 1414.25 |
| G1C4E23B15-42_Breast cancer(D3E) | 1437.88 |
| G1C4E23B15-43_Breast cancer(D3F) | 2848.08 |
| G1C4E23B15-44_Breast cancer(D40) | 1384.94 |
| G1C4E23B15-45_Breast cancer(D42) | 1113.39 |
| G1C4E23B15-46_Breast cancer(D43) | 3013.51 |
| G1C4E23B15-47_Breast cancer(D44) | 1327.16 |
| G1C4E23B15-48_Breast cancer(D45) | 2688.76 |
| G1C4E23B15-49_Breast cancer(D46) | 2352.01 |
| G1C4E30B16-1_2.SK-MES | 0 |
| G1C4E30B16-10_40.HLaC-79 | 0 |
| G1C4E30B16-11_43.H226 | 0 |
| G1C4E30B16-12_45.HCT-116 | 0 |
| G1C4E30B16-13_53.IGROV-1 | 45.56 |
| G1C4E30B16-14_59.MX-1 | 0 |
| G1C4E30B16-15_63.C33A | 0 |
| G1C4E30B16-16_65.Daudi | 616.73 |
| G1C4E30B16-17_71.MV522 | 124.07 |
| G1C4E30B16-18_76.RWP-2 | 94.44 |
| G1C4E30B16-19_77.BON | 0 |
| G1C4E30B16-2_6.MiaPaCa | 0 |
| G1C4E30B16-20_82.H82 | 0 |
| G1C4E30B16-21_86.H69 | 0 |
| G1C4E30B16-22_95.Caki-2 | 73.18 |
| G1C4E30B16-23_100.LNCaP | 184.48 |
| G1C4E30B16-24_101.A549 | 509.8 |
| G1C4E30B16-25_1.DU145 | 0 |
| G1C4E30B16-26_6.OVCAR-3 | 140.33 |
| G1C4E30B16-27_11.HT-29 | 706.61 |
| G1C4E30B16-28_13.DLD-2 | 121.22 |
| G1C4E30B16-29_18.MCF-7 | 0 |
| G1C4E30B16-3_9.H460 | 86.43 |
| G1C4E30B16-4_15.SW620 | 0 |
| G1C4E30B16-5_20.SK-OV-3 | 0 |
| G1C4E30B16-6_23.MDA-231 | 0 |
| G1C4E30B16-7_27.Caki-1 | 68.37 |
| G1C4E30B16-8_31.PC-3 | 0 |
| G1C4E30B16-9_35.LoVo | 0 |
| G1C4I11B20-10_Kidney NAT(10B1) | 1902.61 |
| G1C4I11B20-11_Kidney cancer(10B2) | 1366.4 |
| G1C4I11B20-12_Kidney NAT(10B3) | 1047.89 |
| G1C4I11B20-13_Kidney cancer(10B4) | 2730.94 |
| G1C4I11B20-14_Kidney NAT(10B5) | 719.46 |
| G1C4I11B20-15_Kidney cancer(10B6) | 1002.44 |
| G1C4I11B20-16_Kidney NAT(10B7) | 693.1 |
| G1C4I11B20-17_Kidney cancer(10BA) | 263.94 |
| G1C4I11B20-18_Kidney NAT(10BB) | 327.89 |
| G1C4I11B20-19_Kidney cancer(10C0) | 1142.31 |
| G1C4I11B20-20_Kidney NAT(10C1) | 570.36 |
| G1C4I11B20-21_Kidney cancer(10C4) | 479.11 |
| G1C4I11B20-22_Kidney NAT(10C5) | 8573.84 |
| G1C4I11B20-23_Kidney cancer(10A8) | 2743.33 |
| G1C4I11B20-24_Kidney NAT(10A9) | 558.06 |
| G1C4I11B20-25_Kidney cancer(10AA) | 3181.53 |
| G1C4I11B20-4_Kidney NAT(10AB) | 857.47 |
| G1C4I11B20-5_Kidney cancer(10AC) | 1004.09 |
| G1C4I11B20-6_Kidney NAT(10AD) | 719.41 |
| G1C4I11B20-7_Kidney cancer(10AE) | 2146.52 |
| G1C4I11B20-8_Kidney NAT(10AF) | 2278.13 |
| G1C4I11B20-9_Kidney cancer(10B0) | 3723.24 |
| G1C4I12B21-66_Ardais Lung 4 | 8893.27 |
| G1C4I12B21-67_Ardais Lung 6 | 11517.88 |
| G1C4l12B21-68_Ardais Lung 7 | 15434.12 |
| G1C4l12B21-69_Ardais Lung 10 | 16184.52 |
| G1C4I12B21-70_4169B1 normal lung | 2133.75 |
| G1C4I12B21-71_4267B1 normal lung | 6950.38 |
| G1C4I12B21-72_#689 Control Lung | 7899.8 |
| G1C4I12B21-73_#812 Asthma Lung | 4224.14 |
| G1C4I12B21-74_#1078 Control Lung | 2353.33 |
| G1C4I17B22-10_Lymphoma(9BF) | 11462.97 |
| G1C4I17B22-11_Lymphoma(9D2) | 4997.49 |
| G1C4I17B22-12_Lymphoma(A04) | 7797.29 |
| G1C4I17B22-13_Lymphoma(9DD) | 3909.53 |
| G1C4I17B22-14_Lymphoma(F68) | 1236.36 |
| G1C4I17B22-15_Lymphoma(F6A) | 1004.14 |
| G1C4I17B22-16_Lymphoma(F6B) | 9236.93 |
| G1C4I17B22-17_Lymphoma(F6C) | 4993.33 |
| G1C4I17B22-18_Lymphoma(F6D) | 4111.14 |
| G1C4I17B22-19_Lymphoma(F6E) | 1833.73 |
| G1C4I17B22-20_Lymphoma(F6F) | 647.11 |
| G1C4I17B22-21_Lymphoma(F70) | 2951.11 |
| G1C4I17B22-22_Lymphoma(F71) | 2462.47 |
| G1C4I17B22-23_Lymphoma(F72) | 744.68 |
| G1C4I17B22-24_Lymphoma(F73) | 1217.44 |
| G1C4I17B22-25_Lymphoma(F74) | 1647.31 |
| G1C4I17B22-26_Lymphoma NAT(1002) | 2603.68 |
| G1C4I17B22-28_Lymphoma NAT(1004) | 6720.44 |
| G1C4I17B22-29_Lymphoma NAT(1005) | 7788.32 |
| G1C4I17B22-30_Lymphoma NAT(1007) | 11731.8 |
| G1C4I17B22-32_Lymphoma NAT(1003) | 5973.33 |
| G1C4I17B22-4_Lymphoma(9E3) | 8021.96 |
| G1C4I17B22-5_Lymphoma(9D0) | 1773.69 |
| G1C4I17B22-6_Lymphoma(9E1) | 9636.46 |
| G1C4I17B22-7_Lymphoma(A0D) | 4819.67 |
| G1C4I17B22-8_Lymphoma(9B5) | 3593.18 |
| G1C4I17B22-9_Lymphoma(9D3) | 15739.79 |

Gene expression analysis using CuraChip revealed that this gene was highly expressed in lung, Lymphoma, kidney, liver, and adipose tissues. Therapeutic modulation of this gene, expressed protein and/or use of antibodies or small molecule drugs targeting the gene or gene product would be useful in the treatment of metabolically related diseases, such as obesity obesity, diabetes, hypercholesterolemia and hypertension.

### Other Embodiments

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

1. An isolated polypeptide comprising the mature form of an amino acid sequence selected from the group consisting of SEQ ID NO:2n, wherein n is an integer between 1 and 56.

2. An isolated polypeptide comprising an amino acid sequence selected from the group consisting of SEQ ID NO:2n, wherein n is an integer between 1 and 56.

3. An isolated polypeptide comprising an amino acid sequence which is at least 95% identical to an amino acid sequence selected from the group consisting of SEQ ID NO:2n, wherein n is an integer between 1 and 56.

4. An isolated polypeptide, wherein the polypeptide comprises an amino acid sequence comprising one or more conservative substitutions in the amino acid sequence selected from the group consisting of SEQ ID NO:2n, wherein n is an integer between 1 and 56.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim 1, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator of activity or of latency or of predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO:2n, wherein n is an integer between 1 and 56 or a biologically active fragment thereof.

20. An isolated nucleic acid molecule comprising a nucleic acid sequence selected from the group consisting of SEQ ID NO:2n−1, wherein n is an integer between 1 and 56.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 56.

23. An isolated nucleic acid molecule encoding the mature form of a polypeptide having an amino acid sequence selected from the group consisting of SEQ ID NO:2n, wherein n is an integer between 1 and 56.

24. An isolated nucleic acid molecule comprising a nucleic acid selected from the group consisting of 2n−1, wherein n is an integer between 1 and 56.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 56, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule; and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 56.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO:2n−1, wherein n is an integer between 1 and 56.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
